(19) United States
(12) Patent Application Publication
(10) Pub. No.: US 2025/0129101 A1
(43) Pub. Date: Apr. 24, 2025

(54) Compound, Pharmaceutical Composition Comprising the Same and Method for Treating Cancer Using the Same

(71) Applicants: NATIONAL TSING HUA UNIVERSITY, Hsinchu City (TW); NATIONAL HEALTH RESEARCH INSTITUTES, Miaoli County (TW)

(72) Inventors: Hui-Ching WANG, Hsinchu City (TW); Hsing-Pang HSIEH, Miaoli County (TW); Ching-Chuan KUO, Miaoli County (TW); Yuh-Ju SUN, Hsinchu City (TW); Yuan-Shao PAO, Hsinchu City (TW); Kuan-Ju LIAO, Hsinchu City (TW); Ya-Chia SHIAU, Hsinchu City (TW)

(21) Appl. No.: 18/919,681

(22) Filed: Oct. 18, 2024

Related U.S. Application Data
(60) Provisional application No. 63/591,797, filed on Oct. 20, 2023.

(51) Int. Cl. C07D 513/04; A61K 31/337; A61K 31/35; A61K 31/427; A61K 31/475; A61K 31/519; A61K 31/5377; A61P 35/00

(52) U.S. Cl. CPC C07D 513/04; A61K 31/337; A61K 31/35; A61K 31/427; A61K 31/475; A61K 31/519; A61K 31/5377; A61P 35/00

(57) ABSTRACT

Disclosed is a compound represented by the following formula (I), or a pharmaceutically acceptable salt thereof:

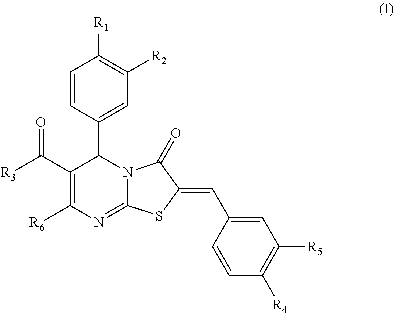

wherein R₁, R₂, R₃, R₄, R₅ and R₆ are defined in the specification. Also disclosed is a pharmaceutical composition comprising the same and a method for treating a cancer using the same.

### CROSS-REFERENCE TO RELATED APPLICATIONS

This application claims the benefit of filing date of U.S. Provisional Application Ser. No. 63/591,797, filed Oct. 20, 2023 under 35 USC § 119(e)(1).

### BACKGROUND OF THE INVENTION

### Field

The present invention relates to a compound, a pharmaceutical composition comprising the same and a method for treating a cancer using the same. More specifically, the present invention relates to a compound as a DHTP analog, a pharmaceutical composition comprising the same and a method for treating a cancer using the same.

### Description of Related Art

Triple-negative breast cancer (TNBC), histologically characterized by a lack of estrogen receptor (ER) and progesterone receptor (PR) as well as human epidermal growth factor receptor-2 (HER2), has the worst clinical outcome among breast cancer subtypes due to the lack of therapeutic targets. Systemic chemotherapy using microtubule targeting agents (MTAs), especially paclitaxel, has been widely used for treatments of patients with stage II and III TNBC, with the general objective response rate (ORR) between 20% to 40% and median progression-free survival (PFS) of 5 months. The overall survival after neoadjuvant chemotherapy of TNBC is low as a result of tumor recurrence with chemoresistance.

The long-term effectiveness of paclitaxel is limited by chemoresistance. Therefore, it is desirable to provide a novel strategy to overcome the above defects.

### SUMMARY OF THE INVENTION

The present invention relates to a compound that can inhibit the growth of tumor cells.

An aspect of the present invention is drawn to compound represented by the following formula (I), or a pharmaceutically acceptable salt thereof:

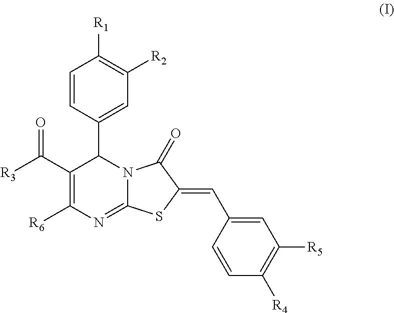

wherein,
one of R₁ and R₂ is H, and the other one of R₁ and R₂ is halogen, unsubstituted or substituted C₁₋₆ alkyl, unsubstituted or substituted C₁₋₆ alkoxy, —NRₐR_{b}, or a N-containing heteroaryl group, wherein Rₐ and R_{b} respectively is H or C₁₋₆ alkyl;
R₃ is —OH, unsubstituted or substituted C₁₋₆ alkyl, or unsubstituted or substituted C₁₋₆ alkoxy;
R₄ is halogen or —O—C₁₋₃ alkylene-R_{c}, wherein R_{c} is H, —CN, —OH, —C(═O)OR_{d}, —C(═O)Rₑ, C₂₋₆ alkynyl or a heteroaryl group; Rd is H, or unsubstituted or substituted C₁₋₆ alkyl; Rₑ is —NR_{f}R_{g}, or a heterocycloalkyl group substituted with —OH, C₁₋₆ alkyl, —NR_{f}R_{g}, cycloalkyl or a heterocycloalkyl group substituted with C₁₋₆ alkyl; wherein R_{f} is H or C₁₋₆ alkyl, and R_{g} is H, unsubstituted or substituted C₁₋₆ alkyl or —SO₂CF₃;
R₅ is H; or R₄, R₅ and carbon atoms attached thereto form a heterocycloalkyl group; and
R₆ is C₁₋₆ alkyl,
with proviso that R₄ is not —OCH₂C(═O)OH when R₁ is Cl, R₂ is H, R₃ is C₃ alkyl, R₆ is methyl, and R₅ is H.

The term “alkyl” herein refers to a straight or branched hydrocarbon group, containing 1-6 carbon atoms (e.g., C₁-C₆, C₁-C₅, C₁-C₄ and C₁-C₃). Examples include methyl, ethyl, n-propyl, i-propyl, n-butyl, i-butyl, and t-butyl.

The term “alkynyl” herein refers to a straight or branched monovalent or bivalent hydrocarbon containing 2-6 carbon atoms (e.g., C₂-C₆, C₂-C₅, C₂-C₄ and C₂-C₃) and one or more triple bonds. Examples of alkynyl include, but are not limited to, ethynyl, ethynylene, 1-propynyl, 1- and 2-butynyl, and 1-methyl-2-butynyl.

The term “cycloalkyl” refers to a saturated and partially unsaturated monocyclic, bicyclic, tricyclic, or tetracyclic hydrocarbon group having 3-12 (e.g., 3-10 and 3-7) carbon atoms. Examples include cyclopropyl, cyclobutyl, cyclopentyl, cyclopentenyl, cyclohexyl, cyclohexenyl, cycloheptyl, and cyclooctyl.

The term “heterocycloalkyl” refers to a nonaromatic 5-8 membered monocyclic, 8-12 membered bicyclic, or 11-14 membered tricyclic ring system having one or more heteroatoms (e.g., O, N, P, and S). Examples include piperazinyl, imidazolidinyl, azepanyl, pyrrolidinyl, dihydrothiadiazolyl, dioxanyl, morpholinyl, tetrahydropuranyl, and tetrahydrofuranyl.

The term “alkoxy” or “alkyloxy” refers to an —O-alkyl group. Examples include methoxy, ethoxy, propoxy, and isopropoxy.

The term “halogen” refers to a fluoro, chloro, bromo, or iodo radical.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include thiophenyl, triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl.

Alkyl, cycloalkyl, heterocycloalkyl, alkoxy, and heteroaryl mentioned herein include both substituted and unsubstituted moieties. Possible substituents on heterocycloalkyl, alkoxy, aryl, and heteroaryl include, but are not limited to, C₁₋₆ alkyl, C₂₋₆ alkenyl, C₂₋₆ alkynyl, C₃₋₁₂ cycloalkyl, C₃₋₁₂ cycloalkenyl, C₁₋₁₂ heterocycloalkyl, C₁₋₁₂ heterocycloalkenyl, C₁₋₆ alkoxy, aryl, aryloxy, heteroaryl, heteroaryloxy, amino, C₁₋₆ alkylamino, C₁₋₂₀ dialkylamino, arylamino, diarylamino, C₁₋₆ alkylsulfonamino, arylsulfonamino, C₁₋₆ alkylimino, arylimino, C₁₋₆ alkylsulfonimino, arylsulfonimino, hydroxyl, halo, thio, C₁₋₆ alkylthio, arylthio, C₁₋₆ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on alkyl include all of the above-recited substituents except C₁₋₆ alkyl. Heterocycloalkyl, aryl, and heteroaryl can also be fused with each other.

In addition to the compounds of formula (I) described above, their pharmaceutically acceptable salts, where applicable, are also covered by the present invention. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

Another aspect of the present invention is a pharmaceutical composition, comprising: one of the compounds of Formula (I) described above or its pharmaceutically acceptable salt thereof; and a pharmaceutically acceptable carrier, excipient or diluent.

The present invention also covers use of such a composition for the manufacture of a medicament for treating a cancer.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques; hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters, fluorocarbons, and/or other solubilizing or dispersing agents known in the art. A composition having an active compound can also be administered in the form of suppositories for rectal administration.

The carrier, the excipient and the diluent in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

Still within the scope of the present invention is a method of treating a cancer.

The method includes administering to a subject in need thereof an effective amount of a compound of formula (I) or a pharmaceutically acceptable salt thereof.

The above-described compounds or a pharmaceutical composition containing one or more of them can be administered to a subject orally, parenterally, by inhalation spray, topically, rectally, nasally, buccally, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

The term “treating”, “treat” or “treatment” refers to application or administration of the compound to a subject with the purpose to cure, alleviate, relieve, alter, remedy, improve, or affect the disease, the symptom, or the predisposition. “An effective amount” refers to the amount of the compound which is required to confer the desired effect on the subject. Effective amounts vary, as recognized by those skilled in the art, depending on route of administration, excipient usage, and the possibility of co-usage with other therapeutic treatments such as use of other active agents.

Other novel features of the disclosure will become more apparent from the following detailed description when taken in conjunction with the accompanying drawings.

### BRIEF DESCRIPTION OF DRAWINGS

FIG. 1A shows the results of Kaplan-Meier survival analysis of breast cancer patients with high or low KIF2C expression, wherein raw data was obtained from the PRECOG database (accession number: Vijver_BreastCancer, n=295).

FIG. 1B shows the results of KIF2C mRNA expressions in different subtypes of breast cancer, wherein data was analyzed from the Breast Invasive Carcinoma data set (TCGA PanCancer Atlas, n=1084, www.cBioPortal.org).

FIG. 1C shows the results of cell viability (% of solvent control) of MDA-MB-231 S and R cells following 72 hr paclitaxel treatment, wherein IC₅₀ indicates the drug concentration required for 50% inhibition of cell viability.

FIG. 1D shows the results of cell viability of 4T1 S and R cells following 72 hr paclitaxel treatment.

FIG. 1E shows the results of western blots of KIF2C and GAPDH in the indicated cell lines with quantitation of three replicates.

FIG. 1F shows the results of western blots of KIF2C and GAPDH in 4T1 or MDA-MB-231 cells transduced with scramble or KIF2C-specific shRNA for 48 hr, wherein cell viability was analyzed following 72 hr paclitaxel treatment.

FIG. 1G shows the results of cell viability of 4T1 S1, R4, and R8 cells following 72 hr paclitaxel treatment.

FIG. 1H shows the results of western blots of GAPDH and KIF2A, 2B, and 2C in 4T1 S1, R4, and R8 cells with quantitation of three replicates.

FIG. 1I shows the results of western blots of GAPDH, Cyclin B1, and KIF2C in interphase (50 μM thymidine synchronized) and mitotic (50 μM thymidine released into 50 μM monastrol) cells among 4T1 S1, R4, and R8 cells, with quantitation of three replicates.

FIG. 1J shows the results of cell proliferation rate of 4T1 S1, R4, and R8 cells over 72 hr.

FIG. 1K shows the results of cell viability of 4T1 R4 and R8 cells following 72 hr treatment with paclitaxel.

FIG. 1L shows the results of wound healing assay in 4T1 S1, R4, and R8 cells with degree of wound closure relative to 4T1 S1.

FIG. 2A shows the results of western blots of KIF2C and tubulin PTMs in 4T1 S and R cells with quantitation of three replicates.

FIG. 2B shows the results of KIF2C activity measured using HEK293 or porcine brain tubulin, with or without 1 mM paclitaxel pre-stabilization.

FIG. 2C shows the results of KIF2C activity measured using tubulin from HEK293, TTLL4 transfected HEK293, or VASH1/2-SVBP transfected HEK293 cells.

FIG. 2D shows the results of KIF2C activity measured using tubulin from HEK293, TTLL4 transfected HEK293, or porcine brain.

FIG. 2E shows the results of western blot of polyE tubulin and total tubulin in 4T1 S1, R4, and R8 cells with or without release from 200 nM paclitaxel treatment for 72 hr with quantitation of three replicates.

FIG. 2F shows the results of western blot of Poly-E tubulin and total tubulin in 4T1 S1 cells with or without 200 nM paclitaxel treatment for 24 hr with quantitation of three replicates.

FIG. 3A shows the results of KIF2C activity measured with tubulin from HEK293 cells with or without in vitro polyglutamylation.

FIG. 3B shows the results of KIF2C activity measured with tubulin purified from 4T1 S1, R4, and R8 cells.

FIG. 3C shows the results of abundance of glutamylated peptides identified on tubulin-ala (tubala) of 4T1 S1, R4, and R8 cells quantified by LC-MS/MS analysis, wherein peptides with indicated glutamylated residues were indicated.

FIG. 3D shows the results of western blots of KIF2A, 2B, 2C, and GAPDH in 4T1 R4 or MDA-MB-231 R cells transduced with scramble or KIF2C-specific shRNA, with quantitation of three replicates.

FIG. 3E shows the results of cell viability of 4T1 R4 or MDA-MB-231 R cells pretreated with shRNA for 48 hr, followed by paclitaxel treatment for 72 hr.

FIG. 4A shows the results of cell viability of MDA-MB-231 cells following treatment of DHTP for 72 hr.

FIG. 4B shows the results of inhibition of KIF2C activity quantitated under treatments of indicated compounds (100 μM).

FIG. 4C shows the results of cell viability of 4T1 and MDA-MB-231 cells following treatment with KIF2C inhibitor C4 for 72 hr.

FIG. 4D shows the results of cell viability of 4T1 R4 and R8 following treatment with a combination of 10 μM C4 and paclitaxel.

FIG. 4E shows the results of cell viability of MDA-MB-231 cells following treatments of indicated compounds for 72 hr.

FIG. 4F shows the emission spectrums of DHTP and 7S9 stock (10 mM) excited with 410 nm wavelength.

FIG. 4G and FIG. 4H shows the results of cell viability of indicated cell lines measured following treatments with DHTP or 7S9 for 72 hr.

FIG. 4I shows the results of the inhibitory effects of DHTP and 7S9 (50 μM) against KIF2A, 2B, and 2C.

FIG. 5 shows the results of KIF2C activity measured in the presence of 1 μM paclitaxel, 1 μM nocodazole, or 100 μM 7S9 as indicated, wherein all data was normalized with the KIF2C activity measured in the presence of porcine brain tubulin and paclitaxel, represented as mean±SEM of three replicates.

FIG. 6A shows the results of cell viability of 4T1 R4 and R8 cells following treatments with paclitaxel alone or a combination with 1 μM 7S9 for 72 hr.

FIG. 6B shows the results of cell viability of 4T1 R4 and R8 cells following treatments with 7S9 alone or a combination treatment with 400 nM paclitaxel for 72 hr.

FIG. 6C shows the results of a colony formation assay conducted to measure anchorage-independent growth of 4T1 S1, R4, and R8 cells in the presence of 400 nM paclitaxel, 1 μM 7S9, or a combination of both, wherein quantification results of colony numbers are shown.

FIG. 6D shows the results of live cell imaging analysis was conducted for 4T1 S1, R4, and R8 cells in the presence of 400 nM paclitaxel, 1 μM 7S9, or a combination of both, wherein quantitation results of mitotic progression time, death in mitosis, and mitotic defects (cytokinesis failure pause death in mitosis) are shown, and all quantitation data represent as mean±SEM from three replicates (t-test ***, P<0.001).

FIG. 7A shows the results tumor volumes of BALB/c mice treated with paclitaxel, 7S9, or a combination of both, wherein BALB/c mice were inoculated subcutaneously with 4T1, R4, or R8 cell suspensions for the progression of tumor nodules. Mice were sorted into 4 groups when the tumor size reached 60-100 mm 3 (day 1) and injected intraperitoneally with solvent control, paclitaxel, 7S9, or a combination of both, the treatment regimen is once per day for 5 consecutive days of administration and 2 days of drug withdrawal (5on/2off) for two consecutive cycles, the number of mice used in each group was shown as indicated n, and tumor volume was measured twice per week during experiments.

FIG. 7B shows the results of cell viability of 4T1 S1, R4, and R8 cells following treatments of docetaxel, ixabepilone, eribulin, or vinorelbine for 72 hr.

FIG. 7C shows the results of cell viability of 4T1 R4 and R8 cells following treatments with indicated MTAs alone or a combination with 2 μM 7S9 for 72 hr.

FIG. 7D shows the results of the correlation between nuclear and cytosolic expression levels of KIF2C and clinical characteristics, including tumor stages and lymph node metastasis stages, was assessed using the Immune Reactivity Scoring System (IRS), wherein the IRS is determined by multiplying the intensity score, which reflects the expression level of KIF2C, with the area score, indicating the proportion of KIF2C-positive cells, and all quantitation data are represented as mean±SEM, t-test *, P<0.05; **, P<0.01; ***, P<0.001.

### DETAILED DESCRIPTION OF THE INVENTION

One embodiment of the present invention is the compound represented by the following formula (I), or a pharmaceutically acceptable salt thereof:

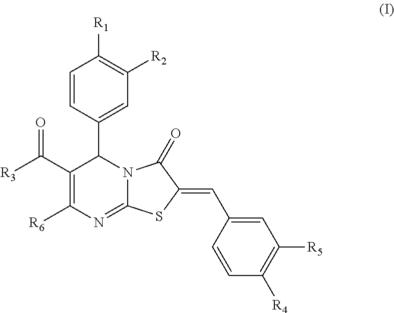

in which each of variables each of variables R₁, R₂, R₃, R₄, R₅ and R₆ are defined in the SUMMARY OF THE INVENTION section.

Another embodiment of the present invention is the compound of the aforesaid embodiment or a pharmaceutically acceptable salt thereof, wherein R₂ may be H, and R₁ may be F, Cl, Br, unsubstituted or substituted C₁₋₆ alkyl, unsubstituted or substituted C₁₋₆ alkoxy, —NRₐR_{b}, or imidazolyl.

Another embodiment of the present invention is the compound of the aforesaid embodiment or a pharmaceutically acceptable salt thereof, wherein R₁ may be Cl or methoxy.

Another embodiment of the present invention is the compound of the aforesaid embodiment or a pharmaceutically acceptable salt thereof, wherein R₃ may be unsubstituted or substituted C₁₋₆ alkoxy.

Another embodiment of the present invention is the compound of the aforesaid embodiment or a pharmaceutically acceptable salt thereof, wherein R₃ may be unsubstituted C₁₋₆ alkoxy.

Another embodiment of the present invention is the compound of the aforesaid embodiment or a pharmaceutically acceptable salt thereof, wherein R₅ may be H.

Another embodiment of the present invention is the compound of the aforesaid embodiment or a pharmaceutically acceptable salt thereof, wherein R₄ may be F or —O—C₁₋₃ alkyl-R_{c}.

Another embodiment of the present invention is the compound of the aforesaid embodiment or a pharmaceutically acceptable salt thereof, wherein R₄ may be —O—CH₂—R_{c}.

Another embodiment of the present invention is the compound of the aforesaid embodiment or a pharmaceutically acceptable salt thereof, wherein R₄ may be —O—CH₂—C(═O)Rₑ.

Another embodiment of the present invention is the compound of the aforesaid embodiment or a pharmaceutically acceptable salt thereof, wherein Rₑ may be a heterocycloalkyl group substituted with —OH, C₁₋₆ alkyl, —NR_{f}R_{g}, cycloalkyl, or a heterocycloalkyl group substituted with C₁₋₆ alkyl.

Another embodiment of the present invention is the compound of the aforesaid embodiment or a pharmaceutically acceptable salt thereof, wherein Rₑ may be

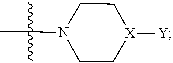

wherein X may be N or C, and Y may be C₁₋₆ alkyl, —NR_{f}R_{g} or a heterocycloalkyl group substituted with C₁₋₆ alkyl, wherein R_{f} and R_{g} may respectively be a C₁₋₆ alkyl.

Another embodiment of the present invention is the compound of the aforesaid embodiment or a pharmaceutically acceptable salt thereof, wherein, X may be N, and Y may be C₁₋₆ alkyl. In one embodiment of the present invention, X may be N, and Y may be ethyl.

Another embodiment of the present invention is the compound of the aforesaid embodiment or a pharmaceutically acceptable salt thereof, wherein X may be C, Y may be —NR_{f}R_{g} or

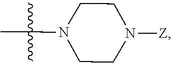

and Z may be C₁₋₆ alkyl; wherein R_{f} and R_{g} may respectively be a C₁₋₆ alkyl. In one embodiment of the present invention, Y may be —N(CH₃)₂ or

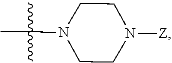

and Z may be methyl.

Another embodiment of the present invention is the compound of the aforesaid embodiment or a pharmaceutically acceptable salt thereof, wherein the compound is a maleic salt thereof.

A further another embodiment of the present invention can be a compound shown in the following Table 1, or a pharmaceutically acceptable salt thereof.

The compounds of the present invention may contain asymmetric or chiral centers, and exist in different stereoisomeric forms. Unless specified otherwise, all stereoisomeric forms of the compounds of the present invention as well as mixtures thereof, including racemic mixtures are within the scope of the present invention. In addition, the compounds of the present invention may also exist in different geometric and positional isomers. For example, both the cis- and trans-forms, as well as mixtures of the compound with a double bond or a fused ring, are also within the scope of the present invention.

Diastereomeric mixtures can be separated into their individual diastereoisomers by any methods, such as by chromatography and/or fractional crystallization. Enantiomers can be separated by use of a chiral HPLC column or by converting the enantiomeric mixture into a diastereomeric mixture by reaction with an appropriate optically active compound to separate the diastereoisomers and convert the individual diastereoisomers into pure enantiomers. The specific stereoisomers may be synthesized by converting one stereoisomer into the other by asymmetric transformation, by using an optically active starting material or by asymmetric synthesis using optically active reagents, catalysts, substrates or solvents.

Also within the present invention is a pharmaceutical composition, comprising: (1) the compound of the present invention, or a pharmaceutically acceptable salt thereof; and (2) a pharmaceutically acceptable carrier, excipient or diluent. The composition may also comprise at least one additional therapeutic agent such as anti-cancer agents. The compound or the pharmaceutically acceptable salt thereof or the composition of the present invention may be used in the manufacture of a medicament of inhibiting the growth of tumor cells or treating cancer. In one embodiment of the present invention, the therapeutic agent may be at least one selected from the group consisting of paclitaxel, docetaxel, ixabepilone, eribulin, and vinorelbine.

Also within the present invention is a method for treating a cancer, which includes the step of administering to the subject in need thereof an effective amount of the compound of the present invention or a pharmaceutically acceptable salt thereof.

Further covered by the present invention a method of inhibiting a growth of tumor cells, which includes the step of administering to a subject in need thereof an effective amount of the compound of the present invention or a pharmaceutically acceptable salt thereof.

In the present invention, the aforesaid subject can be mammal, for example, human.

In the present invention, the compound of the present invention or a pharmaceutically acceptable salt thereof can inhibit the growth of tumor cells to achieve the purpose of treating a cancer. Examples of the cancer include, but are not limited to, gastric cancer, colon cancer, colorectal cancer, breast cancer, lung cancer, prostate cancer, bladder cancer, pancreatic cancer, liver cancer, uterine cancer, cervical cancer, endometrial cancer, esophageal cancer, leukemia, lymphoma, kidney cancer, osteosarcoma, ovarian cancer, skin cancer, small intestine cancer, thymus cancer, thyroid cancer, nervous system cancers, bone cancer, brain cancer, or head and neck cancer. In one embodiment of the present invention, the cancer may be breast cancer.

The compounds or a pharmaceutically acceptable salt thereof of the present invention may be administered in combination with at least one additional therapeutic agent such as anti-cancer agent. The administration formulation can be, for example, (a) a single formulation comprising the compound of the present invention or a pharmaceutically acceptable salt thereof, a pharmaceutically acceptable carrier, excipient or diluent and at least one additional therapeutic agent; or (b) two formulations administered simultaneously or sequentially and in any order, wherein one comprises the compound of the present invention or a pharmaceutically acceptable salt thereof, a pharmaceutically acceptable carrier, excipient or diluent and the other one comprises at least one additional therapeutic agent.

The following embodiments are made to clearly exhibit the above-mentioned and other technical contents, features and/or effects of the present invention. Through the exposition by means of the specific embodiments, people would further understand the technical means and effects the present invention adopts to achieve the above-indicated objectives. Moreover, as the contents disclosed herein should be readily understood and can be implemented by a person skilled in the art, all equivalent changes or modifications which do not depart from the concept of the present invention should be encompassed by the appended claims.

### EXAMPLE

Without further elaboration, it is believed that one skilled in the art can, based on the above description, utilize the present invention to its fullest extent. The following specific examples are therefore to be construed as merely illustrative, and not limitative of the remainder of the disclosure in any way whatsoever. All publications cited herein are incorporated by reference in their entirety.

Described below are the procedures used to synthesize the exemplary compounds of the present invention.

### General Chemistry Methods

All commercial chemicals and solvents are of reagent grade and were used without further purification unless otherwise stated. All reactions were carried out under dry nitrogen or argon atmosphere and were monitored for completion by TLC using Merck 60 F254 silica gel glass-backed plates or aluminum plates; zones were detected visually under UV irradiation (254 nm) or by spraying with potassium permanganate reagent (Aldrich) followed by heating at 80° C. Flash column chromatography was carried out using silica gel (Silicycle SiliaFlash® P60, R12030B, 230-400 mesh). ¹H and ¹³C NMR spectra were recorded with Varian Mercury-300 spectrometers or Bruker 600 MHz AVANCE III spectrometers. Data analysis was done using Mnova software (Mestrelab Research). Chemical shift (δ) was reported in ppm and referenced to solvent residual signals as follows: DMSO-d₆ at 2.50 ppm, CDCl₃ at 7.26 ppm for ¹H NMR; DMSO-d₆ at 39.5 ppm, CDCl₃ at 77.0 ppm for 13 C NMR. Splitting patterns are indicated as follows: s=singlet; d=doublet; m=multiplet. Coupling constants (J) were given in Hertz (Hz). Low-resolution mass spectra (LRMS) data were measured with Agilent Infinity 111290 LC/MS (ESI) systems. High-resolution mass spectra (HRMS) data were measured with Varian 901-MS FT-ICR HPLC/MS-MS system. Purity of the final compounds were determined using ultra performance liquid chromatography (UPLC) system (Waters Acquity UPLC/BSM) equipped with a C18 column (Waters Acquity BEH-C18 1.7 m. 2.1 mm×50 mm) and operating at 25° C. For UPLC system, elution was carried out using acetonitrile as mobile phase A, and water containing 0.1% formic acid and 2 mmol NH4Oac as mobile phase B. Elution conditions: at 0.00 min, phase A 10%+phase B 90%; at 4.15 min, phase A 90%+phase B 10%; at 5.00 min, phase A 10%+phase B 90%; at 6.50 min, phase A 10%+phase B 90%. The flow rate of the mobile phase was 0.6 mL/min, and the injection volume of the sample was 5 μL. Peaks were detected at 254 nm. IUPAC nomenclatures of compounds were obtained with Mnova software (Mestrelab Research).

### Synthesis of 4-(((2Z)-5-(4-chlorophenyl)-7-methyl-3-oxo-6-((propan-2-yloxy)carbonyl)-5H-[1,3]thiazolo[3,2-a]pyrimidin-2(3H)-ylidene)methyl)phenoxy)acetic acid (1)

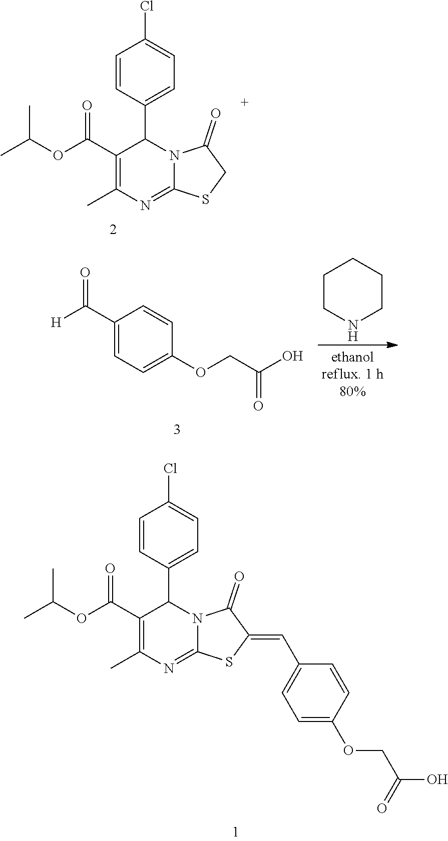 [SMILES: CC1=C(C(=O)OC(C)C)C(c2ccc(Cl)cc2)N2C(=O)CSC2=N1 ; O=Cc1ccc(OCC(=O)O)cc1 ; C1CCNCC1 ; CC1=C(C(=O)OC(C)C)C(c2ccc(Cl)cc2)n2c(s/c(=C\c3ccc(OCC(=O)O)cc3)c2=O)=N1]

Dissolve compound 2 (1.3 g, 3.5 mmol) in 20 ml of ethanol, add compound 3 (0.64 g, 3.5 mmol) and piperidine (0.37 ml, 3.5 mmol), and heat to reflux and react for 1 hour. The ethanol was drained by cycloconcentration, water and 3.0N HCl were added to adjust the pH value to 2.0, and extracted twice with methylene chloride. The organic layer was dehydrated with anhydrous magnesium sulfate, filtered, and concentrated to obtain a crude product. The crude product was subjected to column chromatography using gradient elution (gradient from methanol:ammonia:methylene chloride=1:0.1:50 to methanol:ammonia:dichloromethane=1:0.1:20, R_{f}=0.15, as the eluent), and after purification, yellow solid compound 1 (1.5 g, yield 80%) was obtained. ¹H NMR (400 MHz, DMSO-d₆) δ 7.75 (s, 1H), 7.56 (d, J=9.2 Hz, 2H), 7.42 (d, J=8.4 Hz, 2H), 7.33 (d, J=8.4 Hz, 2H), 7.08 (d, J=9.2 Hz, 2H), 6.00 (s, 1H), 4.84 (p, J=6.2 Hz, 1H), 4.78 (s, 2H), 2.39 (s, 3H), 1.19 (d, J=6.2 Hz, 3H), 0.98 (d, J=6.2 Hz, 3H).

LRMS (ESI) m/z: 527.1 [M+H]⁺.

HRMS (ESI) m/z calcd for C₂₆H₂₃ClN₂O₆S [M+H]⁺=527.1044. found: 527.1040.

### Synthesis of 1-Methylethyl 5-(4-chlorophenyl)-2,3-dihydro-7-methyl-3-oxo-5H-thiazolo[3,2-a]pyrimidine-6-carboxylate (2)

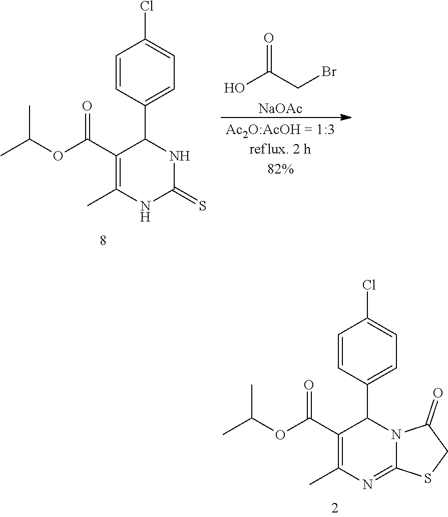 [SMILES: CC1=C(C(=O)OC(C)C)C(c2ccc(Cl)cc2)NC(=S)N1 ; O=C(O)CBr ; CC1=C(C(=O)OC(C)C)C(c2ccc(Cl)cc2)N2C(=O)CSC2=N1 ; "NaOAc"]

Mix compound 8 (1.8 g, 5.5 mmol) with acetic anhydride (6 ml) and acetic acid (18 ml) as solvent, add bromoacetic acid (0.92 g, 6.7 mmol) and sodium acetate (0.45 g, 5.5 mmol), heat to reflux, react for 2 hours, and add saturated sodium bicarbonate aqueous solution. Extract twice with methylene chloride. The organic layer was dehydrated with anhydrous magnesium sulfate, filtered, and concentrated to obtain a crude product. The crude product was subjected to column chromatography (ethyl acetate:n-hexane=1:6, R_{f}=0.21, as the eluent), and after purification, compound 2 (1.7 g, yield 82%) was obtained as a brown oil.

¹H NMR (400 MHz, CDCl₃) δ 7.33-7.26 (m, 4H), 6.01 (s, 1H), 4.94 (p, J=6.2 Hz, 1H), 3.86 (d, J=17.2 Hz, 1H), 3.73 (d, J=17.2 Hz, 1H), 2.48 (s, 3H), 1.21 (d, J=6.2 Hz, 3H), 1.03 (d, J=6.2 Hz, 3H).

¹³C NMR (400 MHz, CDCl₃) δ 170.20, 164.52, 159.51, 152.22, 138.38, 134.39, 129.44, 128.60, 107.79, 77.32, 77.00, 76.68, 67.97, 54.82, 32.26, 22.52, 21.81, 21.43. LRMS (ESI) m/z: 365.1 [M+H]⁺.

### Synthesis of 2-(4-formylphenoxy)acetic acid (3)

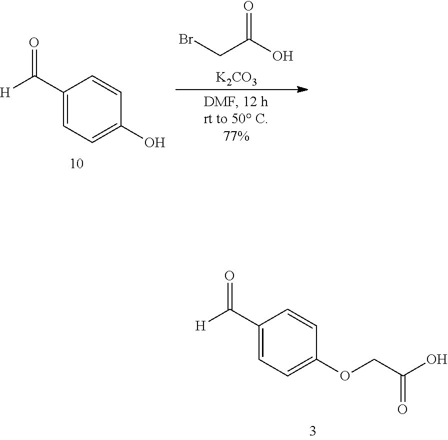

Compound 10 (4-hydroxybenzaldehyde) (1.5 g, 12.3 mmol) was dissolved in 20 ml of dimethylformamide (DMF), and potassium carbonate (8.49 g, 61.5 mmol) was added. After reacting at room temperature for 15 minutes, bromoacetic acid (2.05 g, 14.7 mmol) was dissolved in 15 ml of dimethylformamide followed by adding into the reaction, and the reaction was raised to 50° C. from room temperature. After reaction for 12 hours, add 120 ml of water, slowly drip 6N hydrochloric acid, adjust the pH value to 2, filter and vacuum dry to obtain the crude product. The crude product was purified by ethanol recrystallization, and white solid compound 3 was obtained after filtration and purification (1.7 g, yield 77%).

¹H NMR (400 MHz, DMSO-d₆) δ 9.85 (s, 1H), 7.84 (d, J=8.8 Hz, 2H), 7.09 (d, J=8.8 Hz, 2H), 4.81 (s, 2H).

LRMS (ESI) m/z: 181.2 [M+H]⁺.

### Synthesis of propan-2-yl 3-oxobutanoate (5)

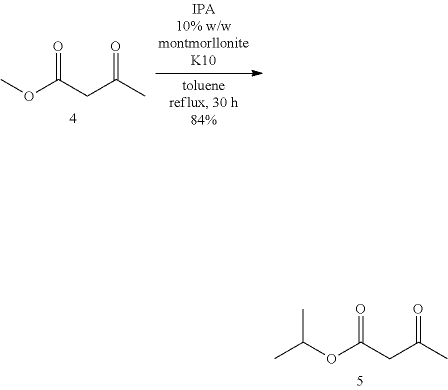

Compound 4 (methyl acetyl acetate) (1.16 g, 10.0 mmol) was dissolved in toluene (15.0 ml), Montmorillonite K10 (0.12 g, 10% w/w) and isopropanol (0.76 ml, 10.0 mmol) were added. Heat to reflux for 30 hours, filter the Montmorillonite K10 with an appropriate amount of diatomaceous earth, remove the toluene by concentrating under reduced pressure, and add water and extract with ethyl acetate. The organic layer was dehydrated with anhydrous magnesium sulfate, filtered, and concentrated to obtain a crude product. The crude product was subjected to column chromatography (ethyl acetate:n-hexane=1:49, R_{f}=0.18, as the eluent), and after purification, a transparent liquid compound 5 (1.2 g, yield 84%) was obtained.

¹H NMR (400 MHz, CDCl₃) δ 5.05 (p, J=6.2 Hz, 1H), 3.40 (s, 2H), 2.25 (s, 3H), 1.25 (d, J=6.2 Hz, 6H).

¹³C NMR (400 MHz, CDCl₃) δ 199.72, 165.82, 88.94, 67.48, 49.14, 28.62, 20.55.

LRMS (ESI) m/z: 145.1 [M+H]⁺.

### Synthesis of propan-2-yl 4-(4-chlorophenyl)-6-methyl-2-sulfanylidene-3,4-dihydro-1H-pyrimidine-5-carboxylate (8)

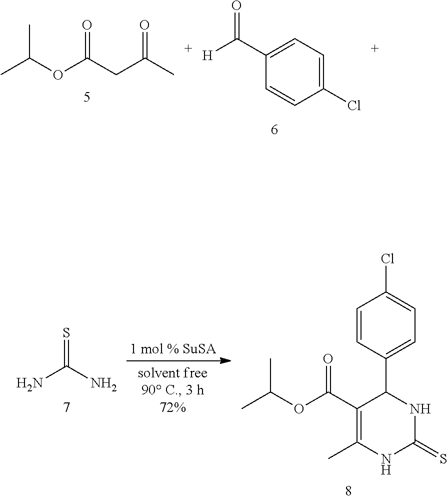

Compound 5 (1.10 g, 7.6 mmol), compound 6 (4-chlorobenzaldehyde) (1.07 g, 7.6 mmol) and compound 7 (thiourea) (0.87 g, 11.4 mmol) were added with SuSA (1 mol %, 13.6 mg), followed by heating to 90° C. After reacting for 3 hours, add water and extract with ethyl acetate. The organic layer was extracted twice with water to remove excess thiourea. The organic layer was dehydrated with anhydrous magnesium sulfate, filtered, and concentrated to obtain crude product. The crude product was added with 20 ml of methanol, purified by recrystallization, and filtered to obtain a white solid product (1.1 g). The filtered filtrate was then subjected to column chromatography (ethyl acetate:n-hexane=1:6, R_{f}=0.2, as the eluent) for purification, and a white solid compound 8 (1.8 g, yield 72%) was obtained by the two purification methods.

¹H NMR (400 MHz, CDCl₃) δ 7.70 (s, 1H), 7.30 (d, J=8.8 Hz, 2H), 7.23 (d, J=8.8 Hz, 2H), 7.18 (s, 1H), 5.37 (s, 1H), 4.96 (p, J=6.2 Hz, 1H), 2.36 (s, 3H), 1.22 (d, J=6.2 Hz, 3H), 1.06 (d, J=6.2 Hz, 3H).

¹³C NMR (400 MHz, DMSO-d₆) δ 174.3, 164.5, 145.2, 142.5, 132.3, 128.5, 128.4, 100.6, 66.9, 53.6, 22.0

LRMS (ESI) m/z: 325.1 [M+H]⁺.

### Synthesis of sodium (Z)-2-(4-((5-(4-chlorophenyl)-6-(isopropoxycarbonyl)-7-methyl-3-oxo-5H-thiazolo[3,2-a]pyrimidin-2(3H)-ylidene)methyl)phenoxy)acetate (11)

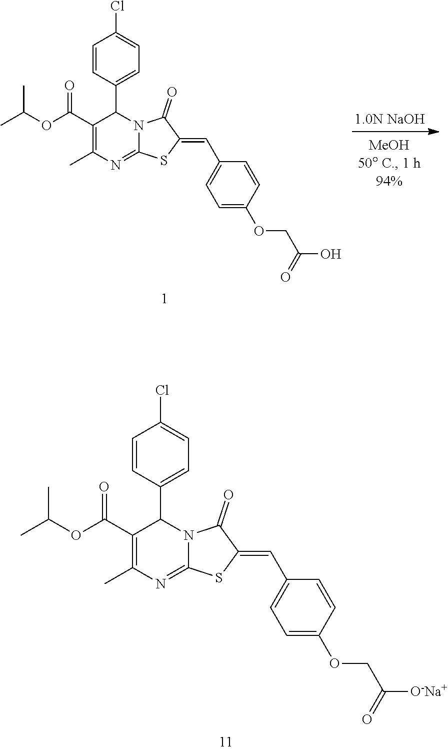

Compound 1 (100 mg, 0.19 mmol) was added to 0.3 ml of 1.0 N NaOH. Since the starting material was not dissolved after adding the aqueous sodium hydroxide solution, 2 ml of methanol was added to help dissolve and the reaction was heated to 50 degrees to dissolve completely. After heating for one hour, leave it at room temperature until the product solid precipitates, and then filter with a small amount of methanol:water=1:1 to remove excess sodium hydroxide in the reaction. The solid was evacuated to obtain compound 11 (98 mg, yield 94%).

¹H NMR (400 MHz, DMSO-d₆) δ 7.71 (s, 1H), 7.50 (d, J=9.2 Hz, 2H), 7.42 (d, J=8.8 Hz, 2H), 7.33 (d, J=8.8 Hz, 2H), 6.95 (d, J=9.2 Hz, 2H), 6.00 (s, 1H), 4.85 (p, J=6.2 Hz, 1H), 4.28 (s, 2H), 2.39 (s, 3H), 1.19 (d, J=6.2 Hz, 3H), 0.99 (d, J=6.2 Hz, 3H).

LRMS (ESI) m/z: 527.1 [M+H]⁺.

### Synthesis of isopropyl (Z)-5-(4-chlorophenyl)-7-methyl-2-(4-(2-morpholino-2-oxoethoxy)benzylidene)-3-oxo-2,3-dihydro-5H-thiazolo[3,2-a]pyrimidine-6-carboxylate (12)

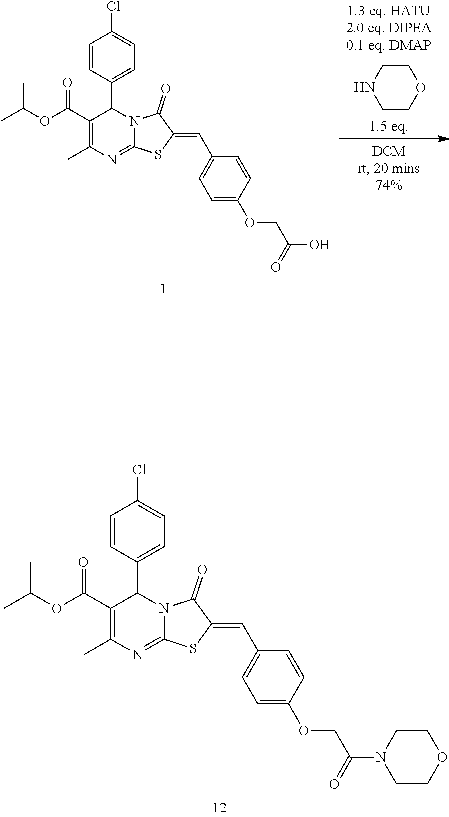 [SMILES: CC1=C(C(=O)OC(C)C)C(c2ccc(Cl)cc2)n2c(s/c(=C\c3ccc(OCC(=O)O)cc3)c2=O)=N1 ; C1COCCN1 ; CC1=C(C(=O)OC(C)C)C(c2ccc(Cl)cc2)n2c(s/c(=C\c3ccc(OCC(=O)N4CCOCC4)cc3)c2=O)=N1]

Compound 1 (105 mg, 0.2 mmol), 1-[bis(dimethylamino)methylene]-1H-1,2,3-triazolo[4,5-b]pyridinium 3-oxid hexafluorophosphate (HATU) (99 mg, 0.26 mmol), N,N-diisopropylethylamine (DIPEA) (0.07 mL, 0.4 mmol), 4-dimethylaminopyridine (DMAP) (2.4 mg, 0.02 mmol) and morpholine (0.04 ml, 0.3 mmol) were dissolved in 3 ml of methylene chloride. After reacting at room temperature for 20 minutes, water was added for extraction. The organic layer was dehydrated with anhydrous magnesium sulfate, filtered, and concentrated to obtain a crude product. The crude product was subjected to column chromatography (methanol:dichloromethane=1:50, R_{f}=0.25, as the eluent), and after purification, a yellow solid compound 12 (200 mg, yield 74%) was obtained.

¹H NMR (300 MHz, CDCl₃) δ 7.72 (s, 1H), 7.44 (d, J=8.7 Hz, 2H), 7.35 (d, J=8.4 Hz, 2H), 7.28 (d, J=8.4 Hz, 2H), 7.03 (d, J=8.7 Hz, 2H), 6.16 (s, 1H), 4.98 (septet, 1H), 4.76 (s, 2H), 3.67-3.57 (m, 8H), 2.54 (s, 3H), 1.24 (d, J=6.3 Hz, 3H), 1.05 (d, J=6.3 Hz, 3H).

LRMS (ESI) m/z: 596.1 [M+H]⁺.

HRMS (ESI) m/z calcd for C₃₀H₃₀ClN₃O₆S [M+H]⁺=596.1622. found: 596.1609.

### Synthesis of propan-2-yl (2Z)-5-(4-chlorophenyl)-7-methyl-2-(4-(2-(4-methylpiperazin-1-yl)-2-oxoethoxy)benzylidene)-3-oxo-2,3-dihydro-5H-[1,3]thiazolo[3,2-a]pyrimidine-6-carboxylate (13)

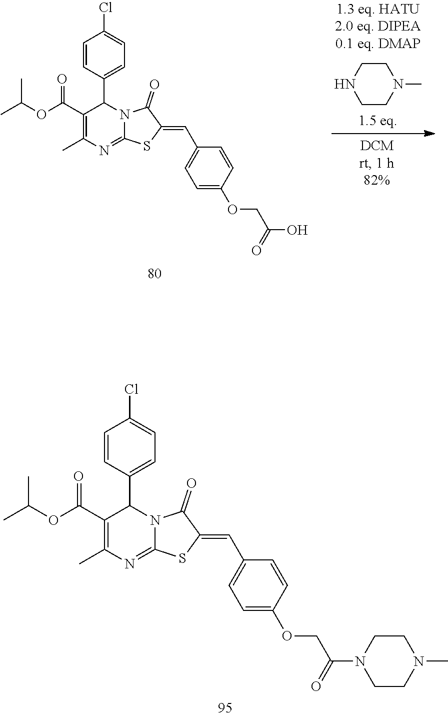

Experimental steps were similar to compound 12. The acid group was activated with HATU, and the amine compound was 1-methylpiperazine. The crude product was subjected to column chromatography (methanol:dichloromethane=1:40, R_{f}=0.31, as the eluent), and after purification, a yellow solid compound 13 was obtained with a yield of 82%.

¹H NMR (400 MHz, CDCl₃) δ 7.69 (s, 1H), 7.43 (d, J=8.8 Hz, 2H), 7.35 (d, J=8.8 Hz, 2H), 7.29-7.26 (m, 2H), 7.03 (d, J=8.8 Hz, 2H), 6.15 (s, 1H), 4.99-4.75 (m, 1H), 4.75 (s, 2H), 3.64 (t, J=4.8 Hz, 2H), 3.56 (t, J=4.8 Hz, 2H), 2.52 (s, 3H), 2.41-2.36 (m, 4H), 2.29 (s, 3H), 1.24 (d, J=6.0 Hz, 3H), 1.05 (d, J=6.0 Hz, 3H). ¹³C NMR (400 MHz, CDCl₃) δ 165.4, 164.9, 164.4, 159.4, 155.8, 152.2, 138.3, 134.1, 132.9, 131.8, 129.3, 128.4, 126.3, 117.2, 115.2, 108.4, 67.8, 66.8, 54.6, 54.1, 45.5, 44.5, 41.5, 22.5, 21.7, 21.3.

LRMS (ESI) m/z: 609.2 [M+H]⁺.

HRMS (ESI) m/z calcd for C₃₁H₃₃ClN₄O₅S [M+H]⁺=609.1938. found: 609.1934.

### Synthesis of propan-2-yl (2Z)-5-(4-chlorophenyl)-2-(4-(2-(4-ethylpiperazin-1-yl)-2-oxoethoxy)benzylidene)-7-methyl-3-oxo-2,3-dihydro-5H-[1,3]thiazolo[3,2-a]pyrimidine-6-carboxylate (14)

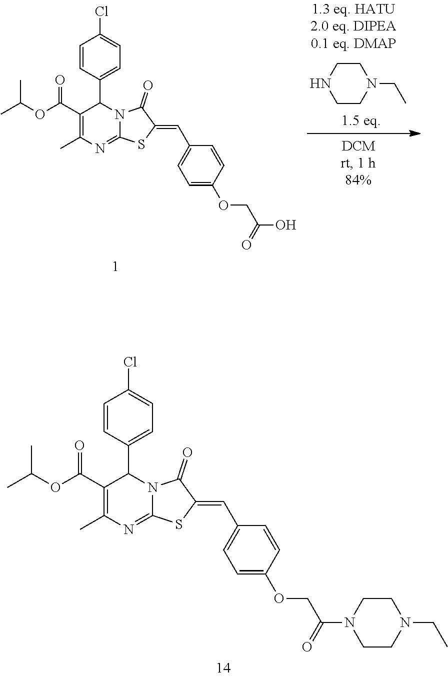

Experimental steps were similar to compound 12. The acid group was activated with HATU, and the amine compound was 1-ethylpiperazine. The crude product was subjected to column chromatography (methanol:dichloromethane=1:40, R_{f}=0.28 as the eluent), and after purification, a yellow solid compound 14 was obtained with a yield of 84%.

¹H NMR (300 MHz, CDCl₃) δ 7.69 (s, 1H), 7.43 (d, J=8.7 Hz, 2H), 7.35 (d, J=8.4 Hz, 2H), 7.27 (d, J=8.4 Hz, 2H), 7.02 (d, J=8.7, 2zH), 6.15 (s, 1H), 4.97 (septet, J=6.3 Hz, 1H), 4.75 (s, 2H), 3.66-3.55 (m, 4H), 2.52 (s, 3H), 2.43-2.40 (m, 6H), 1.24 (d, J=6.3 Hz, 3H), 1.15-1.01 (m, 6H).

LRMS (ESI) m/z: 623.2 [M+H]⁺.

HRMS (ESI) m/z calcd for C₃₂H₃₅ClN₄O₅S [M+H]⁺=623.2095. found: 623.2094.

### Synthesis of isopropyl (Z)-2-(4-(2-amino-2-oxoethoxy)benzylidene)-5-(4-chlorophenyl)-7-methyl-3-oxo-2,3-dihydro-5H-thiazolo[3,2-a]pyrimidine-6-carboxylate (15)

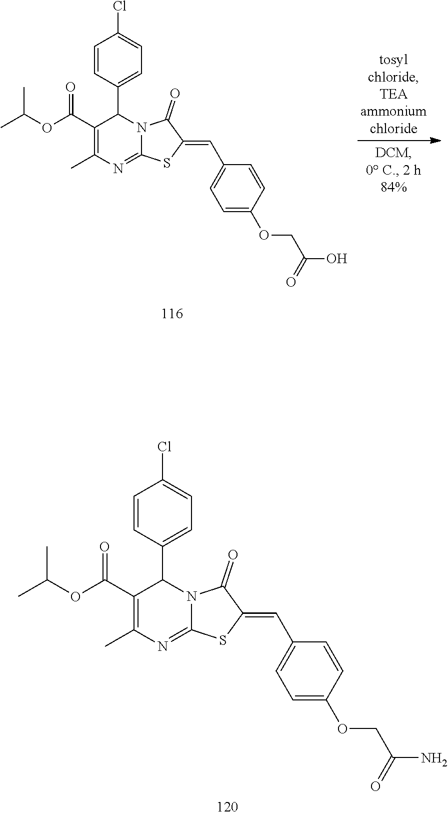 [SMILES: CC1=C(C(=O)OC(C)C)C(c2ccc(Cl)cc2)n2c(s/c(=C\c3ccc(OCC(=O)O)cc3)c2=O)=N1 ; CC1=C(C(=O)OC(C)C)C(c2ccc(Cl)cc2)n2c(s/c(=C\c3ccc(OCC(N)=O)cc3)c2=O)=N1]

Compound 1 (150 mg, 0.28 mmol) was dissolved in 3 ml of methylene chloride, and p-toluenesulfonyl chloride (tosyl chloride) (0.15 ml, 0.3 mmol), triethylamine (TEA) (0.04 ml, 0.3 mmol), and ammonium chloride (2.4 mg, 0.02 mmol) were added under ice bath. After reacting at room temperature for 2 hours, water was added for extraction. The organic layer was dehydrated with anhydrous magnesium sulfate, filtered, and concentrated to obtain a crude product. The crude product was subjected to column chromatography (methanol:dichloromethane=1:30, R_{f}=0.32, as the eluent), and after purification, a yellow solid compound 15 (72 mg, yield 84%) was obtained.

¹H NMR (300 MHz, CDCl₃) δ 7.69 (s, 1H), 7.45 (d, J=8.7 Hz, 2H), 7.35 (d, J=8.4 Hz, 2H), 7.27 (d, J=8.7 Hz, 2H), 7.01 (d, J=8.7 Hz, 2H), 6.47 (brs, 1H), 6.14 (s, 1H), 5.72 (brs, 1H), 5.01-4.92 (m, 1H), 4.54 (s, 2H), 2.52 (s, 3H), 1.23 (d, J=6.3 Hz, 3H), 1.05 (d, J=6.3 Hz, 3H). ¹³C NMR (600 MHz, DMSO-d₆) δ 169.3, 164.4, 164.1, 159.7, 155.7, 151.5, 139.3, 133.1, 133.0, 132.1, 129.6, 128.6, 125.7, 116.6, 115.7, 108.2, 67.7, 66.6, 54.4, 22.4, 21.6, 21.2.

LRMS (ESI) m/z: 526.1 [M+H]⁺.

HRMS (ESI) m/z calcd for C₂₆H₂₄ClN₃O₅S [M+H]⁺=526.1203. found: 526.1316.

### Synthesis of isopropyl (Z)-5-(4-chlorophenyl)-2-(4-(2-(4-isopropylpiperazin-1-yl)-2-oxoethoxy)benzylidene)-7-methyl-3-oxo-2,3-dihydro-5H-thiazolo[3,2-a]pyrimidine-6-carboxylate (16)

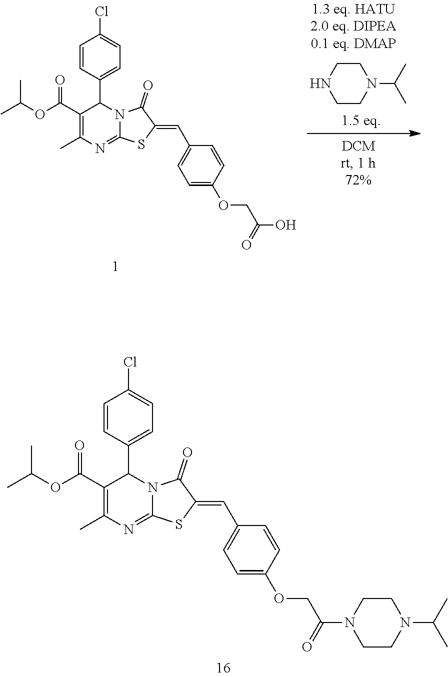

Experimental procedure was similar to compound 12. Acid group was activated with HATU, amine compound was (1-isopropylpiperazine), and crude product was subjected to column chromatography (methanol:dichloromethane=1:40, R_{f}=0.29, as the eluent). After purification, a yellow solid compound 16 was obtained with a yield of 72%.

¹H NMR (400 MHz, CDCl₃) δ 7.69 (s, 1H), 7.43 (d, J=8.8 Hz, 2H), 7.36 (d, J=8.8 Hz, 2H), 7.29-7.26 (m, 2H), 7.03 (d, J=8.8 Hz, 2H), 6.15 (s, 1H), 4.97 (septet, J=6.0 Hz 1H), 4.75 (s, 2H), 3.63 (m, 2H), 3.56 (m, 2H), 2.73-2.69 (m, 1H), 2.52-2.48 (m, 7H), 1.24 (d, J=6.4 Hz, 3H), 1.06-1.01 (m, 9H).

LRMS (ESI) m/z: 637.3 [M+H]⁺.

HRMS (ESI) m/z calcd for C₃₃H₃₇ClN₄O₅S [M+H]⁺=637.2251. found: 637.2247.

### Synthesis of propan-2-yl (2Z)-5-(4-chlorophenyl)-2-(4-(2-(4-cyclohexylpiperazin-1-yl)-2-oxoethoxy)benzylidene)-7-methyl-3-oxo-2,3-dihydro-5H-[1,3]thiazolo[3,2-a]pyrimidine-6-carboxylate (17)

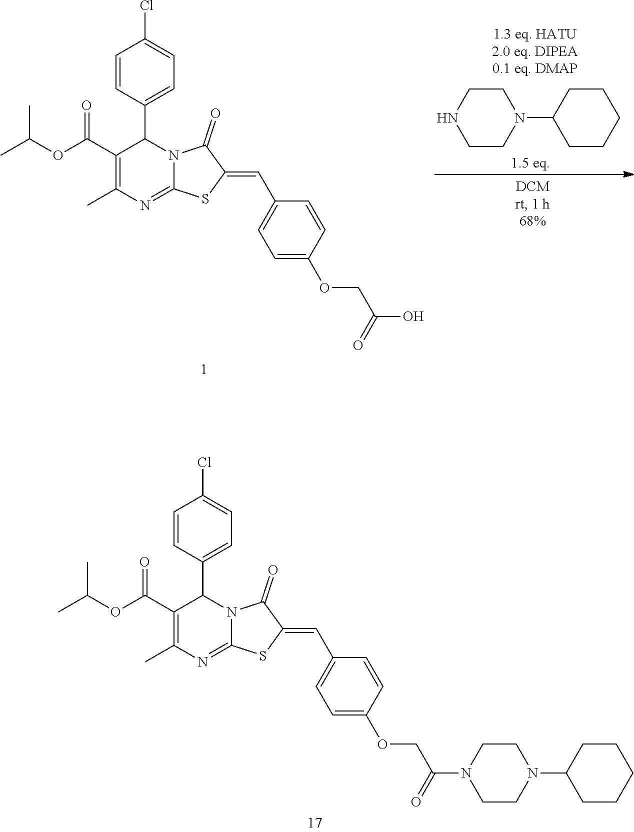 [SMILES: CC1=C(C(=O)OC(C)C)C(c2ccc(Cl)cc2)n2c(s/c(=C\c3ccc(OCC(=O)O)cc3)c2=O)=N1 ; C1CCC(N2CCNCC2)CC1 ; CC1=C(C(=O)OC(C)C)C(c2ccc(Cl)cc2)n2c(s/c(=C\c3ccc(OCC(=O)N4CCN(C5CCCCC5)CC4)cc3)c2=O)=N1]

The experimental procedure was similar to compound 12. HATU was used to activate the acid group. The amine compound was 1-cyclohexylpiperazine. The crude product was analyzed by column chromatography (methanol:dichloromethane=1:40, R_{f}=0.33, as the eluent). After purification, a yellow solid compound 17 was obtained with a yield of 68%.

¹H NMR (300 MHz, CDCl₃) δ 7.69 (s, 1H), 7.42 (d, J=8.4 Hz, 2H), 7.35 (d, J=8.7 Hz, 2H), 7.27 (d, J 8.7 Hz, 2H), 7.02 (d, J 8.7 Hz, 2H), 6.15 (s, 1H), 4.97 (septet, J 6.3 Hz, 1H), 3.62-3.53 (m, 4H), 2.60-2.51 (i, 7H), 2.27-2.26 (m, 1H), 1.81-1.64 (m, 4H), 1.25-1.09 (m, 9H), 1.05 (d, J=6.3 Hz, 3H).

LRMS (ESI) m/z: 677.3 [M+H]⁺.

HRMS (ESI) m/z calcd for C₃₆H₄₁ClN₄O₅S [M+H]⁺=677.2564. found: 677.2579.

### Synthesis of propan-2-yl (2Z)-5-(4-chlorophenyl)-2-(4-(2-(4-(dimethylamino)piperidin-1-yl)-2-oxoethoxy)benzylidene)-7-methyl-3-oxo-2,3-dihydro-5H-[1,3]thiazolo[3,2-a]pyrimidine-6-carboxylate (18)

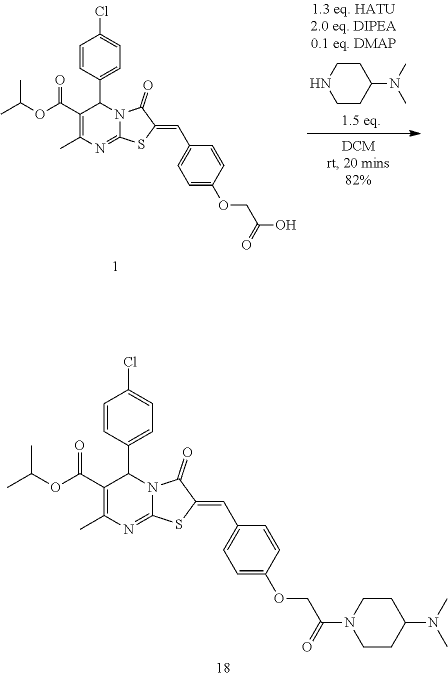

The experimental procedure was similar to compound 12. HATU was used to activate the acid group, and the amine compound was 4-dimethylaminopyridine. The crude product was subjected to column chromatography (methanol:dichloromethane=1:40, R_{f}=0.28, as the eluent). After purification, a yellow solid compound 18 was obtained with a yield of 82%.

¹H NMR (400 MHz, CDCl₃) δ 7.68 (s, 1H), 7.42 (d, J=8.8 Hz, 2H), 7.35 (d, J=8.8 Hz, 2H), 7.27 (d, J=8.8 Hz, 2H), 7.03 (d, J=8.8 Hz, 2H), 6.14 (s, 1H), 4.95 (septet, J=6.0 Hz, 1H), 4.75 (s, 2H), 4.57-4.52 (m, 1H), 4.00-3.95 (m, 1H), 3.08 (t, J=12.0 Hz, 1H), 2.65 (t, J=12.0 Hz, 1H), 2.51 (s, 3H), 2.38 (t, J=10.6 Hz, 1H), 2.27 (s, 6H), 1.88 (t, J=13.2 Hz, 2H), 1.43-1.35 (m, 2H), 1.23 (d, J=6.0 Hz, 3H), 1.05 (d, J=6.0 Hz, 3H).

¹³C NMR (400 MHz, CDCl₃) δ 165.3, 165.3, 164.7, 159.7, 156.1, 152.5, 138.5, 134.4, 133.2, 132.0, 131.9, 129.5, 129.4, 128.6, 128.5, 126.5, 117.5, 115.4, 108.6, 68.0, 67.4, 61.7, 54.8, 44.3, 41.6, 41.4, 29.1, 27.9, 22.7, 21.9, 21.5.

LRMS (ESI) m/z: 637.2 [M+H]⁺.

HRMS (ESI) m/z calcd for C₃₃H₃₇ClN₄O₅S [M+H]⁺=637.2251. found: 637.2207.

### Synthesis of isopropyl (Z)-5-(4-chlorophenyl)-7-methyl-2-(4-(2-(4-(4-methylpiperazin-1-yl)piperidin-1-yl)-2-oxoethoxy)benzylidene)-3-oxo-2,3-dihydro-5H-thiazolo[3,2-a]pyrimidine-6-carboxylate (19)

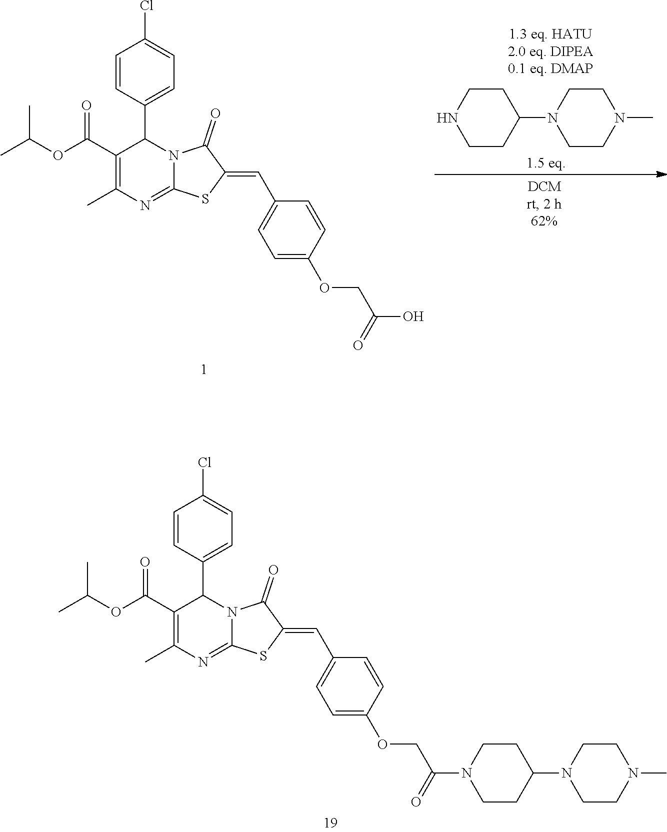 [SMILES: CC1=C(C(=O)OC(C)C)C(c2ccc(Cl)cc2)n2c(s/c(=C\c3ccc(OCC(=O)O)cc3)c2=O)=N1 ; CN1CCN(C2CCNCC2)CC1 ; CC1=C(C(=O)OC(C)C)C(c2ccc(Cl)cc2)n2c(s/c(=C\c3ccc(OCC(=O)N4CCC(N5CCN(C)CC5)CC4)cc3)c2=O)=N1]

The experimental procedure was similar to compound 12. HATU was used to activate the acid group, and the amine compound was 1-methyl-4-(piperidin-4-yl)piperazine. The crude product was purified using column chromatography (methanol:dichloromethane=1:40, R_{f}=0.38 as the eluent) to obtain compound 19, a yellow solid, with a yield of 62%.

¹H NMR (400 MHz, CDCl₃) δ 7.68 (s, 1H), 7.41 (d, J=8.8 Hz, 2H), 7.35 (d, J=8.8 Hz, 2H), 7.27 (d, J=8.8 Hz, 2H), 7.02 (d, J=8.8 Hz, 2H), 6.14 (s, 1H), 4.96 (septet, J=6.2 Hz, 1H), 4.74 (s, 2H), 4.59-4.55 (m, 1H), 4.01-3.97 (m, 1H), 3.08-3.03 (m, 2H), 2.69-2.61 (m, 4H), 2.51 (s, 3H), 2.42 (s, 3H), 1.95-1.85 (m, 3H), 1.57-1.23 (m, 5H), 1.31 (d, J=6.2 Hz, 3H), 1.04 (d, J=6.2 Hz, 3H).

¹³C NMR (400 MHz, CHCl₃) δ 165.2, 165.1, 164.5, 159.6, 155.9, 152.3, 138.4, 134.2, 133.0, 131.9, 129.4, 128.5, 126.4, 117.3, 115.3, 108.5, 67.9, 67.2, 61.1, 54.8, 54.6, 48.2, 45.3, 44.3, 41.4, 28.6, 27.9, 22.6, 21.8, 21.4.

LRMS (ESI) m/z: 692.3 [M+H]⁺.

HRMS (ESI) m/z calcd for C₃₆H₄₂ClN₅O₅S [M+H]⁺=692.2673. found: 692.2661.

### Synthesis of isopropyl (Z)-5-(4-chlorophenyl)-2-(4-(2-(4-hydroxypiperidin-1-yl)-2-oxoethoxy)benzylidene)-7-methyl-3-oxo-2,3-dihydro-5H-thiazolo[3,2-a]pyrimidine-6-carboxylate (20)

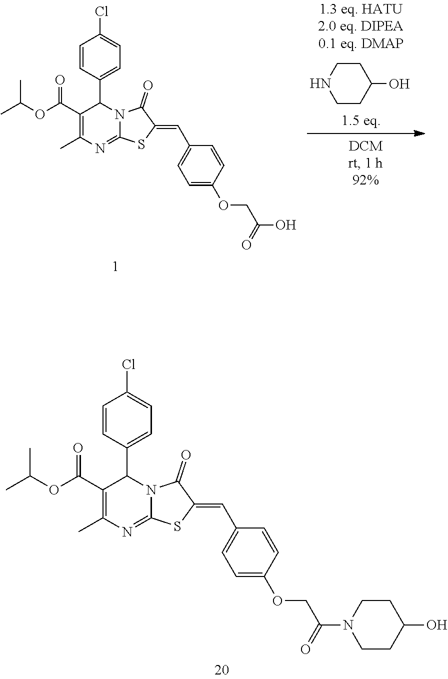 [SMILES: CC1=C(C(=O)OC(C)C)C(c2ccc(Cl)cc2)n2c(s/c(=C\c3ccc(OCC(=O)O)cc3)c2=O)=N1 ; OC1CCNCC1 ; CC1=C(C(=O)OC(C)C)C(c2ccc(Cl)cc2)n2c(s/c(=C\c3ccc(OCC(=O)N4CCC(O)CC4)cc3)c2=O)=N1]

The experimental procedure was similar to that of compound 12. HATU was used to activate the acid group, and the amine compound was 4-piperidin-4-ol. The crude product was subjected to column chromatography (methanol:dichloromethane=1:40, R_{f}=0.22, as the eluent). After purification, a yellow solid compound 20 was obtained with a yield of 92%.

¹H NMR (400 MHz, CDCl₃) δ 7.69 (s, 1H), 7.42 (d, J=9.2 Hz, 2H), 7.35 (d, J=8.8 Hz, 2H), 7.27 (d, J=8.8 Hz, 2H), 7.03 (d, J=9.2 Hz, 2H), 6.14 (s, 1H), 4.97 (septet, J=6.2 Hz, 2H), 4.76 (s, 2H), 4.04-3.77 (m, 4H), 3.35-3.27 (m, 2H), 2.52 (s, 3H), 1.90-1.84 (m, 3H), 1.55-1.46 (m, 3H), 1.24 (d, J=6.2 Hz, 3H), 1.05 (d, J=6.2 Hz, 3H).

¹³C NMR (600 MHz, CDCl₃) δ 165.5, 165.3, 164.7, 159.7, 156.1, 152.5, 138.5, 134.5, 133.2, 132.1, 129.5, 128.7, 126.6, 117.6, 115.4, 108.7, 68.1, 67.5, 66.6, 54.8, 42.3, 39.4, 34.4, 33.7, 22.7, 21.9, 21.6.

LRMS (ESI) m/z: 610.1 [M+H]⁺.

HRMS (ESI) m/z calcd for C₃₁H₃₂ClN₃O₆S [M+H]⁺=610.1779. found: 610.1786.

### Synthesis of isopropyl (Z)-5-(4-chlorophenyl)-2-(4-fluorobenzylidene)-7-methyl-3-oxo-2,3-dihydro-5H-thiazolo[3,2-a]pyrimidine-6-carboxylate (22)

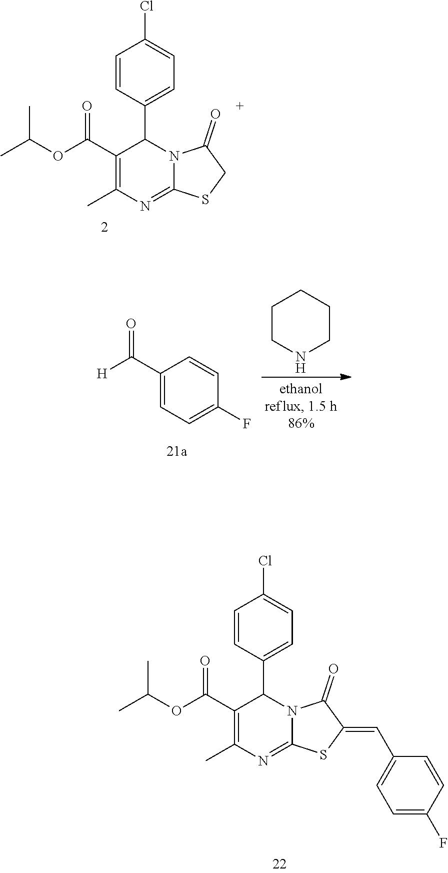 [SMILES: CC1=C(C(=O)OC(C)C)C(c2ccc(Cl)cc2)N2C(=O)CSC2=N1 ; O=Cc1ccc(F)cc1 ; C1CCNCC1 ; CC1=C(C(=O)OC(C)C)C(c2ccc(Cl)cc2)n2c(s/c(=C\c3ccc(F)cc3)c2=O)=N1]

The experimental procedure was similar to that of compound 1. Compound 21a and piperidine were used for condensation reaction. The crude product was subjected to column chromatography (methanol:dichloromethane=1:50, R_{f}=0.2, as the eluent). After purification, a yellow solid compound 22 was obtained with a yield of 86%.

¹H NMR (300 MHz, CDCl₃) δ 7.70 (s, 1H), 7.46 (dd, J=7.8, 5.4 Hz, 2H), 7.36-7.25 (m, 4H), 7.15 (t, J=8.4 Hz, 2H), 6.14 (s, 1H), 5.00-4.92 (m, 1H), 2.51 (s, 3H), 1.23 (d, J=6.9 Hz, 3H), 1.04 (d, J=6.3 Hz, 3H).

LRMS (ESI) m/z: 471.1 [M+H]⁺.

HRMS (ESI) m/z calcd for C₂₄H₂OClFN₂O₃S [M+H]⁺=471.0945. found: 471.0939.

### Synthesis of isopropyl (Z)-5-(4-chlorophenyl)-2-(4-methoxybenzylidene)-7-methyl-3-oxo-2,3-dihydro-5H-thiazolo[3,2-a]pyrimidine-6-carboxylate (23)

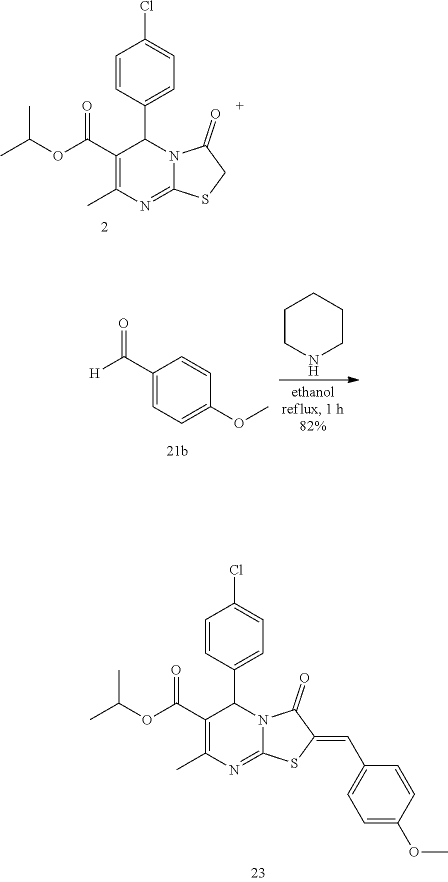

The experimental procedure was similar to that of compound 1. Compound 21b was used for condensation reaction with piperidine. The crude product was subjected to column chromatography (methanol:dichloromethane=1:40, R_{f}=0.22, as the eluent), and a yellow solid compound 23 was obtained after purification, with a yield of 82%.

¹H NMR (300 MHz, CDCl₃) δ 7.70 (s, 1H), 7.42 (d, J=8.7 Hz, 2H), 7.35 (d, J=7.8 Hz, 2H), 7.27 (d, J=8.7 Hz, 2H), 6.97 (d, J=8.4 Hz, 2H), 6.14 (s, 1H), 5.00-4.92 (m, 1H), 3.85 (s, 3H), 2.51 (s, 3H), 1.23 (d, J=6.0 Hz, 3H), 1.04 (d, J=6.3 Hz, 3H).

LRMS (ESI) m/z: 483.1 [M+H]⁺.

HRMS (ESI) m/z calcd for C₂₅H₂₃ClN₂O₄S [M+H]⁺=483.1145. found: 483.1143.

### Synthesis of isopropyl (Z)-2-(benzo[d][1,3]dioxol-5-ylmethylene)-5-(4-chlorophenyl)-7-methyl-3-oxo-2,3-dihydro-5H-thiazolo[3,2-a]pyrimidine-6-carboxylate (24)

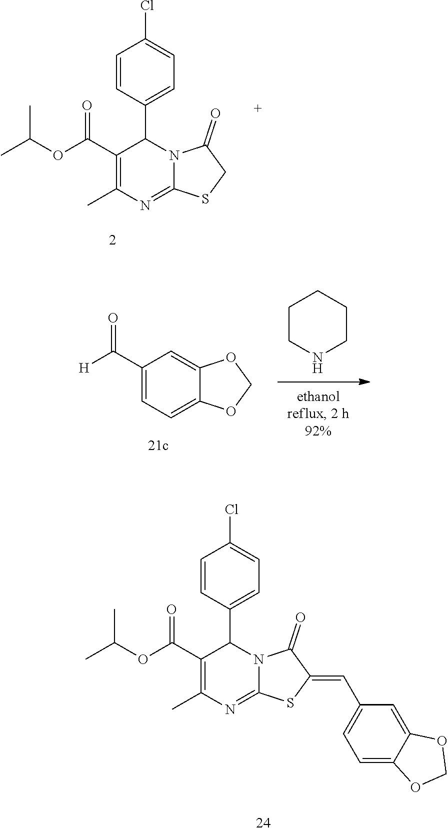 [SMILES: CC1=C(C(=O)OC(C)C)C(c2ccc(Cl)cc2)N2C(=O)CSC2=N1 ; O=Cc1ccc2c(c1)OCO2 ; C1CCNCC1 ; CC1=C(C(=O)OC(C)C)C(c2ccc(Cl)cc2)n2c(s/c(=C\c3ccc4c(c3)OCO4)c2=O)=N1]

The experimental procedure was similar to that of compound 1. Compound 21c is used for condensation reaction with piperidine. The crude product was subjected to column chromatography (methanol:dichloromethane=1:40, R_{f}=0.22, as the eluent). A yellow solid compound 24 was obtained after purification, with a yield of 92%.

¹H NMR (400 MHz, CDCl 3) δ 7.64 (s, 1H), 7.35 (d, J=8.8 Hz, 2H), 7.27 (d, J=8.8 Hz, 2H), 7.01 (dd, J=8.2, 1.8 Hz, 1H), 6.93 (d, J=1.8 Hz, 1H), 6.89 (d, J=8.2 Hz, 1H), 6.14 (s, 1H), 6.05 (s, 2H), 4.97 (septet, J=6.2 Hz, 1H), 2.51 (s, 3H), 1.23 (d, J=6.2 Hz, 4H), 1.05 (d, J=6.2 Hz, 3H).

LRMS (ESI) m/z: 497.1 [M+H]⁺.

HRMS (ESI) m/z calcd for C₂₅H₂₁ClN₂O₅S [M+H]⁺=497.0938. found: 497.0941.

### Synthesis of isopropyl (Z)-5-(4-chlorophenyl)-2-(4-(2-methoxy-2-oxoethoxy)benzylidene)-7-methyl-3-oxo-2,3-dihydro-5H-thiazolo[3,2-a]pyrimidine-6-carboxylate (25)

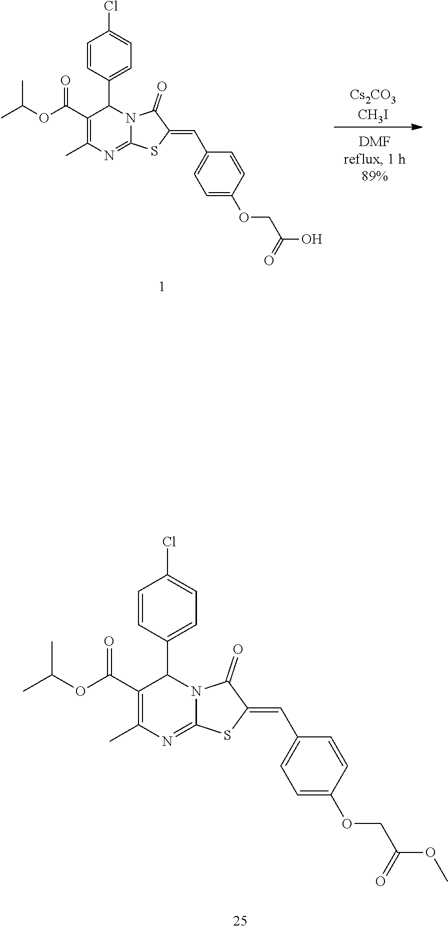 [SMILES: CC1=C(C(=O)OC(C)C)C(c2ccc(Cl)cc2)n2c(s/c(=C\c3ccc(OCC(=O)O)cc3)c2=O)=N1 ; COC(=O)COc1ccc(/C=c2\sc3n(c2=O)C(c2ccc(Cl)cc2)C(C(=O)OC(C)C)=C(C)N=3)cc1]

Compound 1 (159 mg, 0.30 mmol) was dissolved in 5 ml of dimethylformamide, caesium carbonate (197 mg, 0.6 mmol) was added at room temperature, and heated to reflux. Add iodomethane and react for 1 hour. Add water for extraction. The organic layer was dehydrated with anhydrous magnesium sulfate, filtered, and concentrated to obtain a crude product. The crude product was subjected to column chromatography (methanol:dichloromethane=1:30, R_{f}=0.3, as the eluent), and after purification, a yellow solid compound 25 (145 mg, yield 89%) was obtained.

¹H NMR (400 MHz, CDCl₃) δ 7.69 (s, 1H), 7.43 (d, J=9.2 Hz, 2H), 7.35 (d, J=8.8 Hz, 2H), 7.27 (d, J=8.8 Hz, 2H), 6.98 (d, J=9.2 Hz, 2H), 6.14 (s, 1H), 4.97 (septet, J=6.2 Hz, 1H), 4.69 (s, 2H), 3.82 (s, 3H), 2.52 (s, 3H), 1.24 (d, J=6.2 Hz, 3H), 1.05 (d, J=6.2 Hz, 3H).

¹³C NMR (600 MHz, CDCl₃) δ 168.6, 165.3, 164.7, 159.4, 156.1, 152.5, 138.5, 134.5, 133.2, 132.0, 129.6, 128.7, 126.8, 117.8, 115.4, 108.7, 68.1, 65.1, 54.9, 52.4, 22.7, 21.9, 21.6.

LRMS (ESI) m/z: 541.1 [M+H]⁺.

HRMS (ESI) m/z calcd for C₂₇H₂₅ClN₂O₆S [M+H]⁻=541.1145. found: 541.1134.

### Synthesis of 1-methylethyl 5-(4-chlorophenyl)-2-[[4-(2-ethoxy-2-oxoethoxy)phenyl]methylene]-2,3-dihydro-7-methyl-3-oxo-5H-thiazolo[3,2-a]pyrimidine-6-carboxylate (26)

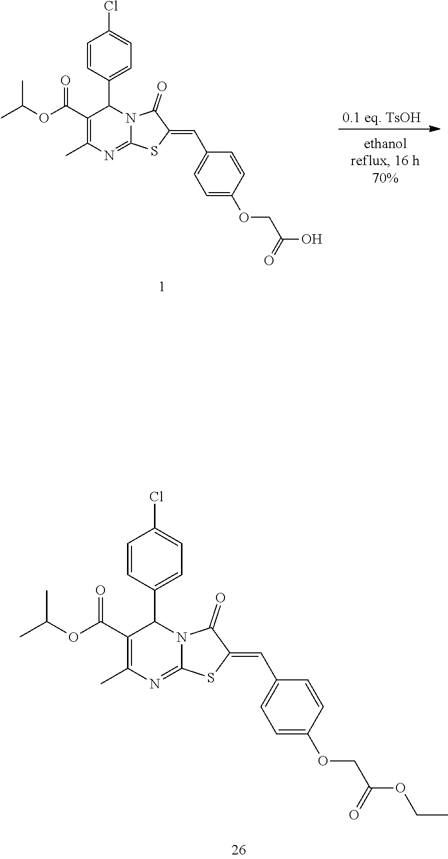 [SMILES: CC1=C(C(=O)OC(C)C)C(c2ccc(Cl)cc2)n2c(s/c(=C\c3ccc(OCC(=O)O)cc3)c2=O)=N1 ; CCOC(=O)COc1ccc(/C=c2\sc3n(c2=O)C(c2ccc(Cl)cc2)C(C(=O)OC(C)C)=C(C)N=3)cc1]

Compound 1 (66 mg, 0.12 mmol) was dissolved in 10 ml of ethanol, added p-toluenesulfonic acid (2.1 mg, 0.01 mmol), heated to reflux, reacted for 16 hours, cycloconcentrated and drained the ethanol. Add water and methylene chloride, extract twice with methylene chloride, the organic layer was dehydrated with anhydrous magnesium sulfate, filtered, and concentrated to obtain a crude product. The crude product was subjected to column chromatography (methanol:methylene chloride=1:50, R_{f}=0.25, as the eluent), and after purification, a yellow solid compound 26 (49 mg, yield 70%) was obtained.

¹H NMR (400 MHz, CDCl₃) δ 7.69 (s, 1H), 7.43 (d, J=9.2 Hz, 2H), 7.35 (d, J=8.8 Hz, 2H), 7.27 (d, J=8.8 Hz, 2H), 6.98 (d, J=9.2 Hz, 2H), 6.14 (s, 1H), 4.97 (septet, J=6.2 Hz, 1H), 4.69 (s, 2H), 3.82 (s, 3H), 2.52 (s, 3H), 1.24 (d, J=6.2 Hz, 3H), 1.05 (d, J=6.2 Hz, 3H).

¹³C NMR (600 MHz, CDCl₃) δ 168.2, 165.3, 164.7, 159.5, 156.1, 152.6, 138.5, 134.5, 133.2, 133.2, 132.0, 129.6, 128.7, 126.7, 117.7, 115.4, 115.4, 108.7, 68.1, 65.2, 61.6, 54.9, 22.7, 22.0, 21.6, 14.1.

LRMS (ESI) m/z: 555.1 [M+H]⁺.

HRMS (ESI) m/z calcd for C₂₈H₂₇ClN₂O₆S [M+H]⁺=555.1357. found: 555.1347.

### Synthesis of isopropyl 4-(4-fluorophenyl)-6-methyl-2-thioxo-1,2,3,4-tetrahydropyrimidine-5-carboxylate (28)

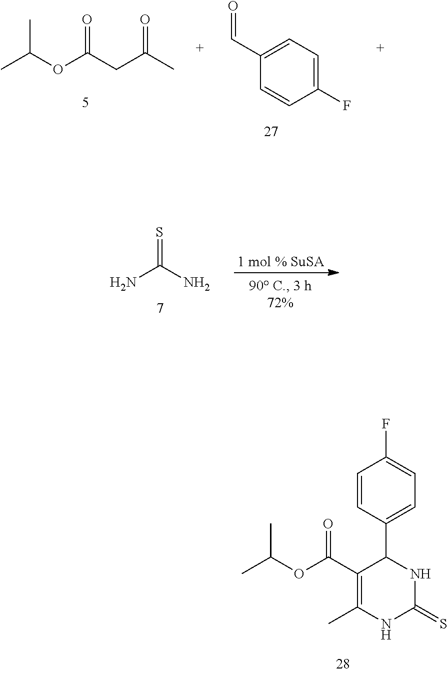

The experimental procedure was similar to that of compound 8. The Biginelli reaction was performed with compound 27 (4-fluorobenzaldehyde). The crude product was subjected to column chromatography (methanol:dichloromethane=1:100, R_{f}=0.3, as the eluent). After purification, a white solid compound 28 was obtained with a yield of 72%.

¹H NMR (400 MHz, CDCl₃) δ 7.72 (s, 1H), 7.30-7.24 (m, 4H), 7.18 (s, 1H), 7.01 (dd, J=8.8, 8.8 Hz, 2H), 5.38 (s, 1H), 4.96 (septet, J=6.2 Hz, 1H), 2.36 (s, 3H), 1.21 (d, J=6.3 Hz, 3H), 1.04 (d, J=6.2 Hz, 3H).

LRMS (ESI) m/z: 309.1 [M+H]⁺.

### Synthesis of isopropyl 5-(4-fluorophenyl)-7-methyl-3-oxo-2,3-dihydro-5H-thiazolo[3,2-a]pyrimidine-6-carboxylate (29)

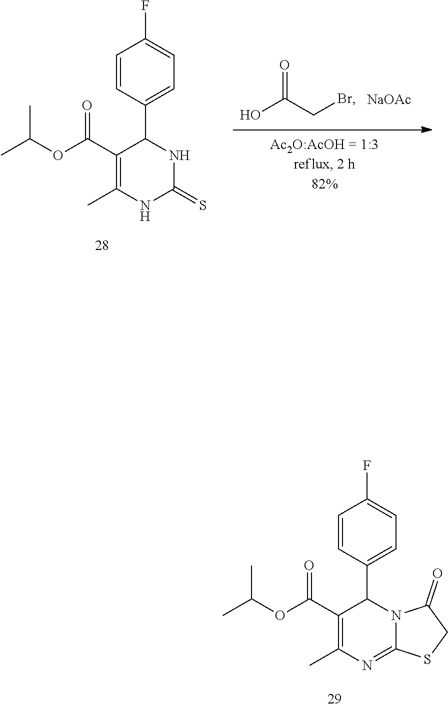 [SMILES: CC1=C(C(=O)OC(C)C)C(c2ccc(F)cc2)NC(=S)N1 ; O=C(O)CBr ; CC1=C(C(=O)OC(C)C)C(c2ccc(F)cc2)N2C(=O)CSC2=N1 ; "NaOAc"]

The experimental procedure was similar to that of compound 2. The crude product was purified by column chromatography (ethyl acetate:n-hexane=1:5, R_{f}=0.2, as the eluent), and reddish brown solid compound 29 was obtained.

¹H NMR (400 MHz, CDCl₃) δ 7.35 (dd, J=8.8, 5.2 Hz, 2H), 6.99 (dd, J=8.8, 8.8 Hz, 2H), 6.02 (s, 1H), 4.94 (septet, J=6.2 Hz, 1H), 3.86 (d, J=17.6 Hz, 1H), 3.74 (d, J=17.6 Hz, 1H), 2.48 (s, 3H), 1.21 (d, J=6.2 Hz, 3H), 1.01 (d, J=6.2 Hz, 3H).

¹³C NMR (600 MHz, CDCl₃) δ 170.3, 164.7, 163.5, 161.9, 159.6, 152.0, 135.9, 130.0, 115.5, 115.4, 108.2, 68.0, 54.9, 32.4, 22.5, 21.9, 21.5.

LRMS (ESI) m/z: 349.1 [M+H]⁺.

### Synthesis of isopropyl (Z)-5-(4-fluorophenyl)-2-(4-hydroxybenzylidene)-7-methyl-3-oxo-2,3-dihydro-5H-thiazolo[3,2-a]pyrimidine-6-carboxylate (31)

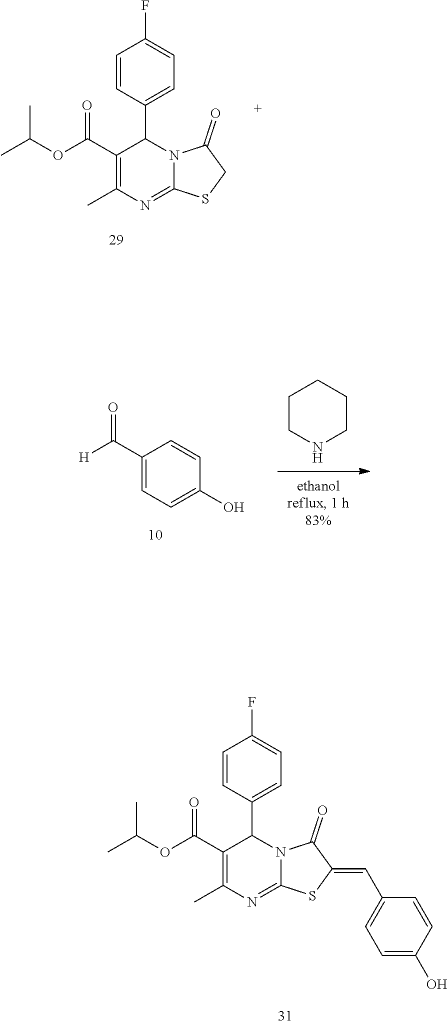 [SMILES: CC1=C(C(=O)OC(C)C)C(c2ccc(F)cc2)N2C(=O)CSC2=N1 ; O=Cc1ccc(O)cc1 ; C1CCNCC1 ; CC1=C(C(=O)OC(C)C)C(c2ccc(F)cc2)n2c(s/c(=C\c3ccc(O)cc3)c2=O)=N1]

The experimental procedure was similar to that of compound 1. Compound 10 was used for condensation reaction with piperidine. The crude product was subjected to column chromatography (methanol:dichloromethane=1:40, R_{f}=0.2, as the eluent). A brown solid compound 31 was obtained after purification, with a yield of 83%.

¹H NMR (400 MHz, CDCl₃) δ 7.70 (s, 1H), 7.71-7.36 (m, 4H), 7.01-6.91 (m, 4H), 6.16 (s, 1H), 4.97 (septet, J=6.4 Hz, 1H), 2.53 (s, 3H), 1.23 (d, J=6.4 Hz, 3H), 1.03 (d, J=6.4 Hz, 3H).

LRMS (ESI) m/z: 453.1 [M+H]⁺.

### Synthesis of (4-(((2Z)-5-(4-fluorophenyl)-7-methyl-3-oxo-6-((propan-2-yloxy)carbonyl)-5H-[1,3]thiazolo[3,2-a]pyrimidin-2(3H)-ylidene)methyl)phenoxy)acetic acid (32)

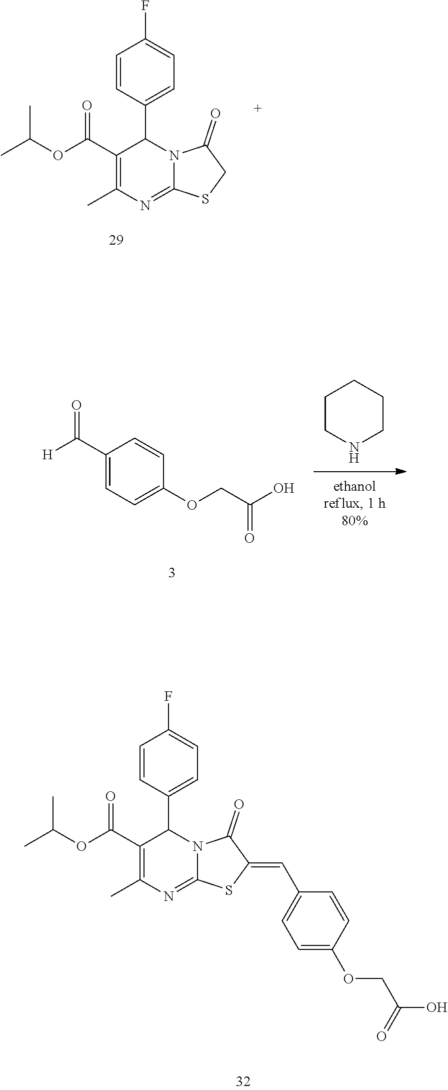 [SMILES: CC1=C(C(=O)OC(C)C)C(c2ccc(F)cc2)N2C(=O)CSC2=N1 ; O=Cc1ccc(OCC(=O)O)cc1 ; C1CCNCC1 ; CC1=C(C(=O)OC(C)C)C(c2ccc(F)cc2)n2c(s/c(=C\c3ccc(OCC(=O)O)cc3)c2=O)=N1]

The experimental procedure was similar to that of compound 1. The crude product was subjected to column chromatography (methanol:ammonium water:dichloromethane=1:0.1:40, R_{f}=0.15, as the eluent). After purification, compound 32 was obtained as a yellow solid with a yield of 80%.

¹H NMR (400 MHz, CDCl 3) δ 7.70 (s, 1H), 7.44 (d, J=9.2 Hz, 2H), 7.39 (dd, J=8.8, 5.2 Hz, 2H), 7.04-6.95 (m, 4H), 6.16 (s, 1H), 4.96 (septet, J=6.2 Hz, 1H), 4.73 (s, 2H), 2.52 (s, 3H), 1.23 (d, J=6.2 Hz, 3H), 1.03 (d, J=6.2 Hz, 3H).

LRMS (ESI) m/z: 511.1 [M+H]⁺.

HRMS (ESI) m/z calcd for C₂₆H₂₃FN₂O₆S [M+H]⁺=511.1339. found: 511.1325.

### Synthesis of 2-chloro-N-methylacetamide (34)

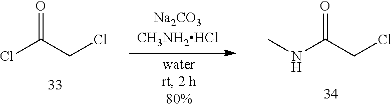

Dissolve methylamine hydrochloride (0.9 g, 0.015 mol) and sodium carbonate (1.68 g, 0.015 mol) in water under ice bath, and slowly drop in compound 33 (2-chloroacetyl chloride) (0.45 g, 0.03 mol). After reacting for 2 hours, compound 34 was obtained without purification after extraction, with a yield of 80%.

¹H NMR (300 MHz, CDCl₃) δ 6.60 (brs, 1H), 4.05 (s, 2H), 2.88 (s, 3H).

### Synthesis of isopropyl (Z)-5-(4-fluorophenyl)-7-methyl-2-(4-(2-(methylamino)-2-oxoethoxy)benzylidene)-3-oxo-2,3-dihydro-5H-thiazolo[3,2-a]pyrimidine-6-carboxylate (35)

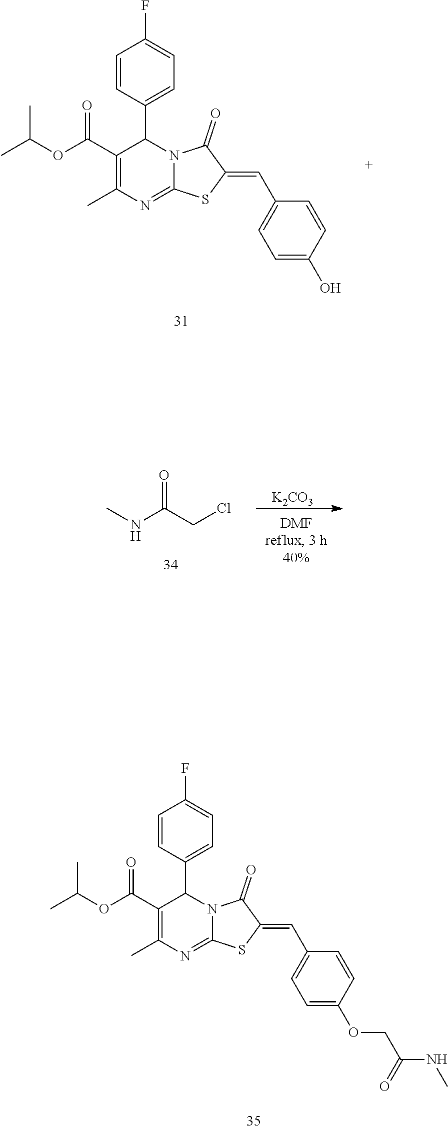

The experimental procedure was similar to compound 3. Compound 31 and potassium carbonate are dissolved in dimethylformamide (DMF). The crude product was subjected to column chromatography using gradient elution (gradient from methanol:ammonia:dichloromethane=1:0.1:40 to methanol:ammonia:dichloromethane=1:0.1:30, R_{f}=0.2, as the eluent), and after purification, a yellow solid compound 35 was obtained, with a yield of 40%.

¹H NMR (300 MHz, DMSO-d₆) δ 8.80 (brs, 1H), 7.75 (s, 1H), 7.58 (d, J=8.7 Hz, 2H), 7.38-7.33 (m, 2H), 7.21-7.10 (m, 4H), 6.02 (s, 1H), 4.85 (septet, J=6.6 Hz, 1H), 4.56 (s, 2H), 2.65 (d, J=4.5 Hz, 3H), 2.39 (s, 3H), 1.16 (d, J=6.6 Hz, 3H), 0.98 (d, J=6.6 Hz, 3H).

LRMS (ESI) m/z: 524.1 [M+H]⁺.

HRMS (ESI) m/z calcd for C₂₇H₂₆FN₃O₅S [M+H]⁺=524.1655. found: 524.1643.

### Synthesis of isopropyl (Z)-5-(4-fluorophenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (36)

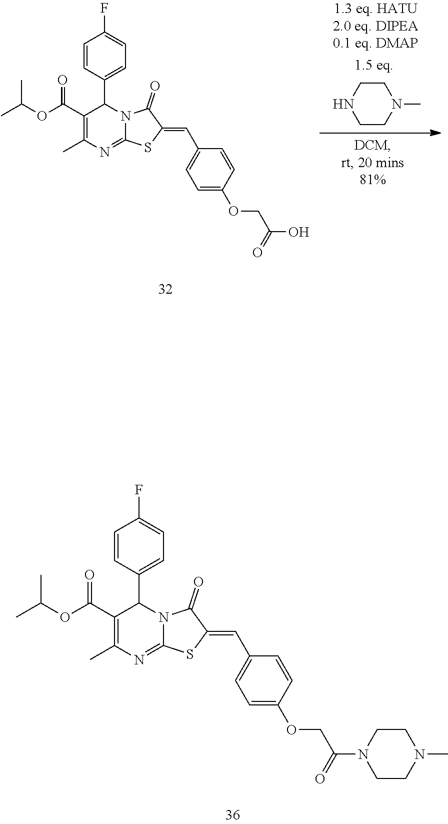

The experimental procedure was similar to that of compound 12. HATU was used to activate the acid group, and the amine compound was 1-methylpiperazine. The crude product was analyzed by column chromatography (methanol:dichloromethane=1:40, R_{f}=0.3, as the eluent). After purification, a yellow solid compound 36 was obtained with a yield of 81%.

¹H NMR (400 MHz, CDCl₃) δ 7.69 (s, 1H), 7.45-7.35 (m, 4H), 7.05-6.94 (m, 4H), 6.16 (s, 1H), 4.96 (septet, J=6.4 Hz, 1H), 4.75 (s, 2H), 3.66-3.55 (m, 4H), 2.52 (s, 3H), 2.42-2.37 (m, 4H), 1.23 (d, J=6.4 Hz, 3H), 1.03 (d, J=6.4 Hz, 3H).

LRMS (ESI) m/z: 593.2 [M+H]⁺.

HRMS (ESI) m/z calcd for C₃₁H₃₃FN₄O₅S [M+H]⁺=593.2234. found: 593.2208.

### Synthesis of propan-2-yl 4-(4-bromophenyl)-6-methyl-2-sulfanylidene-3,4-dihydro-1H-pyrimidine-5-carboxylate (38)

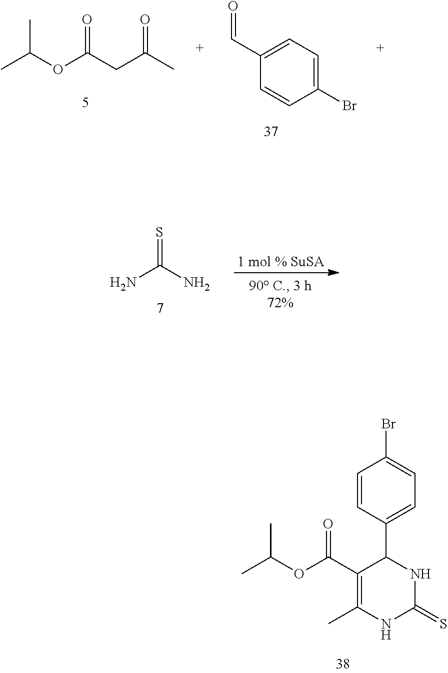

The experimental procedure was similar to that of compound 8. The Biginelli reaction was performed with compound 37 (4-bromobenzaldehyde). The crude product was analyzed by column chromatography (ethyl acetate:n-hexane=1:6, R_{f}=0.23, as the eluent), and after purification, a white solid compound 38 was obtained with a yield of 72%.

¹H NMR (400 MHz, CDCl₃) δ 7.56 (s, 1H), 7.46 (d, J=8.4 Hz, 2H), 7.17 (d, J=8.4 Hz, 2H), 7.01 (s, 1H), 5.36 (s, 1H), 4.97 (septet, J=6.2 Hz, 1H), 2.36 (s, 3H), 1.22 (d, J=6.2 Hz, 3H), 1.07 (d, J=6.2 Hz, 3H).

¹³C NMR (400 MHz, DMSO-d₆) δ 174.2, 164.4, 145.2, 142.9, 131.4, 128.7, 120.8, 100.5, 66.9, 53.6, 21.7, 21.4, 17.1.

LRMS (ESI) m/z: 369.2 [M+H]⁺.

### Synthesis of 1-Methylethyl 5-(4-bromophenyl)-2,3-dihydro-7-methyl-3-oxo-5H-thiazolo[3,2-a]pyrimidine-6-carboxylate (39)

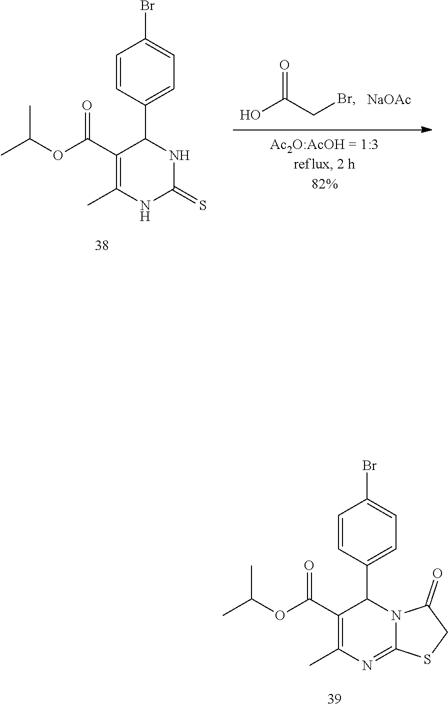 [SMILES: CC1=C(C(=O)OC(C)C)C(c2ccc(Br)cc2)NC(=S)N1 ; O=C(O)CBr ; CC1=C(C(=O)OC(C)C)C(c2ccc(Br)cc2)N2C(=O)CSC2=N1 ; "NaOAc"]

The experimental procedure was similar to that of compound 2. The crude product was purified by column chromatography (ethyl acetate:n-hexane=1:6, R_{f}=0.24, as the eluent), and reddish-brown solid compound 39 was obtained.

¹H NMR (400 MHz, CDCl₃) δ 7.44 (d, J=8.8 Hz, 2H), 7.24 (d, J=8.8 Hz, 2H), 6.00 (s, 1H), 4.94 (septet, J=6.4 Hz, 1H), 3.86 (d, J=17.6 Hz, 1H), 3.74 (d, J=17.6 Hz, 1H), 2.47 (s, 3H), 1.21 (d, J=6.4 Hz, 4H), 1.03 (d, J=6.4 Hz, 3H). ¹³C NMR (400 MHz, CDCl₃) δ 170.1, 164.3, 159.6, 159.5, 152.1, 138.8, 131.7, 131.5, 131.4, 129.8, 129.6, 128.3, 122.5, 107.5, 67.8, 57.8, 54.7, 32.1, 22.4, 22.0, 21.7, 21.6, 21.5, 21.5, 21.3, 18.1.

LRMS (ESI) m/z: 409.1 [M+H]⁺.

### Synthesis of (4-(((2Z)-5-(4-bromophenyl)-7-methyl-3-oxo-6-((propan-2-yloxy)carbonyl)-5H-[1,3]thiazolo[3,2-a]pyrimidin-2(3H)-ylidene)methyl)phenoxy)acetic acid (40)

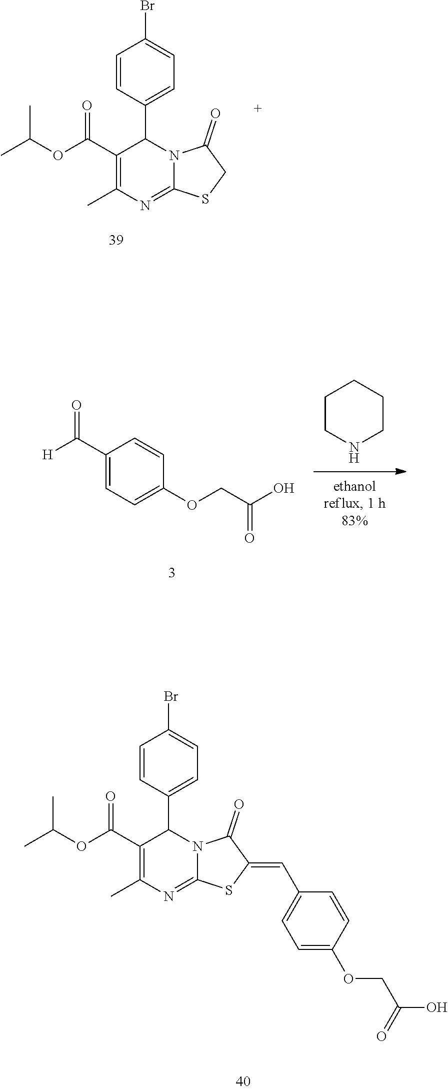 [SMILES: CC1=C(C(=O)OC(C)C)C(c2ccc(Br)cc2)N2C(=O)CSC2=N1 ; O=Cc1ccc(OCC(=O)O)cc1 ; C1CCNCC1 ; CC1=C(C(=O)OC(C)C)C(c2ccc(Br)cc2)n2c(s/c(=C\c3ccc(OCC(=O)O)cc3)c2=O)=N1]

The experimental procedure was similar to that of compound 1 Compound 3 and piperidine were used for a condensation reaction. The crude product was subjected to column chromatography using a gradient elution method (gradient from methanol:ammonia:dichloromethane=1:0.1:40 to methanol:ammonia:methylene chloride=1:0.1:30, R_{f}=0.18, as the eluent), and after purification, a yellow solid compound 40 was obtained with a yield of 83%.

¹H NMR (400 MHz, DMSO-d₆) δ 7.71 (s, 1H), 7.55 (d, J=8.8 Hz, 2H), 7.50 (d, J=8.8 Hz, 2H), 7.26 (d, J=8.8 Hz, 2H), 6.96 (d, J=8.8 Hz, 2H), 5.99 (s, 1H), 4.85 (p, J=6.4 Hz, 1H), 4.30 (s, 2H), 2.38 (s, 3H), 1.19 (d, J=6.4 Hz, 3H), 1.00 (d, J=6.4 Hz, 3H).

LRMS (ESI) m/z: 571.0 [M+H]⁺.

HRMS (ESI) m/z calcd for C₂₆H₂₃BrN₂O₆S [M+H]⁻=569.0382. found: 569.0377.

### Synthesis of isopropyl (Z)-5-(4-bromophenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (41)

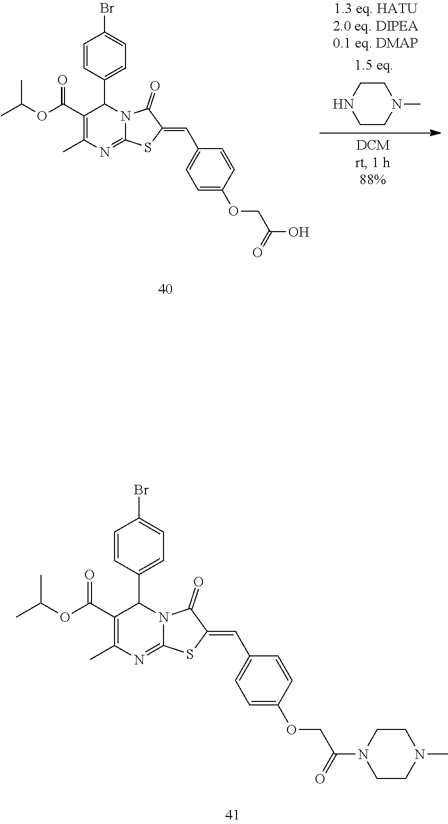

The experimental procedure was similar to compound 12. HATU was used to activate the acid group, and the amine compound was 1-methylpiperazine. The crude product was subjected to column chromatography (methanol:dichloromethane=1:30, R_{f}=0.35, as the eluent). After purification, a yellow solid compound 41 was obtained with a yield of 88%.

¹H NMR (300 MHz, CDCl₃) δ 7.69 (s, 1H), 7.45-7.41 (m, 4H), 7.28-2.26 (m, 2H), 7.03 (d, J=8.1 Hz, 2H), 6.13 (s, 1H), 4.97 (septet, J=6.0 Hz, 1H), 4.75 (s, 2H), 3.72-3.56 (m, 4H), 2.51 (s, 3H), 2.39 (s, 4H), 2.29 (s, 3H), 1.24 (d, J=6.0 Hz, 3H), 1.05 (d, J=6.0 Hz, 3H).

¹³ C NMR (400 MHz, CDCl₃) δ 165.5, 164.8, 164.3, 159.4, 155.8, 152.1, 138.8, 132.9, 131.8, 131.3, 129.5, 126.0, 122.2, 116.9, 115.2, 108.2, 67.8, 66.1, 54.8, 54.5, 54.3, 53.9, 45.4, 44.3, 42.8, 41.5, 38.2, 22.4, 21.6, 21.2, 17.2, 12.2.

LRMS (ESI) m/z: 653.2 [M+H]⁺.

HRMS (ESI) m/z calcd for C₃₁H₃₃BrN₄O₅S [M+H]⁺=653.1433. found: 653.1448.

### Synthesis of isopropyl 4-(4-(1H-imidazol-1-yl)phenyl)-6-methyl-2-thioxo-1,2,3,4-tetrahydropyrimidine-5-carboxylate (43)

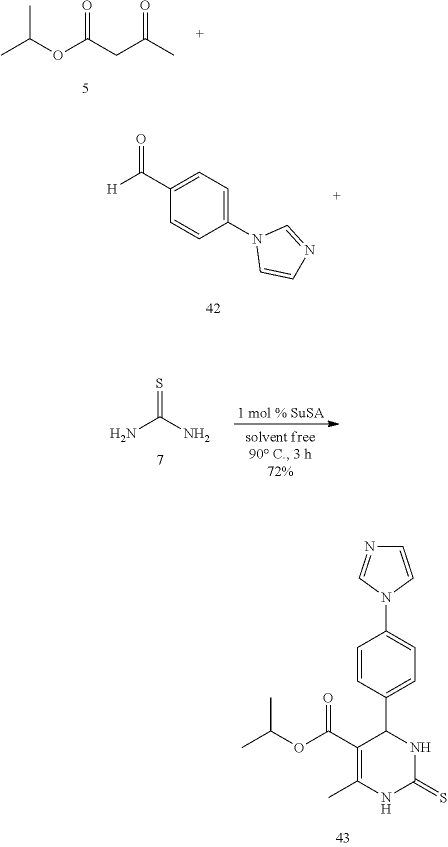

The experimental procedure was similar to that of compound 8. The Biginelli reaction was performed with compound 42 (4-(1H-Imidazol-1-yl)benzaldehyde). The crude product was subjected to column chromatography (ethyl acetate:n-hexane=1:6, R_{f}=0.3, as the eluent). After purification, a white solid compound 43 was obtained with a yield of 72%.

¹H NMR (300 MHz, CDCl₃) δ 8.82 (brs, 1H), 8.37 (brs, 1H), 7.84 (s, 1H), 7.40 (d, J=8.4 Hz, 2H), 7.30 (d, J=8.4 Hz, 2H), 7.22 (s, 1H), 7.18 (s, 1H), 5.44 (s, 1H), 4.97 (septet, J=6.2 Hz, 1H), 2.39 (s, 3H), 1.21 (d, J=6.2 Hz, 3H), 1.07 (d, J=6.2 Hz, 3H).

¹³C NMR (400 MHz, DMSO-d₆) δ 174.4, 164.7, 145.2, 142.3, 136.3, 135.6, 129.9, 128.0, 120.6, 118.1, 100.9, 67.1, 53.7, 21.8, 21.5, 17.3.

LRMS (ESI) m/z: 356.4 [M+H]⁺.

### Synthesis of isopropyl 5-(4-(1H-imidazol-1-yl)phenyl)-7-methyl-3-oxo-2,3-dihydro-5H-thiazolo[3,2-a]pyrimidine-6-carboxylate (44)

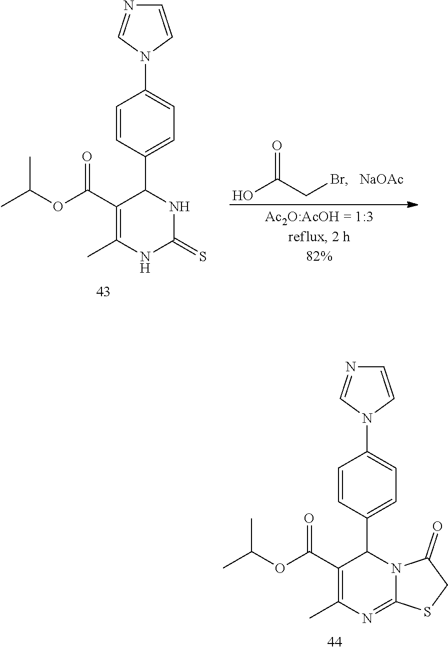

The experimental procedure was similar to compound 2. The crude product was purified using column chromatography (ethyl acetate:n-hexane=1:6, R_{f}=0.2, as the eluent) to obtain compound 44 as a brown oil with a yield of 82%.

¹H NMR (400 MHz, CDCl₃) δ 7.88 (s, 1H), 7.49 (d, J=8.4 Hz, 2H), 7.34 (d, J=8.4 Hz, 2H), 7.24 (s, 1H), 7.20 (s, 1H), 6.07 (s, 1H), 4.96 (septet, J=6.4, 1.0 Hz, 1H), 3.88 (d, J=17.6 Hz, 1H), 3.75 (d, J=17.6 Hz, 1H), 2.49 (s, 3H), 1.22 (d, J=6.4 Hz, 3H), 1.05 (d, J=6.4 Hz, 3H).

LRMS (ESI) m/z: 397.1 [M+H]⁺.

### Synthesis of (Z)-2-(4-((5-(4-(1H-imidazol-1-yl)phenyl)-6-(isopropoxycarbonyl)-7-methyl-3-oxo-5H-thiazolo[3,2-a]pyrimidin-2(3H)-ylidene)methyl)phenoxy)acetic acid (45)

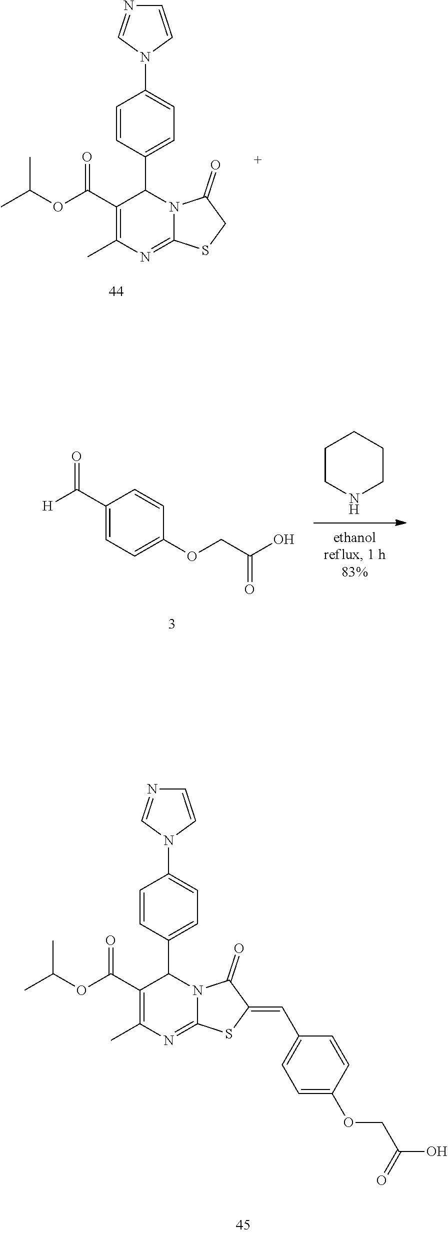 [SMILES: CC1=C(C(=O)OC(C)C)C(c2ccc(-n3ccnc3)cc2)N2C(=O)CSC2=N1 ; O=Cc1ccc(OCC(=O)O)cc1 ; C1CCNCC1 ; CC1=C(C(=O)OC(C)C)C(c2ccc(-n3ccnc3)cc2)n2c(s/c(=C\c3ccc(OCC(=O)O)cc3)c2=O)=N1]

The experimental procedure was similar to that of compound 1. Compound 3 and piperidine were used for a condensation reaction. The crude product was subjected to column chromatography using a gradient elution method (gradient from methanol:ammonia:dichloromethane=1:0.1:30 to methanol:ammonia:methylene chloride=1:0.1:20, R_{f}=0.17, as the eluent), and after purification, a yellow solid compound 45 was obtained with a yield of 83%.

¹H NMR (400 MHz, CDCl₃) δ 7.88 (s, 1H), 7.49 (d, J=8.4 Hz, 2H), 7.34 (d, J=8.4 Hz, 2H), 7.24 (s, 1H), 7.20 (s, 1H), 6.07 (s, 1H), 4.96 (septet, J=6.4, 1.0 Hz, 1H), 3.88 (d, J=17.6 Hz, 1H), 3.75 (d, J=17.6 Hz, 1H), 2.49 (s, 3H), 1.22 (d, J=6.4 Hz, 3H), 1.05 (d, J=6.4 Hz, 3H).

¹³C NMR (400 MHz, DMSO-d₆) δ 170.3, 164.5, 164.3, 160.9, 155.8, 151.6, 139.0, 136.8, 135.6, 133.4, 132.0, 129.9, 129.1, 124.7, 120.6, 118.0, 115.8, 115.5, 108.2, 67.7, 66.9, 54.4, 43.3, 22.5, 22.2, 21.6, 21.3.

LRMS (ESI) m/z: 559.2 [M+H]⁺.

HRMS (ESI) m/z calcd for C₂₉H₂₆N₄O₆S [M+H]⁻=557.1495. found: 557.1482.

### Synthesis of isopropyl (Z)-5-(4-(1H-imidazol-1-yl)phenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (46)

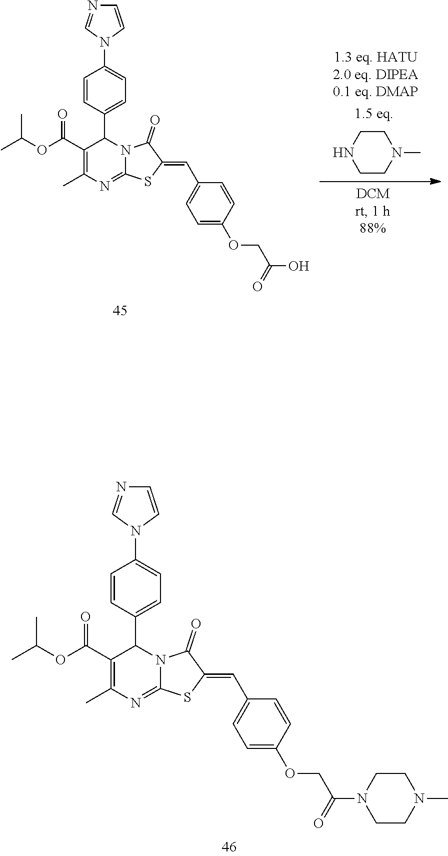

The experimental procedure was similar to compound 12. HATU was used to activate the acid group, and the amine compound was 1-methylpiperazine. The crude product was subjected to column chromatography (methanol:dichloromethane=1:30, R_{f}=0.3, as the eluent). After purification, a yellow solid compound 46 was obtained with a yield of 88%.

¹H NMR (400 MHz, CDCl₃) δ 7.81 (s, 1H), 7.70 (s, 1H), 7.54 (d, J=8.4 Hz, 2H), 7.43 (d, J=8.7 Hz, 2H), 7.33 (d, J=8.4 Hz, 2H), 7.23 (s, 1H), 7.18 (s, 1H), 7.03 (d, J=8.7 Hz, 2H), 6.22 (s, 1H), 4.99 (septet, J=6.3 Hz, 1H), 4.75 (s, 2H), 3.64-3.55 (m, 4H), 2.54 (s, 3H), 2.38 (s, 4H), 2.29 (s, 3H), 1.25 (d, J=6.3 Hz, 3H), 1.08 (d, J=6.3 Hz, 3H).

¹³C NMR (400 MHz, CDCl₃) δ 165.3, 165.0, 164.4, 159.4, 155.8, 152.3, 139.1, 136.9, 135.1, 133.0, 131.8, 130.1, 129.5, 128.7, 127.9, 126.2, 124.9, 121.0, 117.7, 117.1, 115.2, 108.3, 67.9, 66.8, 54.7, 54.6, 54.2, 53.3, 45.6, 44.7, 41.7, 22.5, 21.7, 21.3.

LRMS (ESI) m/z: 641.3 [M+H]⁺.

HRMS (ESI) m/z calcd for C₃₄H₃₆N₆O₅S [M+H]⁺=641.2546. found: 641.2550.

### Synthesis of isopropyl 6-methyl-2-thioxo-4-(p-tolyl)-1,2,3,4-tetrahydropyrimidine-5-carboxylate (48)

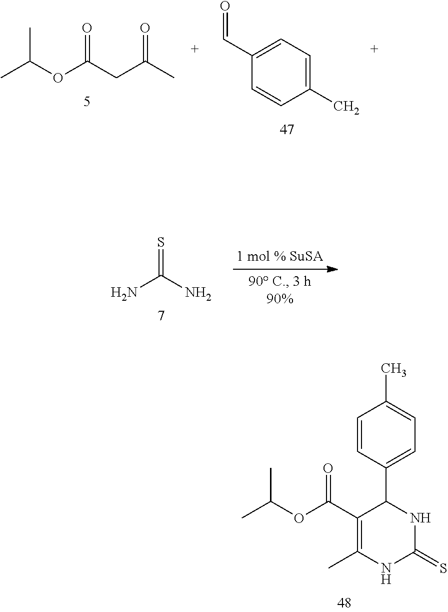

The experimental procedure was similar to that of compound 8. The Biginelli reaction was performed with compound 47 (4-methylbenzaldehyde). The crude product was analyzed by column chromatography (ethyl acetate:n-hexane=1:5, R_{f}=0.3, as the eluent), and after purification, a white solid compound 48 was obtained with a yield of 90%.

¹H NMR (300 MHz, CDCl₃) δ 7.57 (1.05 (d, J=6.4 Hz, 3H). s, 1H), 7.18 (d, J=8.1 Hz, 2H), 7.13 (d, J=8.2 Hz, 2H), 6.98 (s, 1H), 5.36 (s, 1H), 4.95 (septet, J=6.3 Hz, 1H), 2.35 (s, 3H), 2.32 (s, 3H), 1.21 (d, J=6.3 Hz, 3H).

LRMS (ESI) m/z: 305.1 [M+H]⁺.

### Synthesis of isopropyl 7-methyl-3-oxo-5-(p-tolyl)-2,3-dihydro-5H-thiazolo[3,2-a]pyrimidine-6-carboxylate (49)

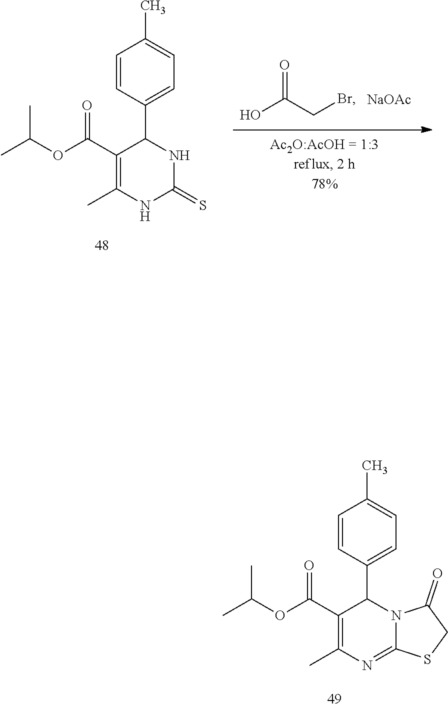 [SMILES: CC1=C(C(=O)OC(C)C)C(c2ccc(C)cc2)NC(=S)N1 ; O=C(O)CBr ; CC1=C(C(=O)OC(C)C)C(c2ccc(C)cc2)N2C(=O)CSC2=N1 ; "NaOAc"]

The experimental procedure was similar to that of compound 2. The crude product was purified using column chromatography (ethyl acetate:n-hexane=1:5, R_{f}=0.21, as the eluent) to obtain red-brown solid compound 49 with a yield of 78%.

¹H NMR (300 MHz, CDCl₃) δ 7.24 (d, J=8.1 Hz, 2H), 7.10 (d, J=8.1 Hz, 2H), 6.00 (s, 1H), 4.94 (septet, J=6.3 Hz, 1H), 3.84 (d, J=17.4 Hz, 1H), 3.71 (d, J=17.5 Hz, 1H), 2.47 (s, 3H), 2.30 (s, 3H), 1.21 (d, J=6.3 Hz, 3H), 1.03 (d, J=6.3 Hz, 3H).

¹³C NMR (400 MHz, CDCl₃) δ 170.3, 164.7, 159.6, 151.5, 138.3, 137.1, 129.0, 127.9, 108.4, 67.8, 55.2, 32.3, 22.4, 21.8, 21.4, 21.0.

LRMS (ESI) m/z: 345.1 [M+H]⁺.

### Synthesis of (Z)-2-(4-((6-(isopropoxycarbonyl)-7-methyl-3-oxo-5-(p-tolyl)-5H-thiazolo[3,2-a]pyrimidin-2(3H)-ylidene)methyl)phenoxy)acetic acid (50)

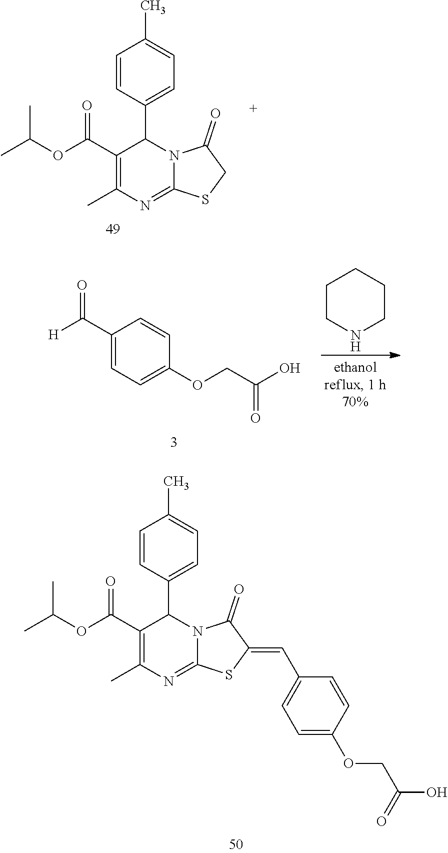 [SMILES: CC1=C(C(=O)OC(C)C)C(c2ccc(C)cc2)N2C(=O)CSC2=N1 ; O=Cc1ccc(OCC(=O)O)cc1 ; C1CCNCC1 ; CC1=C(C(=O)OC(C)C)C(c2ccc(C)cc2)n2c(s/c(=C\c3ccc(OCC(=O)O)cc3)c2=O)=N1]

The experimental steps were similar to compound 1. Piperidine was used for condensation reaction. The crude product was subjected to column chromatography (methanol:dichloromethane=1:50, R_{f}=0.22, as the eluent). After purification, a yellow solid compound 50 was obtained with a yield of 70%.

¹H NMR (300 MHz, CDCl₃) δ 7.70 (s, 1H), 7.44 (d, J=8.7 Hz, 2H), 7.28 (d, J=8.1 Hz, 2H), 7.10 (d, J=8.1 Hz, 2H), 7.00 (d, J=8.7 Hz, 2H), 6.15 (s, 1H), 4.97 (septet, J=6.3 Hz, 1H), 4.73 (s, 2H), 2.39 (s, 3H), 2.17 (s, 3H), 1.24 (d, J=6.3 Hz, 3H), 1.05 (d, J=6.3 Hz, 3H).

¹³C NMR (600 MHz, DMSO-d₆) δ 169.6, 164.4, 164.3, 159.7, 155.8, 150.7, 138.0, 137.5, 133.0, 132.1, 129.1, 127.5, 125.7, 116.7, 115.5, 108.8, 67.6, 64.6, 54.7, 22.2, 21.6, 21.2, 20.7.

LRMS (ESI) m/z: 507.2 [M+H]⁺.

HRMS (ESI) m/z calcd for C₂₇H₂₆N₂O₆S [M+H]⁺=507.1590. found: 507.1593.

### Synthesis of isopropyl (Z)-2-(4-(2-(4-ethylpiperazin-1-yl)-2-oxoethoxy)benzylidene)-7-methyl-3-oxo-5-(p-tolyl)-2,3-dihydro-5H-thiazolo[3,2-a]pyrimidine-6-carboxylate (51)

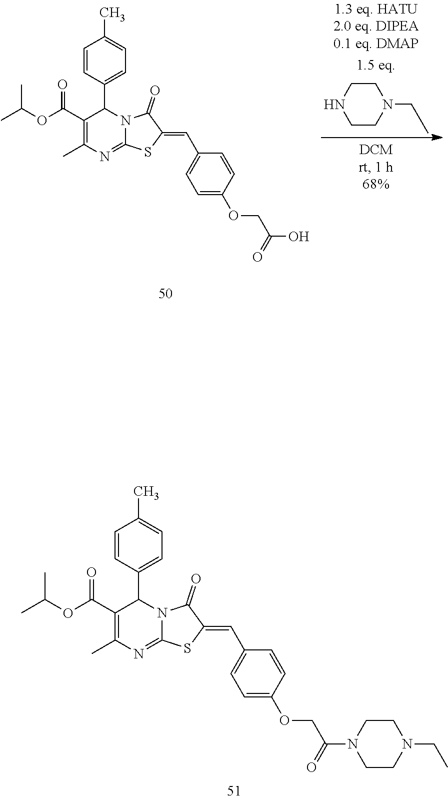

The experimental procedure was similar to that of compound 12. HATU was used to activate the acid group, and the amine compound was 1-ethylpiperazine. The crude product was subjected to column chromatography (methanol:dichloromethane=1:40, R_{f}=0.3, as the eluent), and after purification, a yellow solid compound 51 was obtained with a yield of 68%.

¹H NMR (300 MHz, CDCl₃) δ 7.70 (s, 1H), 7.41 (d, J=8.7 Hz, 2H), 7.29 (d, J=8.1 Hz, 2H), 7.09 (d, J=8.1 Hz, 2H), 7.15 (d, J=8.7 Hz, 2H), 6.14 (s, 1H), 4.96 (septet, J=6.3 Hz, 1H), 4.74 (s, 2H), 3.69-3.62 (m, 4H), 2.51 (s, 3H), 2.49-2.46 (m, 4H), 2.28 (s, 4H), 1.24 (d, J=6.3 Hz, 3H), 1.11 (t, J=6.9 Hz, 3H), 1.05 (d, J=6.3 Hz, 3H).

¹³C NMR (400 MHz, CDCl₃) δ 165.5, 165.4, 165.3, 164.9, 159.4, 156.0, 151.8, 138.3, 137.1, 132.7, 131.9, 129.1, 127.9, 126.7, 126.7, 117.9, 115.3, 109.2, 67.8, 67.2, 55.2, 52.7, 52.1, 52.1, 45.0, 41.9, 22.5, 21.9, 21.4, 21.1, 11.7.

LRMS (ESI) m/z: 603.3 [M+H]⁺.

HRMS (ESI) m/z calcd for C₃₃H₃₈N₄O₅S [M+H]⁺=603.2641. found: 603.2639.

### Synthesis of isopropyl 4-(4-(dimethylamino)phenyl)-6-methyl-2-thioxo-1,2,3,4-tetrahydropyrimidine-5-carboxylate (53)

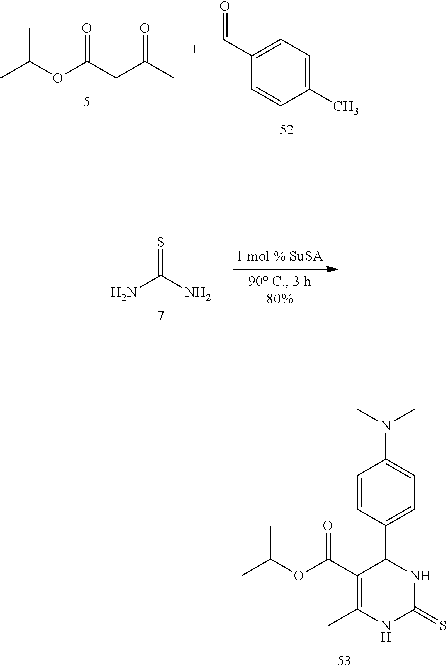

The experimental procedure was similar to that of compound 8. The Biginelli reaction was performed with compound 52 (4-methylbenzaldehyde). The crude product was subjected to column chromatography (methanol:dichloromethane=1:120, R_{f}=0.2, as the eluent) to obtain compound 53, a white solid, with a yield of 80%.

¹H NMR (300 MHz, CDCl₃) δ 7.70 (s, 1H), 7.41 (d, J=8.7 Hz, 2H), 7.29 (d, J=8.1 Hz, 2H), 7.09 (d, J=8.1 Hz, 2H), 7.15 (d, J=8.7 Hz, 2H), 6.14 (s, 1H), 4.96 (septet, J=6.3 Hz, 1H), 4.74 (s, 2H), 3.69-3.62 (m, 4H), 2.51 (s, 3H), 2.49-2.46 (m, 4H), 2.28 (s, 4H), 1.24 (d, J=6.3 Hz, 3H), 1.11 (t, J=6.9 Hz, 3H), 1.05 (d, J=6.3 Hz, 3H).

¹³C NMR (400 MHz, DMSO-d₆) δ 173.9, 164.9, 150.0, 144.1, 131.4, 127.3, 112.1, 101.7, 101.7, 66.8, 53.7, 40.2, 21.8, 21.6, 17.2.

LRMS (ESI) m/z: 334.1 [M+H]⁺.

### Synthesis of isopropyl 5-(4-(dimethylamino)phenyl)-7-methyl-3-oxo-2,3-dihydro-5H-thiazolo[3,2-a]pyrimidine-6-carboxylate (54)

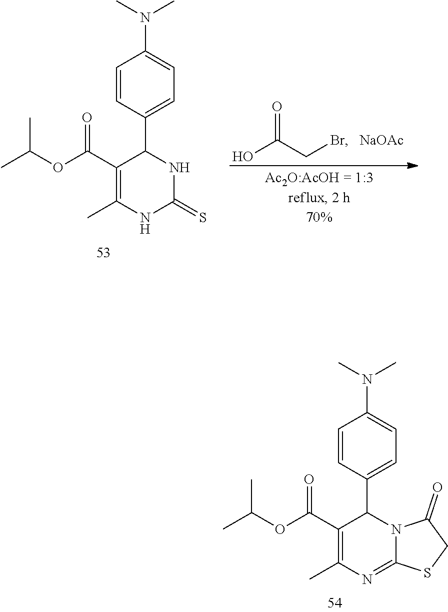

The experimental steps were similar to compound 2. The crude product was purified using column chromatography (ethyl acetate:n-hexane=1:5, R_{f}=0.2, as the eluent) to obtain reddish brown solid compound 54, with a yield of 70%.

¹H NMR (400 MHz, CDCl₃) δ 7.21 (d, J=8.8 Hz, 2H), 6.61 (d, J=8.8 Hz, 2H), 5.96 (s, 1H), 4.93 (septet, J=6.4 Hz, 1H), 3.83 (d, J=17.4 Hz, 1H), 3.71 (d, J=17.4 Hz, 1H), 2.92 (s, 6H), 2.47 (s, 3H), 1.22 (d, J=6.4 Hz, 3H), 1.04 (d, J=6.4 Hz, 3H).

¹³C NMR (400 MHz, CDCl₃) δ 170.3, 164.8, 164.7, 159.3, 150.9, 150.1, 150.1, 128.7, 127.6, 111.5, 111.4, 108.4, 108.4, 67.4, 67.4, 54.8, 54.8, 40.0, 40.0, 32.1, 22.2, 21.6, 21.3.

LRMS (ESI) m/z: 374.1 [M+H]⁺.

### Synthesis of (Z)-2-(4-((5-(4-(dimethylamino)phenyl)-6-(isopropoxycarbonyl)-7-methyl-3-oxo-5H-thiazolo[3,2-a]pyrimidin-2(3H)-ylidene)methyl)phenoxy)acetic acid (55)

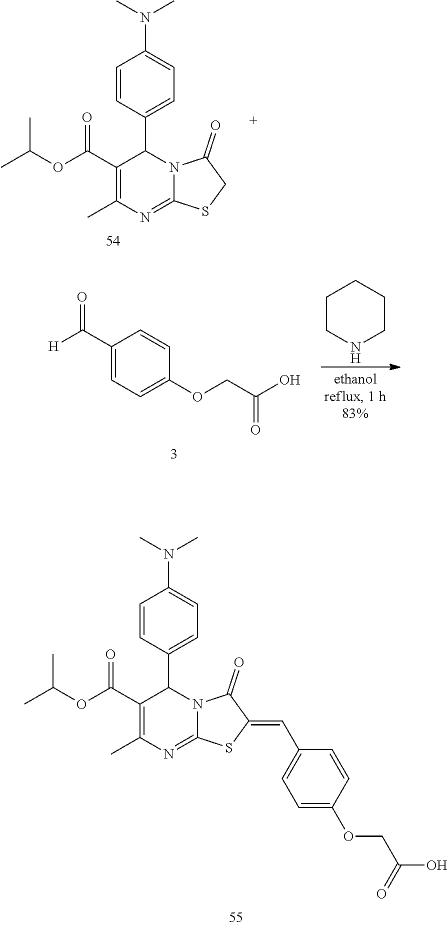 [SMILES: CC1=C(C(=O)OC(C)C)C(c2ccc(N(C)C)cc2)N2C(=O)CSC2=N1 ; O=Cc1ccc(OCC(=O)O)cc1 ; C1CCNCC1 ; CC1=C(C(=O)OC(C)C)C(c2ccc(N(C)C)cc2)n2c(s/c(=C\c3ccc(OCC(=O)O)cc3)c2=O)=N1]

The experimental steps were similar to compound 1. Piperidine was used for condensation reaction. The crude product was subjected to column chromatography (methanol:dichloromethane=1:60, R_{f}=0.2, as the eluent). After purification, a yellow solid compound 55 was obtained with a yield of 83%.

¹H NMR (400 MHz, CDCl₃) δ 7.68 (s, 1H), 7.42 (d, J=9.2 Hz, 2H), 7.24 (d, J=8.4 Hz, 2H), 7.00 (d, J=9.2 Hz, 2H), 6.64 (d, J=8.4 Hz, 2H), 6.09 (s, 1H), 4.96 (septet, J=6.4 Hz, 1H), 4.73 (s, 2H), 2.90 (s, 6H), 2.51 (s, 3H), 1.24 (d, J=6.4 Hz, 3H), 1.06 (d, J=6.4 Hz, 3H).

¹³C NMR (600 MHz, CDCl₃) δ 171.1, 165.4, 165.0, 159.4, 156.7, 150.8, 150.2, 133.2, 132.0, 129.1, 128.6, 126.8, 117.8, 115.4, 112.7, 109.7, 77.2, 77.0, 76.8, 68.0, 65.0, 55.1, 50.8, 40.7, 22.0, 22.0, 21.6, 21.6.

LRMS (ESI) m/z: 536.1 [M+H]⁺.

HRMS (ESI) m/z calcd for C₂₈H₂₉N₃O₆S [M+H]⁻=534.1699. found: 534.1684.

### Synthesis of isopropyl (Z)-5-(4-(dimethylamino)phenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (56)

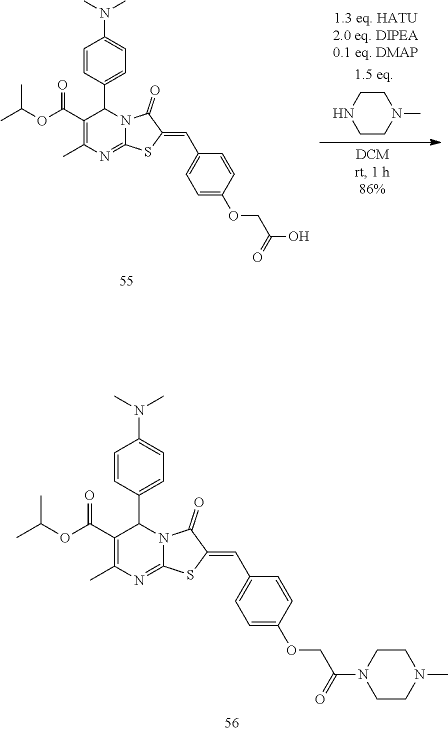

The experimental procedure was similar to that of compound 12. HATU was used to activate the acid group, and the amine compound was 1-methylpiperazine. The crude product was subjected to column chromatography (methanol:dichloromethane=1:50, R_{f}=0.3, as the eluent), and after purification, a yellow solid compound 56 was obtained with a yield of 86%.

¹H NMR (400 MHz, CDCl₃) δ 7.67 (s, 1H), 7.41 (d, J=8.7 Hz, 2H), 7.25 (d, J=8.7 Hz, 2H), 7.01 (d, J=8.7 Hz, 2H), 6.40 (d, J=8.7 Hz, 2H), 6.01 (s, 1H), 4.96 (septet, J=6.0 Hz, 1H), 4.74 (s, 2H), 3.65-3.56 (m, 4H), 2.90 (s, 6H), 2.50 (s, 3H), 2.40-2.38 (m, 4H), 2.29 (s, 3H), 1.24 (d, J=6.0 Hz, 3H), 1.06 (d, J=6.0 Hz, 3H).

¹³C NMR (400 MHz, CDCl₃) δ 165.5, 165.4, 165.1, 159.3, 155.7, 151.2, 150.3, 132.4, 131.8, 128.9, 127.7, 126.7, 118.2, 115.3, 111.8, 109.6, 67.7, 67.2, 55.0, 54.9, 54.4, 45.9, 45.0, 41.9, 40.2, 22.5, 21.9, 21.5.

LRMS (ESI) m/z: 618.3 [M+H]⁺.

HRMS (ESI) m/z calcd for C₃₃H₃₉N₅O₅S [M+H]⁺=618.2750. found: 618.2756.

### Synthesis of isopropyl 4-(4-hydroxyphenyl)-6-methyl-2-thioxo-1,2,3,4-tetrahydropyrimidine-5-carboxylate (58)

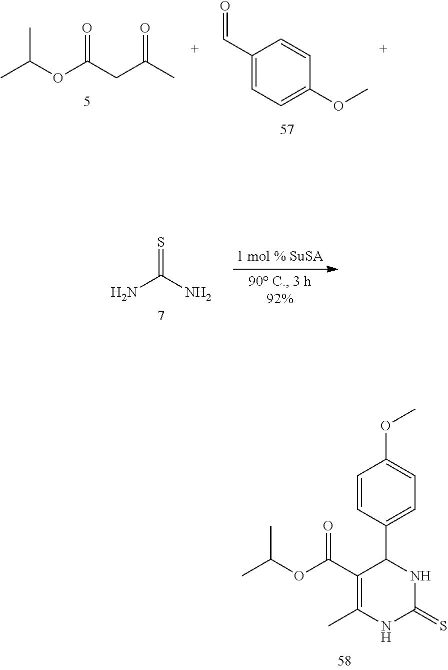

The experimental procedure was similar to that of compound 8. The Biginelli reaction was performed with compound 57 (4-methoxybenzaldehyde). The crude product was subjected to column chromatography (methanol:dichloromethane=1:100, R_{f}=0.3, as the eluent), and after purification, a white solid compound 58 was obtained with a yield of 92%.

¹H NMR (400 MHz, CDCl₃) δ 7.67 (s, 1H), 7.41 (d, J=8.7 Hz, 2H), 7.25 (d, J=8.7 Hz, 2H), 7.01 (d, J=8.7 Hz, 2H), 6.40 (d, J=8.7 Hz, 2H), 6.01 (s, 1H), 4.96 (septet, J=6.0 Hz, 1H), 4.74 (s, 2H), 3.65-3.56 (m, 4H), 2.90 (s, 6H), 2.50 (s, 3H), 2.40-2.38 (m, 4H), 2.29 (s, 3H), 1.24 (d, J=6.0 Hz, 3H), 1.06 (d, J=6.0 Hz, 3H).

LRMS (ESI) m/z: 307.1 [M+H]⁺.

### Synthesis of isopropyl 5-(4-methoxyphenyl)-7-methyl-3-oxo-2,3-dihydro-5H-thiazolo[3,2-a]pyrimidine-6-carboxylate (59)

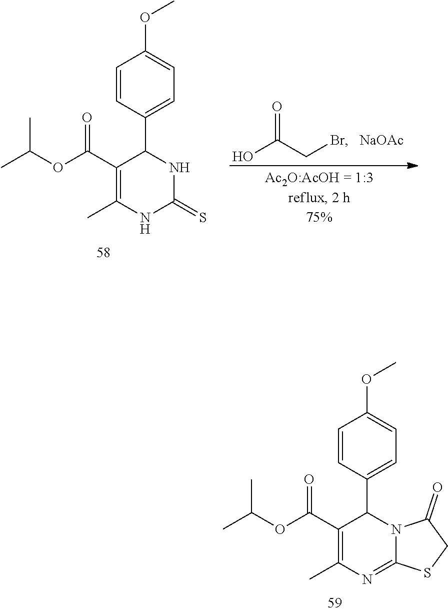

The experimental steps were similar to compound 2. The crude product was purified using column chromatography (ethyl acetate:n-hexane=1:5, R_{f}=0.2, as the eluent) to obtain reddish brown solid compound 59, with a yield of 75%.

¹H NMR (400 MHz, CDCl₃) δ 7.28 (d, J=9.2 Hz, 2H), 6.82 (d, J=9.2 Hz, 2H), 5.99 (s, 1H), 4.94 (septet, J=6.4 Hz, 1H), 3.84 (d, J=17.4 Hz, 1H), 3.77 (s, 3H), 3.72 (d, J=17.4 Hz, 1H), 2.47 (s, 3H), 1.21 (d, J=6.4 Hz, 3H), 1.02 (d, J=6.4 Hz, 3H).

LRMS (ESI) m/z: 361.1 [M+H]⁺.

### Synthesis of (Z)-2-(4-((6-(isopropoxycarbonyl)-5-(4-methoxyphenyl)-7-methyl-3-oxo-5H-thiazolo[3,2-a]pyrimidin-2(3H)-ylidene)methyl)phenoxy)acetic acid (60)

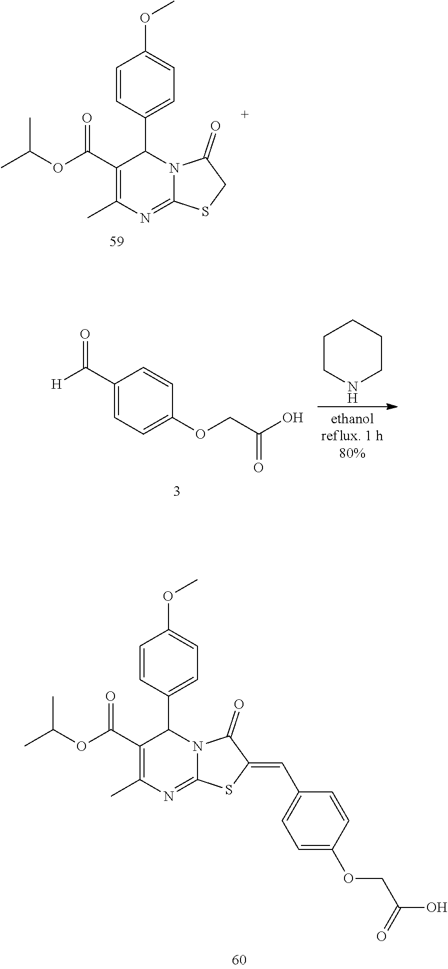 [SMILES: COc1ccc(C2C(C(=O)OC(C)C)=C(C)N=C3SCC(=O)N32)cc1 ; O=Cc1ccc(OCC(=O)O)cc1 ; C1CCNCC1 ; COc1ccc(C2C(C(=O)OC(C)C)=C(C)N=c3s/c(=C\c4ccc(OCC(=O)O)cc4)c(=O)n32)cc1]

The experimental steps were similar to compound 1. Piperidine was used for condensation reaction. The crude product was subjected to column chromatography (methanol:dichloromethane=1:50, R_{f}=0.23, as the eluent). After purification, a yellow solid compound 60 was obtained with a yield of 80%.

¹H NMR (400 MHz, CDCl₃) δ 7.71 (s, 1H), 7.44 (d, J=9.2 Hz, 2H), 7.32 (d, J=8.8 Hz, 2H), 7.01 (d, J=8.98 Hz, 2H), 6.82 (d, J=9.2 Hz, 2H), 6.14 (s, 1H), 4.96 (septet, J=6.4 Hz, 1H), 4.74 (s, 2H), 3.76 (s, 3H), 2.53 (s, 3H), 1.23 (d, J=6.4 Hz, 3H), 1.04 (d, J=6.4 Hz, 3H).

¹³C NMR (600 MHz, CDCl₃) δ 170.4, 165.4, 164.9, 159.7, 159.2, 156.4, 151.4, 133.1, 132.2, 132.1, 129.5, 127.0, 118.0, 115.4, 113.8, 109.5, 68.0, 64.7, 55.2, 55.0, 22.2, 22.0, 21.6.

LRMS (ESI) m/z: 523.1 [M+H]⁺.

HRMS (ESI) m/z calcd for C₂₇H₂₆N₂O₇S [M+H]⁻=521.1383. found: 521.1384.

### Synthesis of isopropyl (Z)-5-(4-methoxyphenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (61)

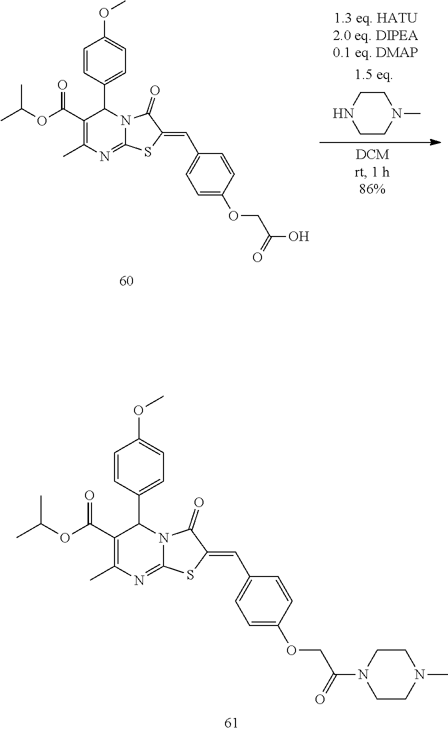

The experimental procedure was similar to that of compound 12. HATU was used to activate the acid group, and the amine compound was 1-methylpiperazine. The crude product was subjected to column chromatography (methanol:dichloromethane=1:30, R_{f}=0.3, as the eluent), and after purification, a yellow solid compound 61 was obtained with a yield of 86%.

¹H NMR (400 MHz, CDCl₃) δ 7.68 (s, 1H), 7.42 (d, J=9.2 Hz, 2H), 7.33 (d, J=8.8 Hz, 2H), 7.02 (d, J=9.2 Hz, 2H), 6.81 (d, J=8.8 Hz, 2H), 6.13 (s, 1H), 4.96 (septet, J=6.4 Hz, 1H), 4.75 (s, 2H), 3.76-3.71 (m, 6H), 2.58 (brs, 3H), 2.51 (s, 3H), 2.43 (brs, 3H), 1.23 (d, J=6.4 Hz, 3H), 1.04 (d, J=6.4 Hz, 3H).

LRMS (ESI) m/z: 605.2 [M+H]⁺.

HRMS (ESI) m/z calcd for C₃₂H₃₆N₄O₆S [M+H]⁺=605.2434. found: 605.2419.

### Synthesis of isopropan-2-yl (2Z)-5-(4-chlorophenyl)-7-methyl-2-(4-(2-(4-(4-methylpiperazin-1-yl)piperidin-1-yl)-2-oxoethoxy)benzylidene)-3-oxo-2,3-dihydro-5H-[1,3]thiazolo[3,2-a]pyrimidine-6-carboxylate (62)

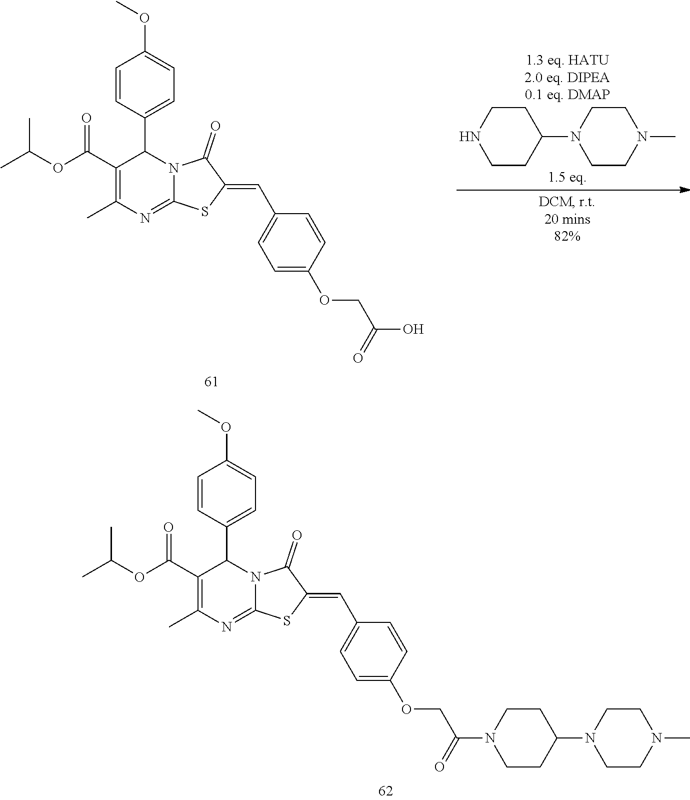 [SMILES: COc1ccc(C2C(C(=O)OC(C)C)=C(C)N=c3s/c(=C\c4ccc(OCC(=O)O)cc4)c(=O)n32)cc1 ; CN1CCN(C2CCNCC2)CC1 ; COc1ccc(C2C(C(=O)OC(C)C)=C(C)N=c3s/c(=C\c4ccc(OCC(=O)N5CCC(N6CCN(C)CC6)CC5)cc4)c(=O)n32)cc1]

The experimental procedure was similar to compound 12. HATU was used to activate the acid group, and the amine compound was 1-methyl-4-(piperidin-4-yl)piperazine. The crude product was purified using column chromatography (methanol:dichloromethane=1:40, R_{f}=0.31, as the eluent) to obtain compound 62, a yellow solid, with a yield of 82%.

¹H NMR (400 MHz, CDCl₃) δ 7.65 (s, 1H), 7.40 (s, 3H), 7.32 (d, J=9.2 Hz, 2H), 7.00-6.80 (m, 2H), 6.80 (d, J=8.8 Hz, 2H), 6.11 (s, 1H), 5.00-4.85 (m, 2H), 4.74 (s, 3H), 3.74 (s, 3H), 3.49 (s, 3H), 3.23 (s, 2H), 3.13-3.04 (m, 2H), 2.85 (s, 4H), 2.50 (s, 4H), 1.23 (d, J=6.4 Hz, 3H), 1.03 (d, J=6.4 Hz, 3H).

LRMS (ESI) m/z: 688.3 [M+H]⁺.

HRMS (ESI) m/z calcd for C₃₇H₄₅N₅O₆S [M+H]⁺=688.3169. found: 688.3169.

### Synthesis of isopropyl 4-(3-chlorophenyl)-6-methyl-2-thioxo-1,2,3,4-tetrahydropyrimidine-5-carboxylate (64)

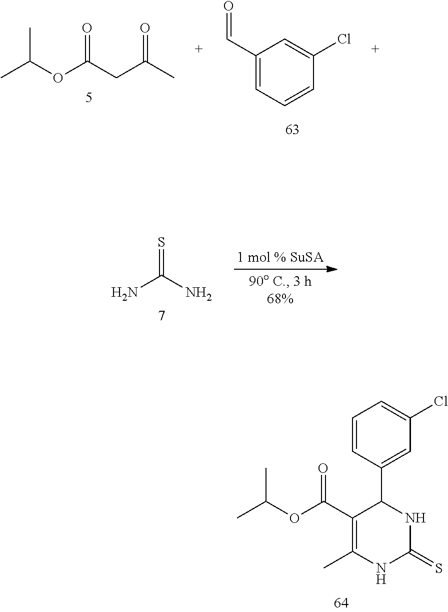

The experimental procedure was similar to that of compound 8. The Biginelli reaction was performed with compound 63 (3-chlorobenzaldehyde). The crude product was subjected to column chromatography (methanol:dichloromethane=1:70, R_{f}=0.4, as the eluent). After purification, white solid compound 64 was obtained with a yield of 68%.

¹H NMR (400 MHz, CDCl₃) δ 8.05 (s, 1H), 7.50 (s, 1H), 7.27-7.14 (m, 4H), 5.35 (s, 1H), 4.97 (septet, J=6.4 Hz, 1H), 2.37 (s, 3H), 1.22 (d, J=6.4 Hz, 2H), 1.06 (d, J=6.4 Hz, 3H).

LRMS (ESI) m/z: 325.1 [M+H]⁺.

### Synthesis of isopropyl 5-(3-chlorophenyl)-7-methyl-3-oxo-2,3-dihydro-5H-thiazolo[3,2-a]pyrimidine-6-carboxylate (65)

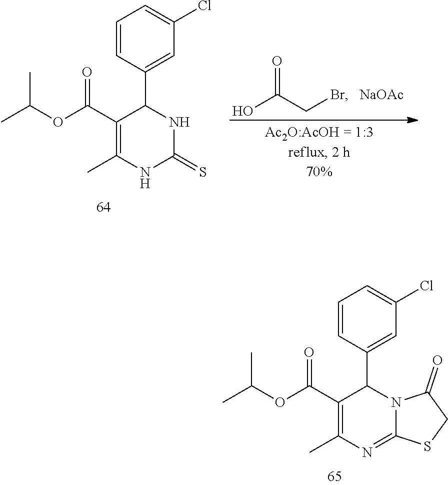

The experimental steps were similar to compound 2. The crude product was purified using column chromatography (ethyl acetate:n-hexane=1:6, R_{f}=0.2, as the eluent) to obtain red-brown solid compound 65, with a yield of 70%.

¹H NMR (300 MHz, CDCl₃) δ 7.34-7.26 (m, 4H), 6.00 (s, 1H), 4.96 (septet, J=6.3 Hz, 1H), 3.87 (d, J=17.4 Hz, 1H), 3.76 (d, J=17.4 Hz, 1H), 2.48 (s, 3H), 1.23 (d, J=6.3 Hz, 3H), 1.04 (d, J=6.3 Hz, 3H).

¹³C NMR (400 MHz, CDCl₃) δ 170.2, 164.5, 159.6, 152.5, 141.9, 134.4, 129.8, 128.8, 128.3, 126.3, 107.8, 68.1, 55.1, 32.3, 22.6, 21.9, 21.5.

LRMS (ESI) m/z: 365.1 [M+H]⁺.

### Synthesis of (Z)-2-(4-((5-(3-chlorophenyl)-6-(isopropoxycarbonyl)-7-methyl-3-oxo-5H-thiazolo[3,2-a]pyrimidin-2(3H)-ylidene)methyl)phenoxy)acetic acid (66)

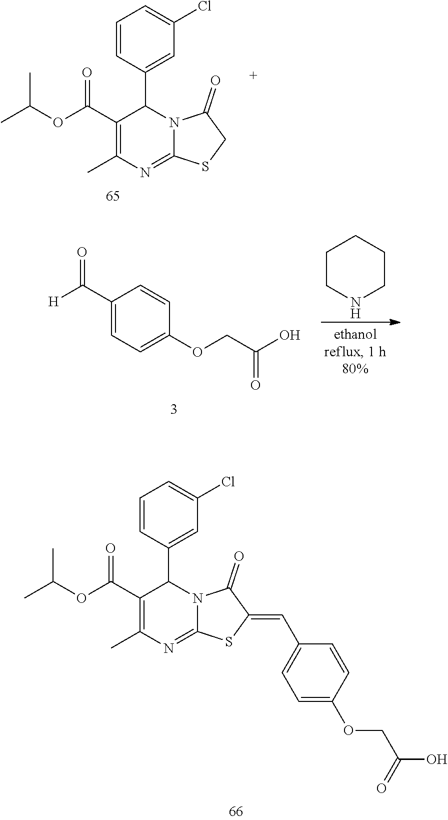 [SMILES: CC1=C(C(=O)OC(C)C)C(c2cccc(Cl)c2)N2C(=O)CSC2=N1 ; O=Cc1ccc(OCC(=O)O)cc1 ; C1CCNCC1 ; CC1=C(C(=O)OC(C)C)C(c2cccc(Cl)c2)n2c(s/c(=C\c3ccc(OCC(=O)O)cc3)c2=O)=N1]

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 66 was obtained with a yield of 80%.

¹H NMR (400 MHz, CD₃OD) δ 7.72 (s, 1H), 7.49 (d, J=8.8 Hz, 2H), 7.38 (s, 1H), 7.33-7.26 (m, 3H), 7.04 (d, J=8.8 Hz, 2H), 6.09 (s, 1H), 4.95 (septet, J=6.4 Hz, 1H), 4.45 (s, 2H), 2.46 (s, 3H), 1.25 (d, J=6.4 Hz, 3H), 1.05 (d, J=6.4 Hz, 3H).

¹³C NMR (400 MHz, DMSO-d₆) δ 170.5, 164.5, 164.1, 161.1, 155.9, 151.9, 142.7, 133.6, 132.9, 132.0, 130.7, 128.5, 127.9, 126.1, 124.5, 115.6, 115.5, 107.9, 67.7, 67.4, 54.6, 22.5, 22.4, 21.6, 21.2.

LRMS (ESI) m/z: 527.1 [M+H]⁺.

HRMS (ESI) m/z calcd for C₂₆H₂₃ClN₂O₆S [M+H]⁺=527.1044. found: 527.1046.

### Synthesis of isopropyl (Z)-5-(3-chlorophenyl)-2-(4-(2-(4-ethylpiperazin-1-yl)-2-oxoethoxy)benzylidene)-7-methyl-3-oxo-2,3-dihydro-5H-thiazolo[3,2-a]pyrimidine-6-carboxylate (67)

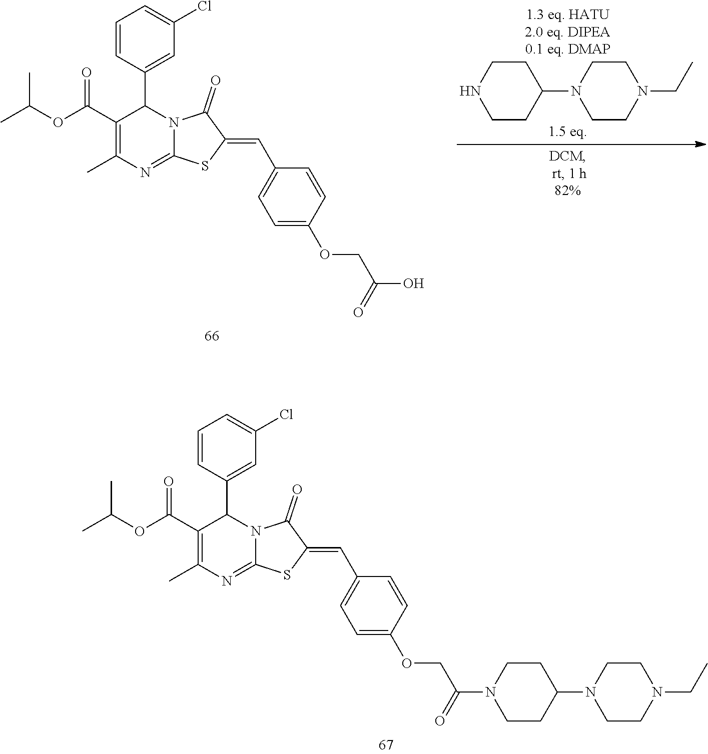 [SMILES: CC1=C(C(=O)OC(C)C)C(c2cccc(Cl)c2)n2c(s/c(=C\c3ccc(OCC(=O)O)cc3)c2=O)=N1 ; CCN1CCN(C2CCNCC2)CC1 ; CCN1CCN(C2CCN(C(=O)COc3ccc(/C=c4\sc5n(c4=O)C(c4cccc(Cl)c4)C(C(=O)OC(C)C)=C(C)N=5)cc3)CC2)CC1]

The experimental procedure was similar to compound 12. HATU was used to activate the acid group, and the amine compound was 1-ethylpiperazine. The crude product was subjected to column chromatography (methanol:dichloromethane=1:40, R_{f}=0.3, as the eluent). After purification, a yellow solid compound 67 was obtained with a yield of 82%.

¹H NMR (300 MHz, CDCl₃) δ 7.70 (s, 1H), 7.43 (d, J=8.4 Hz, 2H), 7.39 (s, 1H), 7.30-7.22 (m, 3H), 7.03 (d, J=8.4 Hz, 2H), 6.13 (s, 1H), 4.98 (septet, J=6.3 Hz, 1H), 4.75 (s, 2H), 3.65-3.55 (m, 4H), 2.52 (s, 3H), 2.45-2.37 (m, 6H), 1.25 (d, J=6.3 Hz, 3H), 1.21-1.04 (m, 6H).

¹³C NMR (400 MHz, CDCl₃) δ 165.2, 165.2, 164.9, 164.8, 164.3, 164.3, 159.5, 155.9, 155.8, 152.4, 152.4, 141.8, 134.0, 134.0, 133.0, 132.9, 131.8, 131.8, 129.6, 128.5, 128.4, 128.1, 126.3, 126.2, 126.0, 117.2, 117.1, 115.2, 108.2, 108.2, 67.8, 67.8, 67.0, 66.9, 54.8, 52.6, 52.0, 51.9, 51.8, 44.9, 44.9, 41.8, 41.8, 22.5, 21.7, 21.7, 21.3, 11.6.

LRMS (ESI) m/z: 623.2 [M+H]⁺.

HRMS (ESI) m/z calcd for C₃₂H₃₅ClN₄O₅S [M+H]⁺=623.2095. found: 623.2094.

### Synthesis of methyl 4-(4-chlorophenyl)-6-methyl-2-thioxo-1,2,3,4-tetrahydropyrimidine-5-carboxylate (72)

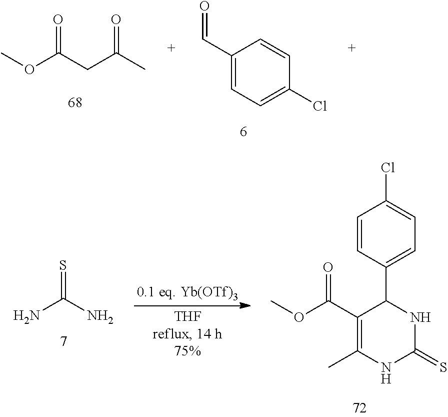

The experimental procedure was similar to that of compound 8. The Biginelli reaction was performed with compound 6 (4-chlorobenzaldehyde). The crude product was subjected to column chromatography (methanol:dichloromethane=1:100, R_{f}=0.2, as the eluent). After purification, a white solid compound 72 was obtained with a yield of 75%.

¹H NMR (300 MHz, CDCl₃) δ 7.79 (s, 1H), 7.30 (d, J=8.7 Hz, 2H), 7.22 (d, J=8.7 Hz, 2H), 5.38 (s, 1H), 3.66 (s, 3H), 2.36 (s, 3H).

LRMS (ESI) m/z: 297.1 [M+H]⁺.

### Synthesis of ethyl 4-(4-chlorophenyl)-6-methyl-2-thioxo-1,2,3,4-tetrahydropyrimidine-5-carboxylate (73)

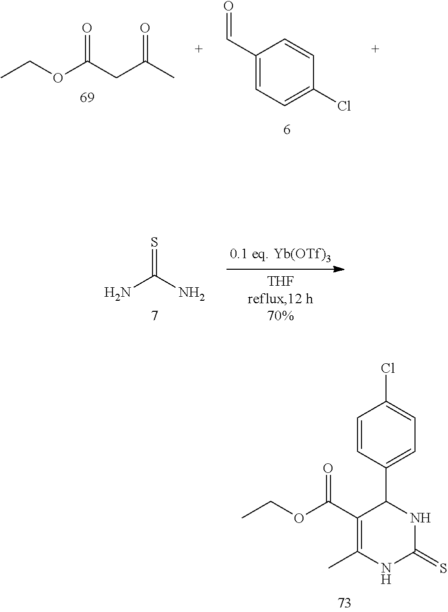

The experimental procedure was similar to that of compound 8. The Biginelli reaction was performed with compound 6 (4-chlorobenzaldehyde). The crude product was subjected to column chromatography (methanol:dichloromethane=1:100, R_{f}=0.21, as the eluent). After purification, a white solid compound 73 was obtained with a yield of 70%.

¹H NMR (300 MHz, CDCl₃) δ 7.81 (s, 1H), 7.30 (d, J=8.7 Hz, 2H), 7.23 (d, J=8.7 Hz, 2H), 5.38 (S, 1H), 4.11 (q, J=7.1 Hz, 2H), 2.36 (s, 3H), 1.19 (t, J=7.1 Hz, 3H).

¹³C NMR (600 MHz, CDCl₃) δ 174.7, 165.0, 142.8, 140.8, 134.3, 129.1, 128.2, 102.7, 60.6, 55.6, 18.5, 14.1.

LRMS (ESI) m/z: 311.1 [M+H]⁺.

### Synthesis of tert-butyl 4-(4-chlorophenyl)-6-methyl-2-thioxo-1,2,3,4-tetrahydropyrimidine-5-carboxylate (74)

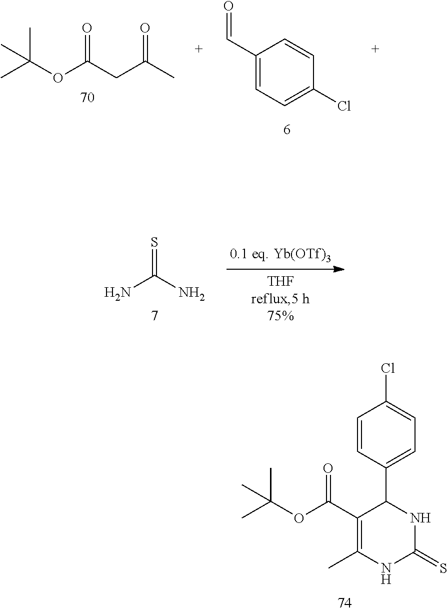

The experimental procedure was similar to that of compound 8. The Biginelli reaction was performed with compound 6 (4-chlorobenzaldehyde). The crude product was subjected to column chromatography (methanol:dichloromethane=1:100, R_{f}=0.3, as the eluent). After purification, a white solid compound 74 was obtained with a yield of 75%.

¹H NMR (300 MHz, CDCl₃) δ 7.95 (s, 1H), 7.39 (d, J=8.7 Hz, 2H), 7.22 (d, J=8.7 Hz, 2H), 5.30 (s, 1H), 2.32 (s, 3H), 1.34 (s, 9H).

¹³C NMR (400 MHz, CDCl₃) δ 173.7, 164.2, 142.3, 140.9, 133.9, 128.8, 128.3, 128.1, 103.6, 81.2, 60.3, 55.4, 28.0, 17.8, 14.1.

LRMS (ESI) m/z: 339.1 [M+H]⁺.

### Synthesis of methyl 5-(4-chlorophenyl)-7-methyl-3-oxo-2,3-dihydro-5H-thiazolo[3,2-a]pyrimidine-6-carboxylate (75)

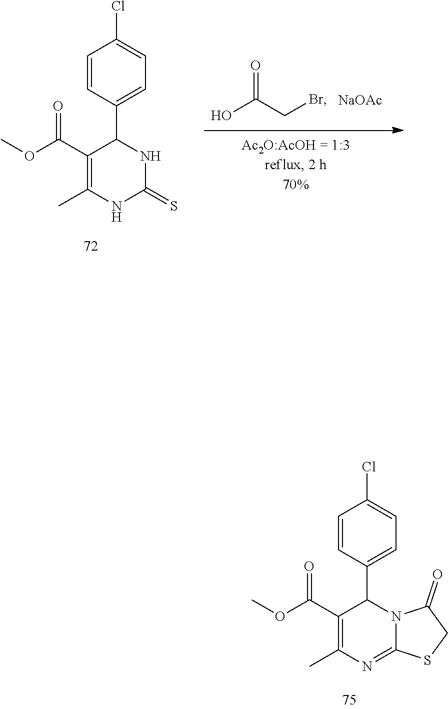 [SMILES: COC(=O)C1=C(C)NC(=S)NC1c1ccc(Cl)cc1 ; O=C(O)CBr ; COC(=O)C1=C(C)N=C2SCC(=O)N2C1c1ccc(Cl)cc1 ; "NaOAc"]

The experimental steps were similar to compound 2. The crude product was purified by column chromatography (ethyl acetate:n-hexane=1:6, R_{f}=0.2, as the eluent). After purification, a reddish-brown solid compound 75 was obtained, with a yield of 70%.

¹H NMR (300 MHz, CDCl₃) δ 7.95 (s, 1H), 7.39 (d, J=8.7 Hz, 2H), 7.22 (d, J=8.7 Hz, 2H), 5.30 (s, 1H), 2.32 (s, 3H), 1.34 (s, 9H).

¹³C NMR (400 MHz, CDCl₃) δ 173.1, 169.8, 164.9, 164.3, 159.9, 159.7, 152.1, 138.1, 133.6, 128.9, 128.8, 128.8, 128.7, 128.4, 128.2, 128.1, 128.0, 127.9, 106.6, 59.8, 59.6, 54.2, 54.1, 54.0, 54.0, 50.9, 50.8, 50.8, 31.8, 22.3, 22.2, 22.1, 22.0, 22.0, 20.2, 20.0, 20.0, 13.5, 13.4, 13.4.

LRMS (ESI) m/z: 337.1 [M+H]⁺.

### Synthesis of ethyl 5-(4-chlorophenyl)-7-methyl-3-oxo-2,3-dihydro-5H-thiazolo[3,2-a]pyrimidine-6-carboxylate (76)

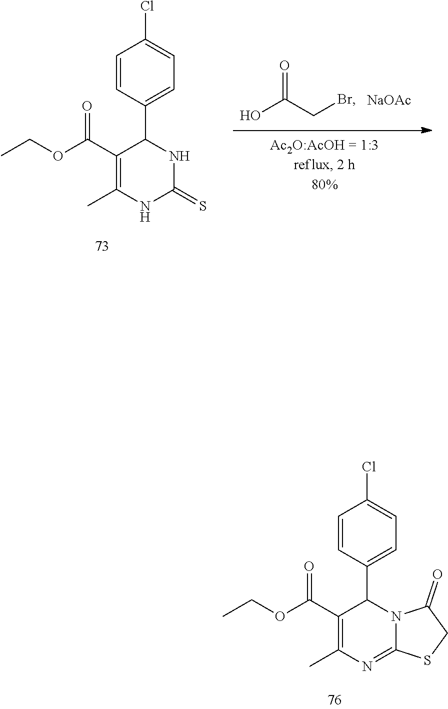 [SMILES: CCOC(=O)C1=C(C)NC(=S)NC1c1ccc(Cl)cc1 ; O=C(O)CBr ; CCOC(=O)C1=C(C)N=C2SCC(=O)N2C1c1ccc(Cl)cc1 ; "NaOAc"]

The experimental procedure was similar to compound 2. The crude product was purified using column chromatography (ethyl acetate:n-hexane=1:7, R_{f}=0.18, as the eluent) to obtain red-brown solid compound 76, with a yield of 80%.

¹H NMR (400 MHz, CDCl₃) δ 7.33-7.26 (m, 4H), 6.02 (s, 1H), 4.08 (q, J=5.4 Hz, 2H), 3.86 (d, J=17.4 Hz, 1H), 3.74 (d, J=17.4 Hz, 1H), 2.48 (s, 3H), 1.17 (t, J=5.4 Hz, 3H).

¹³C NMR (300 MHz, CDCl₃) δ 170.2, 165.1, 159.8, 152.5, 138.4, 134.5, 129.4, 128.8, 128.7, 128.5, 107.6, 60.5, 54.8, 32.3, 22.6, 14.1, 14.0.

LRMS (ESI) m/z: 351.1 [M+H]⁺.

### Synthesis of tert-butyl 5-(4-chlorophenyl)-7-methyl-3-oxo-2,3-dihydro-5H-thiazolo[3,2-a]pyrimidine-6-carboxylate (77)

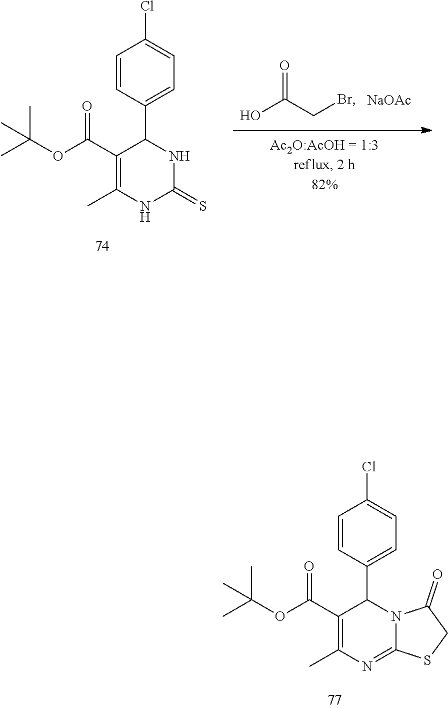 [SMILES: CC1=C(C(=O)OC(C)(C)C)C(c2ccc(Cl)cc2)NC(=S)N1 ; O=C(O)CBr ; CC1=C(C(=O)OC(C)(C)C)C(c2ccc(Cl)cc2)N2C(=O)CSC2=N1 ; "NaOAc"]

The experimental procedure was similar to that of compound 2. The crude product was purified using column chromatography (ethyl acetate:n-hexane=1:6, R_{f}=0.2, as the eluent) to obtain red-brown solid compound 77, with a yield of 82%.

¹H NMR (400 MHz, CDCl₃) δ 7.30-7.27 (m, 4H), 5.96 (s, 1H), 3.85 (d, J=17.4 Hz, 1H), 3.71 (d, J=17.4 Hz, 1H), 2.45 (s, 3H), 1.21 (s, 9H).

¹³C NMR (400 MHz, CDCl₃) δ 169.8, 163.8, 158.7, 151.1, 138.1, 133.8, 129.2, 128.1, 128.1, 108.2, 80.6, 54.6, 31.8, 27.7, 27.6, 27.5, 22.1.

LRMS (ESI) m/z: 379.1 [M+H]⁺.

### Synthesis of (Z)-2-(4-((5-(4-chlorophenyl)-6-(methoxycarbonyl)-7-methyl-3-oxo-5H-thiazolo[3,2-a]pyrimidin-2(3H)-ylidene)methyl)phenoxy)acetic acid (78)

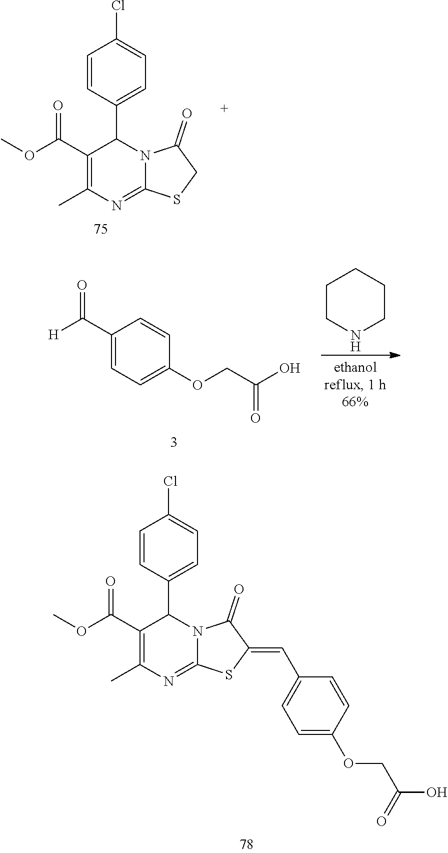 [SMILES: COC(=O)C1=C(C)N=C2SCC(=O)N2C1c1ccc(Cl)cc1 ; O=Cc1ccc(OCC(=O)O)cc1 ; C1CCNCC1 ; COC(=O)C1=C(C)N=c2s/c(=C\c3ccc(OCC(=O)O)cc3)c(=O)n2C1c1ccc(Cl)cc1]

The experimental procedure was similar to that of compound 1. Piperidine was used for condensation reaction. The crude product was subjected to column chromatography (methanol:dichloromethane=1:50, R_{f}=0.2 as the eluent). After purification, a yellow solid compound 78 was obtained with a yield of 66%.

¹H NMR (400 MHz, DMSO-d₆) δ 7.70 (s, 1H), 7.47 (d, J=9.2 Hz, 2H), 7.40 (d, J=8.4 Hz, 2H), 7.31 (d, J=8.4 Hz, 2H), 6.95 (d, J=9.2 Hz, 2H), 6.03 (s, 1H), 4.30 (s, 1H), 3.60 (s, 3H), 2.38 (s, 3H).

¹³C NMR (600 MHz, DMSO-d₆) δ 172.0, 165.3, 164.4, 161.0, 156.0, 152.0, 139.3, 133.6, 133.2, 132.0, 129.4, 129.3, 128.7, 128.6, 124.6, 115.6, 115.5, 107.7, 67.2, 60.2, 54.3, 54.2, 51.5, 22.6, 22.5, 13.9.

LRMS (ESI) m/z: 499.1 [M+H]⁺.

HRMS (ESI) m/z calcd for C₂₄H₁₉ClN₂O₆S [M+H]⁻=497.0574. found: 497.0567.

### Synthesis of (Z)-2-(4-((5-(4-chlorophenyl)-6-(ethoxycarbonyl)-7-methyl-3-oxo-5H-thiazolo[3,2-a]pyrimidin-2(3H)-ylidene)methyl)phenoxy)acetic acid (79)

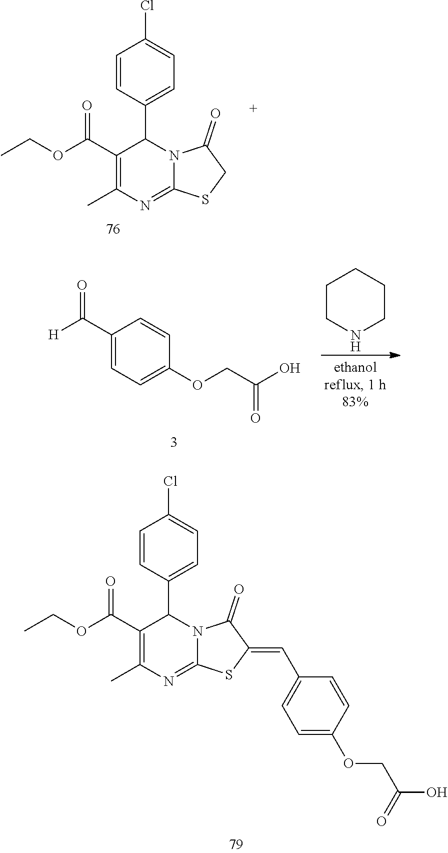 [SMILES: CCOC(=O)C1=C(C)N=C2SCC(=O)N2C1c1ccc(Cl)cc1 ; O=Cc1ccc(OCC(=O)O)cc1 ; C1CCNCC1 ; CCOC(=O)C1=C(C)N=c2s/c(=C\c3ccc(OCC(=O)O)cc3)c(=O)n2C1c1ccc(Cl)cc1]

The experimental procedure was similar to that of compound 1. Piperidine was used for condensation reaction. The crude product was subjected to column chromatography (methanol:dichloromethane=1:51, R_{f}=0.2, as the eluent). After purification, a yellow solid compound 79 was obtained with a yield of 83%.

¹H NMR (400 MHz, CDCl₃) δ 7.70 (s, 1H), 7.44 (d, J=8.8 Hz, 2H), 7.35 (d, J=8.8 Hz, 2H), 7.29-7.26 (m, 2H), 7.01 (d, J=8.8 Hz, 2H), 6.16 (s, 1H), 4.73 (s, 2H), 4.11 (q, J=7.2 Hz, 2H), 2.52 (s, 3H), 1.2 (t, J=7.2 Hz, 3H).

LRMS (ESI) m/z: 513.1 [M+H]⁺.

HRMS (ESI) m/z calcd for C₂₅H₂₁ClN₂O₆S [M+H]⁺=513.0887. found: 513.0890.

### Synthesis of (Z)-2-(4-((6-(tert-butoxycarbonyl)-5-(4-chlorophenyl)-7-methyl-3-oxo-5H-thiazolo[3,2-a]pyrimidin-2(3H)-ylidene)methyl)phenoxy)acetic acid (80)

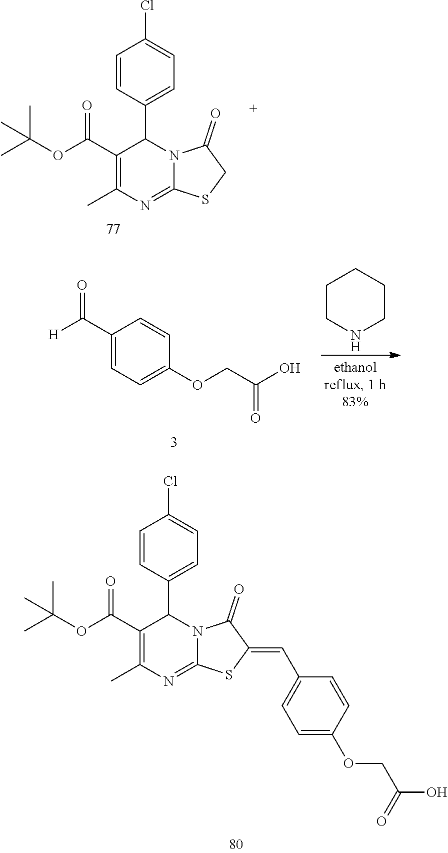 [SMILES: CC1=C(C(=O)OC(C)(C)C)C(c2ccc(Cl)cc2)N2C(=O)CSC2=N1 ; O=Cc1ccc(OCC(=O)O)cc1 ; C1CCNCC1 ; CC1=C(C(=O)OC(C)(C)C)C(c2ccc(Cl)cc2)n2c(s/c(=C\c3ccc(OCC(=O)O)cc3)c2=O)=N1]

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 80 was obtained with a yield of 66%.

¹H NMR (400 MHz, CDCl₃) δ 7.41 (s, 1H), 7.28 (d, J=8.7 Hz, 2H), 7.35 (d, J=8.4 Hz, 2H), 7.23 (d, J=8.4 Hz, 2H), 6.75 (d, J=8.7 Hz, 2H), 6.00 (s, 1H), 4.26 (s, 2H), 2.42 (s, 3H), 1.33 (s, 9H).

¹³C NMR (400 MHz, CDCl₃) δ 174.1, 164.8, 164.3, 159.0, 155.1, 151.1, 138.3, 134.3, 132.2, 131.8, 129.5, 128.5, 126.2, 117.4, 115.0, 109.7, 81.3, 66.5, 55.0, 27.9, 27.8, 27.7, 22.4.

LRMS (ESI) m/z: 541.1 [M+H]⁺.

HRMS (ESI) m/z calcd for C₂₇H₂₅ClN₂O₆S [M+H]⁻=539.1044. found: 539.1042.

### Synthesis of allyl 4-(4-chlorophenyl)-6-methyl-2-thioxo-1,2,3,4-tetrahydropyrimidine-5-carboxylate (84)

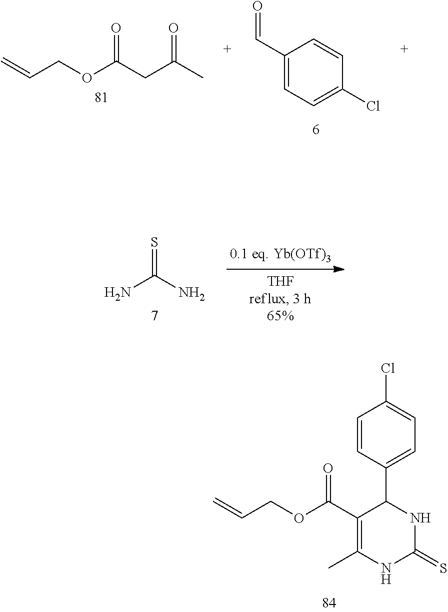

The experimental procedure was similar to that of compound 8. The Biginelli reaction was performed with compound 6 (4-chlorobenzaldehyde). The crude product was subjected to column chromatography (methanol:dichloromethane=1:100, R_{f}=0.3, as the eluent). After purification, a white solid compound 84 was obtained with a yield of 64%.

¹H NMR (400 MHz, CDCl₃) δ 7.77 (s, 1H), 7.30 (d, J=8.4 Hz, 2H), 7.23 (d, J=8.4 Hz, 4H), 5.88-5.73 (m, 1H), 5.22-5.12 (m, 2H), 4.59-4.53 (m, 2H), 2.37 (s, 3H).

LRMS (ESI) m/z: 323.1 [M+H]⁺.

### Synthesis of benzyl 4-(4-chlorophenyl)-6-methyl-2-thioxo-1,2,3,4-tetrahydropyrimidine-5-carboxylate (85)

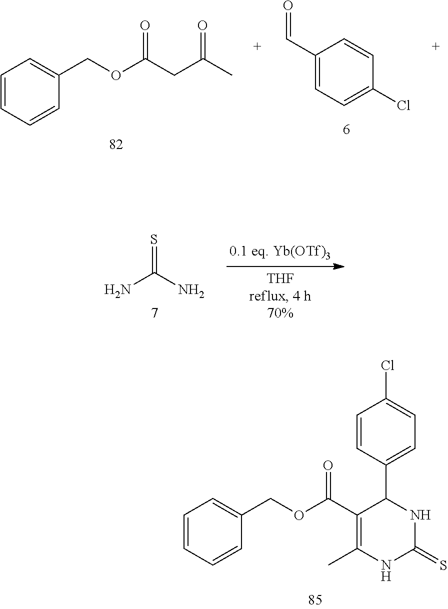

The experimental procedure was similar to that of compound 8. The Biginelli reaction was performed with compound 6 (4-chlorobenzaldehyde). The crude product was subjected to column chromatography (methanol:dichloromethane=1:80, R_{f}=0.2, as the eluent). After purification, a white solid compound 85 was obtained with a yield of 70%.

¹H NMR (400 MHz, CDCl₃) δ 7.56 (brs, 1H), 7.40-7.33 (m, 3H), 7.34-7.28 (m, 3H), 7.24 (s, 1H), 7.19-7.09 (m, 3H), 6.98 (brs, 1H), 5.38 (s, 1H), 5.18-4.97 (m, 2H), 4.71 (d, J=5.9 Hz, 1H), 2.37 (s, 3H).

¹³C NMR (400 MHz, CDCl₃) δ 174.1, 164.8, 143.7, 143.1, 140.7, 135.4, 134.2, 129.0, 128.5, 128.3, 128.2, 102.6, 102.1, 66.4, 60.5, 55.3, 18.3, 14.1.

LRMS (ESI) m/z: 373.2 [M+H]⁺.

### Synthesis of 1-(4-(4-chlorophenyl)-6-methyl-2-thioxo-1,2,3,4-tetrahydropyrimidin-5-yl)-3-methylbutan-1-one (86)

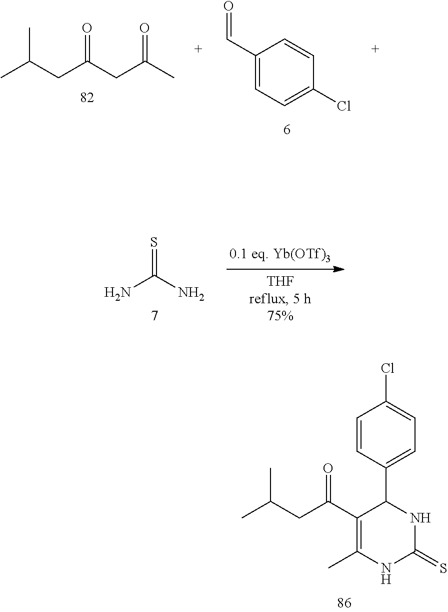

The experimental procedure was similar to that of compound 8. The Biginelli reaction was performed with compound 6 (4-chlorobenzaldehyde). The crude product was subjected to column chromatography (methanol:dichloromethane=1:50, R_{f}=0.4, as the eluent). After purification, a white solid compound 86 was obtained with a yield of 75%.

¹H NMR (300 MHz, CDCl₃): δ 7.68 (s, 1H), 7.32 (d, J=8.7 Hz, 2H), 7.21 (d, J=8.7 Hz, 2H), 5.46 (s, 1H), 2.33-2.04 (m, 6H), 0.83 (s, 3H), 0.77 (s, 3H).

¹³C NMR (400 MHz, CDCl₃) δ 197.8, 173.3, 141.5, 139.9, 134.1, 129.0, 128.2, 111.5, 54.9, 50.5, 24.5, 22.4, 22.2, 18.8.

LRMS (ESI) m/z: 323.1 [M+H]⁺.

### Synthesis of allyl 5-(4-chlorophenyl)-7-methyl-3-oxo-2,3-dihydro-5H-thiazolo[3,2-a]pyrimidine-6-carboxylate (87)

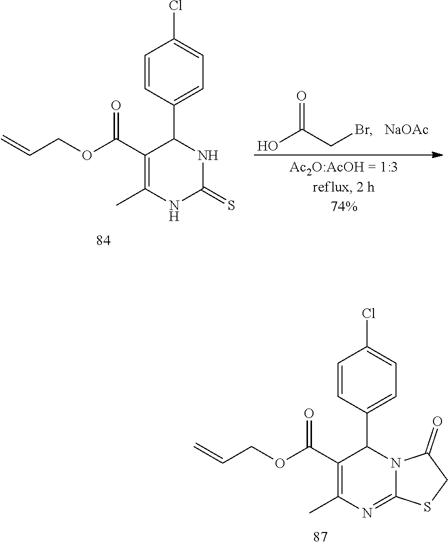

The experimental procedure was similar to that of compound 2. The crude product was purified using column chromatography (ethyl acetate:n-hexane=1:6, R_{f}=0.2, as the eluent) to obtain red-brown solid compound 87, with a yield of 74%.

¹H NMR (400 MHz, CDCl₃) δ 7.35-7.27 (m, 4H), 6.05 (s, 1H), 5.88-5.65 (m, 1H), 5.22-5.11 (m, 2H), 4.54 (d, J=5.4 Hz, 2H), 3.92-3.67 (m, 2H), 2.49 (s, 3H).

LRMS (ESI) m/z: 363.1 [M+H]⁺.

### Synthesis of benzyl 5-(4-chlorophenyl)-7-methyl-3-oxo-2,3-dihydro-5H-thiazolo[3,2-a]pyrimidine-6-carboxylate (88)

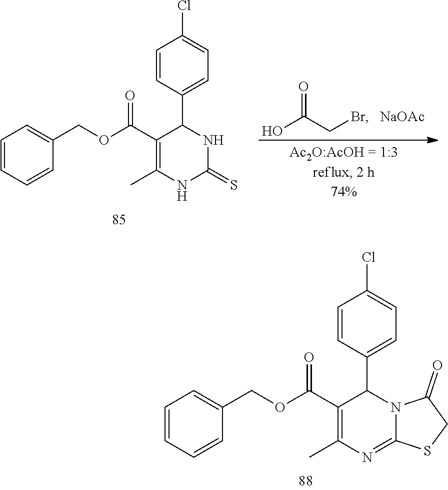

The experimental procedure was similar to that of compound 2. The crude product was purified using column chromatography (ethyl acetate:n-hexane=1:5, R_{f}=0.2, as the eluent) to obtain red-brown solid compound 88, with a yield of 68%.

¹H NMR (400 MHz, CDCl₃) δ 7.33-7.30 (m, 4H), 7.26-7.13 (m, 2H), 6.04 (s, 1H), 5.15 (d, J=12.3 Hz, 1H), 5.03 (d, J=12.3 Hz, 1H), 3.83 (d, J=17.6 Hz, 1H), 3.71 (d, J=17.6 Hz, 1H), 2.53 (s, 3H).

¹³C NMR (400 MHz, CDCl₃) δ 170.1, 164.8, 160.0, 153.2, 138.1, 135.3, 134.4, 129.4, 128.6, 128.3, 128.1, 128.1, 128.0, 107.1, 66.2, 54.7, 32.2, 22.7.

LRMS (ESI) m/z: 413.1 [M+H]⁺.

### Synthesis of 5-(4-chlorophenyl)-7-methyl-6-(3-methylbutanoyl)-5H-thiazolo[3,2-a]pyrimidin-3(2H)-one (89)

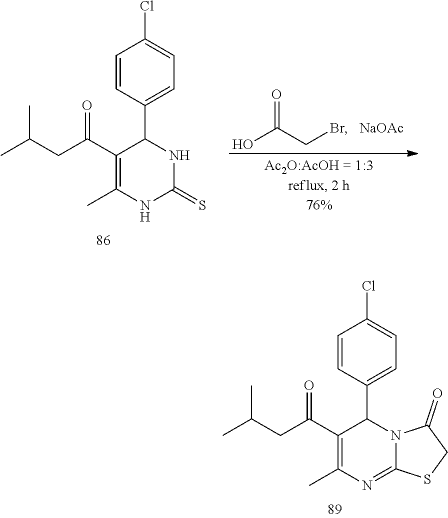

The experimental procedure was similar to that of compound 2. The crude product was purified using column chromatography (ethyl acetate:n-hexane=1:8, R_{f}=0.22, as the eluent) to obtain red-brown solid compound 89, with a yield of 76%.

¹H NMR (300 MHz, CDCl₃) δ 7.30-7.23 (m, 4H), 6.07 (s, 1H), 3.83 (d, J=17.4 Hz, 2H), 3.70 (d, J=17.4 Hz, 2H), 2.43-2.01 (m, 7H), 0.74 (s, 3H), 0.72 (s, 3H).

LRMS (ESI) m/z: 363.1 [M+H]⁺.

### Synthesis of (Z)-2-(4-((6-((allyloxy)carbonyl)-5-(4-chlorophenyl)-7-methyl-3-oxo-5H-thiazolo[3,2-a]pyrimidin-2(3H)-ylidene)methyl)phenoxy)acetic acid (90)

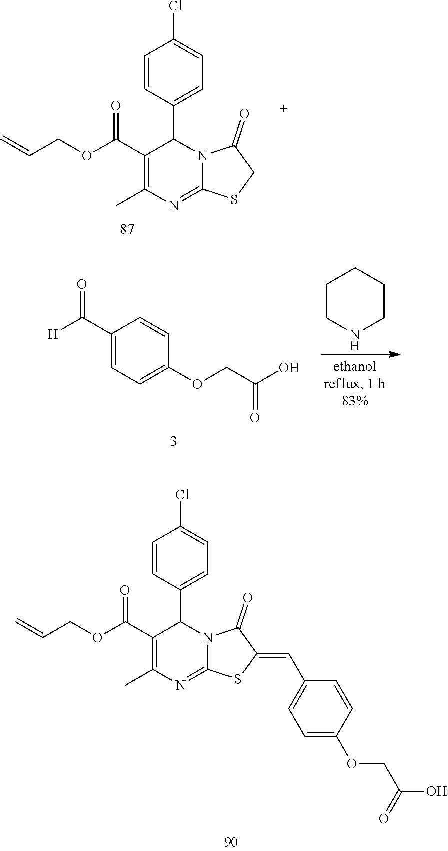 [SMILES: C=CCOC(=O)C1=C(C)N=C2SCC(=O)N2C1c1ccc(Cl)cc1 ; O=Cc1ccc(OCC(=O)O)cc1 ; C1CCNCC1 ; C=CCOC(=O)C1=C(C)N=c2s/c(=C\c3ccc(OCC(=O)O)cc3)c(=O)n2C1c1ccc(Cl)cc1]

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 90 was obtained with a yield of 83%.

¹H NMR (400 MHz, CDCl₃) δ 7.70 (s, 1H), 7.44 (d, J=8.8 Hz, 2H), 7.35 (d, J=8.6 Hz, 2H), 7.28 (s, 2H), 7.01 (d, J=8.8 Hz, 2H), 6.18 (s, 1H), 5.81 (ddd, J=16.6, 11.1, 5.6 Hz, 1H), 5.23-5.12 (m, 2H), 4.74 (s, 2H), 4.56 (s, 2H), 2.53 (s, 3H).

¹³C NMR (600 MHz, CDCl₃) δ 172.7, 165.0, 159.3, 156.7, 152.8, 138.2, 134.6, 133.4, 132.0, 131.7, 129.4, 128.8, 128.8, 126.5, 118.4, 117.4, 115.3, 108.3, 65.3, 65.3, 60.7, 54.8, 54.7, 44.6, 22.6, 22.5, 22.4, 14.0.

LRMS (ESI) m/z: 525.1 [M+H]⁺.

### Synthesis of (Z)-2-(4-((6-((benzyloxy)carbonyl)-5-(4-chlorophenyl)-7-methyl-3-oxo-5H-thiazolo[3,2-a]pyrimidin-2(3H)-ylidene)methyl)phenoxy)acetic acid (91)

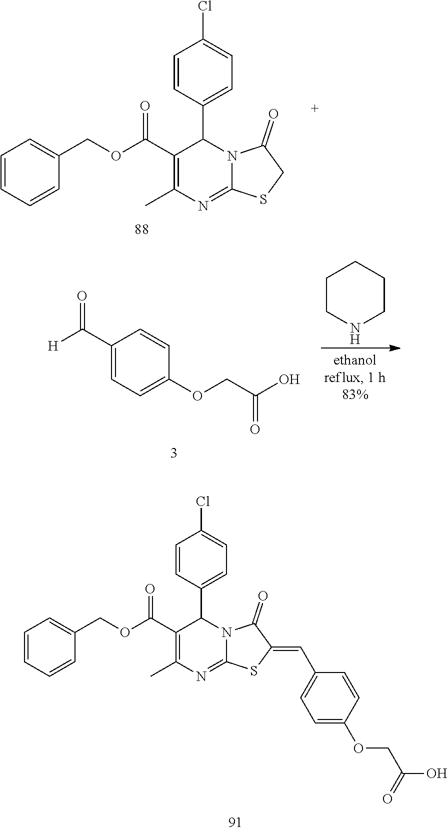 [SMILES: CC1=C(C(=O)OCc2ccccc2)C(c2ccc(Cl)cc2)N2C(=O)CSC2=N1 ; O=Cc1ccc(OCC(=O)O)cc1 ; C1CCNCC1 ; CC1=C(C(=O)OCc2ccccc2)C(c2ccc(Cl)cc2)n2c(s/c(=C\c3ccc(OCC(=O)O)cc3)c2=O)=N1]

The experimental procedure was similar to that of compound 1. Piperidine was used for condensation reaction. The crude product was subjected to column chromatography (methanol:dichloromethane=1:40, R_{f}=0.18, as the eluent). After purification, a yellow solid compound 91 was obtained with a yield of 83%.

¹H NMR (400 MHz, CDCl₃) δ 7.59 (s, 1H), 7.36-7.28 (m, 2H), 7.27-7.20 (m, 4H), 7.18-7.12 (m, 2H), 7.06 (dd, J=6.2, 2.8 Hz, 2H), 6.94-6.85 (m, 2H), 6.08 (s, 1H), 5.14-4.85 (m, 2H), 4.42 (s, 2H), 2.46 (s, 3H).

LRMS (ESI) m/z: 575.1 [M+H]⁺.

### Synthesis of (Z)-2-(4-((5-(4-chlorophenyl)-7-methyl-6-(3-methylbutanoyl)-3-oxo-5H-thiazolo[3,2-a]pyrimidin-2(3H)-ylidene)methyl)phenoxy)acetic acid (92)

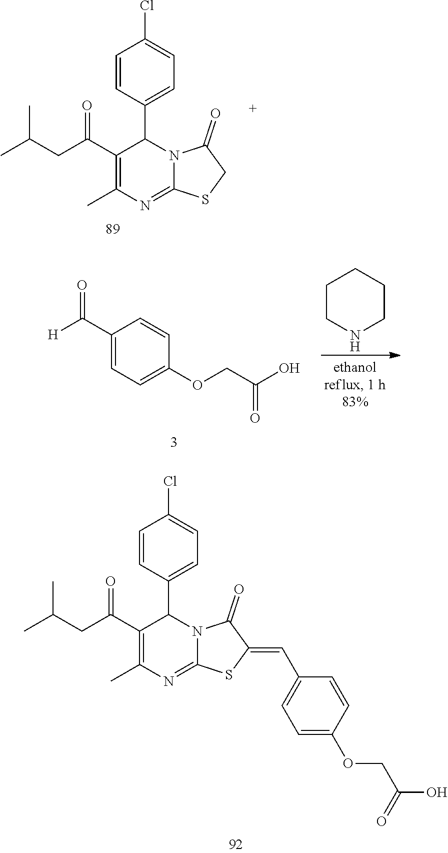 [SMILES: CC1=C(C(=O)CC(C)C)C(c2ccc(Cl)cc2)N2C(=O)CSC2=N1 ; O=Cc1ccc(OCC(=O)O)cc1 ; C1CCNCC1 ; CC1=C(C(=O)CC(C)C)C(c2ccc(Cl)cc2)n2c(s/c(=C\c3ccc(OCC(=O)O)cc3)c2=O)=N1]

The experimental procedure was similar to that of compound 1 Piperidine was used for condensation reaction. The crude product was subjected to column chromatography (methanol:dichloromethane=1:50, R_{f}=0.2, as the eluent). After purification, a yellow solid compound 92 was obtained with a yield of 83%.

¹H NMR (400 MHz, CDCl₃) δ 7.56 (s, 1H), 7.27 (d, J=8.7 Hz, 2H), 7.26-7.18 (m, 4H), 6.86 (d, J=6.6 Hz, 2H), 6.15 (s, 1H), 4.46 (s, 2H), 2.43-1.46 (m, 6H), 0.71 (s, 3H), 0.70 (s, 3H).

¹³C NMR (300 MHz, CDCl₃) δ 199.4, 173.3, 164.7, 159.5, 155.0, 148.4, 137.6, 134.3, 132.8, 131.7, 129.3, 128.6, 125.8, 117.5, 116.8, 115.1, 66.1, 54.4, 53.3, 51.0, 44.1, 24.4, 23.1, 22.3, 22.1, 22.0.

LRMS (ESI) m/z: 525.1 [M+H]+.

### Synthesis of compound 93

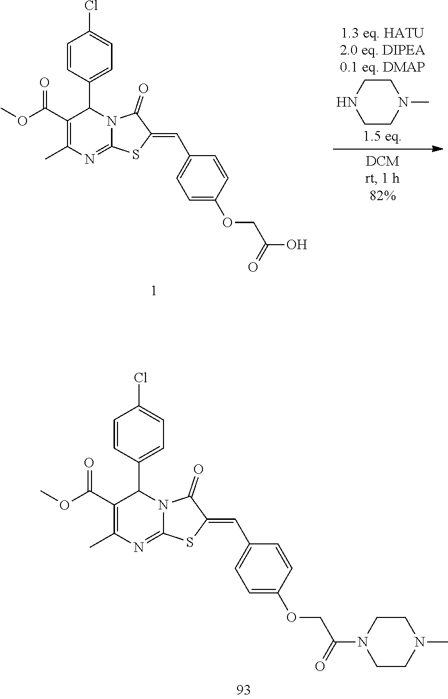

The experimental procedure was similar to that of compound 12. HATU was used to activate the acid group, and the amine compound was 1-methylpiperazine. The crude product was analyzed by column chromatography (methanol:dichloromethane=1:40, R_{f}=0.31, as the eluent). After purification, a yellow solid compound 93 was obtained with a yield of 82%.

¹H NMR (400 MHz, CDCl₃) δ 7.70 (s, 1H), 7.42 (d, J=8.8 Hz, 2H), 7.34 (d, J=8.8 Hz, 2H), 7.27 (d, J=8.8 Hz, 2H), 7.03 (d, J=8.8 Hz, 2H), 6.16 (s, 1H), 4.75 (s, 2H), 3.66 (s, 3H), 3.64-3.55 (m, 4H), 2.52 (s, 3H), 2.42-2.36 (m, 4H), 2.29 (s, 3H).

¹³C NMR (400 MHz, CDCl₃) δ 165.5, 165.2, 164.8, 159.4, 156.2, 152.7, 138.2, 134.1, 133.1, 131.8, 129.0, 128.5, 126.2, 117.1, 115.2, 107.9, 66.9, 54.7, 54.3, 54.2, 51.3, 45.6, 44.7, 41.7, 22.6.

LRMS (ESI) m/z: 581.2 [M+H]⁺.

HRMS (ESI) m/z calcd for C₂₉H₂₉ClN₄O₅S [M+H]⁺=581.1625. found: 581.1618.

### Synthesis of Compound 94

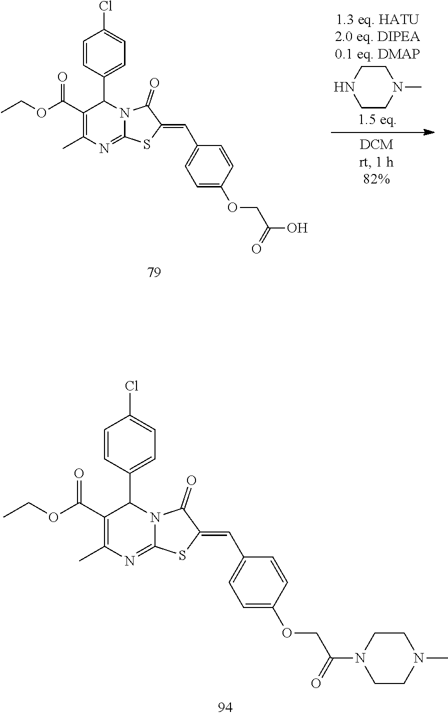

The experimental procedure was similar to that of compound 12. HATU was used to activate the acid group, and the amine compound was 1-methylpiperazine. The crude product was subjected to column chromatography (methanol:dichloromethane=1:50, R_{f}=0.3, as the eluent). After purification, a yellow solid compound 94 was obtained with a yield of 82%.

¹H NMR (400 MHz, CDCl₃) δ 7.65 (s, 1H), 7.39 (d, J=8.8 Hz, 2H), 7.34 (d, J=8.4 Hz, 2H), 7.27 (d, J=8.4 Hz, 2H), 7.00 (d, J=8.7 Hz, 2H), 6.14 (s, 1H), 4.74 (s, 2H), 4.10 (q, J=6.6 Hz, 2H), 3.57 (s, 3H), 2.50 (brs, 8H), 1.86 (t, J=6.6 Hz, 3H).

LRMS (ESI) m/z: 595.1 [M+H]⁺.

HRMS (ESI) m/z calcd for C₃₀H₃₁ClN₄O₅S [M+H]⁺=595.1782. found: 595.1772.

### Synthesis of compound 95

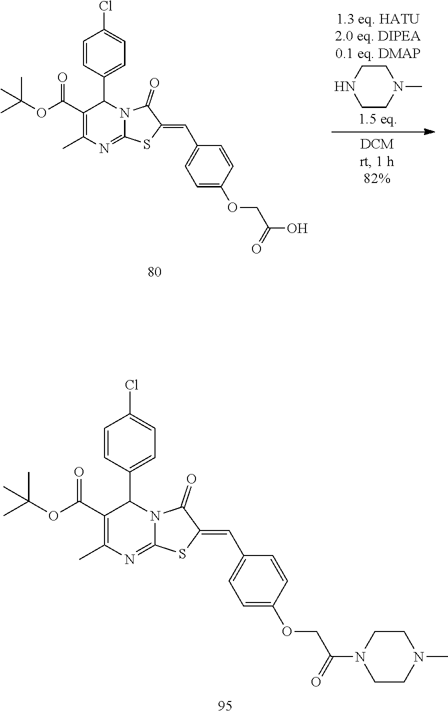

The experimental procedure was similar to that of compound 12. HATU was used to activate the acid group, and the amine compound was 1-methylpiperazine. The crude product was analyzed by column chromatography (methanol:dichloromethane=1:40, R_{f}=0.33, as the eluent). After purification, a yellow solid compound 95 was obtained with a yield of 82%.

¹H NMR (400 MHz, CDCl₃) δ 7.67 (s, 1H), 7.42 (d, J=8.4 Hz, 2H), 7.35 (d, J=8.4 Hz, 2H), 7.28 (d, J=8.4 Hz, 2H), 7.02 (d, J=8.8 Hz, 2H), 6.01 (s, 1H), 4.75 (s, 2H), 3.58-3.55 (m, 4H), 2.49 (s, 3H), 2.43-2.37 (m, 4H), 2.30 (s, 3H), 1.35 (s, 9H).

¹³C NMR (400 MHz, CDCl₃) δ 165.3, 164.7, 164.0, 159.2, 156.6, 155.0, 151.1, 138.8, 138.1, 133.8, 132.5, 131.6, 129.2, 128.1, 126.0, 117.0, 115.0, 109.2, 106.5, 66.2, 54.7, 54.6, 54.6, 54.3, 53.9, 45.3, 44.2, 42.6, 41.4, 39.3, 27.6, 22.2, 17.2, 12.1.

LRMS (ESI) m/z: 623.2 [M+H]⁺.

HRMS (ESI) m/z calcd for C₃₂H₃₅ClN₄O₅S [M+H]⁺=623.2095. found: 623.2087.

### Synthesis of allyl (Z)-5-(4-chlorophenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (96)

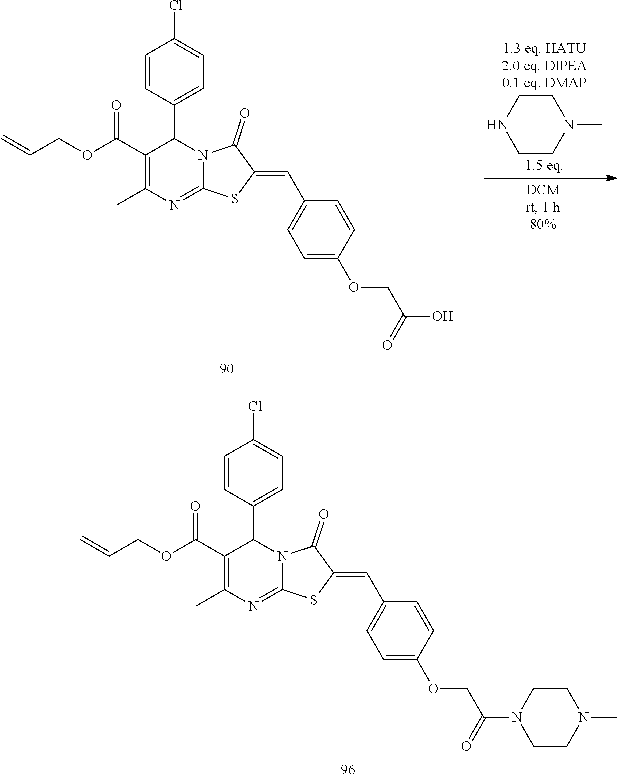

The experimental procedure was similar to that of compound 12. HATU was used to activate the acid group, and the amine compound was 1-methylpiperazine. The crude product was analyzed by column chromatography (methanol:dichloromethane=1:40, R_{f}=0.31, as the eluent). After purification, a yellow solid compound 96 was obtained with a yield of 80%.

¹H NMR (400 MHz, CDCl₃) δ 7.69 (s, 1H), 7.43 (d, J=8.8 Hz, 2H), 7.35 (d, J=8.4 Hz, 2H), 7.28 (d, J=8.4 Hz, 2H), 7.03 (d, J=8.8 Hz, 2H), 6.18 (s, 1H), 5.87-5.74 (m, 1H), 5.21-5.13 (m, 2H), 4.75 (s, 2H), 4.56 (dd, J=5.8, 1.1 Hz, 2H), 3.68-3.57 (m, 4H), 2.96 (d, J=0.5 Hz, 1H), 2.88 (d, J=0.6 Hz, 1H), 2.53 (s, 3H), 2.42 (t, J=5.4 Hz, 4H), 2.31 (s, 3H).

¹³C NMR (600 MHz, CDCl₃) δ 165.6, 165.2, 165.1, 159.7, 156.6, 153.5, 138.3, 135.5, 134.5, 133.4, 132.1, 129.5, 128.8, 128.5, 128.2, 128.2, 126.6, 117.5, 115.5, 108.0, 107.0, 67.3, 66.4, 55.5, 54.9, 54.7, 54.4, 45.9, 45.0, 43.5, 41.9, 40.0, 39.5, 22.9, 12.4.

LRMS (ESI) m/z: 607.2 [M+H]⁺.

HRMS (ESI) m/z calcd for C₃₁H₃₁ClN₄O₅S [M+H]⁺=607.1782. found: 607.1794.

### Synthesis of benzyl (Z)-5-(4-chlorophenyl)-7-methyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (97)

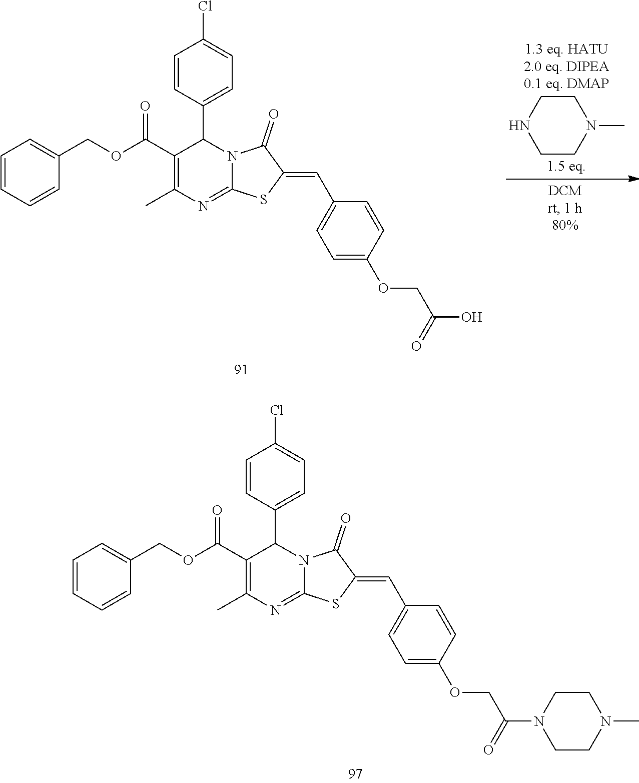

The experimental procedure was similar to that of compound 12. HATU was used to activate the acid group, and the amine compound was 1-methylpiperazine. The crude product was analyzed by column chromatography (methanol:dichloromethane=1:40, R_{f}=0.31, as the eluent). After purification, a yellow solid compound 97 was obtained with a yield of 80%.

¹H NMR (400 MHz, CDCl₃) δ 7.65 (s, 1H), 7.40 (d, J=8.4 Hz, 2H), 7.34-7.26 (m, 4H), 7.26-7.18 (m, 3H), 7.16-7.09 (m, 2H), 7.00 (d, J=8.3 Hz, 2H), 6.14 (s, 1H), 5.20-4.94 (m, 3H), 4.74 (s, 2H), 2.52 (s, 3H).

LRMS (ESI) m/z: 657.2 [M+H]⁺.

HRMS (ESI) m/z calcd for C₃₅H₃₃ClN₄O₅S [M+H]⁺=657.1938. found: 657.1933.

### Synthesis of (Z)-5-(4-chlorophenyl)-7-methyl-6-(3-methylbutanoyl)-2-(4-(2-(4-methylpiperazin-1-yl)-2-oxoethoxy)benzylidene)-5H-thiazolo[3,2-a]pyrimidin-3(2H)-one (98)

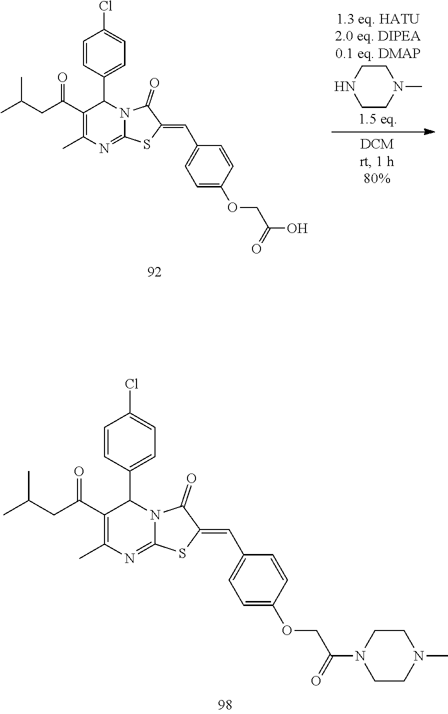

The experimental procedure was similar to compound 12. HATU was used to activate the acid group, and the amine compound was 1-methylpiperazine. The crude product was subjected to column chromatography (methanol:dichloromethane=1:80, R_{f}=0.25, as the eluent). After purification, a yellow solid compound 98 was obtained with a yield of 80%.

¹H NMR (400 MHz, CDCl₃) δ 7.70 (s, 1H), 7.42 (d, J=8.8 Hz, 2H), 7.35 (d, J=8.8 Hz, 2H), 7.28 (d, J=8.8 Hz, 2H), 7.03 (d, J=8.8 Hz, 2H), 6.23 (s, 1H), 4.75 (s, 2H), 3.64-3.55 (m, 4H), 2.43 (s, 3H), 2.41-2.31 (m, 4H), 2.29 (s, 3H), 2.10 (septet, J=6.4 Hz, 1H), 0.79-0.76 (m, 6H).

¹³C NMR (400 MHz, CDCl₃) δ 199.6, 165.4, 165.1, 159.5, 155.2, 148.8, 137.7, 134.5, 133.1, 131.9, 129.5, 129.5, 128.8, 128.8, 126.4, 117.6, 117.3, 115.3, 67.1, 54.8, 54.6, 54.6, 54.3, 51.2, 45.8, 45.8, 44.9, 41.8, 24.7, 24.6, 23.3, 23.3, 22.4, 22.4, 22.3, 22.3.

LRMS (ESI) m/z: 607.3 [M+H]⁺.

HRMS (ESI) m/z calcd for C₃₂H₃₅ClN₄O₄S [M+H]⁺=607.2146. found: 607.2155.

### Synthesis of ethyl (Z)-5-(4-chlorophenyl)-2-(4-(2-(4-cyclohexylpiperazin-1-yl)-2-oxoethoxy)benzylidene)-7-methyl-3-oxo-2,3-dihydro-5H-thiazolo[3,2-a]pyrimidine-6-carboxylate (99)

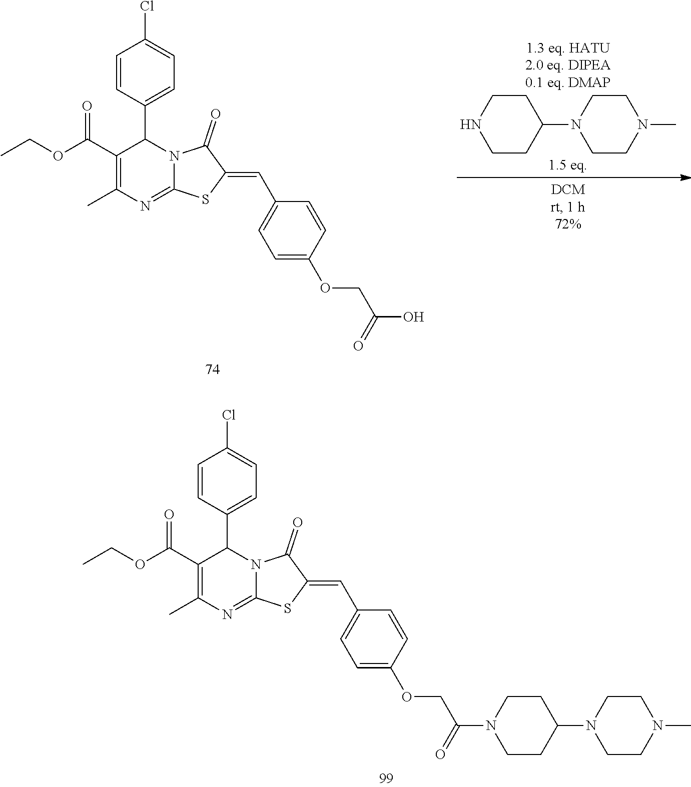 [SMILES: CCOC(=O)C1=C(C)N=c2s/c(=C\c3ccc(OCC(=O)O)cc3)c(=O)n2C1c1ccc(Cl)cc1 ; CN1CCN(C2CCNCC2)CC1 ; CCOC(=O)C1=C(C)N=c2s/c(=C\c3ccc(OCC(=O)N4CCC(N5CCN(C)CC5)CC4)cc3)c(=O)n2C1c1ccc(Cl)cc1]

The experimental procedure was similar to compound 12. HATU was used to activate the acid group, and the amine compound was 1-methyl-4-(piperidin-4-yl)piperazine. The crude product was purified using column chromatography (methanol:dichloromethane=1:40, R_{f}=0.31, as the eluent) to obtain compound 99, a yellow solid, with a yield of 72%.

¹H NMR (400 MHz, CDCl₃) δ 7.69 (s, 1H), 7.42 (d, J=9.2 Hz, 2H), 7.35 (d, J=8.8 Hz, 2H), 7.26 (d, J=8.8 Hz, 2H), 7.02 (d, J=8.9 Hz, 2H), 6.16 (s, 1H), 4.74 (d, J=4.5 Hz, 2H), 4.56 (d, J=13.3 Hz, 1H), 4.10 (qd, J=7.1, 1.3 Hz, 2H), 3.98 (d, J=13.5 Hz, 1H), 3.07 (t, J=12.3 Hz, 1H), 2.62 (d, J=22.0 Hz, 6H), 2.34 (s, 3H), 1.90 (t, J=14.3 Hz, 2H), 1.40 (ddt, J=18.3, 10.9, 5.5 Hz, 2H), 1.19 (t, J=7.1 Hz, 3H).

LRMS (ESI) m/z: 678.2 [M+H]⁺.

HRMS (ESI) m/z calcd for C₃₅H₄₀ClN₅O₅S [M+H]⁺=678.2517. found: 678.2517.

### Synthesis of isopropyl 3-oxopentanoate (101)

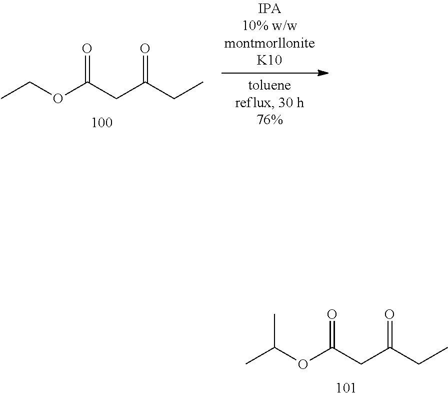

The experimental procedure was similar to that of compound 5. The crude product was purified by column chromatography (ethyl acetate:n-hexane=1:8, R_{f}=0.26, as the eluent) to obtain compound 101, a transparent liquid, with a yield of 76%.

¹H NMR (400 MHz, CDCl₃) δ 5.12-4.98 (m, 1H), 3.40 (s, 2H), 2.56 (q, J=7.3 Hz, 2H), 1.25 (d, J=6.3 Hz, 8H), 1.12-1.01 (m, 3H).

¹³C NMR (400 MHz, CDCl₃) δ 203.0, 166.5, 88.0, 68.0, 48.9, 35.7, 21.2, 7.1. LRMS (ESI) m/z: 159.2 [M+H]⁺.

### Synthesis of 1-(4-(4-chlorophenyl)-6-ethyl-2-thioxo-1,2,3,4-tetrahydropyrimidin-5-yl)-3-methylbutan-1-one (102)

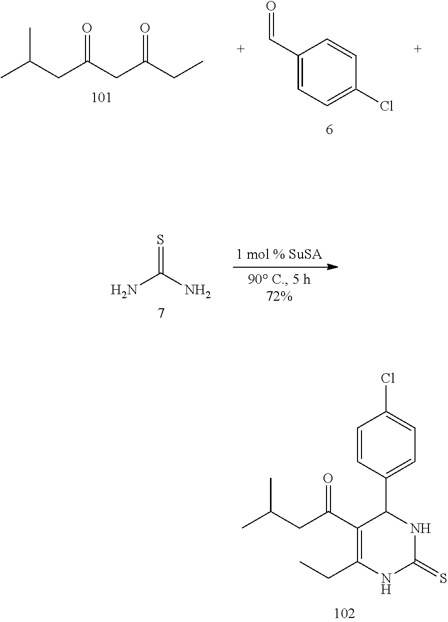

The experimental procedure was similar to that of compound 8. The Biginelli reaction was performed with compound 6 (4-chlorobenzaldehyde). The crude product was subjected to column chromatography (methanol:dichloromethane=1:120, R_{f}=0.3, as the eluent). After purification, a white solid compound 102 was obtained with a yield of 72%.

¹H NMR (400 MHz, CDCl₃) δ 7.74 (s, 1H), 7.30 (d, J=8.8 Hz, 2H), 7.22 (d, J=8.8 Hz, 2H), 5.36 (s, 1H), 4.96 (septet, J=6.4 Hz, 1H), 2.81-2.67 (m, 2H), 1.27-1.18 (m, 6H), 1.07 (s, 3H), 1.06 (s, 3H) ¹³C NMR (400 MHz, CDCl₃) δ 174.2, 164.2, 148.4, 140.9, 134.0, 128.9, 128.9, 128.8, 128.2, 128.1, 102.0, 68.1, 55.2, 55.1, 24.7, 21.8, 21.8, 21.5, 12.5.

LRMS (ESI) m/z: 337.1 [M+H]⁺.

### Synthesis of 5-(4-chlorophenyl)-7-ethyl-6-(3-methylbutanoyl)-5H-thiazolo[3,2-a]pyrimidin-3(2H)-one (103)

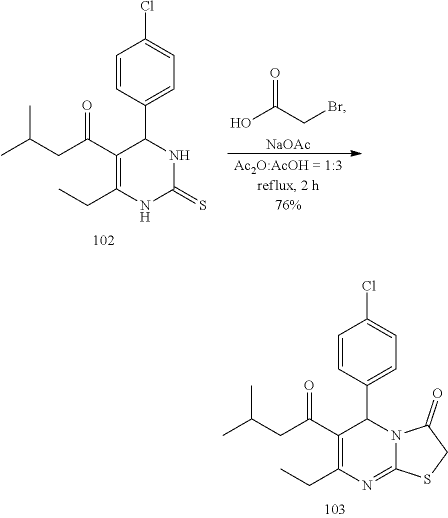 [SMILES: CCC1=C(C(=O)CC(C)C)C(c2ccc(Cl)cc2)NC(=S)N1 ; O=C(O)CBr ; CCC1=C(C(=O)CC(C)C)C(c2ccc(Cl)cc2)N2C(=O)CSC2=N1 ; "NaOAc"]

The experimental procedure was similar to that of compound 2. The crude product was purified by column chromatography (ethyl acetate:n-hexane=1:6, R_{f}=0.2) to obtain reddish brown solid compound 103, with a yield of 76%.

¹H NMR (400 MHz, CDCl₃) δ 7.29-7.23 (m, 4H), 5.98 (s, 1H), 4.92 (septet, J=6.4 Hz, 1H), 3.85-3.76 (m, 2H), 1.07 (d, J=6.4 Hz, 3H), 1.06 (d, J=6.4 Hz, 3H)

¹³C NMR (400 MHz, CDCl₃) δ 173.3, 169.8, 163.7, 159.7, 156.6, 138.1, 133.7, 129.0, 128.3, 128.0, 127.1, 106.5, 67.3, 54.2, 31.8, 27.9, 21.2, 21.0, 20.8, 20.0, 11.8.

LRMS (ESI) m/z: 377.1 [M+H]⁺.

### Synthesis of (Z)-2-(4-((5-(4-chlorophenyl)-7-ethyl-6-(isopropoxycarbonyl)-3-oxo-5H-thiazolo[3,2-a]pyrimidin-2(3H)-ylidene)methyl)phenoxy)acetic acid (104)

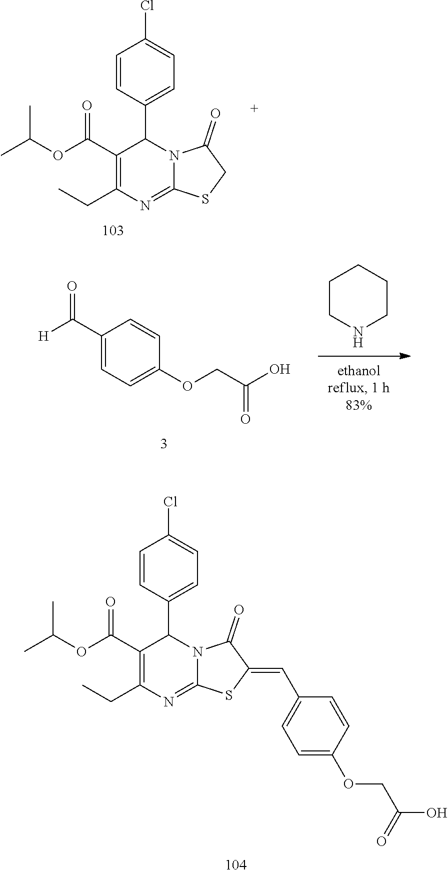 [SMILES: CCC1=C(C(=O)OC(C)C)C(c2ccc(Cl)cc2)N2C(=O)CSC2=N1 ; O=Cc1ccc(OCC(=O)O)cc1 ; C1CCNCC1 ; CCC1=C(C(=O)OC(C)C)C(c2ccc(Cl)cc2)n2c(s/c(=C\c3ccc(OCC(=O)O)cc3)c2=O)=N1]

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 104 was obtained with a yield of 83%.

¹H NMR (400 MHz, CDCl₃) δ 7.67 (s, 1H), 7.39 (d, J=8.7 Hz, 2H), 7.34 (d, J=8.4 Hz, 2H), 7.28 (d, J=8.7 Hz, 2H), 6.98 (d, J=8.4 Hz, 2H), 6.14 (s, 1H), 5.05-4.86 (m, 1H), 4.49 (s, 2H), 1.27-1.14 (m, 8H), 1.06 (s, 3H).

LRMS (ESI) m/z: 541.1 [M+H]⁺.

### Synthesis of isopropyl (Z)-5-(4-chlorophenyl)-7-ethyl-2-(4-(2-(4-methylpiperazin-1-yl)-2-oxoethoxy)benzylidene)-3-oxo-2,3-dihydro-5H-thiazolo[3,2-a]pyrimidine-6-carboxylate (105)

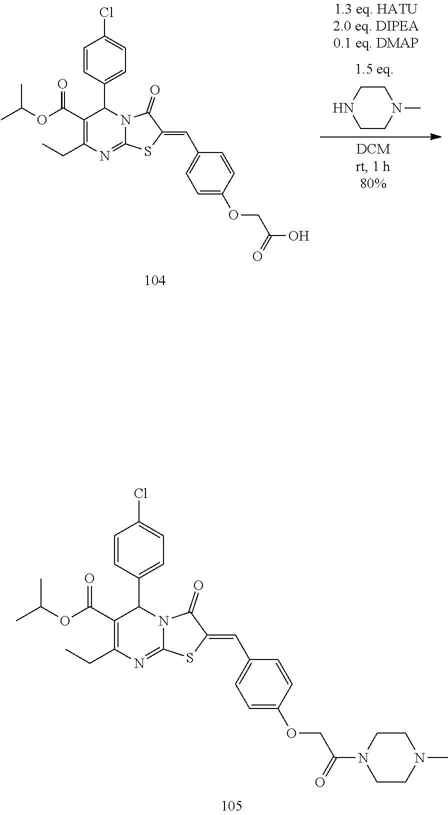

The experimental procedure was similar to that of compound 12. HATU was used to activate the acid group, and the amine compound was 1-methylpiperazine. The crude product was analyzed by column chromatography (methanol:dichloromethane=1:40, R_{f}=0.31, as the eluent). After purification, a yellow solid compound 105 was obtained with a yield of 80%.

¹H NMR (400 MHz, CDCl₃) δ 7.68 (s, 1H), 7.43 (d, J=8.8 Hz, 2H), 7.34 (d, J=8.4 Hz, 2H.), 7.27 (d, J=8.8 Hz, 2H), 7.02 (d, J=8.8 Hz, 2H), 6.14 (s, 1H), 4.97 (septet, J=1.6 Hz, 1H), 4.74 (s, 2H), 3.69-3.61 (m, 4H), 2.96-2.82 (m, 2H), 2.45 (s, 4H), 2.33 (s, 3H), 1.28-1.22 (m, 6H), 1.06 (d, J=3.2 Hz, 3H).

¹³C NMR (400 MHz, CDCl₃) δ 165.5, 165.2, 164.4, 159.5, 157.4, 156.2, 138.5, 134.4, 133.0, 132.9, 132.0, 129.5, 128.6, 126.6, 117.6, 115.4, 115.3, 108.0, 68.0, 67.2, 54.9, 54.7, 54.7, 54.4, 45.9, 45.8, 45.0, 41.9, 28.6, 21.8, 21.8, 21.5, 21.4, 12.4, 12.3.

LRMS (ESI) m/z: 623.2 [M+H]⁺.

HRMS (ESI) m/z calcd for C₃₂H₃₅ClN₄O₅S [M+H]⁺=623.2095. found: 623.2114.

### Synthesis of methyl 2-(4-formylphenoxy)-2-methylpropanoate (106a)

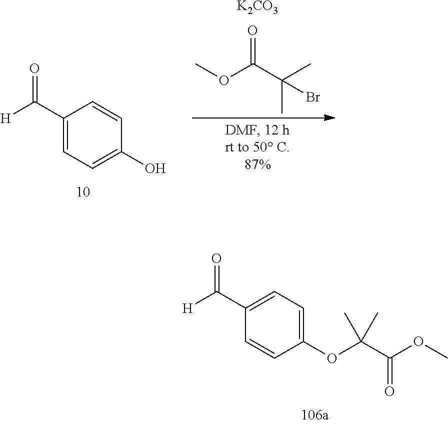

Compound 10 (4-hydroxybenzaldehyde) (0.8 g, 6.1 mmol) was dissolved in 12 ml of dimethylformamide (DMF), and potassium carbonate (4.3 g, 30.2 mmol) was added. After reacting at room temperature for 15 minutes, methyl 2-bromo-2-methylpropanoate (1.2 ml, 8.0 mmol) were dissolved in 5 ml of dimethylformamide and added into the reaction. The reaction was raised from room temperature to 50° C. After 12 hours of reaction, the crude product was purified using column chromatography (ethyl acetate:n-hexane=1:8, R_{f}=0.22, as the eluent), and the product was transparent liquid compound 106a, yield 87%.

¹H NMR (400 MHz, CDCl₃) δ 9.88 (s, 1H), 7.79 (d, J=8.8 Hz, 2H), 6.88 (d, J=8.8 Hz, 2H), 3.76 (s, 3H), 1.67 (s, 6H).

¹³C NMR (600 MHz, CDCl₃) δ 190.6, 174.0, 160.8, 131.5, 130.3, 117.6, 79.4, 52.6, 25.3.

LRMS (ESI) m/z: 223.2 [M+H]⁺.

### Synthesis of 2-(4-formylphenoxy)-2-methylpropanoic acid (106b)

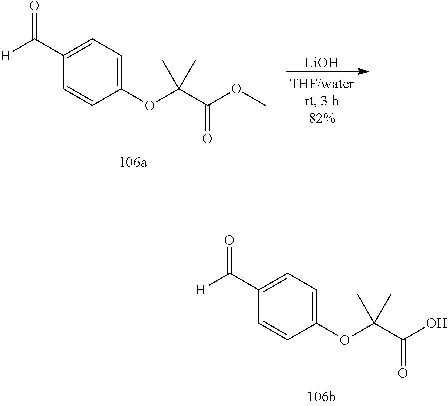 [SMILES: COC(=O)C(C)(C)Oc1ccc(C=O)cc1 ; "LiOH" ; CC(C)(Oc1ccc(C=O)cc1)C(=O)O]

Compound 106a (1.1 ml, 7.8 mmol) was dissolved in THF/Water=1:1, and LiOH (194 mg, 8.1 mmol) was dissolved in water and dropped into the reaction bottle. After the starting material was consumed, the solvent was concentrated and removed. After extraction with DCM, 3.0N HCl was added dropwise to the aqueous layer to adjust the pH value to 2. The crude product was subjected to column chromatography (ethyl acetate:n-hexane=1:4, R_{f}=0.15, as the eluent). After purification, white solid compound 106b was obtained with a yield of 82%.

¹H NMR (400 MHz, CDCl₃) δ 9.88 (s, 1H), 7.79 (d, J=8.8 Hz, 2H), 6.88 (d, J=8.8 Hz, 2H), 3.76 (s, 3H), 1.67 (s, 6H).

¹³C NMR (600 MHz, CDCl₃) δ 190.9, 160.4, 131.6, 118.5, 79.4, 25.3.

LRMS (ESI) m/z: 209.1 [M+H]⁺.

### Synthesis of (Z)-2-(4-((5-(4-chlorophenyl)-6-(isopropoxycarbonyl)-7-methyl-3-oxo-5H-thiazolo[3,2-a]pyrimidin-2(3H)-ylidene)methyl)phenoxy)-2-methylpropanoic acid (107)

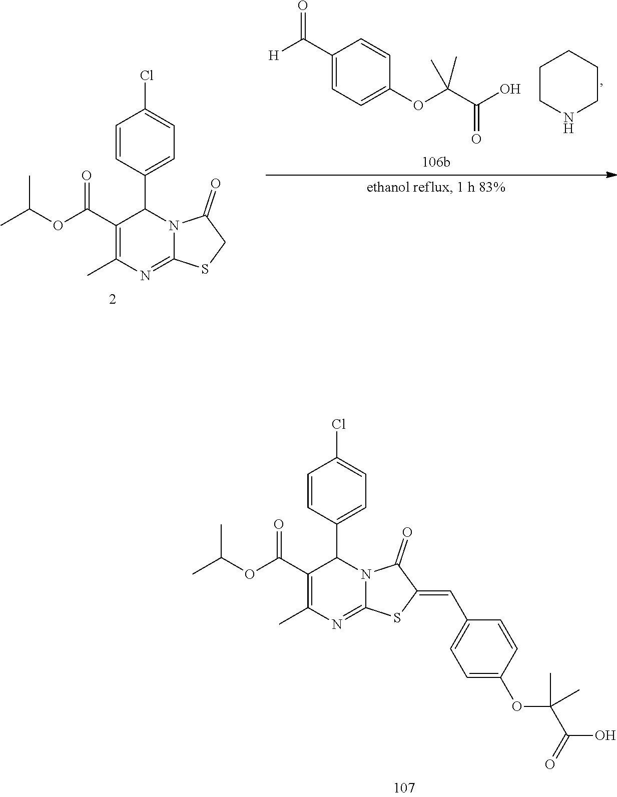

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 107 was obtained with a yield of 83%.

¹H NMR (400 MHz, DMSO-d₆) δ 7.72 (s, 1H), 7.54 (d, J=9.2 Hz, 2H), 7.42 (d, J=8.8 Hz, 3H), 7.33 (d, J=8.8 Hz, 3H), 6.93 (d, J=9.2 Hz, 2H), 6.01 (s, 1H), 4.85 (septet, J=6.4 Hz, 1H), 2.39 (s, 3H), 1.56 (s, 6H), 1.19 (d, J=6.4 Hz, 3H), 0.99 (d, J=6.4 Hz, 3H).

LRMS (ESI) m/z: 555.1 [M+H]⁺.

HRMS (ESI) m/z calcd for C₂₈H₂₇ClN₂O₆S [M+H]⁻=553.1200. found: 553.1195.

### Synthesis of isopropyl (Z)-5-(4-chlorophenyl)-7-methyl-2-(4-((2-methyl-1-(4-methylpiperazin-1-yl)-1-oxopropan-2-yl)oxy)benzylidene)-3-oxo-2,3-dihydro-5H-thiazolo[3,2-a]pyrimidine-6-carboxylate (108)

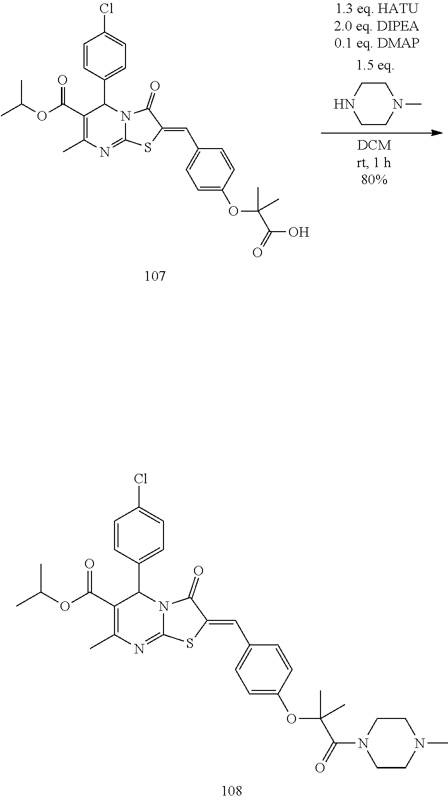

The experimental procedure was similar to that of compound 12. HATU was used to activate the acid group, and the amine compound was 1-methylpiperazine. The crude product was analyzed by column chromatography (methanol:dichloromethane=1:40, R_{f}=0.31, as the eluent). After purification, a yellow solid compound 108 was obtained with a yield of 80%.

¹H NMR (400 MHz, CDCl₃) δ 7.66 (s, 1H), 7.40 (d, J=8.9 Hz, 2H), 7.33 (d, J=8.5 Hz, 2H), 7.25 (dd, J=7.2, 1.4 Hz, 3H), 7.04-6.94 (m, 2H), 6.12 (s, 1H), 5.27 (s, 1H), 4.94 (ddd, J=10.8, 7.1, 5.2 Hz, 1H), 4.73 (s, 2H), 3.58 (dt, J=30.0, 5.1 Hz, 4H), 2.49 (d, J=1.1 Hz, 3H), 2.27 (s, 3H), 1.21 (d, J=6.2 Hz, 4H), 1.03 (d, J=6.2 Hz, 3H).

LRMS (ESI) m/z: 637.2 [M+H]⁺.

HRMS (ESI) m/z calcd for C₃₃H₃₇ClN₄O₅S [M+H]⁺=659.2071. found: 659.2071.

### Synthesis of ethyl (Z)-5-(4-chlorophenyl)-2-(4-(2-(4-ethylpiperazin-1-yl)-2-oxoethoxy)benzylidene)-7-methyl-3-oxo-2,3-dihydro-5H-thiazolo[3,2-a]pyrimidine-6-carboxylate (109)

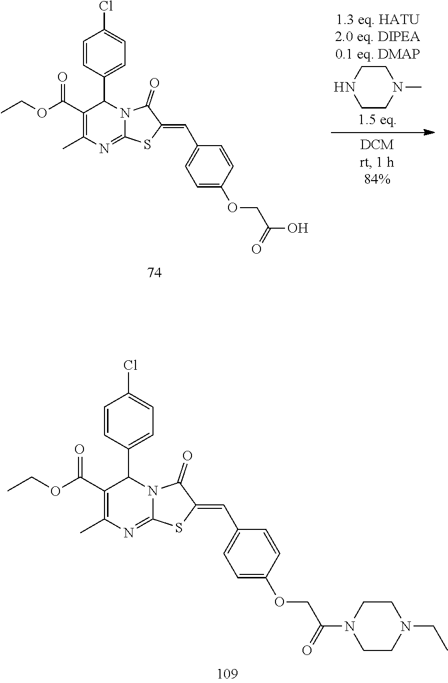

The experimental procedure was similar to compound 12. HATU was used to activate the acid group, and the amine compound was 1-ethylpiperazine. The crude product was subjected to column chromatography (methanol:dichloromethane=1:40, R_{f}=0.31, as the eluent). After purification, a yellow solid compound 109 was obtained with a yield of 84%.

¹H NMR (300 MHz, CDCl₃) δ 7.69 (s, 1H), 7.43 (d, J=8.7 Hz, 2H), 7.35 (d, J=8.7 Hz, 2H), 7.29-7.26 (m, 2H), 7.03 (d, J=8.7 Hz, 2H), 6.16 (s, 1H), 4.75 (s, 1H), 4.11 (q, J=6.9 Hz, 2H), 3.65-3.57 (m, 4H), 2.46-2.37 (m, 2H), 1.20 (t, J=7.2 Hz, 3H), 1.08 (t, J=7.2 Hz, 3H).

LRMS (ESI) m/z: 609.2 [M+H]⁺.

HRMS (ESI) m/z calcd for C₃₂H₃₅ClN₄O₅S [M+H]⁺=609.0999. found: 609.1937.

### Synthesis of ethyl (Z)-5-(4-chlorophenyl)-2-(4-(2-methoxy-2-oxoethoxy)benzylidene)-7-methyl-3-oxo-2,3-dihydro-5H-thiazolo[3,2-a]pyrimidine-6-carboxylate (110)

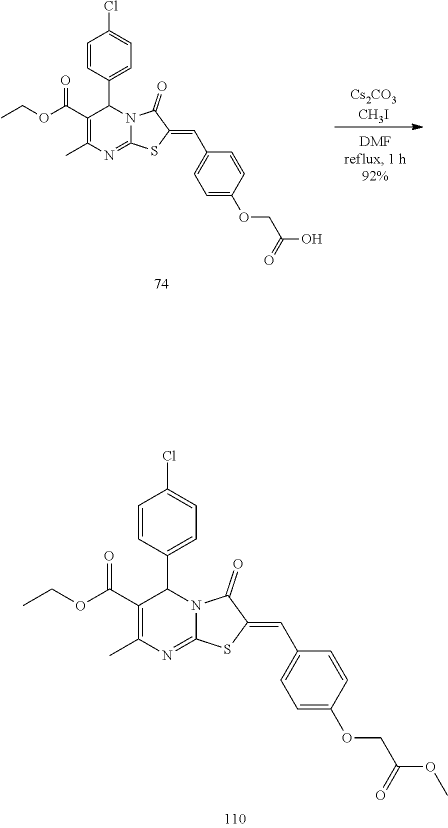 [SMILES: CCOC(=O)C1=C(C)N=c2s/c(=C\c3ccc(OCC(=O)O)cc3)c(=O)n2C1c1ccc(Cl)cc1 ; CCOC(=O)C1=C(C)N=c2s/c(=C\c3ccc(OCC(=O)OC)cc3)c(=O)n2C1c1ccc(Cl)cc1]

The experimental procedure was similar to that of compound 25. After hydrogen extraction with cesium carbonate, methyl iodide was added and heated to reflux for substitution reaction. The crude product was analyzed by column chromatography (methanol:dichloromethane=1:30, R_{f}=0.4). After purification, a yellow solid compound 110 was obtained with a yield of 92%.

¹H NMR (400 MHz, CDCl₃) δ 7.69 (s, 1H), 7.43 (d, J=8.8 Hz, 2H), 7.35 (d, J=8.4 Hz, 2H), 7.28 (d, J=8.4 Hz, 2H), 6.98 (d, J=8.8 Hz, 2H), 6.16 (s, 1H), 4.69 (s, 2H), 4.11 (q, J=7.1 Hz, 3H), 3.82 (s, 3H), 2.52 (d, J=1.0 Hz, 3H), 1.20 (t, J=7.1 Hz, 4H).

LRMS (ESI) m/z: 527.1 [M+H]⁺.

HRMS (ESI) m/z calcd for C₂₆H₂₃ClN₂O₆S [M+H]⁺=527.1044. found: 527.1001.

### Synthesis of 4-((2H-tetrazol-5-yl)methoxy)benzaldehyde (111)

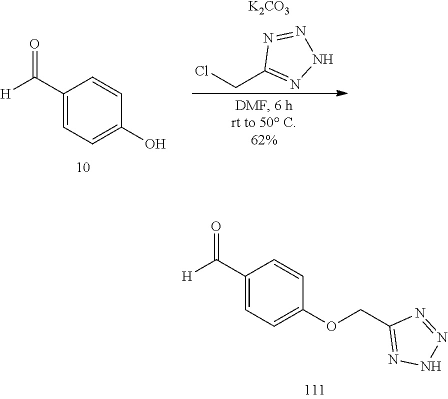 [SMILES: "K2CO3" ; O=Cc1ccc(O)cc1 ; ClCc1nn[nH]n1 ; O=Cc1ccc(OCc2nn[nH]n2)cc1]

The experimental procedure was similar to compound 3. The crude product was purified using column chromatography (methanol:dichloromethane=1:50, R_{f}=0.2, as the eluent) to obtain compound 111, a white solid, with a yield of 62%.

¹H NMR (400 MHz, CDCl₃) δ 7.69 (s, 1H), 7.43 (d, J=8.8 Hz, 2H), 7.35 (d, J=8.4 Hz, 2H), 7.28 (d, J=8.4 Hz, 2H), 6.98 (d, J=8.8 Hz, 2H), 6.16 (s, 1H), 4.69 (s, 2H), 4.11 (q, J=7.1 Hz, 3H), 3.82 (s, 3H), 2.52 (d, J=1.0 Hz, 3H), 1.20 (t, J=7.1 Hz, 4H).

LRMS (ESI) m/z: 205.1 [M+H]⁺.

### Synthesis of 4-(prop-2-yn-1-yloxy)benzaldehyde (112)

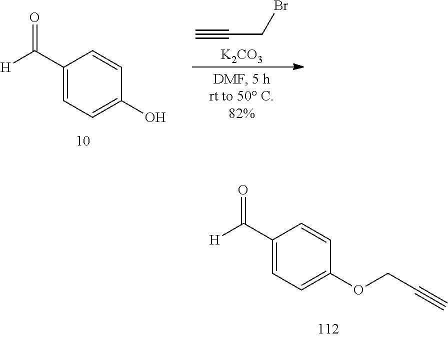

The experimental steps were similar to compound 3. The crude product was purified using column chromatography (methanol:dichloromethane=1:80, R_{f}=0.2, as the eluent) to obtain compound 112, a white solid, with a yield of 82%.

¹H NMR (400 MHz, CDCl 3) δ 9.91 (s, 1H), 7.86 (d, J=8.8 Hz, 2H), 7.10 (d, J=8.8 Hz, 2H), 4.79 (s, 2H), 2.57 (s, 1H).

¹³C NMR (600 MHz, CDCl 3) δ 190.7, 162.3, 131.9, 130.6, 115.2, 76.3, 55.9.

LRMS (ESI) m/z: 161.2 [M+H]⁺.

### Synthesis of 2-(4-formylphenoxy)acetonitrile (113)

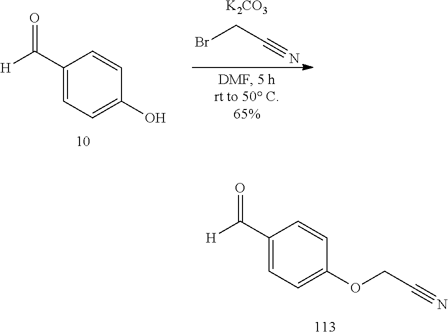

The experimental procedure was similar to compound 3. The crude product was purified using column chromatography (methanol:dichloromethane=1:70, R_{f}=0.2, as the eluent) to obtain compound 113, a white solid, with a yield of 65%.

¹H NMR (400 MHz, CDCl₃) δ 9.95 (s, 1H), 7.92 (d, J=8.8 Hz, 2H), 7.11 (d, J=8.8 Hz, 2H), 4.87 (s, 2H).

¹³C NMR (600 MHz, CDCl₃) δ 190.5, 160.9, 132.0, 131.7, 115.0, 114.3, 53.2.

LRMS (ESI) m/z: 162.1 [M+H]⁺.

### Synthesis of 4-(3-hydroxypropoxy)benzaldehyde (114)

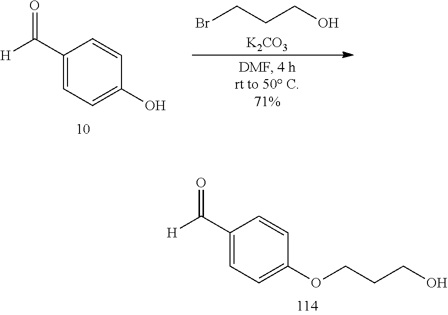

The experimental procedure was similar to compound 3. The crude product was purified using column chromatography (methanol:dichloromethane=1:80, R_{f}=0.2, as the eluent) to obtain compound 114, a white solid, with a yield of 71%.

¹H NMR (400 MHz, CDCl₃) δ 9.95 (s, 1H), 7.92 (d, J=8.8 Hz, 2H), 7.11 (d, J=8.8 Hz, 2H), 4.87 (s, 2H).

¹³C NMR (600 MHz, CDCl₃) δ 190.9, 190.8, 190.8, 163.9, 132.0, 129.8, 114.7, 65.5, 59.5, 31.8.

LRMS (ESI) m/z: 181.1 [M+H]⁺.

### Synthesis of isopropyl (Z)-2-(4-((2H-tetrazol-5-yl)methoxy)benzylidene)-5-(4-chlorophenyl)-7-methyl-3-oxo-2,3-dihydro-5H-thiazolo[3,2-a]pyrimidine-6-carboxylate (115)

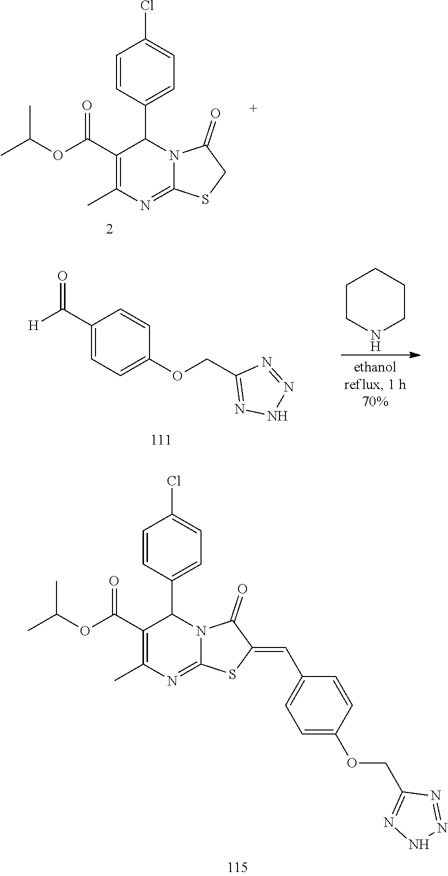 [SMILES: CC1=C(C(=O)OC(C)C)C(c2ccc(Cl)cc2)N2C(=O)CSC2=N1 ; O=Cc1ccc(OCc2nn[nH]n2)cc1 ; C1CCNCC1 ; CC1=C(C(=O)OC(C)C)C(c2ccc(Cl)cc2)n2c(s/c(=C\c3ccc(OCc4nn[nH]n4)cc3)c2=O)=N1]

The experimental procedure was similar to that of compound 1. Piperidine was used for condensation reaction. The crude product was subjected to column chromatography (methanol:dichloromethane=1:50, R_{f}=0.2, as the eluent). After purification, a yellow solid compound 115 was obtained with a yield of 70%.

¹H NMR (400 MHz, CDCl₃) δ 7.63 (s, 1H), 7.38 (d, J=8.8 Hz, 2H), 7.35 (d, J=8.4 Hz, 2H), 7.28 (d, J=8.4 Hz, 2H), 7.03 (d, J=8.8 Hz, 2H), 6.13 (s, 1H), 5.53 (s, 2H), 4.98 (septet, J=6.4 Hz, 1H), 2.53 (s, 3H), 1.24 (d, J=6.4 Hz, 3H), 1.06 (d, J=6.4 Hz, 3H).

LRMS (ESI) m/z: 551.1 [M+H]⁺.

HRMS (ESI) m/z calcd for C₂₆H₂₃ClN₆O₄S [M+H]⁻=549.1112. found: 549.1107.

### Synthesis of isopropyl (Z)-5-(4-chlorophenyl)-7-methyl-3-oxo-2-(4-(prop-2-yn-1-yloxy)benzylidene)-2,3-dihydro-5H-thiazolo[3,2-a]pyrimidine-6-carboxylate (116)

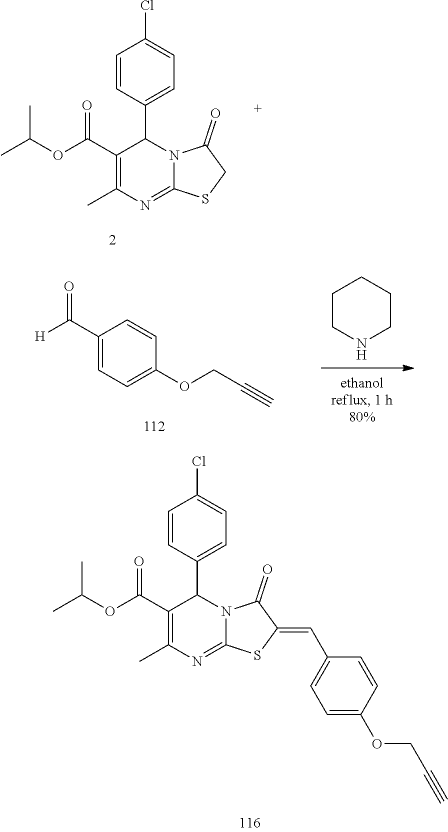 [SMILES: CC1=C(C(=O)OC(C)C)C(c2ccc(Cl)cc2)N2C(=O)CSC2=N1 ; C#CCOc1ccc(C=O)cc1 ; C1CCNCC1 ; C#CCOc1ccc(/C=c2\sc3n(c2=O)C(c2ccc(Cl)cc2)C(C(=O)OC(C)C)=C(C)N=3)cc1]

The experimental procedure was similar to that of compound 1. Piperidine was used for condensation reaction. The crude product was subjected to column chromatography (methanol:dichloromethane=1:80, R_{f}=0.2, as the eluent). After purification, a yellow solid compound 116 was obtained with a yield of 80%.

¹H NMR (400 MHz, CDCl₃) δ 7.70 (s, 1H), 7.44 (d, J=9.2 Hz, 2H), 7.35 (d, J=8.8 Hz, 2H), 7.27 (d, J=8.8 Hz, 2H), 7.06 (d, J=9.2 Hz, 2H), 6.15 (s, 1H), 4.97 (septet, J=6.4 Hz, 1H), 4.75 (s, 2H), 2.56 (t, J=2.4 Hz, 1H), 2.52 (s, 3H), 1.24 (d, J=6.4 Hz, 3H), 1.05 (d, J=6.4 Hz, 3H).

LRMS (ESI) m/z: 507.1 [M+H]⁺.

HRMS (ESI) m/z calcd for C₂₇H₂₃ClN₂O₄S [M+H]⁺=507.1145. found: 507.1139.

### Synthesis of isopropyl (Z)-5-(4-chlorophenyl)-2-(4-(cyanomethoxy)benzylidene)-7-methyl-3-oxo-2,3-dihydro-5H-thiazolo[3,2-a]pyrimidine-6-carboxylate (117)

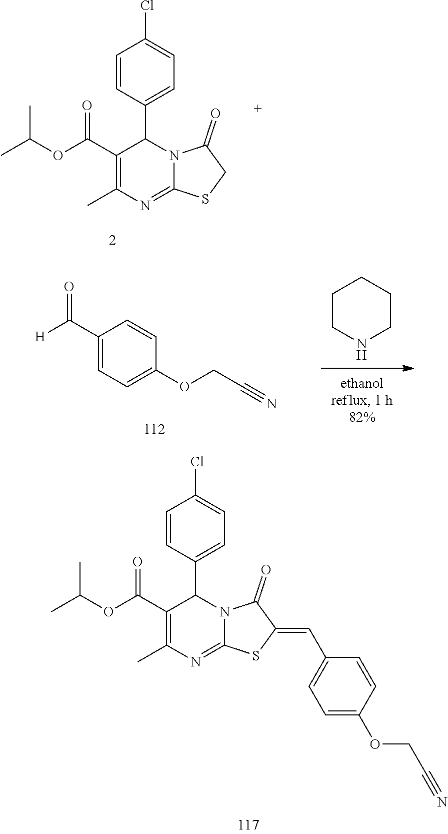 [SMILES: CC1=C(C(=O)OC(C)C)C(c2ccc(Cl)cc2)N2C(=O)CSC2=N1 ; N#CCOc1ccc(C=O)cc1 ; C1CCNCC1 ; CC1=C(C(=O)OC(C)C)C(c2ccc(Cl)cc2)n2c(s/c(=C\c3ccc(OCC#N)cc3)c2=O)=N1]

The experimental procedure was similar to that of compound 1. Piperidine was used for condensation reaction. The crude product was subjected to column chromatography (methanol:dichloromethane=1:70, R_{f}=0.2, as the eluent). After purification, a yellow solid compound 117 was obtained with a yield of 82%.

¹H NMR (400 MHz, CDCl₃) δ 7.70 (s, 1H), 7.44 (d, J=9.2 Hz, 2H), 7.35 (d, J=8.8 Hz, 2H), 7.27 (d, J=8.8 Hz, 2H), 7.06 (d, J=9.2 Hz, 2H), 6.15 (s, 1H), 4.97 (septet, J=6.4 Hz, 1H), 4.75 (s, 2H), 2.56 (t, J=2.4 Hz, 1H), 2.52 (s, 3H), 1.24 (d, J=6.4 Hz, 3H), 1.05 (d, J=6.4 Hz, 3H).

¹³C NMR (600 MHz, CDCl₃) δ 165.2, 164.7, 157.8, 155.8, 152.4, 138.4, 134.6, 132.5, 132.1, 129.6, 128.8, 128.1, 118.9, 115.6, 114.4, 108.9, 68.2, 54.9, 53.3, 22.7, 22.0, 21.6.

LRMS (ESI) m/z: 508.1 [M+H]⁺.

HRMS (ESI) m/z calcd for C₂₆H₂₂ClN₃O₄S [M+H]⁺=508.1098. found: 508.1091.

### Synthesis of isopropyl (Z)-5-(4-chlorophenyl)-2-(4-(3-hydroxypropoxy)benzylidene)-7-methyl-3-oxo-2,3-dihydro-5H-thiazolo[3,2-a]pyrimidine-6-carboxylate (118)

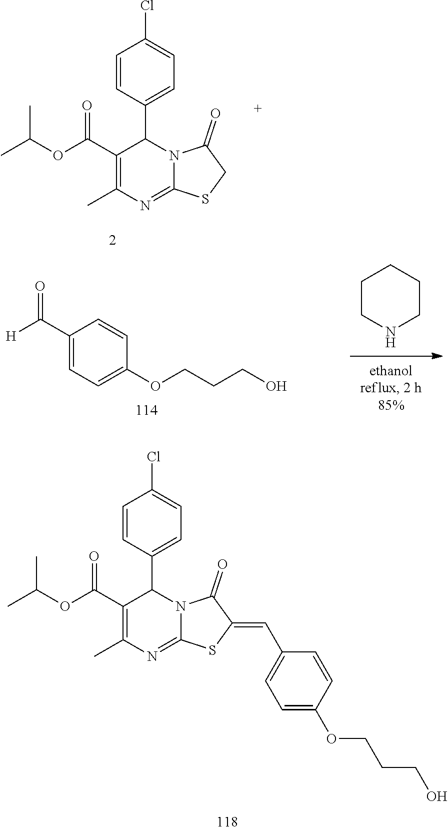 [SMILES: CC1=C(C(=O)OC(C)C)C(c2ccc(Cl)cc2)N2C(=O)CSC2=N1 ; O=Cc1ccc(OCCCO)cc1 ; C1CCNCC1 ; CC1=C(C(=O)OC(C)C)C(c2ccc(Cl)cc2)n2c(s/c(=C\c3ccc(OCCCO)cc3)c2=O)=N1]

The experimental procedure was similar to that of compound 1. Piperidine was used for condensation reaction. The crude product was subjected to column chromatography (methanol:dichloromethane=1:50, R_{f}=0.23, as the eluent). After purification, a yellow solid compound 118 was obtained with a yield of 85%.

¹H NMR (400 MHz, CDCl₃) δ 7.70 (s, 1H), 7.44 (d, J=9.2 Hz, 2H), 7.35 (d, J=8.8 Hz, 2H), 7.27 (d, J=8.8 Hz, 2H), 7.06 (d, J=9.2 Hz, 2H), 6.15 (s, 1H), 4.97 (septet, J=6.4 Hz, 1H), 4.75 (s, 2H), 2.56 (t, J=2.4 Hz, 1H), 2.52 (s, 3H), 1.24 (d, J=6.4 Hz, 3H), 1.05 (d, J=6.4 Hz, 3H).

LRMS (ESI) m/z: 527.1 [M+H]⁺.

HRMS (ESI) m/z calcd for C₂₇H₂₇ClN₂O₅S [M+H]⁺=549.1227. found: 549.1227.

### Synthesis of isopropyl (Z)-2-(4-((1H-1,2,3-triazol-5-yl)methoxy)benzylidene)-5-(4-chlorophenyl)-7-methyl-3-oxo-2,3-dihydro-5H-thiazolo[3,2-a]pyrimidine-6-carboxylate (120)

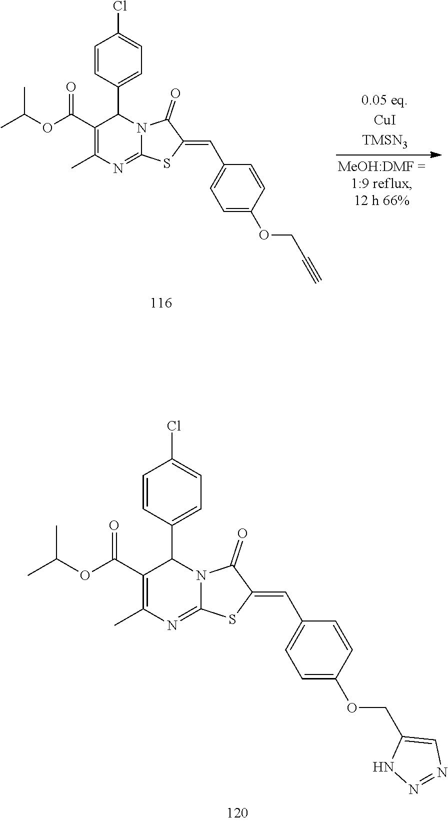

Compound 116 (360 mg, 0.7 mmol) and CuI (6.1 mg, 0.03 mmol) of 0.05 equivalents of Cu (I) were added into TMSN₃ (0.14 ml, 1.05 mmol) to perform click reaction under catalysis to obtain 1,2,3-triazole structural compound 120. The crude product was purified using column chromatography (methanol:dichloromethane=1:50, R_{f}=0.2, as the eluent) to obtain compound 120, a yellow solid, with a yield of 66%.

¹H NMR (400 MHz, CDCl₃) δ 7.70 (s, 1H), 7.44 (d, J=9.2 Hz, 2H), 7.35 (d, J=8.8 Hz, 2H), 7.27 (d, J=8.8 Hz, 2H), 7.06 (d, J=9.2 Hz, 2H), 6.15 (s, 1H), 4.97 (septet, J=6.4 Hz, 1H), 4.75 (s, 2H), 2.56 (t, J=2.4 Hz, 1H), 2.52 (s, 3H), 1.24 (d, J=6.4 Hz, 3H), 1.05 (d, J=6.4 Hz, 3H).

LRMS (ESI) m/z: 550.1 [M+H]⁺.

HRMS (ESI) m/z calcd for C₂₇H₂₄ClN₅O₄S [M+H]⁺=550.1316. found: 550.1315.

### Synthesis of isopropyl (Z)-5-(4-chlorophenyl)-7-methyl-3-oxo-2-(4-(2-oxo-2-((trifluoromethyl)sulfonamido)ethoxy)benzylidene)-2,3-dihydro-5H-thiazolo[3,2-a]pyrimidine-6-carboxylate (121)

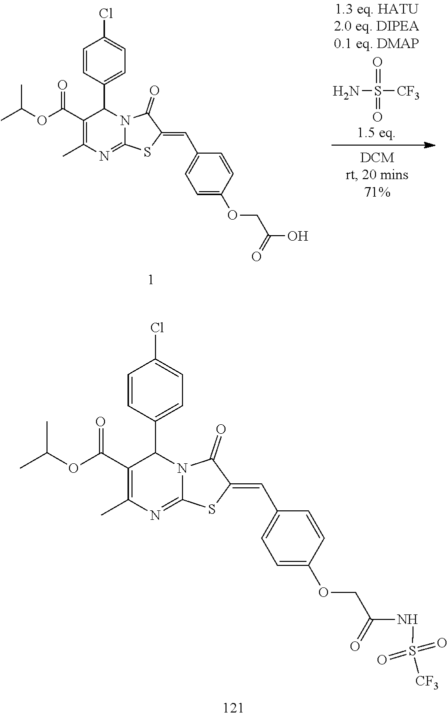 [SMILES: CC1=C(C(=O)OC(C)C)C(c2ccc(Cl)cc2)n2c(s/c(=C\c3ccc(OCC(=O)O)cc3)c2=O)=N1 ; NS(=O)(=O)C(F)(F)F ; CC1=C(C(=O)OC(C)C)C(c2ccc(Cl)cc2)n2c(s/c(=C\c3ccc(OCC(=O)NS(=O)(=O)C(F)(F)F)cc3)c2=O)=N1]

The experimental procedure was similar to that of compound 12. HATU was used to activate the acid group. The crude product was purified by column chromatography (methanol:dichloromethane=1:40, R_{f}=0.31) to obtain compound 121, a yellow solid with yield of 71%.

¹H NMR (400 MHz, CDCl₃) δ 8.07 (s, 1H), 7.69 (s, 1H), 7.42 (d, J=9.2 Hz, 2H), 7.35 (d, J=8.8 Hz, 2H), 7.28 (d, J=8.8 Hz, 2H), 6.98 (d, J=9.2 Hz, 2H), 6.15 (s, 1H), 4.97 (septet, J=6.4 Hz, 1H), 4.72 (s, 2H), 2.52 (s, 3H), 1.24 (d, J=6.4 Hz, 3H), 1.05 (d, J=6.4 Hz, 3H).

LRMS (ESI) m/z: 658.1 [M+H]⁺.

HRMS (ESI−) m/z calcd for C₂₇H₂₃ClF₃N₃O₇S₂ [M−H]⁻=656.0540. found: 656.0534.

### Synthesis of 2,2′-((1E,1′E)-(((1S,2S)-1,2-Diphenylethane-1,2-diyl)bis(azanylylidene))bis(methanylylidene))diphenol (123)

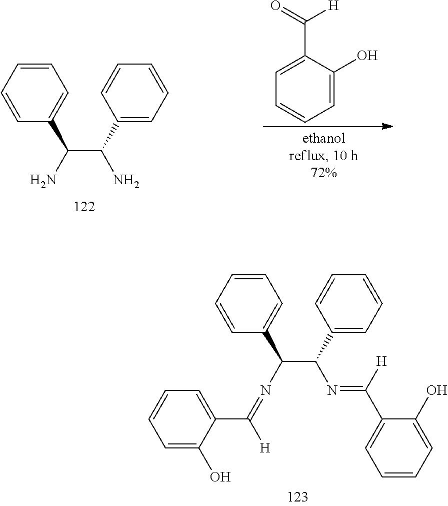 [SMILES: N[C@@H](c1ccccc1)[C@@H](N)c1ccccc1 ; O=Cc1ccccc1O ; Oc1ccccc1C=N[C@@H](c1ccccc1)[C@@H](/N=C/c1ccccc1O)c1ccccc1]

Compound 122 (0.5 g, 2.5 mmol) and salicylaldehyde (0.61 g, 5.0 mmol) were dissolved in 15 ml of ethanol, heated to reflux for 10 hours, and the crude product was subjected to column chromatography (ethyl acetate:n-hexane=1:9, R_{f}=0.14, as the eluent). After purification, a white solid compound 123 was obtained with a yield of 72%.

¹H NMR (400 MHz, CDCl₃) δ 8.07 (s, 1H), 7.69 (s, 1H), 7.42 (d, J=9.2 Hz, 2H), 7.35 (d, J=8.8 Hz, 2H), 7.28 (d, J=8.8 Hz, 2H), 6.98 (d, J=9.2 Hz, 2H), 6.15 (s, 1H), 4.97 (septet, J=6.4 Hz, 1H), 4.72 (s, 2H), 2.52 (s, 3H), 1.24 (d, J=6.4 Hz, 3H), 1.05 (d, J=6.4 Hz, 3H).

LRMS (ESI) m/z: 421.2 [M+H]⁺.

### Synthesis of 2,2′-[[(1S,2S)-1,2-Diphenyl-1,2-ethanediyl]bis(iminomethylene)]bis[phenol](124)

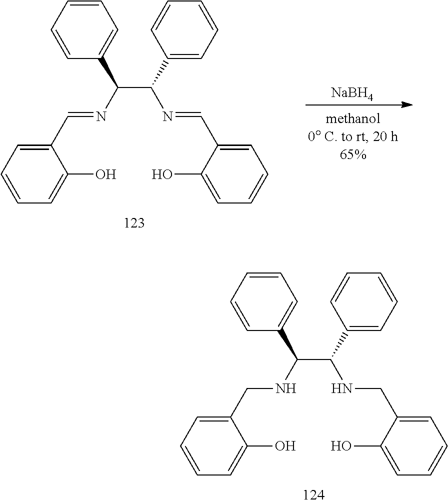 [SMILES: Oc1ccccc1/C=N/[C@@H](c1ccccc1)[C@@H](/N=C/c1ccccc1O)c1ccccc1 ; "NaBH4" ; Oc1ccccc1CN[C@@H](c1ccccc1)[C@@H](NCc1ccccc1O)c1ccccc1]

Compound 123 (100 mg, 0.23 mmol) was dissolved in methanol, NaBH₄ (45 mg, 1.19 mmol) was slowly added in an ice bath, and the reaction was carried out for 20 hours. The crude product was analyzed by column chromatography (ethyl acetate:n-hexane=1:5, R_{f}=0.21, as the eluent). After purification, a white solid compound 124 was obtained with a yield of 65%.

¹H NMR (400 MHz, CDCl₃) δ 7.27-6.81 (m, 18H), 4.04 (s, 2H), 3.87 (d, J=13.2 Hz, 2H), 3.61 (d, J=13.2 Hz, 2H).

¹³C NMR (400 MHz, CDCl₃) δ 157.4, 137.3, 137.3, 128.6, 128.4, 128.1, 127.7, 127.6, 122.2, 122.1, 119.0, 116.0, 66.2, 49.6.

LRMS (ESI) m/z: 425.1 [M+H]⁺.

### Synthesis of 2,2′-[[(1S,2S)-1,2-Diphenyl-1,2-ethanediyl]bis(iminomethylene)]bis[phenol](125)

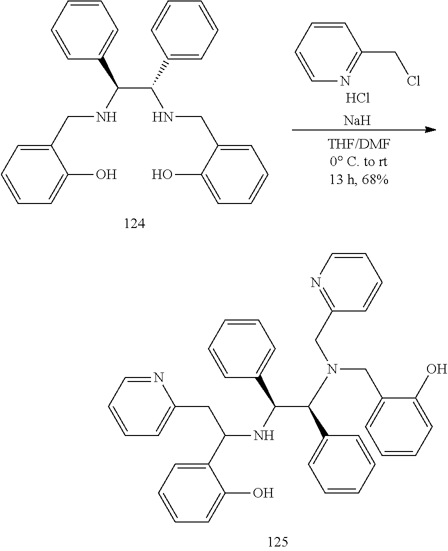

Compound 124 (0.1 g, 0.25 mmol) was dissolved in a solution of THF/DMF=1:1, and 60% w/w NaH (48 mg, 1 mmol) was slowly added under an ice bath. After reacting for about 20 minutes, 2-chloromethyl-pyridine (82 mg, 0.5 mmol) was added, followed by reaction for 13 hours. The crude product was analyzed by column chromatography (ethyl acetate:n-hexane=1:9, R_{f}=0.14, as the eluent). After purification, a brown oily compound 125 was obtained with a yield of 68%.

¹H NMR (400 MHz, CDCl₃) δ 8.67-8.65 (m, 2H), 7.88-7.67 (m, 3H), 7.46-7.34 (m, 9H), 7.11-6.90 (m, 3H), 5.15 (s, 4H), 4.25 (s, 4H), 3.83 (s, 2H), 3.12 (s, 2H), 3.05 (s, 2H).

¹³C NMR (400 MHz, CDCl₃) δ 155.90, 155.51, 148.18, 137.55, 136.34, 130.00, 128.17, 127.44, 126.78, 125.45, 121.95, 120.55, 120.24, 110.89, 69.33, 66.33, 45.92.

LRMS (ESI) m/z: 607.1 [M+H]⁺.

### Synthesis of 2,2′-[[(1S,2S)-1,2-Diphenyl-1,2-ethanediyl]bis(iminomethylene)]bis[phenol](130)

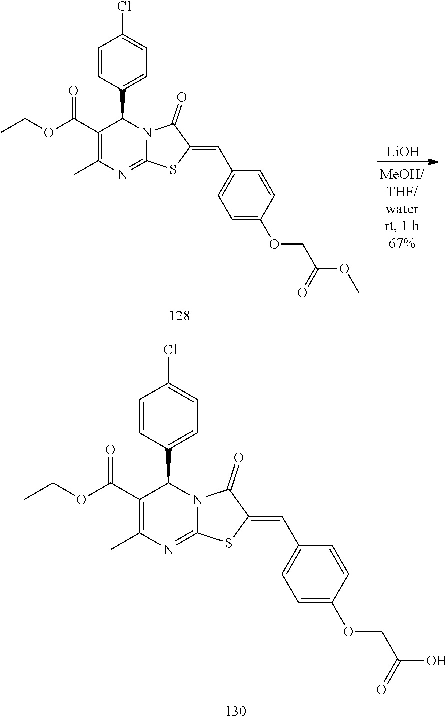

The experimental procedure was similar to that of compound 106b. The crude product was subjected to column chromatography using a gradient elution method (gradient from methanol:ammonia:methylene chloride=1:0.1:50 to methanol:ammonia:methylene chloride=1:0.1:20, R_{f}=0.15, as the eluent), and after purification, a yellow solid compound 130 was obtained with a yield of 67%.

¹H NMR (400 MHz, DMSO-d₆) δ 9.24 (d, J=2.0 Hz, 1H), 7.76 (s, 2H), 7.57 (d, J=9.0 Hz, 2H), 7.41 (dd, J=11.0, 8.5 Hz, 4H), 7.33 (d, J=8.6 Hz, 3H), 7.08 (d, J=8.9 Hz, 2H), 6.03 (d, J=1.2 Hz, 1H), 5.14 (s, 1H), 4.78 (s, 3H), 3.98 (q, J=7.1 Hz, 3H), 2.39 (dd, J=3.6, 1.1 Hz, 4H), 2.30-2.21 (m, 3H), 1.10-1.05 (m, 4H).

LRMS (ESI) m/z: 531.1 [M+H]⁺.

HRMS (ESI) m/z calcd for C₂₅H₂₁ClN₂O₆S [M+H]⁻=511.0731. found: 511.0734.

### Synthesis of 2,2′-[[(1S,2S)-1,2-Diphenyl-1,2-ethanediyl]bis(iminomethylene)]bis[phenol](131)

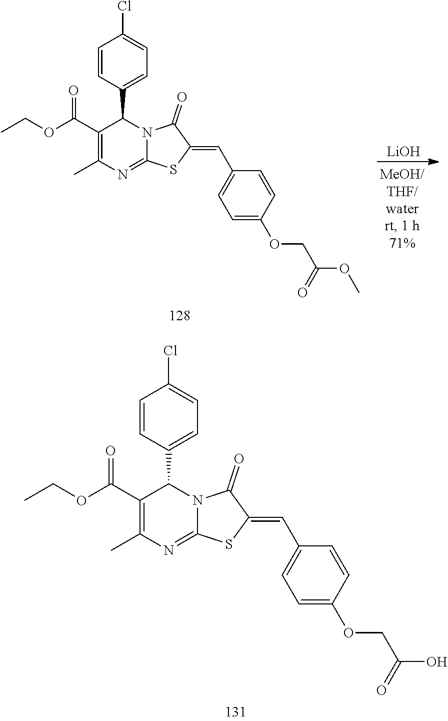

The experimental procedure was similar to that of compound 106b. The crude product was subjected to column chromatography using a gradient elution method (gradient from methanol:ammonia:methylene chloride=1:0.1:50 to methanol:ammonia:methylene chloride=1:0.1:20, R_{f}=0.15, as the eluent), and after purification, a yellow solid compound 131 was obtained with a yield of 71%.

¹H NMR (400 MHz, DMSO-d₆) δ 9.25 (s, 1H), 7.39 (d, J=8.8 Hz, 3H), 7.24 (d, J=8.4 Hz, 2H), 7.04 (d, J=8.4 Hz, 2H), 6.73 (d, J=8.8 Hz, 2H), 6.04 (s, 1H), 5.14 (s, 1H), 3.98 (q, J=7.2 Hz, 4H), 2.24 (s, 3H), 1.10 (t, J=7.2 Hz, 3H).

LRMS (ESI) m/z: 531.1 [M+H]⁺.

HRMS (ESI) m/z calcd for C₂₅H₂₁ClN₂O₆S [M+H]⁻=511.0731. found: 511.0724.

### Synthesis of potassium (Z)-2-(4-((5-(4-chlorophenyl)-6-(isopropoxycarbonyl)-7-methyl-3-oxo-5H-thiazolo[3,2-a]pyrimidin-2(3H)-ylidene)methyl)phenoxy)acetate (132)

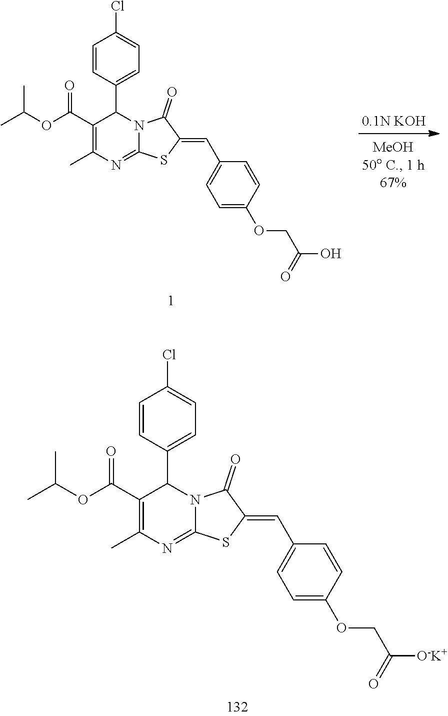

The experimental procedure was similar to that of compound 11. After purification by crystallization from methanol, compound 132 was obtained as a yellow solid with a yield of 67%.

¹H NMR (300 MHz, DMSO-d₆) δ 7.71 (s, 1H), 7.49 (d, J=9.0 Hz, 2H), 7.41 (d, J=8.7 Hz, 2H), 7.32 (d, J=8.47 Hz, 2H), 6.91 (d, J=9.0 Hz, 2H), 6.00 (s, 1H), 4.87-4.823 (m, 1H), 4.16 (s, 2H), 2.38 (s, 3H), 1.19 (d, J=6.3 Hz, 3H), 0.99 (d, J=6.3 Hz, 3H).

LRMS (ESI) m/z: 527.1 [M+H]⁺.

### Synthesis of isopropyl (Z)-5-(4-chlorophenyl)-2-(4-(2-(4-ethylpiperazin-1-yl)-2-oxoethoxy)benzylidene)-7-methyl-3-oxo-2,3-dihydro-5H-thiazolo[3,2-a]pyrimidine-6-carboxylate maleate (133)

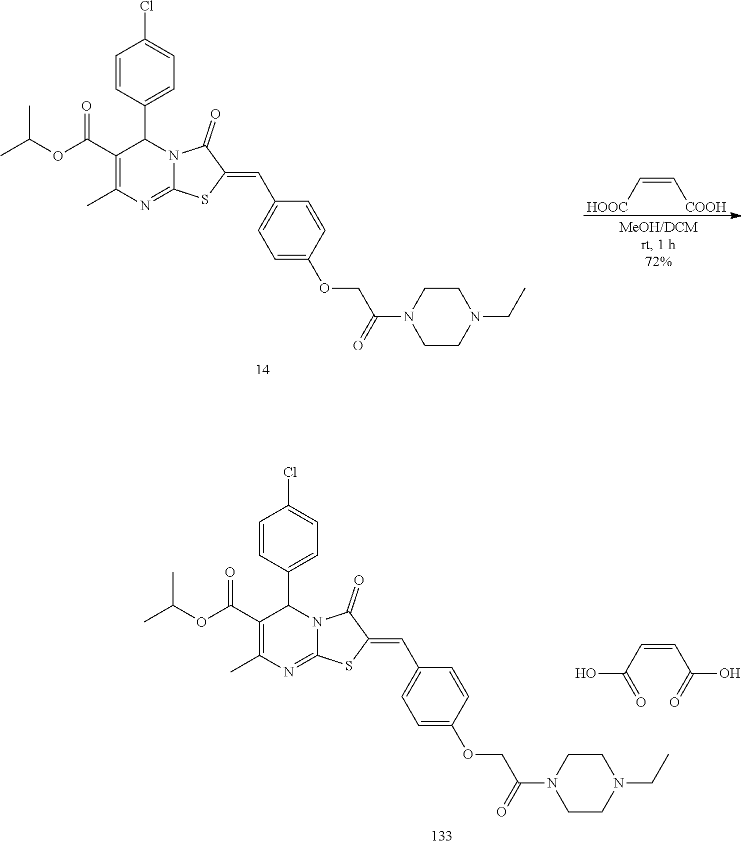

Dissolve compound 14 in DCM, add a little methanol to aid dissolution, add a corresponding amount of maleic acid and react at room temperature for 1 hour to form maleate compound 133 (maleic salt). The crude product was recrystallized from methanol and purified to obtain compound 133 as a yellow solid with a yield of 72%.

¹H NMR (400 MHz, CDCl₃) δ 7.70 (s, 1H), 7.44 (d, J=8.8 Hz, 2H), 7.35 (d, J=8.4 Hz, 2H), 7.28 (d, J=8.4 Hz, 2H), 7.03 (d, J=8.8 Hz, 2H), 6.39 (s, 2H), 6.15 (s, 1H), 4.97 (septet, J=6.4 Hz, 1H), 4.80 (s, 2H), 3.13 (q, J=7.3 Hz, 2H), 2.52 (s, 3H), 1.37 (t, J=7.3 Hz, 3H), 1.24 (d, J=6.4 Hz, 3H), 1.05 (d, J=6.4 Hz, 3H).

LRMS (ESI) m/z: 623.2 [M+H]⁺.

### Synthesis of isopropyl (Z)-5-(4-chlorophenyl)-2-(4-(2-(4-(dimethylamino)piperidin-1-yl)-2-oxoethoxy)benzylidene)-7-methyl-3-oxo-2,3-dihydro-5H-thiazolo[3,2-a]pyrimidine-6-carboxylate maleate (134)

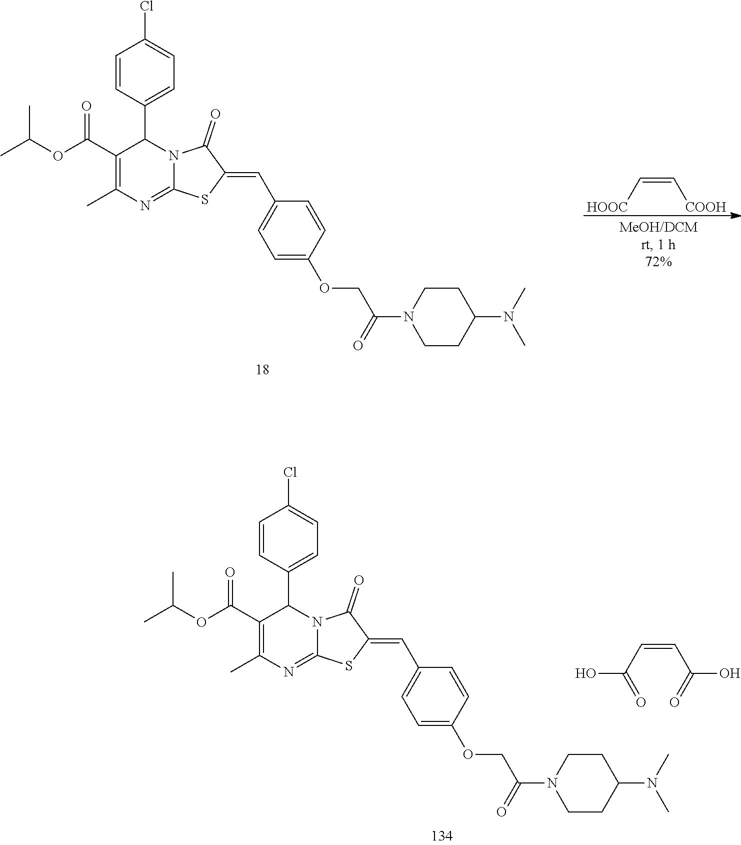

The experimental procedure was similar to that of compound 133. The crude product was recrystallized from methanol and purified to obtain compound 134 as a yellow solid with a yield of 72%.

¹H NMR (400 MHz, DMSO-d₆) δ 7.75 (s, 1H), 7.56 (d, J=8.8 Hz, 2H), 7.43 (d, J=8.4 Hz, 2H), 7.33 (d, J=8.4 Hz, 2H), 7.08 (d, J=8.8 Hz, 2H), 6.08 (s, 2H), 6.01 (s, 1H), 4.97 (s, 2H), 4.85 (septet, J=6.4 Hz, 1H), 4.46 (d, J=13.2 Hz, 1H), 3.95 (d, J=13.2 Hz, 1H), 3.06 (d, J=13.2 Hz, 2H), 2.75 (s, 7H), 2.59 (t, J=12.4 Hz, 2H), 2.39 (s, 3H), 2.01 (d, J=11.9 Hz, 2H), 1.63 (d, J=12.6 Hz, 1H), 1.43 (d, J=12.4 Hz, 2H), 1.19 (d, J=6.4 Hz, 3H), 0.99 (d, J=6.4 Hz, 3H).

LRMS (ESI) m/z: 637.1 [M+H]⁺.

### Synthesis of isopropyl (Z)-5-(4-chlorophenyl)-7-methyl-2-(4-(2-(4-(4-methylpiperazin-1-yl)piperidin-1-yl)-2-oxoethoxy)benzylidene)-3-oxo-2,3-dihydro-5H-thiazolo[3,2-a]pyrimidine-6-carboxylate maleate (135)

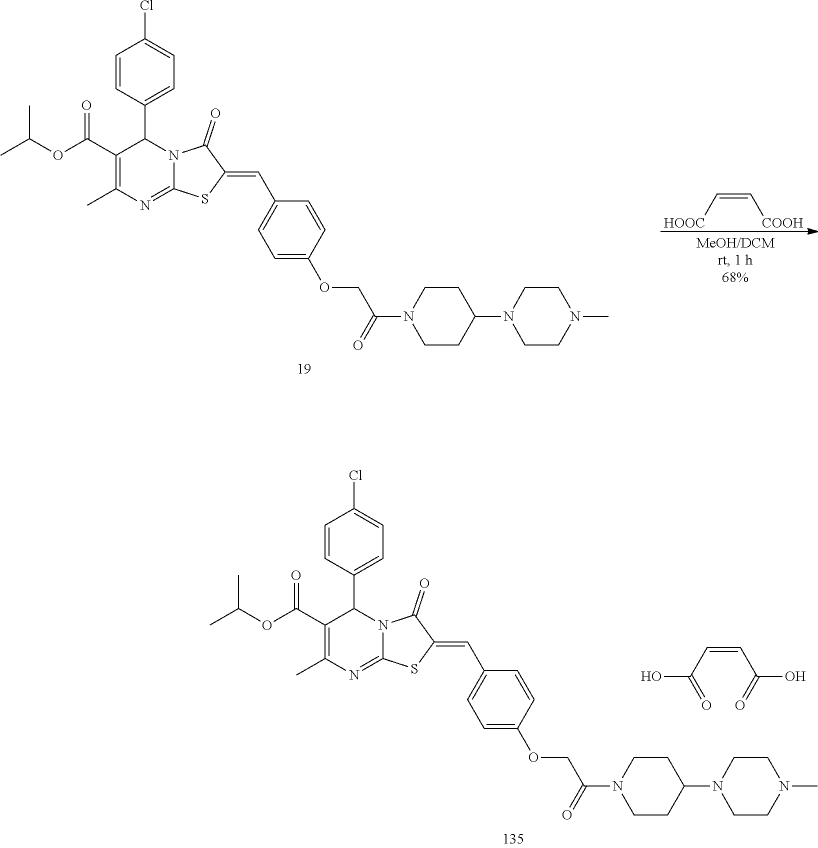

The experimental procedure was similar to that of compound 133. The crude product was recrystallized from methanol and purified to obtain compound 135 as a yellow solid with a yield of 68%.

¹H NMR (400 MHz, DMSO-d₆) δ 7.75 (s, 1H), 7.56 (d, J=8.8 Hz, 2H), 7.42 (d, J=8.4 Hz, 2H), 7.33 (d, J=8.4 Hz, 2H), 7.07 (d, J=8.8 Hz, 2H), 6.19 (s, 2H), 6.01 (s, 1H), 4.96 (s, 2H), 4.90-4.79 (m, 1H), 4.34 (d, J=12.0 Hz, 1H), 3.86 (d, J=12.0 Hz, 1H), 3.02 (t, J=12.0 Hz, 6H), 2.71 (s, 3H), 2.39 (s, 3H), 1.84 (s, 2H), 1.50-1.23 (m, 3H), 1.19 (d, J=6.4 Hz, 3H), 0.99 (d, J=6.4 Hz, 3H).

LRMS (ESI) m/z: 692.1 [M+H]⁺.

The in vitro validation and cell validation of the compounds of the present invention were performed as follows.

The in vitro validation was performed by measuring KIF2C kinesin activity using malachite green-based phosphate colorimetric assay (BioVision). In short, 400 nM recombinant kinesin protein was incubated with 300 nM microtubules in a 200 μL reaction buffer (1 μM paclitaxel, 75 mM KCl, 1 mM MgCl2, and 600 nM ATP) for 30 min at 37° C. The microtubules from porcine brain tubulin (Cytoskeleton) or purified from HEK293 cells in Free-style medium (293Fs) were dissolved in the BRB80 buffer (80 mM PIPES pH 6.8, 1 mM EGTA, 1 mM MgCl2, 1 mM GTP, 1 mM DTT, and 10% DMSO) at 37° C. for 2 hr. Phosphates released from kinesin-mediated ATP hydrolysis was measured by adding 30 μL of the phosphate colorimetric reagent (BioVision), incubated for 10 min, and then measured with absorbance at 650 nm. The EC50 indicated the concentration (μM) of indicated compounds that inhibits 50% of KIF2C activity. The cell validation was performed by measuring cytotoxicity in MDA-MB-231 cells. The cells were cultured in a 96-well plate at a confluence of 2000 cells/well with indicates compounds for specific days. When the time was up, the culture medium in the plate was aspirated and replaced with a prepared CellTiter-Glo reagent (Promega)/DMEM mixture (1:1 mix of CellTiter-Glo reagent and serum-supplemented DMEM). After incubation for 10 min, the luminescence was measured with an ELISA plate reader to determine the cell viability. The IC50 value indicates the concentration (μM) of indicated compounds with 50% of cytotoxicity.

The results are shown in the following Table 1.

TABLE 1

| | | In vitro validation | Cell validation |
|---|---|---|---|
| | | (EC50 >100 - | (IC50 >50 - |
| | | <100, >50 + | <50, >20 + |
| Compound | | <50, >20 ++ | <20, >10 ++ |
| name/ | | <20, >10 +++ | <10, >5 +++ |
| number | Compound structure | <10 ++++) | <5 ++++) |
| 1S0 (1) | 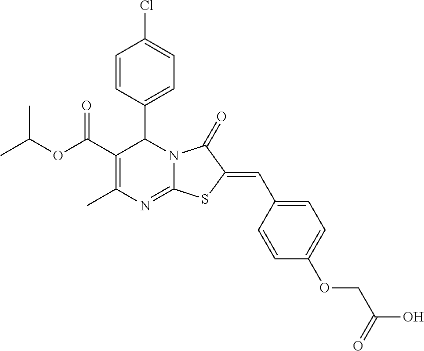 [SMILES: CC1=C(C(=O)OC(C)C)C(c2ccc(Cl)cc2)n2c(s/c(=C\c3ccc(OCC(=O)O)cc3)c2=O)=N1] | + + + + | − |
| 2S0 (22) | 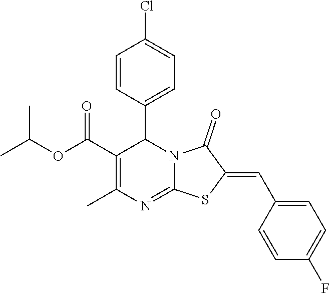 [SMILES: CC1=C(C(=O)OC(C)C)C(c2ccc(Cl)cc2)n2c(s/c(=C\c3ccc(F)cc3)c2=O)=N1] | − | − |
| 3S0 (23) | 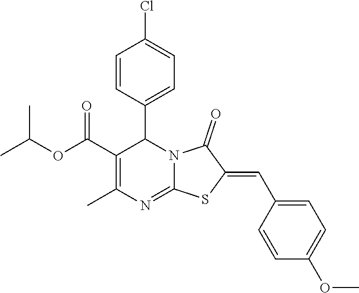 [SMILES: COc1ccc(/C=c2\sc3n(c2=O)C(c2ccc(Cl)cc2)C(C(=O)OC(C)C)=C(C)N=3)cc1] | − | − |
| 4S0 (26) | 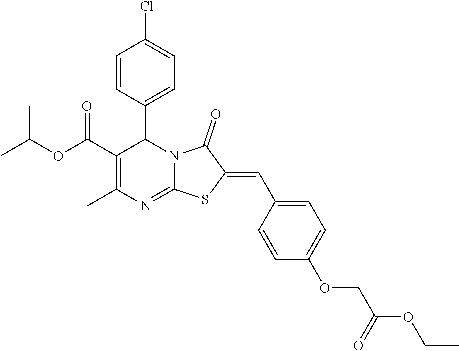 [SMILES: CCOC(=O)COc1ccc(/C=c2\sc3n(c2=O)C(c2ccc(Cl)cc2)C(C(=O)OC(C)C)=C(C)N=3)cc1] | − | − |
| 5S0 (12) | 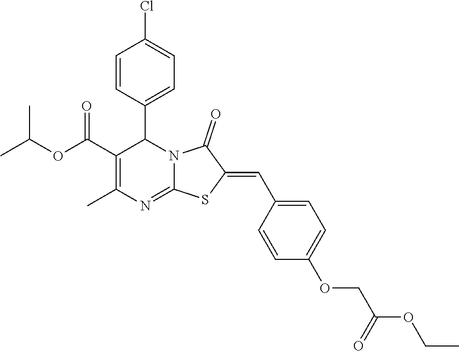 [SMILES: CCOC(=O)COc1ccc(/C=c2\sc3n(c2=O)C(c2ccc(Cl)cc2)C(C(=O)OC(C)C)=C(C)N=3)cc1] | − | − |
| 6S0 (13) | 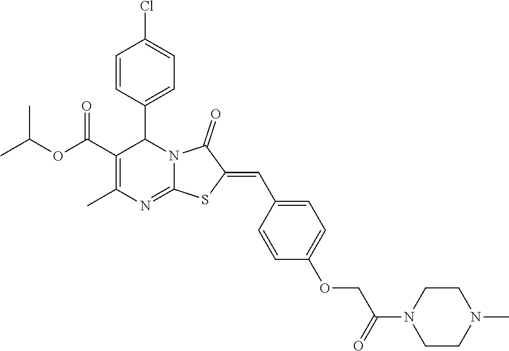 [SMILES: CC1=C(C(=O)OC(C)C)C(c2ccc(Cl)cc2)n2c(s/c(=C\c3ccc(OCC(=O)N4CCN(C)CC4)cc3)c2=O)=N1] | + | + + |
| 7S9 (133) | 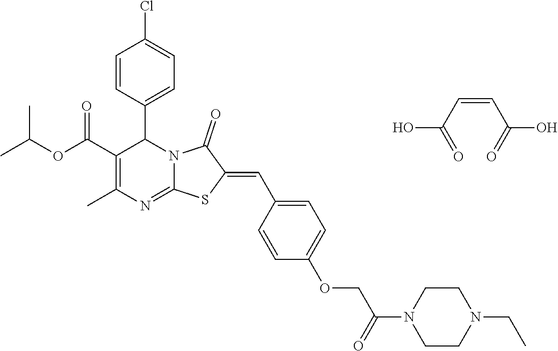 [SMILES: CCN1CCN(C(=O)COc2ccc(/C=c3\sc4n(c3=O)C(c3ccc(Cl)cc3)C(C(=O)OC(C)C)=C(C)N=4)cc2)CC1 ; O=C(O)/C=C\C(=O)O] | + + | + + + |
| 8S0 (15) | 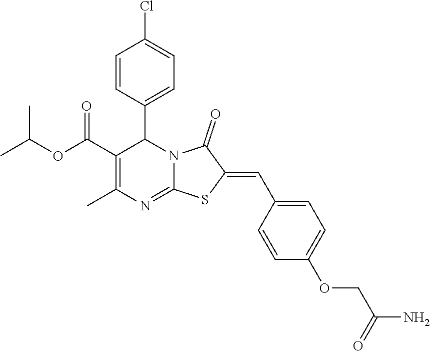 [SMILES: CC1=C(C(=O)OC(C)C)C(c2ccc(Cl)cc2)n2c(s/c(=C\c3ccc(OCC(N)=O)cc3)c2=O)=N1] | − | − |
| 9S0 (32) | 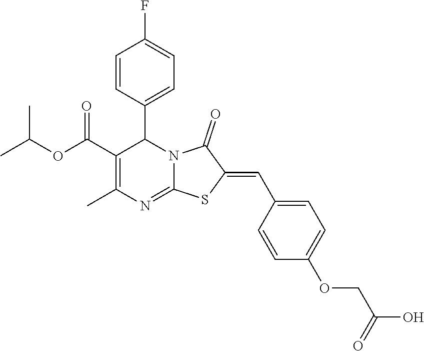 [SMILES: CC1=C(C(=O)OC(C)C)C(c2ccc(F)cc2)n2c(s/c(=C\c3ccc(OCC(=O)O)cc3)c2=O)=N1] | + + | − |
| 10S0 (35) | 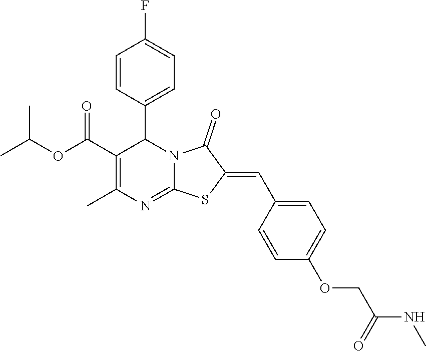 [SMILES: CNC(=O)COc1ccc(/C=c2\sc3n(c2=O)C(c2ccc(F)cc2)C(C(=O)OC(C)C)=C(C)N=3)cc1] | − | − |
| 11S0 (16) | 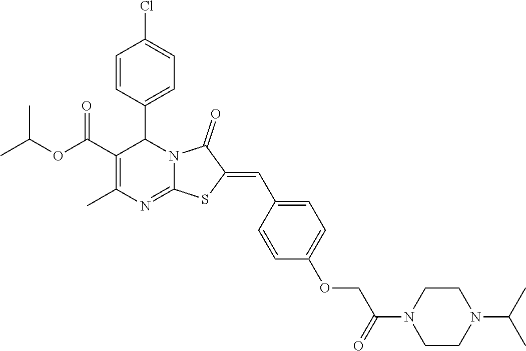 [SMILES: CC1=C(C(=O)OC(C)C)C(c2ccc(Cl)cc2)n2c(s/c(=C\c3ccc(OCC(=O)N4CCN(C(C)C)CC4)cc3)c2=O)=N1] | − | + + |
| 12S0 (79) | 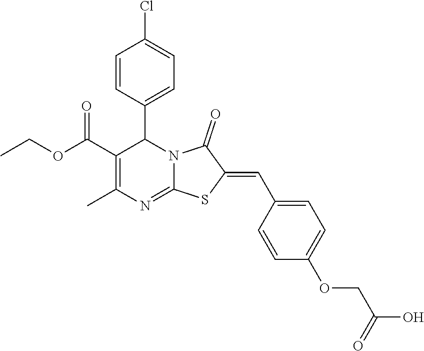 [SMILES: CCOC(=O)C1=C(C)N=c2s/c(=C\c3ccc(OCC(=O)O)cc3)c(=O)n2C1c1ccc(Cl)cc1] | + + + | − |
| 13S0 | 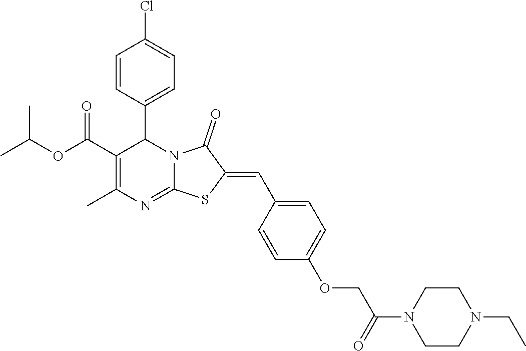 [SMILES: CCN1CCN(C(=O)COc2ccc(/C=c3\sc4n(c3=O)C(c3ccc(Cl)cc3)C(C(=O)OC(C)C)=C(C)N=4)cc2)CC1] | + | + + |
| 14S0 (78) | 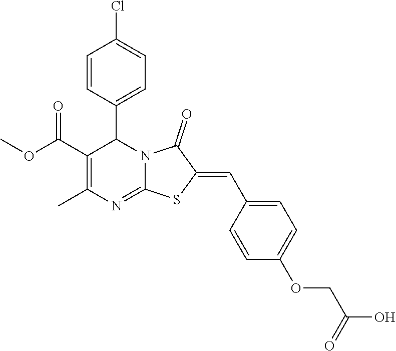 [SMILES: COC(=O)C1=C(C)N=c2s/c(=C\c3ccc(OCC(=O)O)cc3)c(=O)n2C1c1ccc(Cl)cc1] | + | − |
| 15S0 (66) | 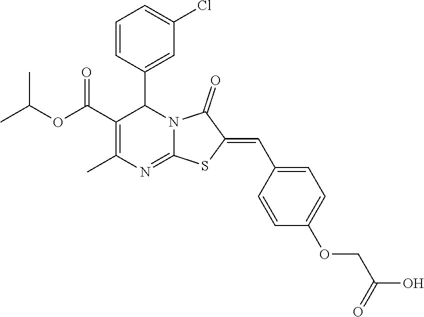 [SMILES: CC1=C(C(=O)OC(C)C)C(c2cccc(Cl)c2)n2c(s/c(=C\c3ccc(OCC(=O)O)cc3)c2=O)=N1] | − | − |
| 16S0 (67) | 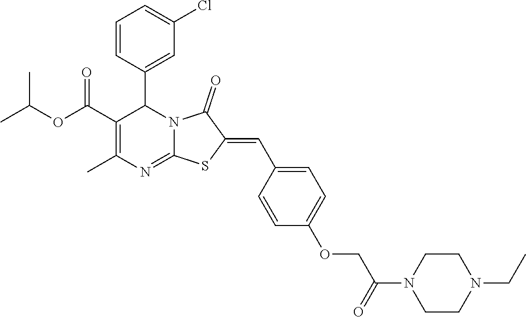 [SMILES: CCN1CCN(C(=O)COc2ccc(/C=c3\sc4n(c3=O)C(c3cccc(Cl)c3)C(C(=O)OC(C)C)=C(C)N=4)cc2)CC1] | + | + + |
| 17S0 (50) | 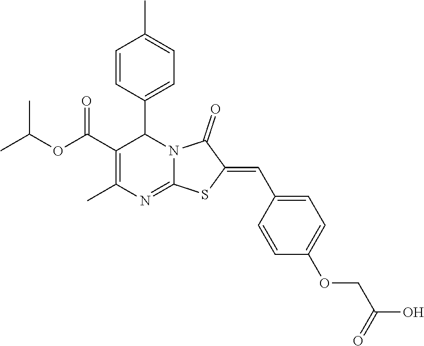 [SMILES: CC1=C(C(=O)OC(C)C)C(c2ccc(C)cc2)n2c(s/c(=C\c3ccc(OCC(=O)O)cc3)c2=O)=N1] | − | − |
| 18S0 (51) | 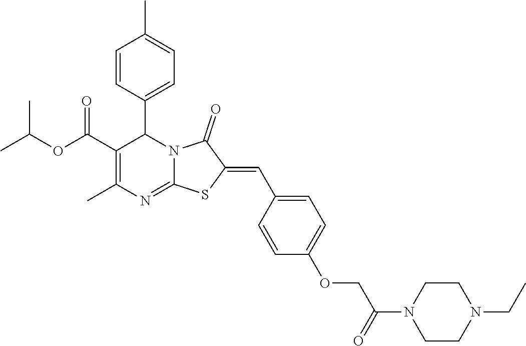 [SMILES: CCN1CCN(C(=O)COc2ccc(/C=c3\sc4n(c3=O)C(c3ccc(C)cc3)C(C(=O)OC(C)C)=C(C)N=4)cc2)CC1] | + | + + |
| 19S0 (56) | 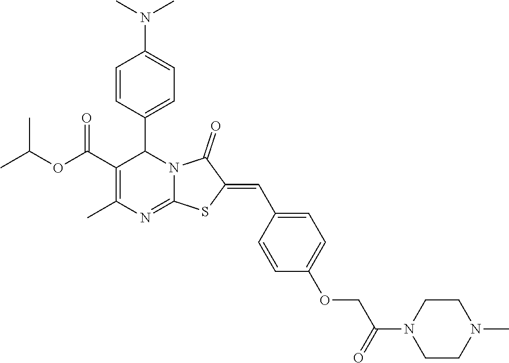 [SMILES: CC1=C(C(=O)OC(C)C)C(c2ccc(N(C)C)cc2)n2c(s/c(=C\c3ccc(OCC(=O)N4CCN(C)CC4)cc3)c2=O)=N1] | + + | + + |
| 20S0 (41) | 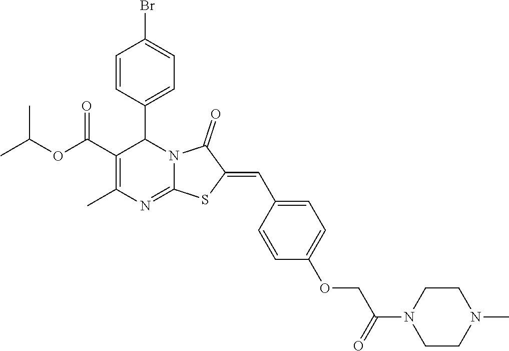 [SMILES: CC1=C(C(=O)OC(C)C)C(c2ccc(Br)cc2)n2c(s/c(=C\c3ccc(OCC(=O)N4CCN(C)CC4)cc3)c2=O)=N1] | + | + + + |
| 21S0 (46) | 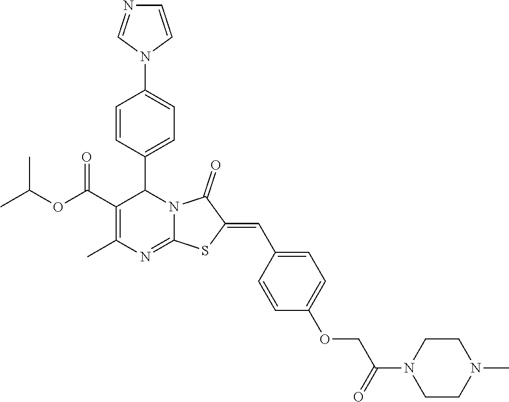 [SMILES: CC1=C(C(=O)OC(C)C)C(c2ccc(-n3ccnc3)cc2)n2c(s/c(=C\c3ccc(OCC(=O)N4CCN(C)CC4)cc3)c2=O)=N1] | + | + + |
| 22S0 (17) | 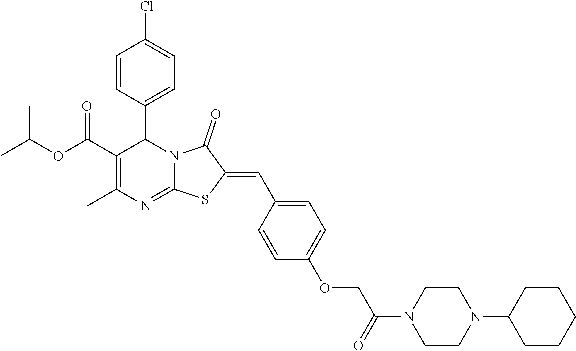 [SMILES: CC1=C(C(=O)OC(C)C)C(c2ccc(Cl)cc2)n2c(s/c(=C\c3ccc(OCC(=O)N4CCN(C5CCCCC5)CC4)cc3)c2=O)=N1] | − | − |
| 23S0 (18) | 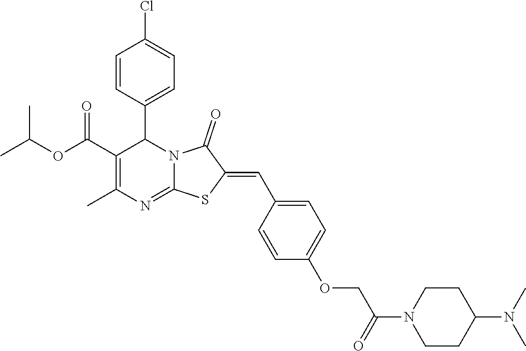 [SMILES: CC1=C(C(=O)OC(C)C)C(c2ccc(Cl)cc2)n2c(s/c(=C\c3ccc(OCC(=O)N4CCC(N(C)C)CC4)cc3)c2=O)=N1] | + + | + + + |
| 23S9 (134) | 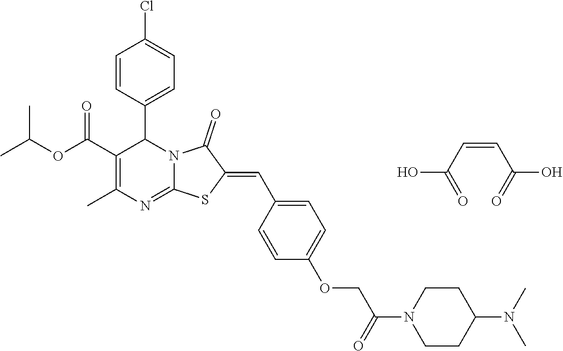 [SMILES: CC1=C(C(=O)OC(C)C)C(c2ccc(Cl)cc2)n2c(s/c(=C\c3ccc(OCC(=O)N4CCC(N(C)C)CC4)cc3)c2=O)=N1 ; O=C(O)/C=C\C(=O)O] | + + | + + + |
| 24S0 (19) | 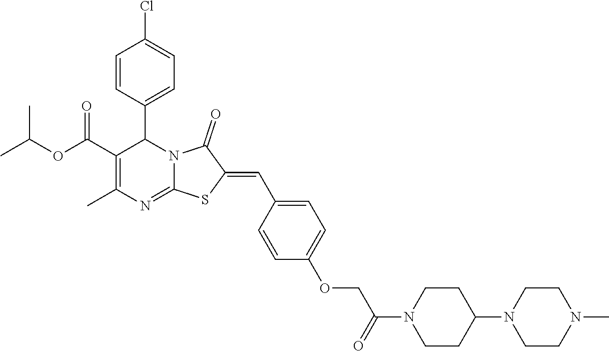 [SMILES: CC1=C(C(=O)OC(C)C)C(c2ccc(Cl)cc2)n2c(s/c(=C\c3ccc(OCC(=O)N4CCC(N5CCN(C)CC5)CC4)cc3)c2=O)=N1] | + + | + + + + |
| 24S9 (135) | 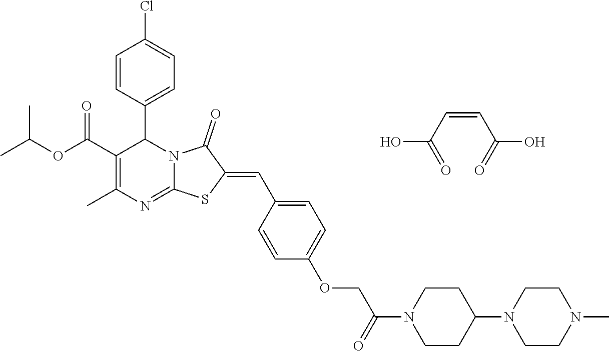 [SMILES: CC1=C(C(=O)OC(C)C)C(c2ccc(Cl)cc2)n2c(s/c(=C\c3ccc(OCC(=O)N4CCC(N5CCN(C)CC5)CC4)cc3)c2=O)=N1 ; O=C(O)/C=C\C(=O)O] | + + | + + + + |
| 25S0 (95) | 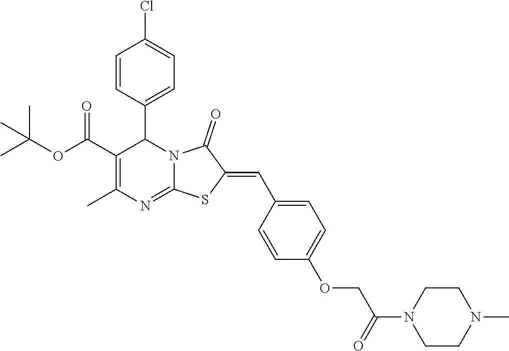 [SMILES: CC1=C(C(=O)OC(C)(C)C)C(c2ccc(Cl)cc2)n2c(s/c(=C\c3ccc(OCC(=O)N4CCN(C)CC4)cc3)c2=O)=N1] | − | + + |
| 26S0 (93) | 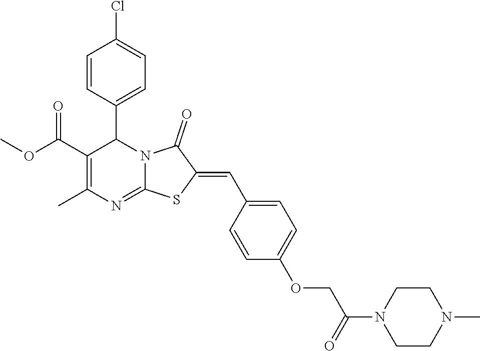 | + | + + |
| 27S0 (98) | 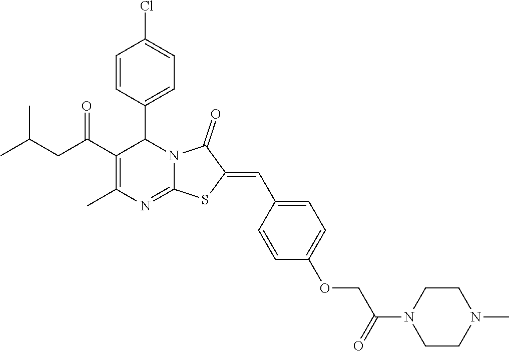 [SMILES: CC1=C(C(=O)CC(C)C)C(c2ccc(Cl)cc2)n2c(s/c(=C\c3ccc(OCC(=O)N4CCN(C)CC4)cc3)c2=O)=N1] | + | + + |
| 28S0 (105) | 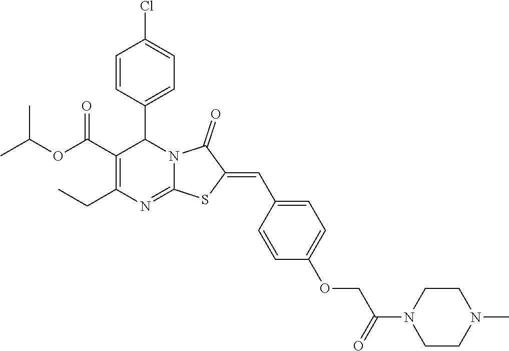 [SMILES: CCC1=C(C(=O)OC(C)C)C(c2ccc(Cl)cc2)n2c(s/c(=C\c3ccc(OCC(=O)N4CCN(C)CC4)cc3)c2=O)=N1] | − | + |
| 28S9 | 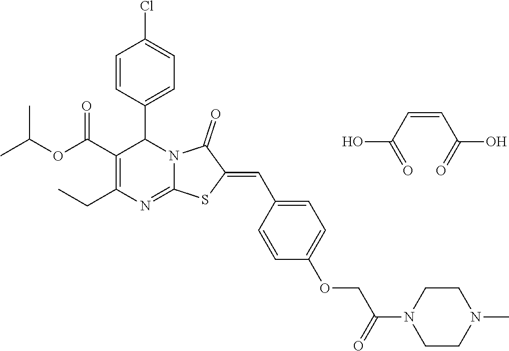 [SMILES: CCC1=C(C(=O)OC(C)C)C(c2ccc(Cl)cc2)n2c(s/c(=C\c3ccc(OCC(=O)N4CCN(C)CC4)cc3)c2=O)=N1 ; O=C(O)/C=C\C(=O)O] | − | + |
| 29S0 (97) | 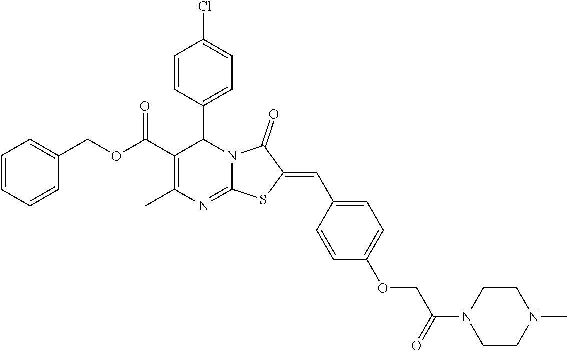 [SMILES: CC1=C(C(=O)OCc2ccccc2)C(c2ccc(Cl)cc2)n2c(s/c(=C\c3ccc(OCC(=O)N4CCN(C)CC4)cc3)c2=O)=N1] | + | + + + |
| 30S0 (96) | 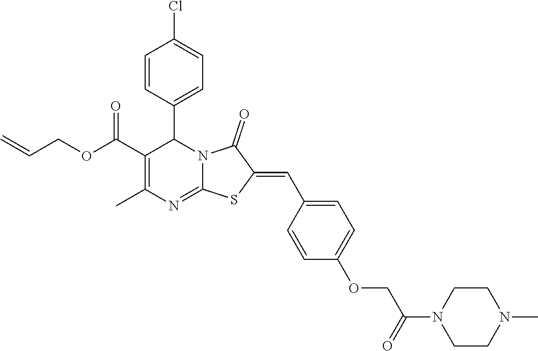 [SMILES: C=CCOC(=O)C1=C(C)N=c2s/c(=C\c3ccc(OCC(=O)N4CCN(C)CC4)cc3)c(=O)n2C1c1ccc(Cl)cc1] | − | + + |
| 31S0 (20) | 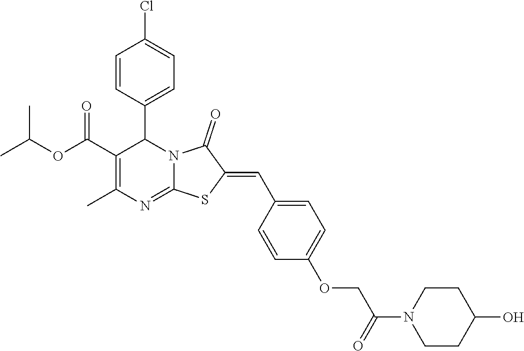 [SMILES: CC1=C(C(=O)OC(C)C)C(c2ccc(Cl)cc2)n2c(s/c(=C\c3ccc(OCC(=O)N4CCC(O)CC4)cc3)c2=O)=N1] | − | − |
| 32S0 (36) | 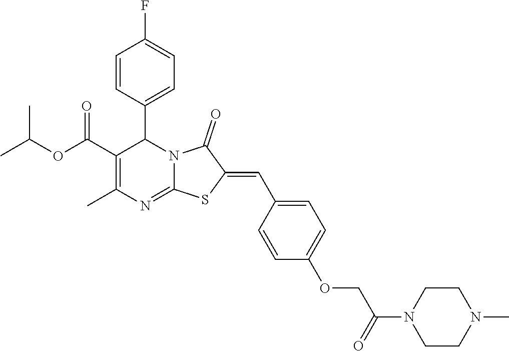 [SMILES: CC1=C(C(=O)OC(C)C)C(c2ccc(F)cc2)n2c(s/c(=C\c3ccc(OCC(=O)N4CCN(C)CC4)cc3)c2=O)=N1] | − | + + |
| 33S0 (115) | 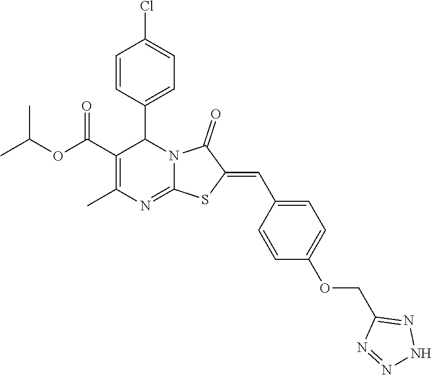 [SMILES: CC1=C(C(=O)OC(C)C)C(c2ccc(Cl)cc2)n2c(s/c(=C\c3ccc(OCc4nn[nH]n4)cc3)c2=O)=N1] | + + + | − |
| 34S0 (24) | 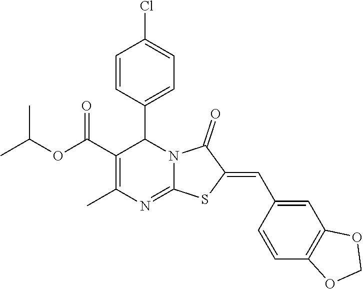 [SMILES: CC1=C(C(=O)OC(C)C)C(c2ccc(Cl)cc2)n2c(s/c(=C\c3ccc4c(c3)OCO4)c2=O)=N1] | − | − |
| 35S0 (110) | 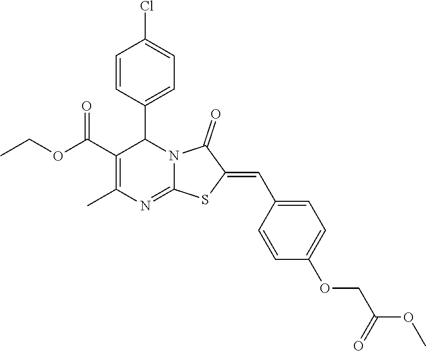 [SMILES: CCOC(=O)C1=C(C)N=c2s/c(=C\c3ccc(OCC(=O)OC)cc3)c(=O)n2C1c1ccc(Cl)cc1] | − | + |
| 36S0 (116) | 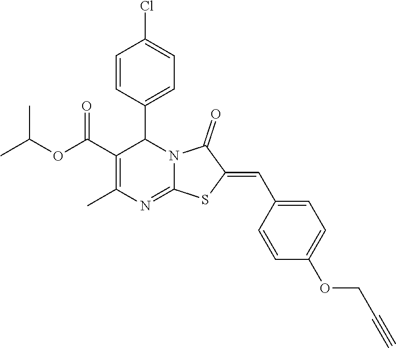 [SMILES: C#CCOc1ccc(/C=c2\sc3n(c2=O)C(c2ccc(Cl)cc2)C(C(=O)OC(C)C)=C(C)N=3)cc1] | − | − |
| 37S0 (120) | 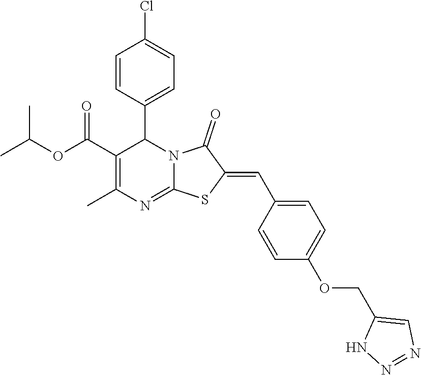 [SMILES: CC1=C(C(=O)OC(C)C)C(c2ccc(Cl)cc2)n2c(s/c(=C\c3ccc(OCc4cnn[nH]4)cc3)c2=O)=N1] | − | − |
| 38S0 (117) | 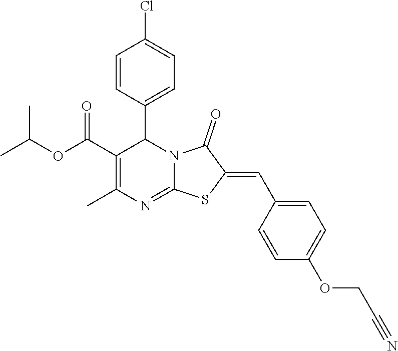 [SMILES: CC1=C(C(=O)OC(C)C)C(c2ccc(Cl)cc2)n2c(s/c(=C\c3ccc(OCC#N)cc3)c2=O)=N1] | − | − |
| 39S0 (118) | 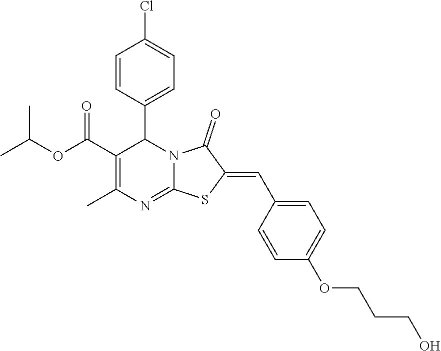 [SMILES: CC1=C(C(=O)OC(C)C)C(c2ccc(Cl)cc2)n2c(s/c(=C\c3ccc(OCCCO)cc3)c2=O)=N1] | − | − |
| 40S0 (40) | 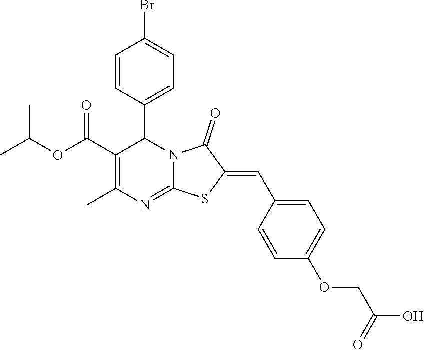 [SMILES: CC1=C(C(=O)OC(C)C)C(c2ccc(Br)cc2)n2c(s/c(=C\c3ccc(OCC(=O)O)cc3)c2=O)=N1] | + + + | − |
| 41S0 (60) | 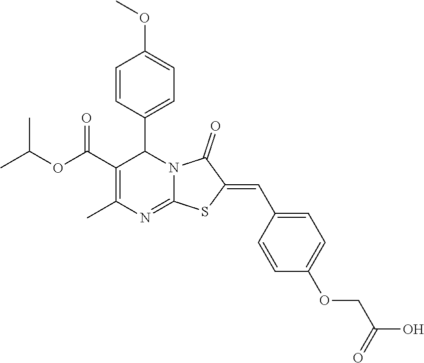 [SMILES: COc1ccc(C2C(C(=O)OC(C)C)=C(C)N=c3s/c(=C\c4ccc(OCC(=O)O)cc4)c(=O)n32)cc1] | + + | − |
| 42S0 (61) | 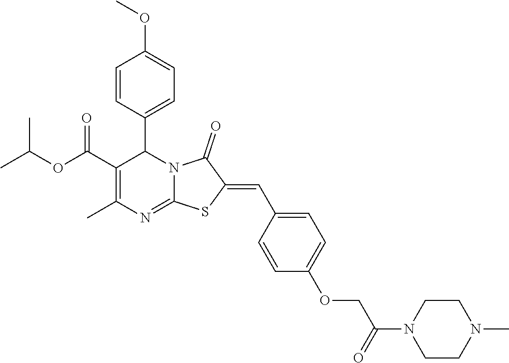 [SMILES: COc1ccc(C2C(C(=O)OC(C)C)=C(C)N=c3s/c(=C\c4ccc(OCC(=O)N5CCN(C)CC5)cc4)c(=O)n32)cc1] | − | + |
| 43S0 (62) | 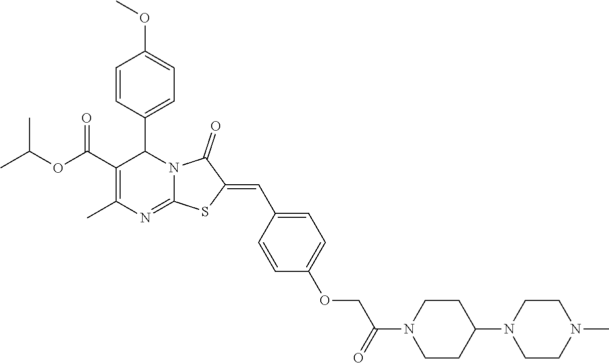 [SMILES: COc1ccc(C2C(C(=O)OC(C)C)=C(C)N=c3s/c(=C\c4ccc(OCC(=O)N5CCC(N6CCN(C)CC6)CC5)cc4)c(=O)n32)cc1] | + | + + + + |
| 44S0 (128) | 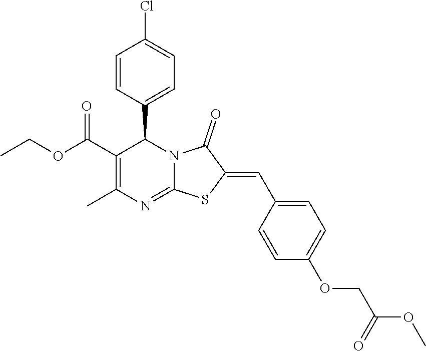 [SMILES: CCOC(=O)C1=C(C)N=c2s/c(=C\c3ccc(OCC(=O)OC)cc3)c(=O)n2[C@@H]1c1ccc(Cl)cc1] | − | − |
| 45S0 (129) | 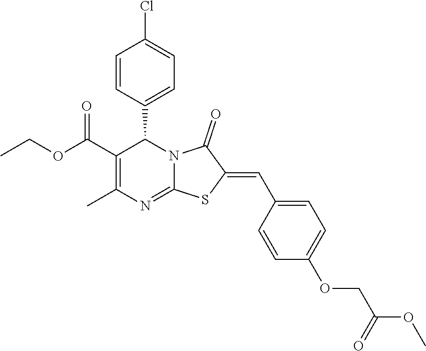 [SMILES: CCOC(=O)C1=C(C)N=c2s/c(=C\c3ccc(OCC(=O)OC)cc3)c(=O)n2[C@H]1c1ccc(Cl)cc1] | − | − |
| 46S0 (25) | 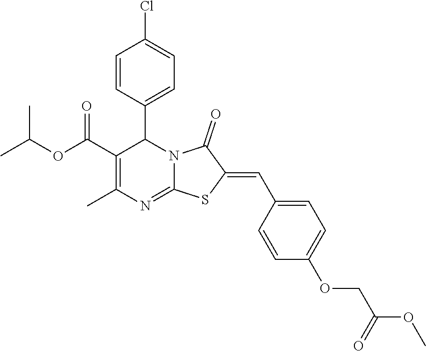 [SMILES: COC(=O)COc1ccc(/C=c2\sc3n(c2=O)C(c2ccc(Cl)cc2)C(C(=O)OC(C)C)=C(C)N=3)cc1] | − | − |
| 47S0 (107) | 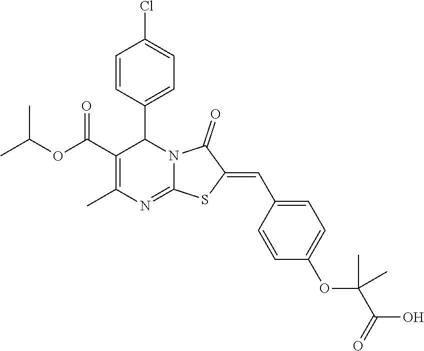 [SMILES: CC1=C(C(=O)OC(C)C)C(c2ccc(Cl)cc2)n2c(s/c(=C\c3ccc(OC(C)(C)C(=O)O)cc3)c2=O)=N1] | − | − |
| 48S0 (108) | 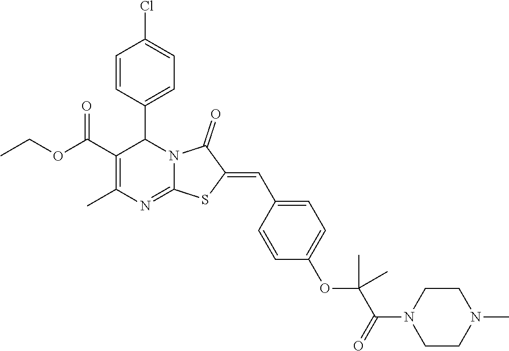 [SMILES: CCOC(=O)C1=C(C)N=c2s/c(=C\c3ccc(OC(C)(C)C(=O)N4CCN(C)CC4)cc3)c(=O)n2C1c1ccc(Cl)cc1] | − | + |
| 49S0 (94) | 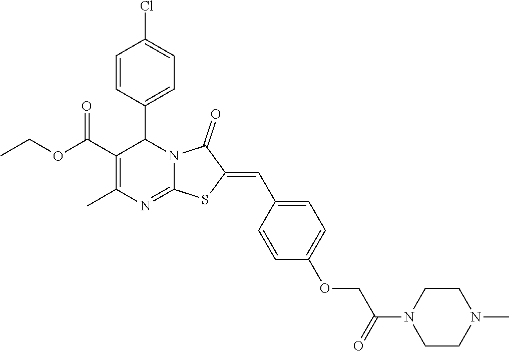 [SMILES: CCOC(=O)C1=C(C)N=c2s/c(=C\c3ccc(OCC(=O)N4CCN(C)CC4)cc3)c(=O)n2C1c1ccc(Cl)cc1] | + | + + |
| 50S0 (99) | 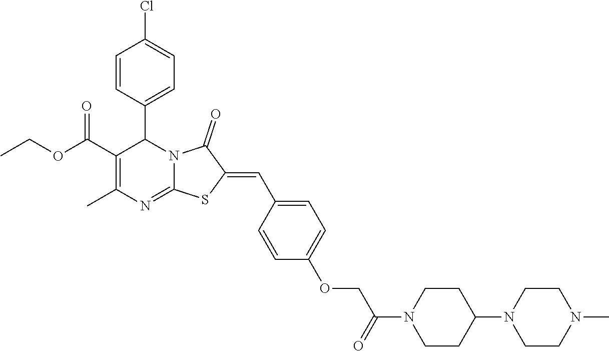 [SMILES: CCOC(=O)C1=C(C)N=c2s/c(=C\c3ccc(OCC(=O)N4CCC(N5CCN(C)CC5)CC4)cc3)c(=O)n2C1c1ccc(Cl)cc1] | + + | + + + + |
| 51S0 (55) | 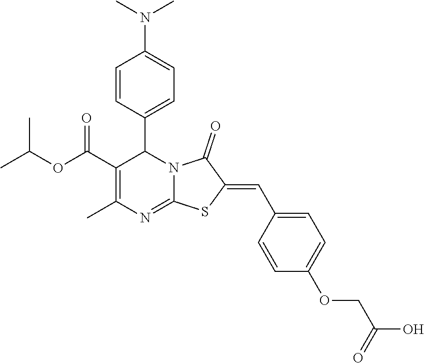 [SMILES: CC1=C(C(=O)OC(C)C)C(c2ccc(N(C)C)cc2)n2c(s/c(=C\c3ccc(OCC(=O)O)cc3)c2=O)=N1] | + | − |
| 52S0 (45) | 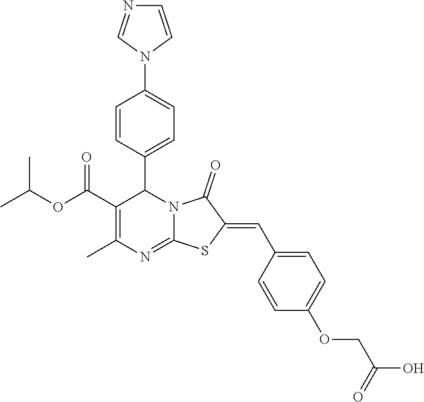 [SMILES: CC1=C(C(=O)OC(C)C)C(c2ccc(-n3ccnc3)cc2)n2c(s/c(=C\c3ccc(OCC(=O)O)cc3)c2=O)=N1] | − | − |
| 53S0 (80) | 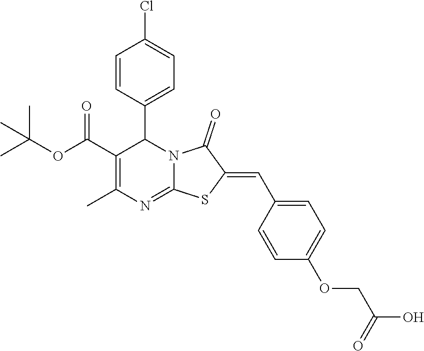 [SMILES: CC1=C(C(=O)OC(C)(C)C)C(c2ccc(Cl)cc2)n2c(s/c(=C\c3ccc(OCC(=O)O)cc3)c2=O)=N1] | + | − |
| 54S0 (130) | 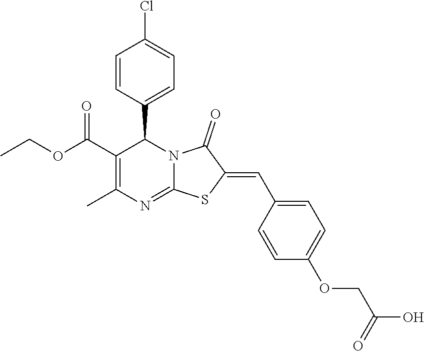 [SMILES: CCOC(=O)C1=C(C)N=c2s/c(=C\c3ccc(OCC(=O)O)cc3)c(=O)n2[C@@H]1c1ccc(Cl)cc1] | + | − |
| 55S0 (131) | 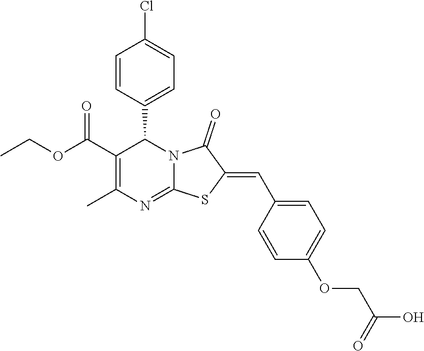 [SMILES: CCOC(=O)C1=C(C)N=c2s/c(=C\c3ccc(OCC(=O)O)cc3)c(=O)n2[C@H]1c1ccc(Cl)cc1] | − | − |
| 56S0 (121) | 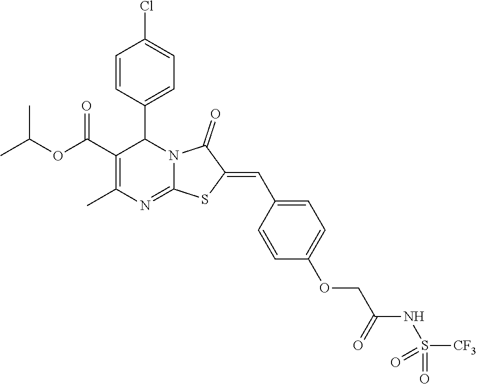 [SMILES: CC1=C(C(=O)OC(C)C)C(c2ccc(Cl)cc2)n2c(s/c(=C\c3ccc(OCC(=O)NS(=O)(=O)C(F)(F)F)cc3)c2=O)=N1] | − | − |
| 136 | 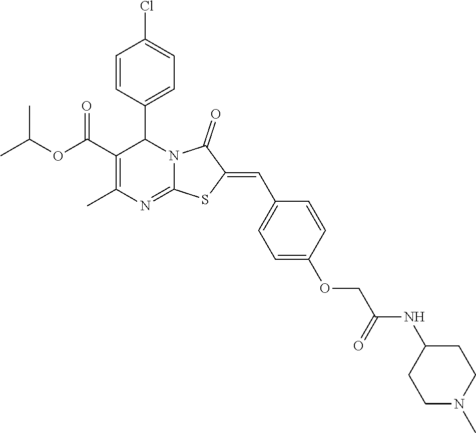 [SMILES: CC1=C(C(=O)OC(C)C)C(c2ccc(Cl)cc2)n2c(s/c(=C\c3ccc(OCC(=O)NC4CCN(C)CC4)cc3)c2=O)=N1] | − | − |
| 137 | 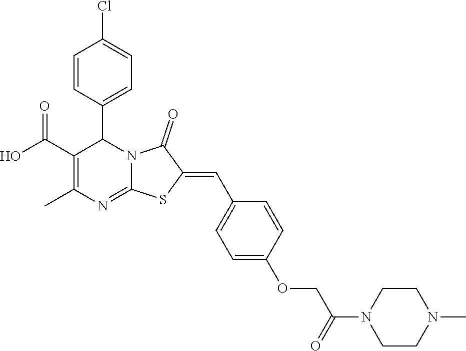 [SMILES: CC1=C(C(=O)O)C(c2ccc(Cl)cc2)n2c(s/c(=C\c3ccc(OCC(=O)N4CCN(C)CC4)cc3)c2=O)=N1] | + + | − |
| 138 | 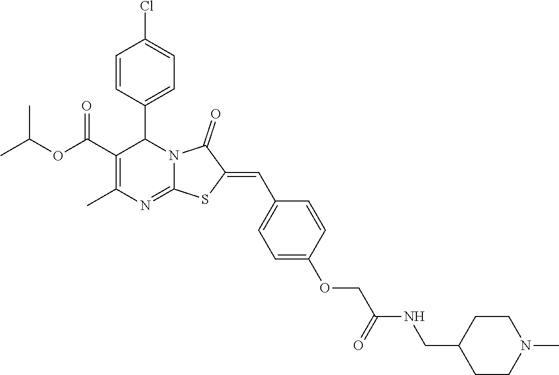 [SMILES: CC1=C(C(=O)OC(C)C)C(c2ccc(Cl)cc2)n2c(s/c(=C\c3ccc(OCC(=O)NCC4CCN(C)CC4)cc3)c2=O)=N1] | + | + |
| 139 | 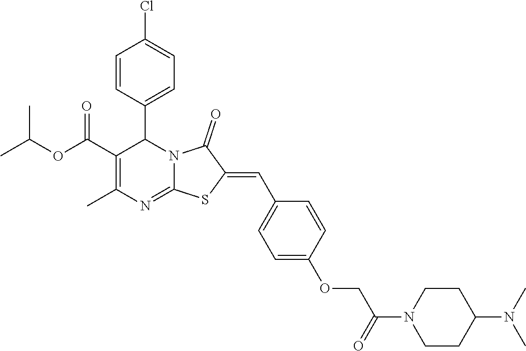 [SMILES: CC1=C(C(=O)OC(C)C)C(c2ccc(Cl)cc2)n2c(s/c(=C\c3ccc(OCC(=O)N4CCC(N(C)C)CC4)cc3)c2=O)=N1] | + | + |
| 140 | 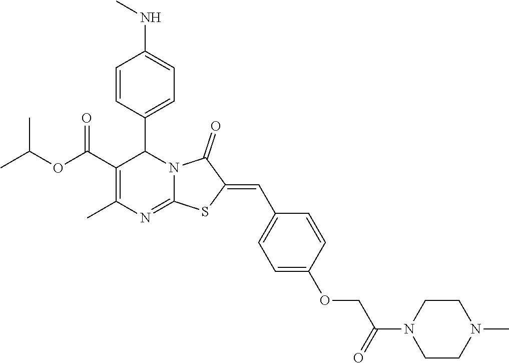 [SMILES: CNc1ccc(C2C(C(=O)OC(C)C)=C(C)N=c3s/c(=C\c4ccc(OCC(=O)N5CCN(C)CC5)cc4)c(=O)n32)cc1] | + | + |
| 141 | 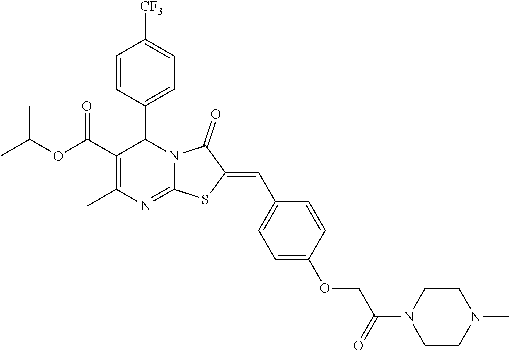 [SMILES: CC1=C(C(=O)OC(C)C)C(c2ccc(C(F)(F)F)cc2)n2c(s/c(=C\c3ccc(OCC(=O)N4CCN(C)CC4)cc3)c2=O)=N1] | + + | + |
| 142 | 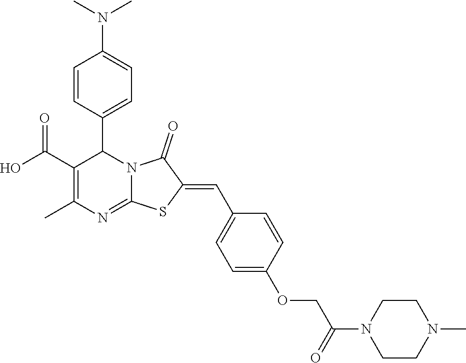 [SMILES: CC1=C(C(=O)O)C(c2ccc(N(C)C)cc2)n2c(s/c(=C\c3ccc(OCC(=O)N4CCN(C)CC4)cc3)c2=O)=N1] | + | − |
| 143 | 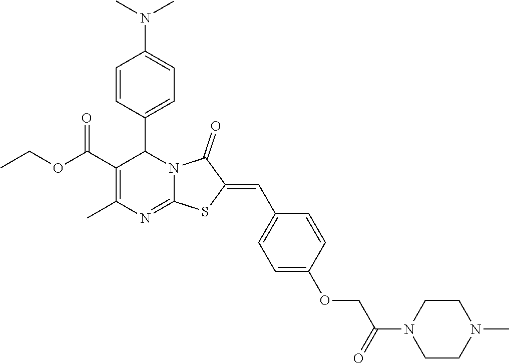 [SMILES: CCOC(=O)C1=C(C)N=c2s/c(=C\c3ccc(OCC(=O)N4CCN(C)CC4)cc3)c(=O)n2C1c1ccc(N(C)C)cc1] | + | + |
| 144 | 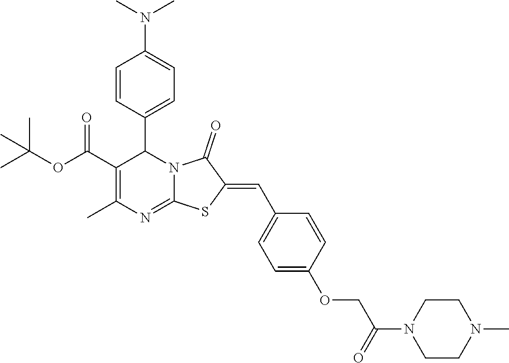 [SMILES: CC1=C(C(=O)OC(C)(C)C)C(c2ccc(N(C)C)cc2)n2c(s/c(=C\c3ccc(OCC(=O)N4CCN(C)CC4)cc3)c2=O)=N1] | + | + |
| 145 | 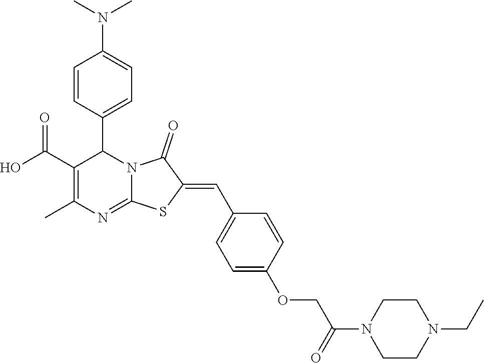 [SMILES: CCN1CCN(C(=O)COc2ccc(/C=c3\sc4n(c3=O)C(c3ccc(N(C)C)cc3)C(C(=O)O)=C(C)N=4)cc2)CC1] | + + | − |
| 146 | 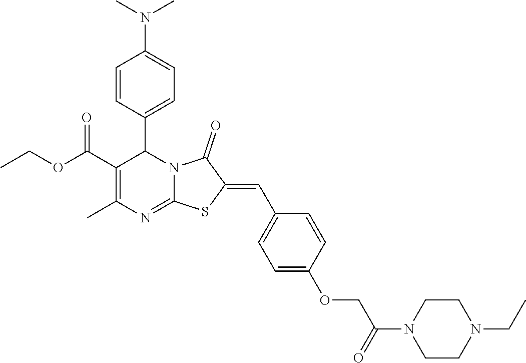 [SMILES: CCOC(=O)C1=C(C)N=c2s/c(=C\c3ccc(OCC(=O)N4CCN(CC)CC4)cc3)c(=O)n2C1c1ccc(N(C)C)cc1] | + + | + |
| 147 | 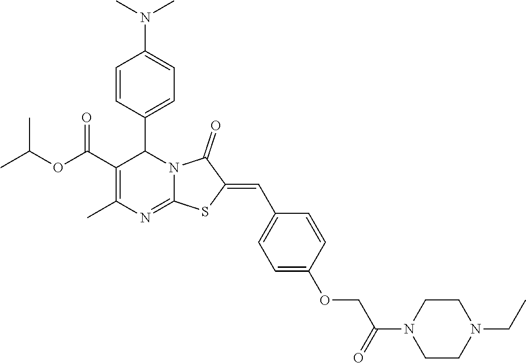 [SMILES: CCN1CCN(C(=O)COc2ccc(/C=c3\sc4n(c3=O)C(c3ccc(N(C)C)cc3)C(C(=O)OC(C)C)=C(C)N=4)cc2)CC1] | + + | + + |
| 148 | 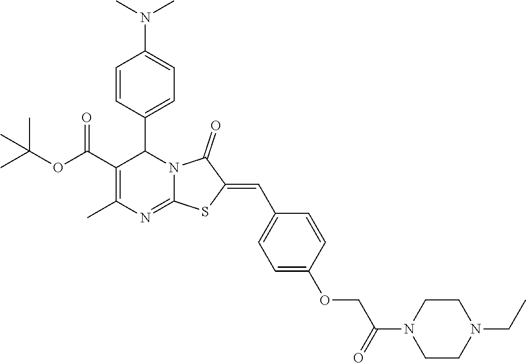 [SMILES: CCN1CCN(C(=O)COc2ccc(/C=c3\sc4n(c3=O)C(c3ccc(N(C)C)cc3)C(C(=O)OC(C)(C)C)=C(C)N=4)cc2)CC1] | + + | + + |
| 149 | 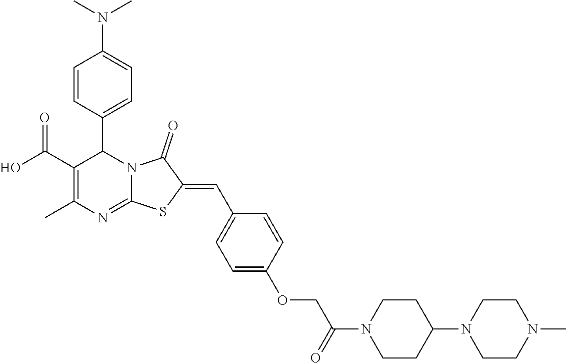 [SMILES: CC1=C(C(=O)O)C(c2ccc(N(C)C)cc2)n2c(s/c(=C\c3ccc(OCC(=O)N4CCC(N5CCN(C)CC5)CC4)cc3)c2=O)=N1] | + + | − |
| 150 | 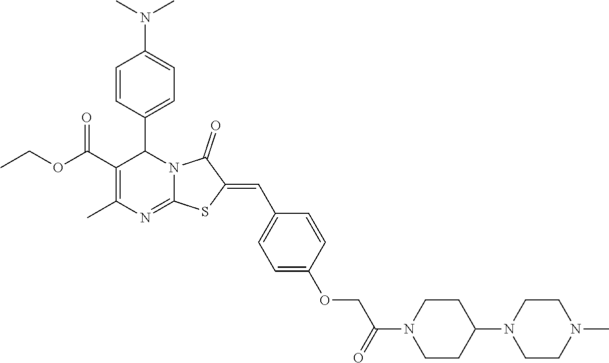 [SMILES: CCOC(=O)C1=C(C)N=c2s/c(=C\c3ccc(OCC(=O)N4CCC(N5CCN(C)CC5)CC4)cc3)c(=O)n2C1c1ccc(N(C)C)cc1] | + | + |
| 151 | 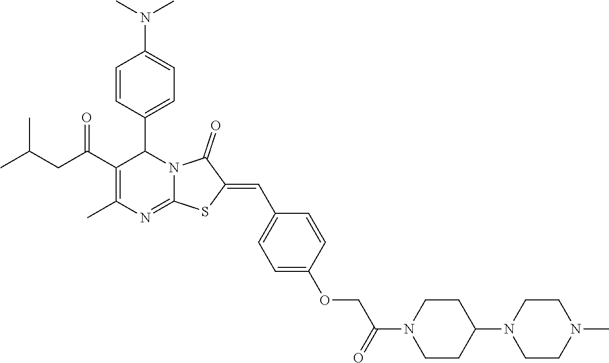 [SMILES: CC1=C(C(=O)CC(C)C)C(c2ccc(N(C)C)cc2)n2c(s/c(=C\c3ccc(OCC(=O)N4CCC(N5CCN(C)CC5)CC4)cc3)c2=O)=N1] | + | + |
| 152 | 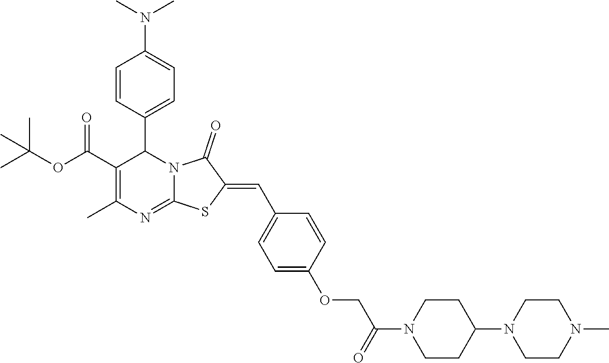 [SMILES: CC1=C(C(=O)OC(C)(C)C)C(c2ccc(N(C)C)cc2)n2c(s/c(=C\c3ccc(OCC(=O)N4CCC(N5CCN(C)CC5)CC4)cc3)c2=O)=N1] | + | + |

The experimental procedures of derivatives 136-152 were similar to that of compound 133. The LCMS data are shown in the following Table 1-1.

TABLE 1-1

| Compound name/number | LRMS (ESI) m/z |
|---|---|
| 136 | 623.1 [M + H]⁺ |
| 137 | 567.3 [M + H]⁺ |
| 138 | 637.1 [M + H]⁺ |
| 139 | 637.1 [M + H]⁺ |
| 140 | 604.2 [M + H]⁺ |
| 141 | 643.1 [M + H]⁺ |
| 142 | 576.2 [M + H]⁺ |
| 143 | 604.3 [M + H]⁺ |
| 144 | 632.1 [M + H]⁺ |
| 145 | 590.2 [M + H]⁺ |
| 146 | 618.2 [M + H]⁺ |
| 147 | 632.2 [M + H]⁺ |
| 148 | 646.3 [M + H]⁺ |
| 149 | 659.3 [M + H]⁺ |
| 150 | 687.3 [M + H]⁺ |
| 151 | 701.3 [M + H]⁺ |
| 152 | 715.1 [M + H]⁺ |

### Cell Culture

MCF-7, MDA-MB-231, MDA-MB-468, Hs578T, 4T1, and HeLa cell lines stably expressing H2B-mCherry and eGFP-tubulin (kindly provided by Dr. Anthony A. Hyman, Max-Planck-Institute for Molecular Cell Biology and Genetics, Dresden, Germany) were cultured in high-glucose Dulbecco's Modified Eagle Medium (DMEM). SK-BR-3 cells were cultured in McCoy's Modified 5a Medium. Hs578T cells were cultured in high-glucose DMEM supplemented with an additional 0.01 mg/mL bovine insulin. MDA-MB-231 R cells were maintained with an additional 10 nM paclitaxel, while 4T1 R4 and R8 cells were maintained with 200 nM paclitaxel. All mentioned media were supplemented with 10% fetal bovine serum (Gibco) and 1% Penicillin/Streptomycin (Corning), and cells were incubated at 37° C. in a humidified atmosphere containing 5% CO₂. HEK293 cells were cultured in HE300 medium (GMEP incorporated, Japan) and maintained in suspension culture with 125 rpm and 8% CO₂.

### Western Blot Analysis

To detect the various endogenous protein levels in cancer cell lines, the cells were harvested by trypsin and lysed in RIPA buffer (1% NP-40, 0.1% SDS, 0.5% sodium deoxycholate, 150 mM NaCl, 50 mM Tris-HCl, pH 7.4) supplemented with 1× protease inhibitor cocktail (Roche). The lysing samples were incubated on ice for 30 min and scraped every 10 min, followed by 13000 rpm centrifugation at 4° C. for 15 min. The supernatants were collected for protein quantification by Bradford assay, by which the protein concentrations were determined with spectrophotometer measurements. The protein samples were mixed with a 2× Laemmli sample buffer containing 3-Me and then incubated at 100° C. for 15 min before loading to 10% SDS gel. After electrophoresis, the samples were transferred to the PVDF membrane (Millipore) by 100V for 1 hr. The membrane was incubated with primary antibodies at 4° C. overnight. After washing three times with phosphate buffer saline with 0.05% Tween 20 (PBST) every 10 min, the PVDF membrane was then incubated with horseradish peroxidase (HRP)-conjugated secondary antibodies (GE Healthcare) for 30 min at room temperature. Finally, protein bands on washed PVDF membrane were visualized using modified ECL reagent (ddH₂O, 100 nM Tris-HCl pH8.0, 200 μM p-coumaric acid, 1.25 mM luminal, 0.001% H₂O₂) and detected by ImageQuant LAS 4000 digital imaging system (GE Healthcare).

### Immunofluorescence Staining

Cells grown on coverslips were washed with PBS and then incubated with PTEMF fixation solution (20 mM PIPES pH 6.8, 0.2% Triton X-100 10 mM EGTA, and 4% formaldehyde) at room temperature for 15 min. The fixation buffer was aspirated and replaced with PBST at room temperature for 10 min. The coverslips were then washed with PBST and incubated with primary antibodies at room temperature for 1 hr. After washing three times with PBST (2, 2, and 5 min), the AlexaFlour-conjugated secondary antibodies were used to recognize primary antibodies (at room temperature, for 30 min). Finally, the coverslips were washed another three times (2, 2, and 5 min), rinsed with ddH₂O, and mounted with VECTASHIELD Antifade Mounting Medium (Vector). Images were acquired with corresponding laser wavelengths by Leica DMI6000 microscope with an HCX PL FL 63×/1.4 NA oil objective lens and Andor Neo sCMOS camera, which were all processed by MetaMorph software (Molecular Devices, LLC. San Jose, CA, USA).

### Cell Viability Assay

The cancer cells were cultured in a 96-well plate at a confluence of 1000-2000 cells/well with indicated drugs for specific days. When the time was up, the culture medium in the plate was aspirated and replaced with a prepared CellTiter-Glo reagent/DMEM mixture (1:1 mix of CellTiter-Glo reagent and serum-supplemented DMEM). After incubation for 10 min, the luminescence (proportional to the number of living cells) was measured with an ELISA plate reader to determine the cell viability.

### Time-Lapse Imaging Microscopy

To investigate the role of KIF2C in cell cycle progression, 4T1 cells were cultured in the 4-well chamber slide with about 50% confluence. The culture medium was replaced with a CO₂-independent medium (Gibco) supplemented with 10% fetal bovine serum (Biological Industries) and 1% penicillin-streptomycin (Biowest). Then, each well of the chamber slide was treated with indicated drugs, such as DMSO, KIF2C inhibitors, or chemotherapy drugs. After that, the chamber slide was maintained in the microscope stage incubator with the supplement of humidified air with 5% CO₂ at 37° C. Multiple-positional time-lapse imaging was performed using an automated Leica DM16000 inverted microscope equipped with HCX PL FL 20×/0.4 and EMCCD camera (Andor Luca R, Belfast, UK). Sequential differential interference contrast (DIC) images of cells obtained at indicated intervals were further analyzed using Metamorph software (Molecular Devices).

For the microtubule distribution analysis, HeLa cell line stably expressing H2B-mCherry and eGFP-tubulin (a kind gift from Anthony A. Hyman, Max-Planck-Institute for Molecular Cell Biology and Genetics, Dresden, Germany) were treated with KIF2C inhibitors (10 μM DHTP or 10 μM 7S9) during imaging. Live-cell imaging was conducted on a Nikon T1 inverted fluorescence microscope (Nikon) with a 60× oil objective (Nikon), a Prime camera (Photometrics), and 37° C., 5% CO₂ heat stage (Live Cell Instrument). Microtubule morphological changes were imaged at 3-min intervals. Images were obtained using ImageJ software processed with Gaussian Blur to remove the noise.

### Colony Formation Assay

To assess the long-term survival of breast cancer cell lines following the treatment of KIF2C knockdown or inhibition, the cells trypsinized into single-cell suspension were homogeneously seeded in 6-well cell culture plates at a density of 5000 cells per dish, supplemented with 2× complete medium (50% 4×DMEM, 20% FBS, 2% penicillin-streptomycin, ddH₂O, and 2× glutaMAX™), agarose (0.6% for bottom agar layer; 0.3% for top cell layer), ddH₂O, and DMEM. After incubation for 24 hr, cells were treated with indicated drugs, such as DMSO, KIF2C inhibitors, or chemotherapy drugs, and then cultured at 37° C. incubator in an atmosphere of humidified air with 5% CO₂ for a further 14-30 days. When the cell colonies grew to appropriate sizes, the culture mediums were replaced with PBS for washing. After that, the plates were stained with 10% crystal violet and analyzed using Image J.

### In Vitro Kinesin Assay

In vitro kinesin activity was measured using malachite green-based phosphate colorimetric assay (BioVision). In short, 400 nM recombinant kinesin protein was incubated with 300 nM microtubules in a 200 μL reaction buffer (1 μM paclitaxel, 75 mM KCl, 1 mM MgCl2, and 600 nM ATP) for 30 min at 37° C. The microtubules from porcine brain tubulin (Cytoskeleton) or purified from HEK293 cells in Free-style medium (293Fs) were dissolved in the BRB80 buffer (80 mM PIPES pH 6.8, 1 mM EGTA, 1 mM MgCl₂, 1 mM GTP, 1 mM DTT, and 10% DMSO) at 37° C. for 2 hr. Phosphates released from kinesin-mediated ATP hydrolysis was measured by adding 30 μL of the phosphate colorimetric reagent (BioVision), incubated for 10 min, and then measured with absorbance at 650 nm.

### Wound Healing Assay

Cells were seeded into a 4-well chamber slide and incubated overnight to reach a confluent monolayer culture. The wound was performed mechanically by generating a break with a pipet tip over the monolayer cells. The degree of cell migration was quantified after 72 hr post-wounding. Images were acquired with corresponding laser wavelengths by Leica DM16000 inverted microscope equipped with HCX PL FL 100×/NA1.40 objective and EMCCD camera (Andor Luca R, Belfast, UK) and further analyzed by Image J.

### Flow Cytometry Analysis

To quantify the cell membrane penetration ability of different DHTP analogs, 4T1 cells were collected by trypsin after 2 hr of drug treatment and then transferred into microcentrifuge tubes. 4T1 cells were then resuspended into the FACS buffer (1% FBS, 2 mM EDTA, and 0.05% NaN₃ in PBS). After two times of FACS buffer rinsing, these samples were detected by flow cytometry (Cyto FLEX™ FLOW Cytometer). The KO525 (405 nm laser; 525 nm filter) channel was used to detect DHTP and 7S9.

### Production of Recombinant Kinesin-13 Family Proteins

Constructs of sN+M regions of KIF2C (216-598 a.a.), KIF2A (181-562 a.a.) and KIF2B (171-553 a.a.) with C-terminal 6×His tag were subcloned into pET-28a vector and expressed in BL21 (DE3) E. coli cells (Novartis) cultured in Luria-Bertani medium supplemented with 50 ug/mL kanamycin at 37° C. After induction with 0.8 mM IPTG for 18 hr at 20° C., the cells were pelleted by centrifugation at 6000 rpm for 30 min at 10° C. The collected pellets were resuspended in 30 mL of buffer A (20 mM Tris-HCl pH 7.5, 500 mM NaCl, 10 mM imidazole, 1% CHAPS, and 1 mM PMSF) and next subjected to a high-pressure homogenizer (EmulsiFlex-C3) for cell lysis. After removing the cell debris by centrifugation at 13000 rpm for 30 min at 10° C., the recombinant Kinesin-13 in the supernatant was purified using nickel affinity chromatography. Firstly, the supernatant was applied to the Ni-NTA column (GE Healthcare) pre-equilibrated with buffer A. Secondly, the column was washed with 10× column volumes of buffer A to remove the unwanted proteins. The washed column was then eluted with 10× column volumes of buffer containing increasing concentrations of imidazole (40, 100, 300, 1000 mM), and the majority of the recombinant Kinesin-13 protein was eventually obtained in a fraction of 300 mM imidazole. The fractions containing the recombinant Kinesin-13 were subsequently concentrated in the buffer B (20 mM Tris-HCl pH 7.5, 10 mM imidazole, 50 mM NaCl, 2 mM DTT) using an Amicon® Ultra filter (Millipore) and subjected to cation exchange column (Capto S, GE Healthcare) for further purification. The recombinant Kinesin-13 was then eluted in elution buffer (20 mM Tris-HCl pH 7.5 and 2 mM DTT) containing 400 mM of NaCl and stored at −80° C. for long-term storage.

### Tubulin Purification

To purify tubulin from HEK293 cells, a 500 mL suspension culture of HEK293 cells at a concentration of 2×10⁶ cells/mL was harvested by centrifugation at 1700 g for 15 min. Transient transfections of tubulin modifying enzymes (polyglutamylase TTLL4, deglutamylase CCP5, and carboxypeptidase VASH1/2-VBP) were carried out using polyethylenimine transfection. After 72 hr post-transfection, the transfected HEK293 cells were harvested. For tubulin purification from 4T1 S1, R4, and R8 cells, at least 1×10⁸ cells were harvested. Purification of a/0-tubulin was conducted following previously established protocols.

### In Vitro Microtubule Depolymerization Assay

To visualize microtubule depolymerization, 5 μM porcine brain microtubules (Cytoskeleton) were prepared in a reaction buffer containing 500 nM SiR-Tubulin (Cytoskeleton), 500 nM paclitaxel, 75 mM KCl, 1 mM MgCl₂. Additional purified KIF2C (0.5 ng/μl), ATP (0.75 mM), and 7S9 (50 μM) were included in a typical 10 μL reaction, as indicated, loaded onto a p-Slide (Ibidi, 81506) and incubated at 37° C. for 30 min. Images were acquired using the Leica DMI6000 microscope with an HCX PL FL 20×/0.4 and Andor Neo sCMOS camera, which were all processed by MetaMorph software (Molecular Devices, LLC. San Jose, CA, USA).

### KIF2C-Tubulin Affinity Pull-Down Assay

To explore the interaction strength between KIF2C and tubulin, we mixed 10 pg recombinant KIF2C with tubulin (approximate molar ratio=1:2) in reaction buffer (75 mM KCl, 1 mM MgCl₂, and 600 nM ATP) with or without 1 μM paclitaxel and incubated at 37° C. for 30 min. The mixture was subjected to KIF2C pull-down with 50 μL of pre-equilibrated Ni-NTA resin. Residual tubulin that remained interacting with KIF2C was resolved by SDS-PAGE followed by Coomassie brilliant blue staining or western blotting.

### Immunohistochemical Analysis of Breast Cancer Tissue Arrays

Formalin-fixed, paraffin-embedded human breast tissue microarrays (BC081120d, BR2082c, BR1921c, BC081116d) were acquired from Biomax, Inc. KIF2C protein expression was evaluated through immunohistochemical staining using the anti-KIF2C antibody (ab70536; Abcam), following standard IHC staining procedures. The IHC staining results were scored independently by a pathologist from the pathology core lab at NHRI and three investigators, who were blinded to the clinical data. The correlation between nuclear and cytosolic expression levels of KIF2C and clinical characteristics, including tumor stages and lymph node metastasis stages, was assessed using the Immune Reactivity Scoring System (IRS). The IRS was calculated by multiplying the intensity of KIF2C staining (score, 0-4) by the area score of KIF2C-positive cells (4, 51-100%; 3, 31-50%; 2, 11-30%; 1, 10%; 0, 0%), which resulted in values ranging from 0 to 16.

Lentivirus-Mediated shRNA Interference

The breast cancer cell lines were seeded in 10-cm dishes with 50%-70% confluence and infected with recombinant lentiviruses carrying shKIF2C (C6 Core Lab, Academia Sinica, Taipei, Taiwan) and 8 μg/mL polybrene. After incubation at 37° C. for three days, the cells were subjected to time-lapse imaging or cell viability assay.

### In Vivo Tumor Growth Inhibition in 4T1-Derived Syngeneic Models

Female BALB/c mice (From BioLASCO, Taiwan) were 9-10 weeks old and had a body weight (BW) range of 18-22 g in the study. The mice were housed at the National Health Research Institute (NHRI) Animal Center in accordance with the Institutional Animal Care and Use Committee procedures and guidelines. The 4T1-derived cells, including 4T1, 4T1 R4, and 4T1 R8, used for implantation were harvested during log phase growth and resuspended in phosphate-buffered saline. Each mouse was injected s.c. in the right flank with 5×10⁵ cells. After tumor size reached 60-100 mm 3 (D1), mice were divided into groups and received treatment with vehicle or different compounds alone or in combinations. Tumor size, in mm 3, was measured twice per week and calculated from Tumor volume=(w 2×1)/2, where w=width and 1=length in mm of the tumor. The values of tumor size were expressed as mean±SEM.

Liquid Chromatography with Tandem Mass Spectrometry

Sample preparation and data processing procedures were detailed in the supplementary methods. For proteome analysis, LC-MS/MS analysis was performed with a Thermo Scientific™ UltiMate™ 3000 RSLCnano system (Thermo Fisher Scientific) coupled to an Orbitrap Eclipse™ Tribrid™ Mass Spectrometer (Thermo Fisher Scientific). From each digested peptide sample, ˜0.2 g was injected onto the Thermo Scientific™ PepMap™ C18 25 cm×75 m ID column (Thermo Fisher Scientific) with a 6-24% ACN gradient in 0.1% FA 20 over 83 min at a flow rate of 300 nL/min and total for 120 min per LC run. For data-dependent-acquisition (DDA) mode, the spectra of full MS scan (m/z 375-1500) were acquired in the Orbitrap mass analyzer at 120000 resolution for a maximum injection time of 246 ms with an AGC target value of 4e5. Cycle time was set at 3 s with an isolation window of 1.4 Th and dynamic exclusion time was set to 20 s. Precursors were fragmented by HCD using a normalized collision energy of 30% and analyzed using the Orbitrap at 30000 resolution for a maximum injection time of 54 ms with AGC target value of 5e4.

For PTM analysis, ˜0.5 g peptides were injected onto the Thermo Scientific™ PepMap™ C18 25 cm×75 μm ID column (Thermo Fisher Scientific) with a 6-24% ACN gradient in 0.1% FA over 51 min at a flow rate of 300 nL/min and total for 80 min per LC run. For DDA mode, the spectra of full MS scan (m/z 375-1500) were acquired in the Orbitrap mass analyzer at 120,000 resolution for a maximum injection time of 246 ms with an AGC target value of 4e5. Cycle time was set at 3 s with an isolation window of 1.4 Th and dynamic exclusion time was set to 20 s. Precursors were fragmented by HCD using a normalized collision energy of 30% and analyzed using the Orbitrap at 30000 resolution for a maximum injection time of 54 ms with AGC target value of 5e4. The scan range (m/z) was set to 110-2000.

### Statistical Analysis

At least three independent experiments were carried out for each experimental setting. The preprocessing results were analyzed using MATLAB, R language, and Microsoft Excel, as indicated. The student's t-test evaluated the statistical results, where p-value <0.05 was considered statistically significant, and then graphically displayed as mean±SEM (standard error of the mean).

### Expression of KIF2C in Breast Cancer

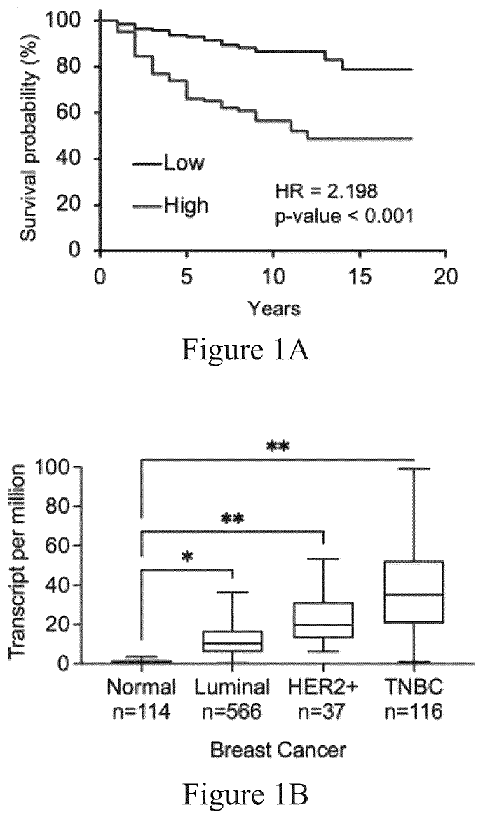

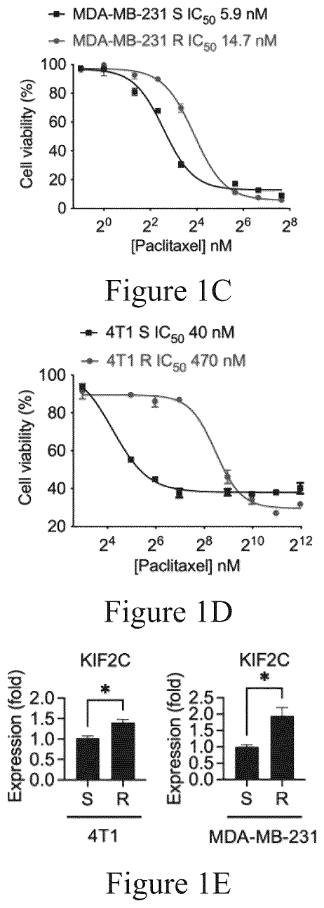

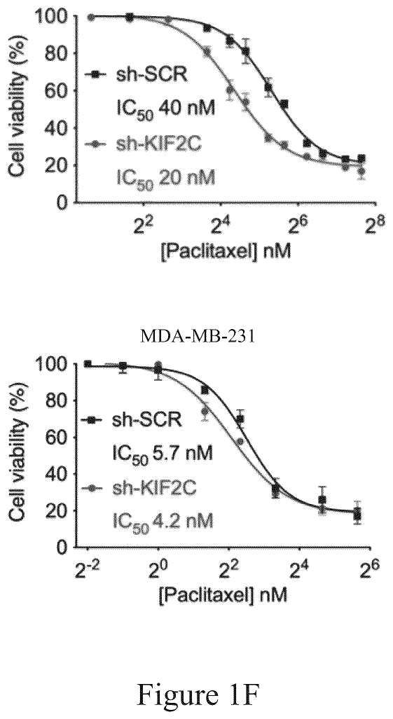

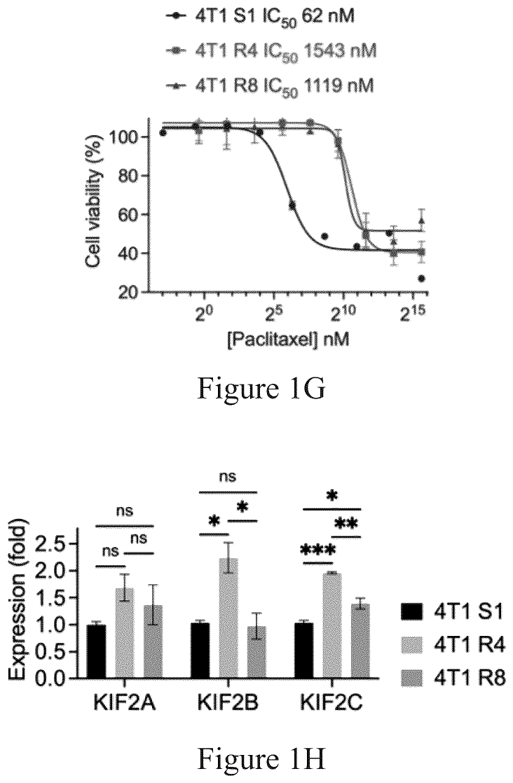

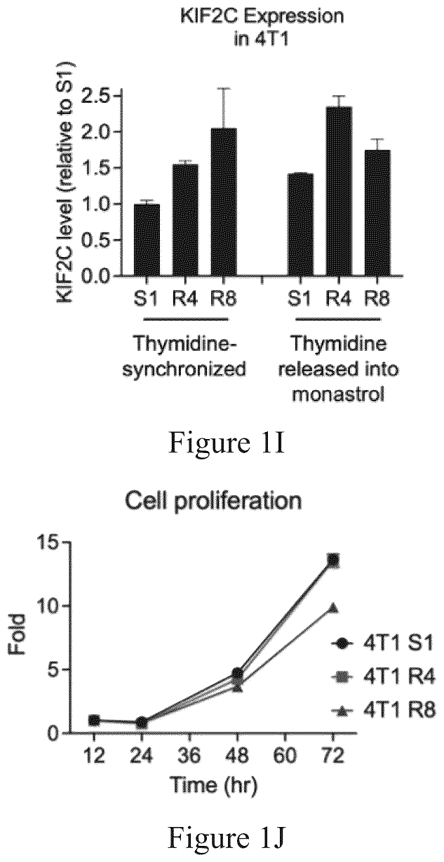

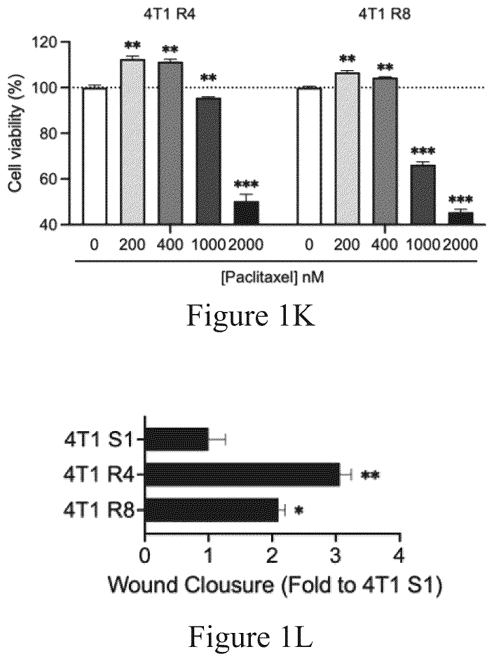

In this study, three clinical datasets (GSE25066 n=508, GSE41998 n=295, and GSE32646 n=123) were included in the systems biology analysis to identify differentially expressed genes in TNBC. KIF2C was the only target that fulfilled the following criteria: (1) over-expression in all subtypes of TNBC versus non-TNBC with p-value <0.05 and fold change >1.3-fold; (2) prognosis filter in the PRECOG survival z-score >2; (3) low abundance expression in normal tissues as analyzed from GTEx dataset from THE HUMAN PROTEIN ATLAS. Kaplan-Meier plot revealed that a higher KIF2C expression was associated with poor survival of breast cancer patients (FIG. 1A). KIF2C is a member of the kinesin-13 family, sharing a similar functional motor domain with the other two kinesin-13 family members, KIF2A and KIF2B. Based on the GTEx dataset, KIF2A is ubiquitously expressed in most tissues, whereas KIF2B and KIF2C are detected predominantly on the testis. We explored the TCGA database among different breast cancer subtypes. KIF2C expression is elevated in all types of breast cancers, and the highest level of KIF2C is detected in TNBC (FIG. 1B). Similarly, we classified breast cancer subtypes of the GTEx dataset, and KIF2C expression was elevated in all types of TNBC when compared to non-TNBC. Taken together, these data indicate that KIF2C is differentially expressed in TNBC among other breast cancer types.

To validate KIF2C expression in TNBC, we confirmed that KIF2C protein was high in TNBC cell lines (MDA-MB-231, MDA-MB-468, and Hs578T) when compared to non-TNBC lines (MCF-7 and SK-BR-3). Notably, depletion of KIF2C by shRNA significantly reduced cell survival of TNBC cell lines (data not shown). We analyzed the mitotic progression of MDA-MB-231 cells by time-lapse imaging. Significant mitotic defects were detected when depleted with KIF2C, with 44% showing cytokinesis failure and 9% showing mitotic arrest (data not shown). Taken together, KIF2C is essential for the cell survival and mitotic progression of TNBC.

### Elevation of KIF2C Increased Chemoresistance

To investigate whether KIF2C is involved in the development of chemoresistance, we established chemoresistant TNBC cell lines by gradually increasing the dosage of paclitaxel, which eventually led to a 2.5- and 11.8-fold increase in chemoresistance in MDA-MB-231 R (FIG. 1C) and mouse 4T1 R cells (FIG. 1D). In both cases, KIF2C expression levels were significantly increased in the resistant cells (FIG. 1E). The depletion of KIF2C by shRNA increased paclitaxel sensitivity by 2 and 1.4-fold in 4T1 and MDA-MB-231 cells, respectively (FIG. 1F). In the following studies, we focus on studying 4T1-resistant clones, which showed a profound resistance against paclitaxel and thus could be applied in the syngeneic mice tumorigenesis model. We selected single clones, including one representative sensitive clone (S1) and two resistant clones (R4 and R8), in the following studies. The IC₅₀ values of paclitaxel were 62, 1543, and 1119 nM for S1, R4, and R8 cells, respectively (FIG. 1G). The expression levels of KIF2A, KIF2B, and KIF2C in these cell lines were compared using Western blotting. KIF2C was upregulated in both R4 and R8 cells, with R4 cells exhibiting a higher level of KIF2C than R8 cells. In contrast, KIF2B was upregulated only in R4 cells, while KIF2A levels remained consistent across three cell lines (FIG. 1H). To explore if KIF2C upregulation is associated with cell proliferation, we compared KIF2C expressions in interphase (thymidine synchronized) and mitotic (thymidine released into monastrol) cells. As expected, KIF2C expression was higher in mitotic cells across all samples. In thymidine synchronized cells, KIF2C levels were higher in R4 and R8 cells compared to S1 cells (FIG. 1I). We noted that S1 and R4 cells had similar proliferation rates, while R8 cells proliferated slightly slower (FIG. 1J). Thus, the induction of KIF2C in R4 and R8 cells was likely independent of cell proliferation. Interestingly, R4 and R8 cells had slightly higher proliferation in the presence of 200-400 nM of paclitaxel, and cell viability decreased at concentrations over 1000 nM (FIG. 1K). In the following experiments, we constantly cultured R4 and R8 cells with 200 nM paclitaxel to maintain the chemoresistance. Morphologically, R4 and R8 cells displayed spindle-shaped morphology, whereas S1 cells displayed epithelial-like morphology (data not shown). KIF2C upregulation was shown to promote cancer cell migration and invasion by regulating microtubule dynamics. We found that cell motility of R4 and R8 were at least 2-fold higher than S1 cells, with R4 cells migrating faster than R8 cells (FIG. 1L). Taken together, we identified KIF2C as a differentially expressed gene in TNBC, and its upregulation correlated with chemoresistance and increased cell migration.

Paclitaxel Facilitates KIF2C Activity when Tubulin is Polyglutamylated

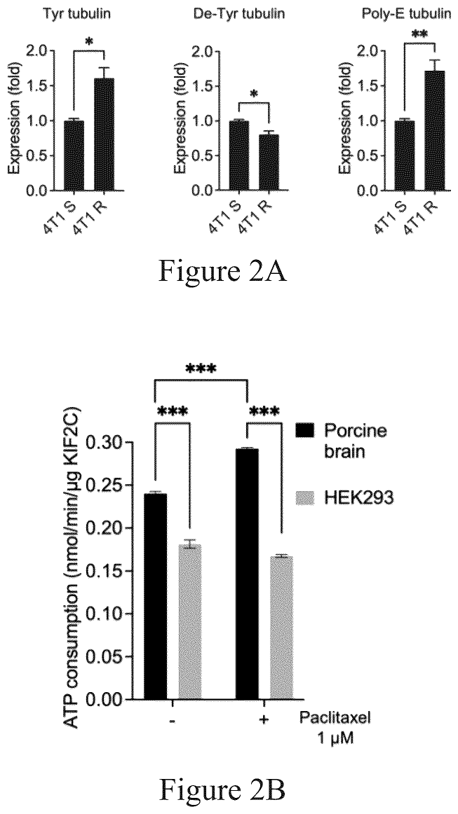

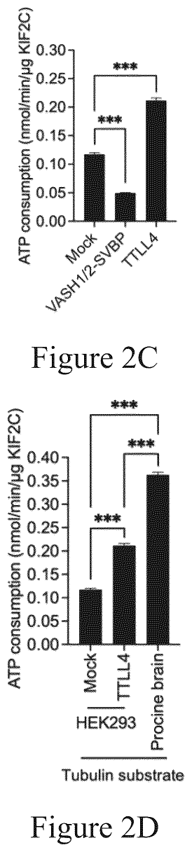

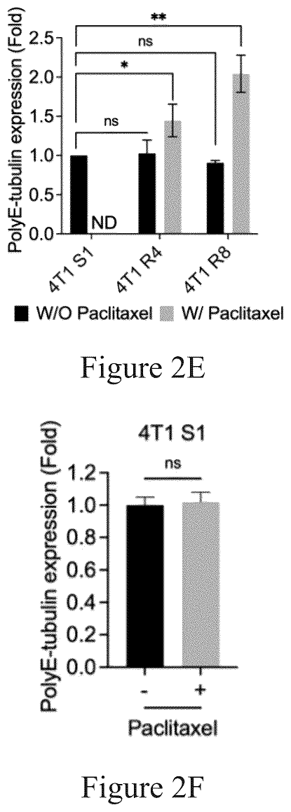

Tubulin tyrosination and polyglutamylation are known to modulate the KIF2C activity. Tyrosination is located at the C-terminus end of a-tubulin, whereas polyglutamylation can occur in both a- and P-tubulin. We detected increased tyrosinated and poly-E tubulins in 4T1 R cells (FIG. 2A). To study whether and how tubulin PTMs modulate KIF2C activity, we produced recombinant proteins containing minimal function domains of KIF2A, 2B, and 2C using the pET-28a E. coli expression system and developed an in vitro kinesin assay using tubulin purified from porcine brain as the substrate. In this assay, KIF2C hydrolyzed ATP at 0.2 nM per min per microgram of KIF2C, slightly higher than that of KIF2A and KIF2B (data not shown). We next compared KIF2C kinesin activity in reactions containing tubulin purified from porcine brain or human HEK293 cells. Compared to HEK293 cells, tubulin purified from porcine brain contained higher levels of poly-E tubulin and de-tyrosinated tubulin (data not shown). The overall KIF2C activity was higher in the reaction containing porcine brain tubulin than HEK293 tubulin. Most importantly, the addition of paclitaxel further increased KIF2C activity when using porcine brain tubulin as the substrate (FIG. 2B). These results implied that KIF2C is able to depolymerize microtubules in the presence of paclitaxel.

To clarify the interplay between tubulin tyrosination and polyglutamylation, we transfected HEK293 cells with VASH1/2-SVBP and TTLL4. Overexpression of VASH1/2-SVBP increased de-tyrosinated tubulin with a mild reduction of poly-E tubulin, whereas TTLL4 increased poly-E tubulin robustly (data not shown). We found that prior expression of VASH1/2-SVBP for 48 hr largely suppressed tubulin polyglutamylation mediated by TTLL4 (data not shown), indicating that tubulin tyrosination is a prerequisite of subsequent polyglutamylation. Notably, KIF2C activity was reduced with de-tyrosinated tubulin purified from VASH1/2-SVBP-transfected cells and was enhanced with poly-E enriched tubulin purified from TTLL4-transfected cells (FIG. 2C). Accordingly, KIF2C favors poly-E enriched tubulin as the substrate.

To investigate the molecular interaction between KIF2C and tubulin, we incubated recombinant KIF2C with porcine brain or HEK293 tubulins at a molar ratio 1:2 and then subjected to the affinity pull-down assay. The affinity of porcine brain tubulin to KIF2C is approximately 2-fold higher than HEK293 tubulin (data not shown). Noted that porcine brain may contain different tubulin isotypes from humans and mice, we compared KIF2C binding affinity with tubulin purified from HEK293 cells. KIF2C interaction was reduced by 80% with tubulin purified from VASH1/2-SVBP transfected cells (data not shown). We also compared tubulin PTMs in HEK293 cells transfected with CCP5 and TTLL4. Expression of CCP5 slightly reduced poly-E tubulins, whereas TTLL4 significantly increased poly-E tubulin (data not shown). KIF2C activity was enhanced when using tubulin purified from TTLL4-transfected cells, but to a lesser extent when using porcine brain tubulin as the substrate (FIG. 2D). We confirmed that tubulin purified from TTLL4 enhanced binding affinity with KIF2C, and the strongest interaction was detected with porcine brain tubulin, which contained the highest level of tubulin polyglutamylation (data not shown). Thus, KIF2C activity can be modulated by increasing tubulin polyglutamylation, which serves as a preferential substrate of KIF2C, especially in the presence of paclitaxel (FIG. 2D). In this study, R4 and R8 cells were maintained with 200 nM paclitaxel. We found that the removal of paclitaxel reduced poly-E tubulin in R4 and R8 cells (FIG. 2E). Interestingly, treatment of paclitaxel in S1 cells did not increase poly-E tubulin expression (FIG. 2F). Accordingly, paclitaxel-mediated induction of tubulin polyglutamylation is specific for chemoresistant cells.

Given that changes in tubulin isotype compositions may contribute to paclitaxel resistance, we performed liquid chromatography with tandem mass spectrometry (LC-MS/MS) analysis. In all cases, the overall tubulin abundance was consistent among chemosensitive and chemoresistant 4T1 and MDA-MB-231 cells (data not shown). In MDA-MB-231 cells, we identified three types of a tubulins (1a, 1c, and 4a) and five types of 3 tubulins (2a, 3, 4a, 4b, and 6) (data not shown). Noted that tubulin b4a was increased in MDA-MB-231 R cells. In 4T1 cells, we detected four types of a tubulins (1a, 1c, 4a, and 8) and four types of 3 tubulins (2a, 4b, 5, and 6), which were consistent among 4T1 S1, R4, and R8 cells (data not shown). These results indicated that both tubulin isotype composition and polyglutamylation were involved in the development of paclitaxel resistance in MDA-MB-231 cells, whereas tubulin polyglutamylation likely played a predominant role in 4T1 chemoresistance.

Electrostatic interaction between the positively charged motor domain of kinesin and the negatively charged C terminus of tubulin, known as the E-hooks, was shown to increase the processivity and speed of kinesin. We analyzed the published crystal structure (PDB code: 10 5MIO), which contains the KIF2C motor domain and the α/β-tubulin heterodimer. In this structure, the tubulin-KIF2C interaction interface is mediated by electrostatic interactions, with negative charges on α- or β-tubulin interacting with positively charged residues on KIF2C, e.g., Glu420 of β-tubulin interacts with Arg540 of KIF2C. Note that the E-hooks and poly-E were not included in the tubulin heterodimer displayed in the crystal. We superimposed the C-terminus tail of β-tubulin with polyglutamylation on this structure and found that the poly-E tail was adjacent to a series group of positive-charged residues on KIF2C. We proposed that tubulin polyglutamylation enhances KIF2C binding via increased electrostatic interaction. To confirm this, we conducted an affinity pull-down assay with escalating ionic strength. At 300 mM KCl, the interaction between KIF2C and poly-E low tubulin from HEK293 was lost, while 66% of poly-E enriched tubulin from porcine brain still bound to KIF2C (data not shown). Taken together, these results indicated that tubulin polyglutamylation may strengthen electrostatic interaction with KIF2C and thus facilitate microtubule depolymerization, even in the presence of paclitaxel.

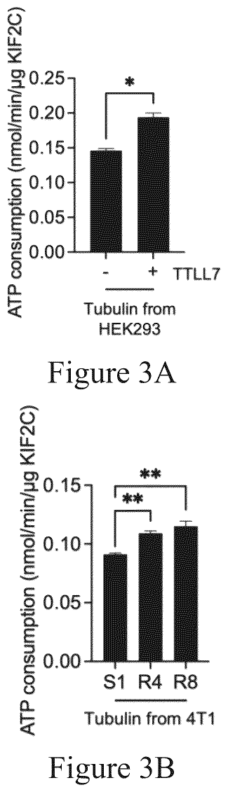

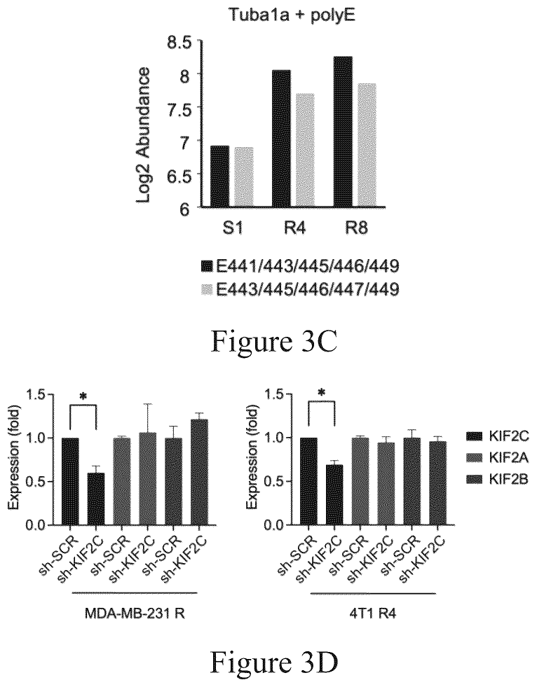

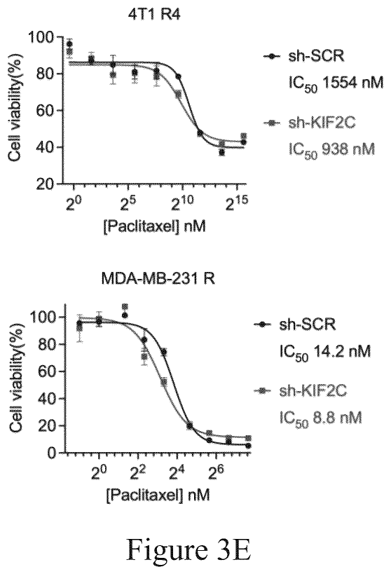

To further characterize the effect of tubulin polyglutamylation, we performed the in vitro polyglutamylation on HEK293 tubulin using recombinant TTLL7. As expected, treatment of TTLL7 increased tubulin polyglutamylation by 55%, and employing TTLL7-treated tubulin enhanced KIF2C activity by 26% (FIG. 3A). The KIF2C pull-down assay confirmed an increase in KIF2C binding affinity with TTLL7-treated tubulin (data not shown). Similarly, we confirmed that tubulin from R4 and R8 cells contained higher levels of poly-E tubulin and enhanced KIF2C binding activity (FIG. 3B). We next performed LC-MS/MS analysis on tubulin purified from 4T1 cells and identified polyglutamylated sites at the C-terminus of a-tubulin tail, including E441, E443, E445, E446, E447, and E449. Noted that glutamylated peptides identified from LC-MS/MS all contained Y451 residue. This results confirmed our finding that tubulin tyrosination is a prerequisite of polyglutamylation (data not shown). Here we showed that the extracted peak area of polyglutamylated peptides on the tubulin C-terminal tail were significantly upregulated in R4 and R8 cells, compared to S1 cells (FIG. 3C).

Finally, we depleted KIF2C with lentiviruses carrying shRNA of KIF2C and detected an approximately 1.6-fold increased chemosensitivity in MDA-MB-231 R and 4T1 R4 cells (FIG. 3E). The expression levels of KIF2A and KIF2B were not altered upon KIF2C depletion (FIG. 3D), implying that there is no compensatory mechanism for the expression of kinesin-13 family members.

### Development of Novel KIF2C Inhibitors

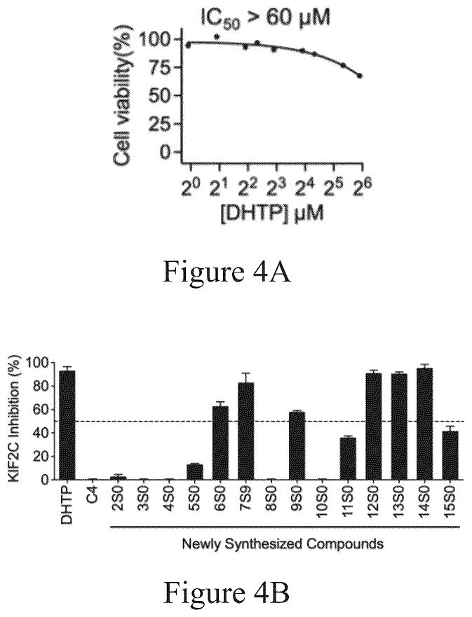

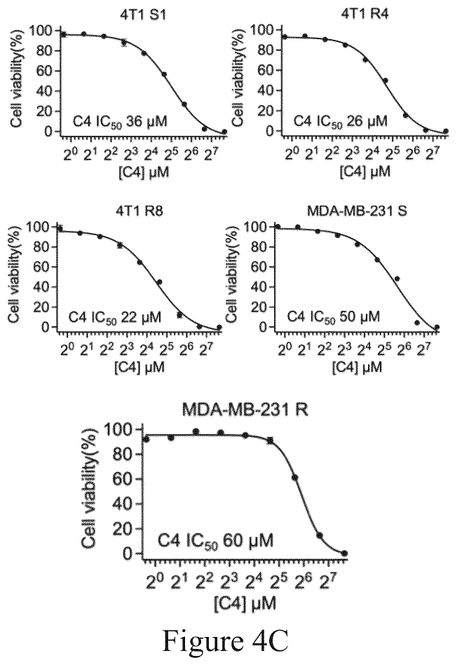

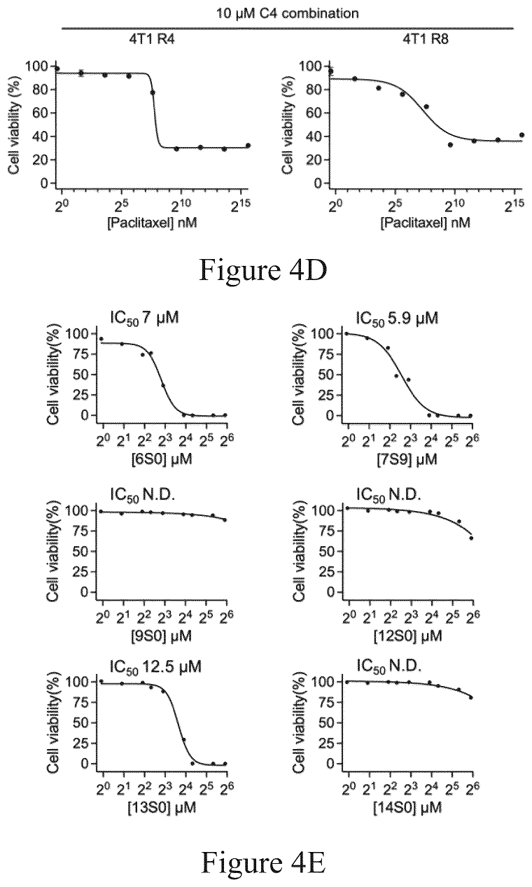

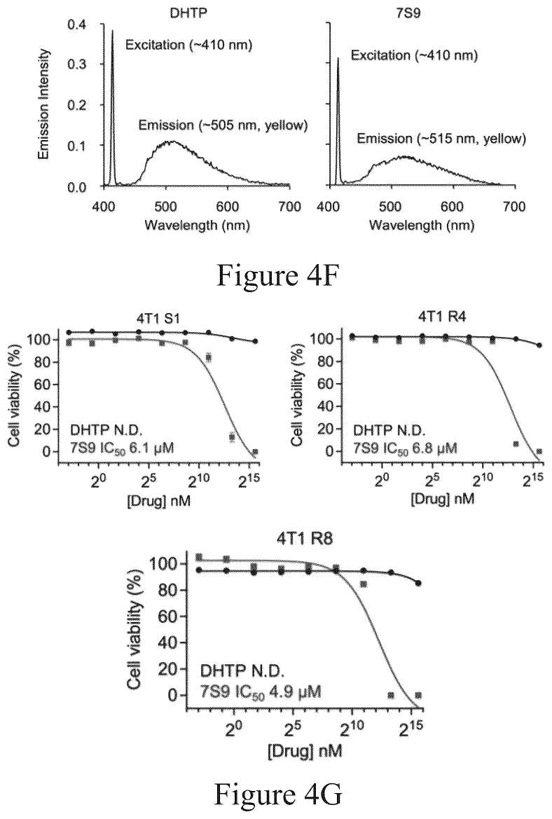

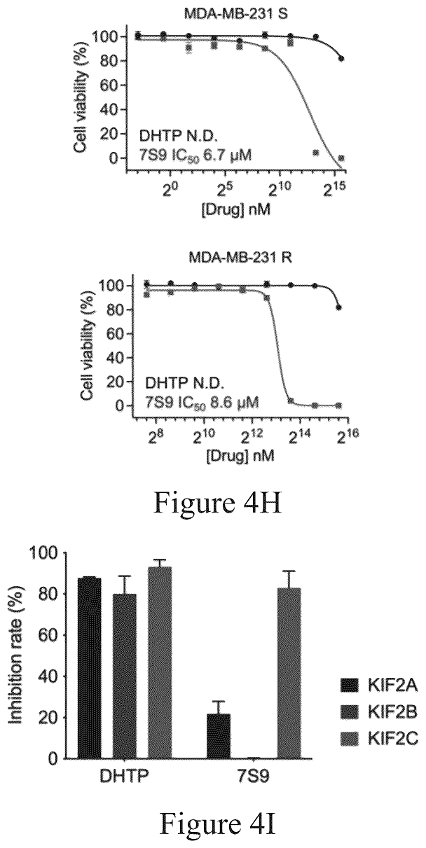

DHTP is an allosteric inhibitor of kinesin-13 family proteins. We found that DHTP was not cytotoxic to MDA-MB-231 cells (FIG. 4A) but could inhibit KIF2C kinesin activity (FIG. 4B). Recently, another KIF2C inhibitor, C4, was identified using a conformation-based FRET analysis. We found that C4 could not inhibit KIF2C in our kinesin assay (FIG. 4B) but was cytotoxic to 4T1 and MDA-MB-231 cells, albeit at high molar ranges (FIG. 4C). R4 and R8 cells were slightly more sensitive to C4 than S1 cells, whereas the C4 IC₅₀ in MDA-MB-231 S and R cells were similar (FIG. 4C). The combination of a sublethal dose of C4 (10 μM) increased paclitaxel sensitivity in R4 and R8 cells by approximately 7 folds, with the combination index (CI) of 0.53 for R4 and 0.59 for R8 cells (FIG. 4D and the following Table 2). In Table 2, the combination index (CI) was calculated by dividing the IC₅₀ values of a single paclitaxel treatment by the C4 combination estimated. These results implied that KIF2C inhibitors have a synergistic effect with paclitaxel in TNBC cells. To acquire a more potent KIF2C inhibitor, we synthesized a series of DHTP derivatives by introducing various solubilizing groups to the carboxylic acid or replacing the chloride with fluoride. Among them, six candidates inhibited KIF2C activity by at least 50% at a concentration of 100 μM (FIG. 4B). Compounds 6S0, 7S9, and 13S0 showed cytotoxicity on MDA-MB-231 cells (FIG. 4E). As compound 7S9 is the most potent inhibitor of KIF2C, we continued follow-up studies with 7S9. The chemical structure of 7S9 is different from DHTP by replacing its carboxyl group with the N-ethyl piperazine group. DHTP and 7S9 share similar spectrum profiles, with significant absorbance at 410 nm and emitting a yellow color at around 510 nm wavelength (FIG. 4F). We noted that cells treated with 7S9 were yellowish, whereas cells treated with DHTP were not (data not shown). Following flow cytometry analysis, DHTP-treated cells showed no fluorescence signals, whereas 7S9-treated cells displayed a significant fluorescence signal at 510 nm emission, indicating that 7S9 has a good cell-penetrating property (FIG. 4J). 7S9 displayed apparent cytotoxicity on MDA-MB-231 R, MDA-MB-231 S, 4T1 S1, R4, and R8 cells, with IC₅₀ values between 4.9-8.6 μM. In striking contrast, DHTP was not cytotoxic to these cell lines (FIG. 4G and FIG. 4H).

TABLE 2
Paclitaxel IC₅₀ (nM)

| | Single | C4 combination | Fold change | CI |
|---|---|---|---|---|
| 4T1 R4 | 1543 | 217 | 7.1 | 0.53 |
| 4T1 R8 | 1119 | 157 | 7.1 | 0.59 |

To demonstrate the specificity of 7S9, we confirmed that 7S9 exhibited selective inhibition against KIF2C, while DHTP inhibited all three members of the kinesin-13 family (FIG. 4I). Additionally, treatment with 7S9 did not increase cytotoxicity in MDA-MB-231 cells upon depletion of KIF2C, in the presence or absence of paclitaxel (data not shown). These results confirmed that the cytotoxicity induced by 7S9 is specific to the presence of KIF2C. Taken together, we conclude that 7S9 is a potent, selective, and cell-penetrating inhibitor of KIF2C.

### 7S9 Blocks KIF2C-Mediated Microtubule Depolymerization

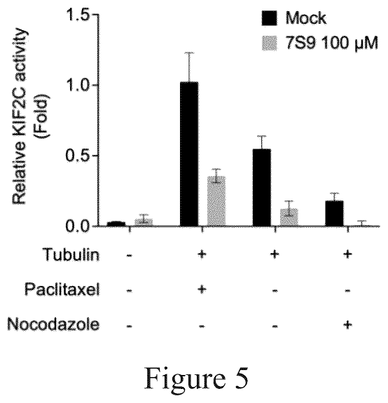

Here we explore if 7S9 may inhibit KIF2C activity in the presence of paclitaxel. In vitro kinesin assay of KIF2C was performed with high concentration (1 μM) paclitaxel or nocodazole for over-stabilized or depolymerized microtubules, respectively. We found that KIF2C activity was largely reduced in the reaction containing nocodazole, indicating that free-form tubulins were not favorable substrates of KIF2C (FIG. 5). Notably, 7S9 could successfully inhibit KIF2C activity in the presence of paclitaxel, indicating that the inhibitory effect of 7S9 was still intact when paclitaxel was present.

To explore the interplay between 7S9 and KIF2C, we performed molecular docking with Autodock Vina. We compared tubulin-bound (Blue, PDB code: 5MIO) and free-form KIF2C (Green, PDB code: 2HEH) and found significant conformational changes in KIF2C upon binding to the tubulin dimer, particularly at the α4 and α5 helices, which are situated at the binding interface with tubulin dimers. We identified two adjacent 7S9 binding sites within the pocket region of the α4 and α5 helix residues. These sites are situated at the central region of the KIF2C-tubulin interface. Notably, binding site 1 is exclusively exposed to tubulin-bound KIF2C (5MIO) and not to the free-form KIF2C (2HEH). Consequently, we propose that 7S9 may intercalate into the KIF2C-tubulin complex, thereby impeding the conformational changes in KIF2C necessary for its release from tubulin. To test this possibility, we performed the KIF2C-tubulin pull-down assay and demonstrated that the molecular interaction between KIF2C and tubulin was increased by 7S9 in a dose-dependent manner (data not shown).

To confirm if KIF2C-mediated microtubule depolymerization can be blocked by 7S9, we labeled microtubules with a fluorescent SiR-tubulin dye and imaged the integrity of microtubules under fluorescence microscopy. The addition of recombinant KIF2C and ATP successfully depolymerized microtubules in 30 min. This effect was completely blocked by 7S9 (data not shown). To study the impact of 7S9 in living cells, we explored intracellular microtubule distribution in HeLa cells expressing H2B-mCherry and eGFP-tubulin. The treatment of paclitaxel induced a significant accumulation of bundled microtubules at the cell periphery. In contrast, 7S9 induced puncta formation of tubulins in the cytoplasm. Combined treatment of paclitaxel and 7S9 induced mixed phenotypes of tubulins with both bundled and puncta tubulins (data not shown). In live cell imaging, 7S9-induced tubulin aggregation was initially detected at the microtubule plus ends, where KIF2C-mediated microtubule depolymerization took place, and then tubulin aggregation into distinct puncta in the cytoplasm. In contrast, such tubulin aggregation phenotype was not detected in cells treated with DHTP (data not shown). Finally, we showed that the depletion of KIF2C or the treatment of 7S9 did not change the level of poly-E tubulin in 4T1 R4 cells (data not shown).

In 4T1 S1 cells, bundled microtubules were detected in the presence of paclitaxel (data not shown). In striking contrast, bundled microtubules were not observed in 4T1 R4 and R8 cells (data not shown). These results indicated that general microtubule dynamics were intact in chemoresistant cells in the presence of paclitaxel. In R4 and R8 cells, treatment of 7S9 alone induced puncta microtubules. When combined with paclitaxel, 7S9 induced significant bundled microtubules in the cytoplasm (data not shown), resembling the reversal of microtubule phenotype in paclitaxel-treated S1 cells (data not shown). Taken together, these results indicated that 7S9 inhibits KIF2C activity by disrupting its dissociation cycle from microtubules and thereby re-sensitizes microtubules to paclitaxel in chemoresistant cells.

### Inhibition of KIF2C Increased Chemosensitivity in Paclitaxel-Resistant Cells

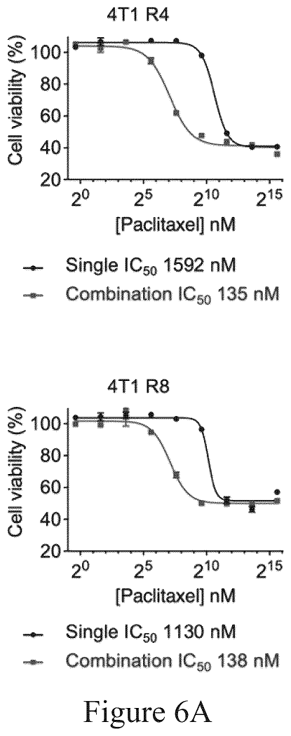

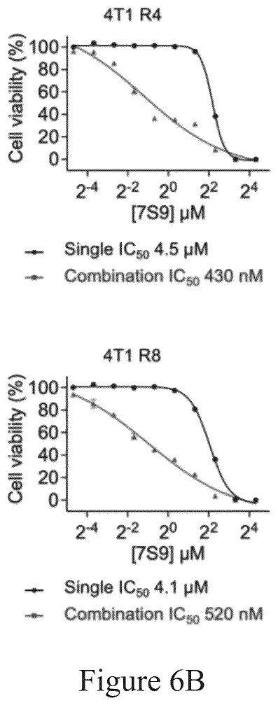

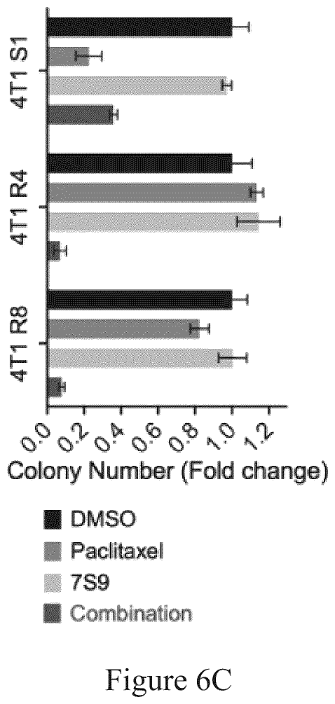

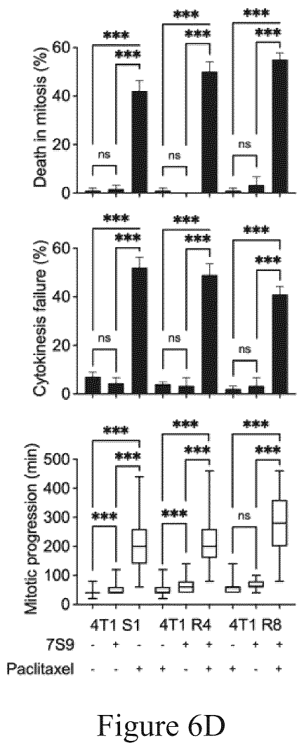

To clarify if 7S9 may be applied to overcome chemoresistance, we measured the cytotoxicity of paclitaxel in the presence of a sublethal dose of 7S9 (1 μM). Strikingly, the combined treatment of 7S9 reduced the IC₅₀ of 4T1 R4 and R8 cells by 11.8 and 8.2-fold, respectively (FIG. 6A). Along the same line, the presence of a non-lethal dose of paclitaxel (400 nM) increased cell sensitivity to 7S9 by 10.5 and 8-fold, respectively, for R4 and R8 cells (FIG. 6B). To study the long-term effect of the combination therapy, we performed the colony formation assay. For S1 cells, treatment of 400 nM paclitaxel suppressed colony formation by 80%, and combined treatment of 1 μM 7S9 did not further suppress colony formation. For R4 and R8 cells, suppression of colony formation was detected only with combined treatment of 7S9 and paclitaxel (FIG. 6C).

To study how mitotic progression is affected by 7S9, we performed time-lapse imaging. Paclitaxel significantly prolonged mitotic progression time and increased cytokinesis failure in S1 cells but not in R4 and R8 cells (data not shown). Approximately 40% of S1 cells died in mitosis when treated with paclitaxel. For R4 and R8 cells, combined treatment of 1 μM 7S9 and 400 nM paclitaxel induced significant defects in mitosis, including prolonged mitotic progression time, cytokinesis failure, and mitotic cell death (FIG. 6D). Noted that these mitotic defects detected in R4 and R8 cells resembled those found in S1 cells when treated with paclitaxel alone. Taken together, these results indicated that the treatment of 7S9 and paclitaxel may induce synthetic lethality in chemoresistant cells.

### Combination Treatment of 7S9 Augments the Anti-Tumor Effect of Paclitaxel

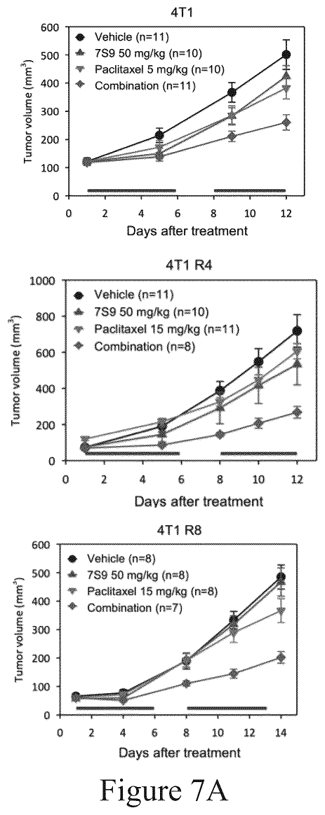

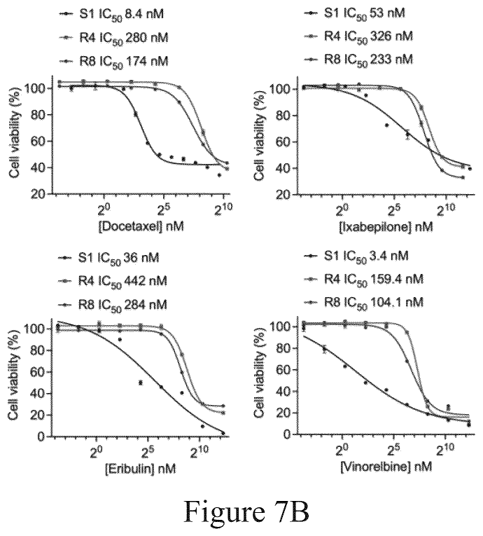

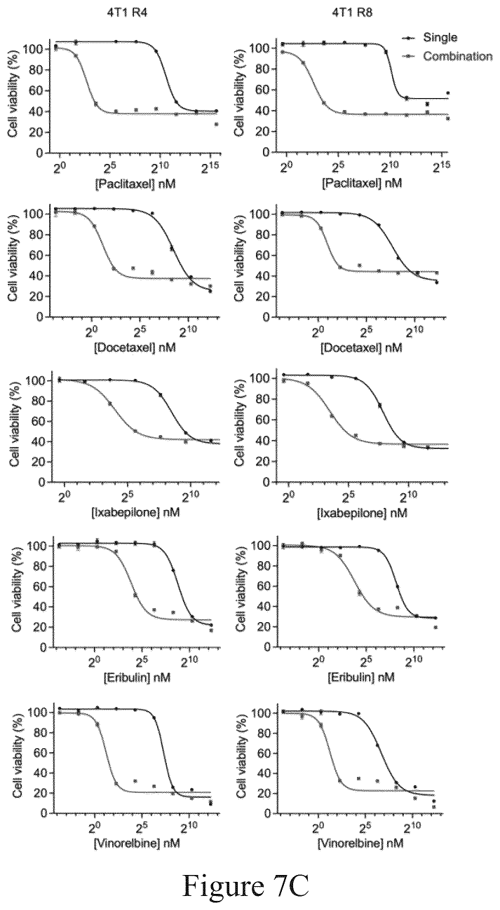

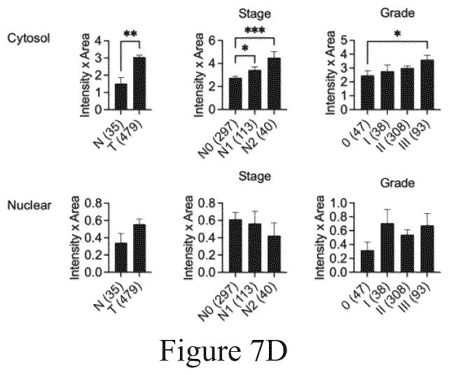

To explore the anti-tumor effect of 7S9, BALB/c mice were inoculated subcutaneously with 5×10⁵ of 4T1 cells. After tumor size reached 60-100 mm 3, mice were treated with paclitaxel and 7S9 as indicated. For 4T1 cells, tumor growth inhibition was detected at 20.3%, 31.3%, and 62.6% in mice treated with 7S9 (50 mg/kg), paclitaxel (5 mg/kg), and the combination, respectively (FIG. 7A). For R4 and R8 cells, 15 mg/kg paclitaxel was used. For R4 cells, tumor growth inhibition was detected at 29%, 41.7%, and 70.2% in mice treated with 7S9 (50 mg/kg), paclitaxel (15 mg/kg), and the combination. For R8 cells, tumor growth inhibition was prominently detected with 66.2% in the combination group (FIG. 7A). Taken together, 7S9 induced synergistic anti-tumor effects in both chemosensitive and chemoresistant cells when combined with paclitaxel.

### Inhibition of KIF2C Reversed Cross-Resistance Against MTAs

We next explored if 7S9 showed synergistic effects when combined with other commonly used MTAs, including docetaxel, ixabepilone, eribulin, and vinorelbine. Strikingly, at least a 5-fold increase in IC₅₀ against these MTAs was detected in R4 and R8 cells compared with S1 cells (FIG. 7B), implying that R4 and R8 cells had developed cross-resistance against general MTAs. Remarkably, the presence of a sublethal dose of 2 μM 7S9 significantly increased the chemosensitivity of R4 and R8 cells against all types of MTAs (FIG. 7C). We then calculated the combination index using the typical Chou and Talalay method. Among these MTAs, the highest synergistic cytotoxic effect of 7S9 was detected with taxane derivatives (docetaxel and paclitaxel), with an increased sensitivity by over 100-fold and a combination index (CI) below 0.5. For other MTAs, at least a 20-fold increase of chemosensitivity by 7S9 was detected in R4 and R8 cells, with CI between 0.46-0.53 (see the following Table 3). In Table 3, IC₅₀ of indicated MTAs when treated alone (single) or the combination with 2 μM 7S9 were shown. The fold change in chemosensitivity was calculated by dividing the IC 50 values of a single treatment by that of the 7S9 combination, and the combination index (CI) was estimated. Taken together, 7S9 displayed a synergistic effect with major types of MTAs in chemoresistant cells. Accordingly, inhibition of KIF2C may be applied to combat the cross-resistance of TNBC.

TABLE 3

| | | | |
|---|---|---|---|
| Single | Combination | Fold change | CI |

4T1 R4 IC₅₀ (nM)

| | | | | |
|---|---|---|---|---|
| Paclitaxel | 1543 | 6.4 | 241 | 0.45 |
| Docetaxel | 358 | 2.2 | 163 | 0.45 |
| Ixabepilone | 358 | 15.9 | 24 | 0.49 |
| Eribulin | 442 | 14.5 | 30 | 0.48 |
| Vinorelbine | 159 | 2.3 | 69 | 0.46 |

4T1 R8 IC₅₀ (nM)

| | | | | |
|---|---|---|---|---|
| Paclitaxel | 1119 | 6.1 | 183 | 0.49 |
| Docetaxel | 208 | 1.9 | 109 | 0.50 |
| Ixabepilone | 234 | 10.9 | 21 | 0.53 |
| Eribulin | 284 | 13.6 | 21 | 0.54 |
| Vinorelbine | 104 | 2.4 | 43 | 0.51 |

### KIF2C is a Potential Biomarker for Breast Cancer Progression

To further validate the role of KIF2C in breast cancer, we performed immunohistochemistry staining of KIF2C in four different tissue arrays with a collection of breast cancer tissues from 482 patients. Representative KIF2C expressions on tumors and normal tissues were shown (data not shown), and KIF2C was present in both cell nucleus and cytoplasm. Quantitatively, nuclear KIF2C expression was relatively higher in tumor tissues than in normal tissues but showed no correlation with tumor progression, as defined by the onset of lymph node metastasis (N1 and N2) and the stage of tumors (Stage 0, I, II, and III) (FIG. 7D). Notably, cytoplasmic KIF2C was significantly increased in tumor tissues and displayed positive correlations with lymph node metastasis and tumor progressions (FIG. 7D). Accordingly, cytoplasmic KIF2C may represent a suitable biomarker for tumor progression of breast cancer.

To investigate whether KIF2C can be used as an independent predictive biomarker of breast cancer, we performed the receiver operating characteristic (ROC) curve analysis as the binary classification of KIF2C expression levels in tumor versus non-tumor samples. Based on the results of tissue arrays, the “area under the curve” (AUC) values of cytoplasmic KIF2C were calculated as 0.67 with a P value <0.001, suggesting that cytoplasmic KIF2C can be used as a biomarker to distinguish breast cancer from adjacent normal tissues (data not shown). ROC curve analysis was also performed on the TCGA PanCancer Atlas dataset, and the AUC value of KIF2C mRNA expression was 0.97, with a P value <0.0001 (data not shown). These data implied that both the mRNA and protein levels of KIF2C have the predictive value to indicate breast cancer from normal tissues. We also analyzed KIF2C expression in the GSE25066 dataset, which contains classified information on different breast cancer subtypes. Strikingly, elevated levels of KIF2C mRNA could be used to distinguish TNBC from other breast cancer types (AUC=0.92, p<0.0001) (data not shown). Taken together, these analyses demonstrated that KIF2C is an indicative prognostic biomarker of breast cancer, most prominent in TNBC.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the disclosure to adapt it to various usages and conditions. Thus, other embodiments are also within the claims.

1. A compound represented by the following formula (I), or a pharmaceutically acceptable salt thereof: 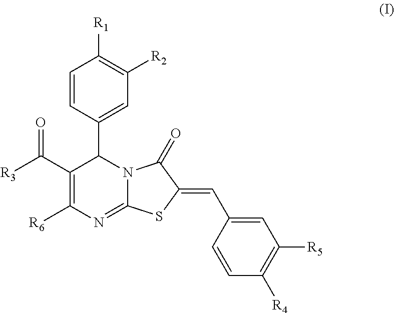
wherein,
one of R₁ and R₂ is H, and the other one of R₁ and R₂ is halogen, unsubstituted or substituted C₁₋₆ alkyl, unsubstituted or substituted C₁₋₆ alkoxy, —NRₐR_{b}, or a N-containing heteroaryl group, wherein Rₐ and R_{b} respectively is H or C₁₋₆ alkyl;
R₃ is —OH, unsubstituted or substituted C₁₋₆ alkyl, or unsubstituted or substituted C₁₋₆ alkoxy;
R₄ is halogen or —O—C₁₋₃ alkylene-R_{c}, wherein R_{c} is H, —CN, —OH, —C(═O)OR_{d}, —C(═O)Rₑ, C₂₋₆ alkynyl or a heteroaryl group; Rd is H, or unsubstituted or substituted C₁₋₆ alkyl; Rₑ is —NR_{f}R_{g}, or a heterocycloalkyl group substituted with —OH, C₁₋₆ alkyl, —NR_{f}R_{g}, cycloalkyl or a heterocycloalkyl group substituted with C₁₋₆ alkyl; wherein R_{f} is H or C₁₋₆ alkyl, and R_{g} is H, unsubstituted or substituted C₁₋₆ alkyl or —SO₂CF₃;
R₅ is H; or R₄, R₅ and carbon atoms attached thereto form a heterocycloalkyl group; and
R₆ is C₁₋₆ alkyl, with proviso that R₄ is not —OCH₂C(═O)OH when R₁ is Cl, R₂ is H, R₃ is C₃ alkyl, R₆ is methyl, and R₅ is H.

2. The compound of claim 1, wherein R₂ is H, and R₁ is F, Cl, Br, unsubstituted or substituted C₁₋₆ alkyl, unsubstituted or substituted C₁₋₆ alkoxy, —NRₐR_{b}, or imidazolyl.

3. The compound of claim 2, wherein R₁ is Cl or methoxy.

4. The compound of claim 1, wherein R₃ is unsubstituted or substituted C₁₋₆ alkoxy.

5. The compound of claim 1, wherein R₃ is unsubstituted C₁₋₆ alkoxy.

6. The compound of claim 1, wherein R₅ is H.

7. The compound of claim 1, wherein R₄ is F or —O—C₁₋₃ alkyl-R_{c}.

8. The compound of claim 7, wherein R₄ is —O—CH₂—R_{c}.

9. The compound of claim 8, wherein R₄ is —O—CH₂—C(═O)Rₑ.

10. The compound of claim 9, wherein Rₑ is a heterocycloalkyl group substituted with —OH, C₁₋₆ alkyl, —NR_{f}R_{g}, cycloalkyl, or a heterocycloalkyl group substituted with C₁₋₆ alkyl.

11. The compound of claim 10, wherein Rₑ is 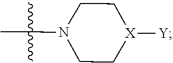
wherein X is N or C, and Y is C₁₋₆ alkyl, —NR_{f}R_{g} or a heterocycloalkyl group substituted with C₁₋₆ alkyl, wherein R_{f} and R_{g} respectively is a C₁₋₆ alkyl.

12. The compound of claim 11, wherein X is N, and Y is C₁₋₆ alkyl.

13. The compound of claim 12, wherein X is N, and Y is ethyl.

14. The compound of claim 11, wherein X is C, Y is —NR_{f}R_{g} or 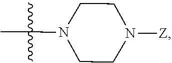
and Z is C₁₋₆ alkyl; wherein R_{f} and R_{g} respectively is a C₁₋₆ alkyl.

15. The compound of claim 14, wherein Y is —N(CH₃)₂ or 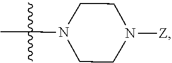
and Z is methyl.

16. The compound of claim 1, which is a maleic salt thereof.

17. The compound of claim 1, which is any one selected from the group consisting of:

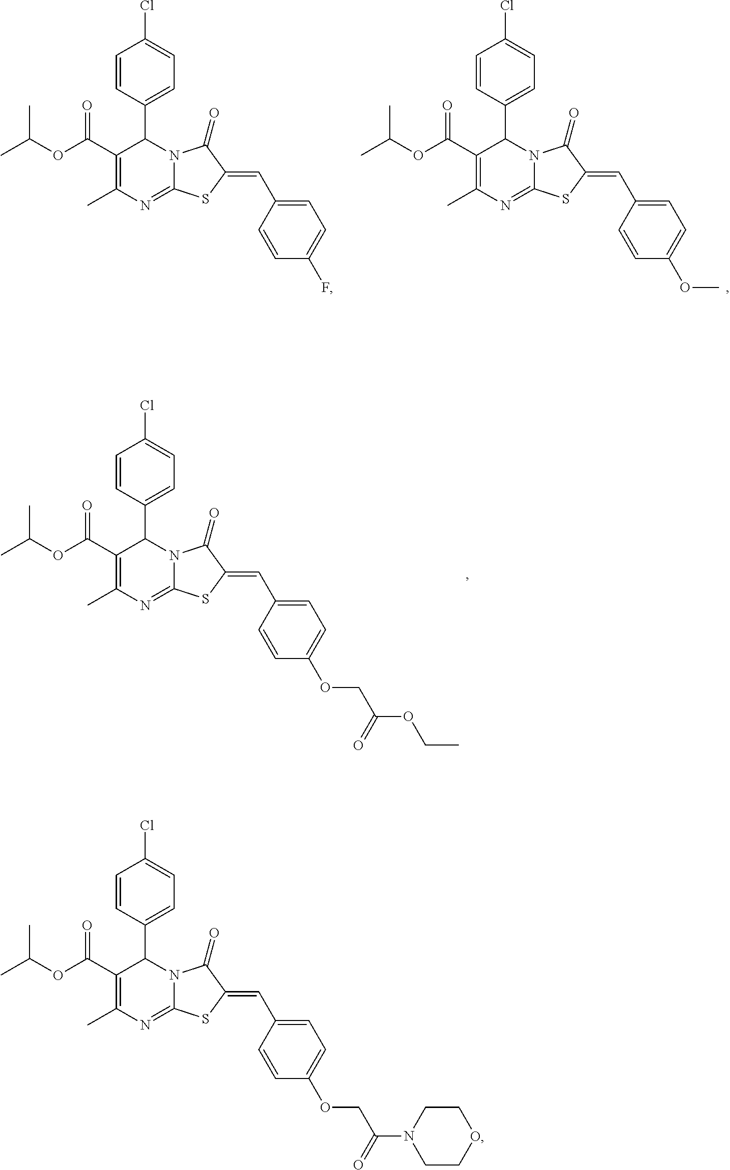

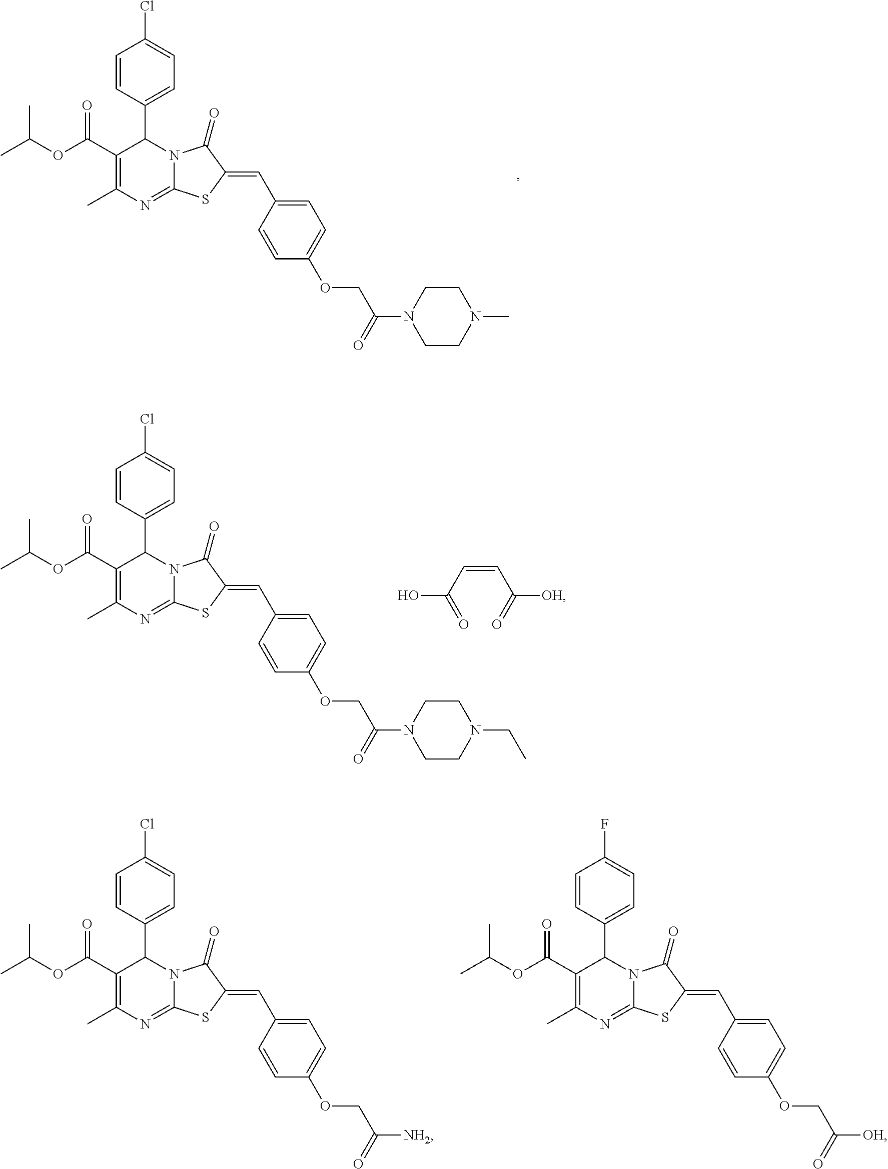

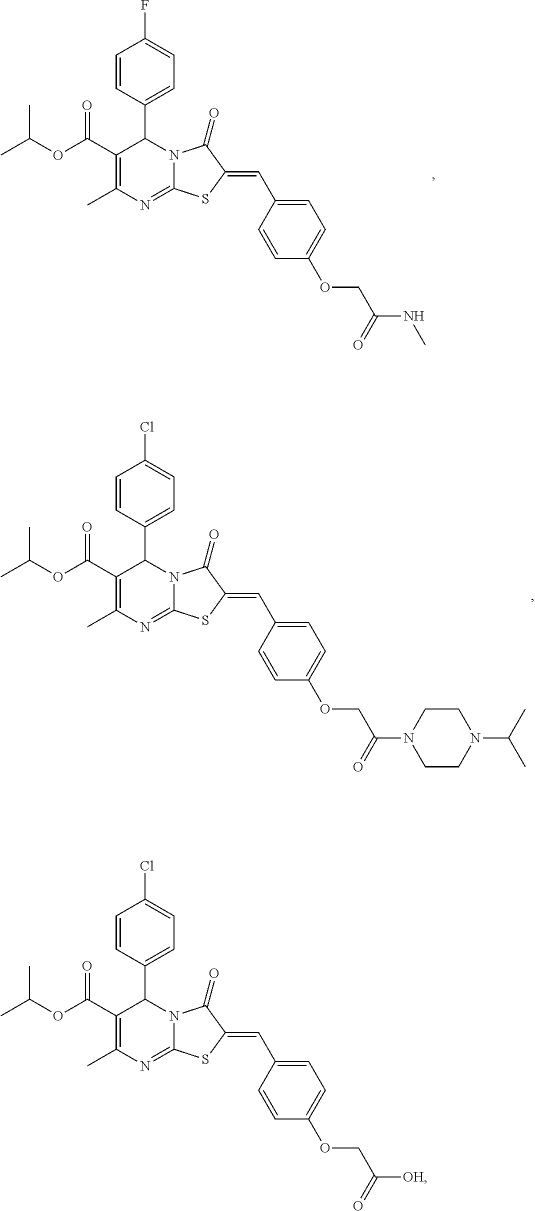

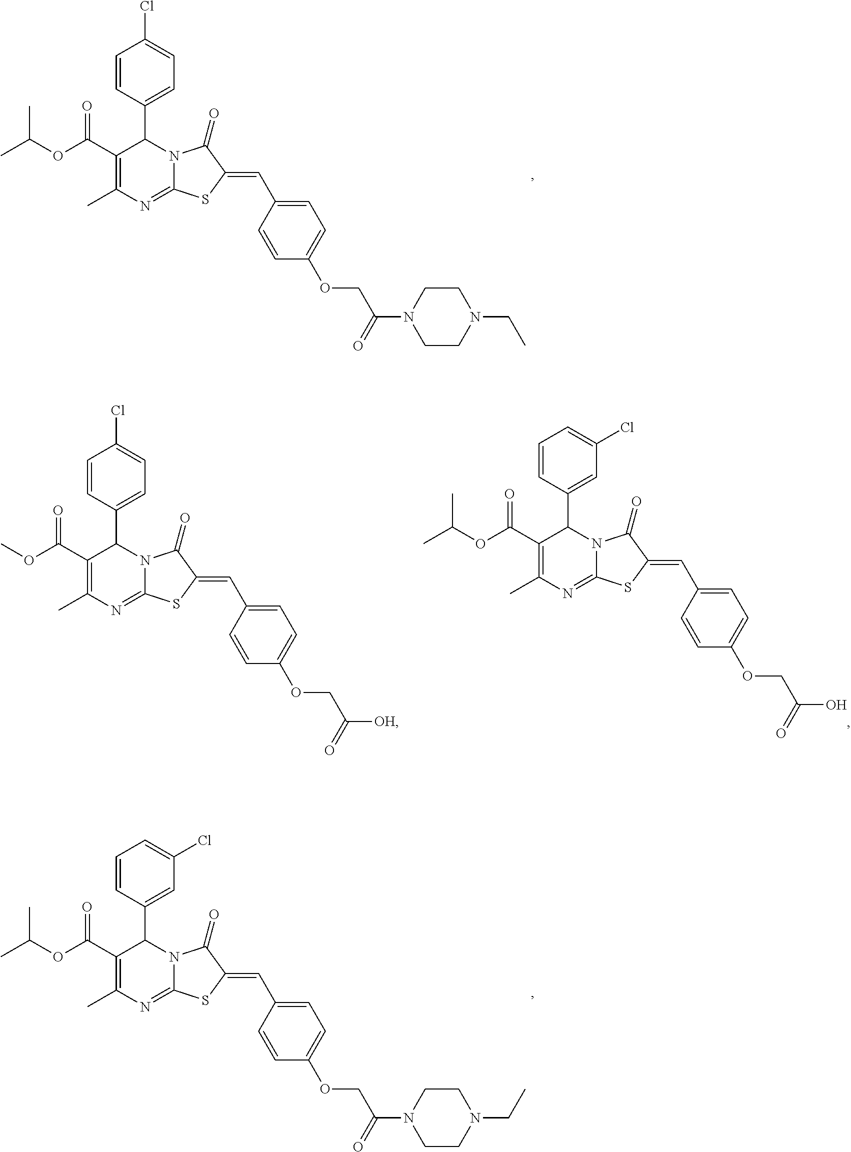

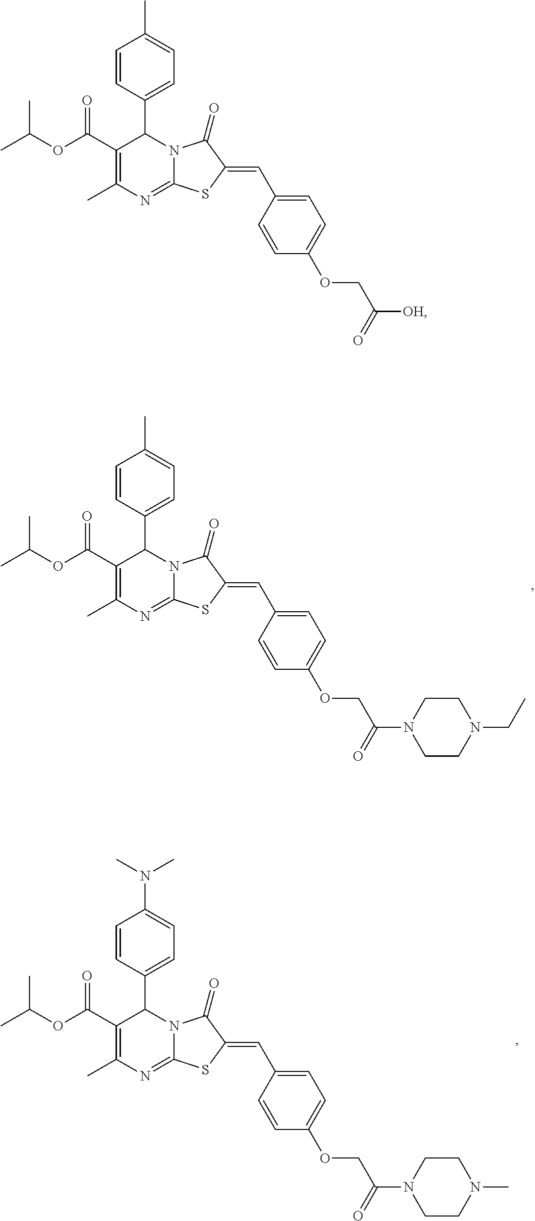

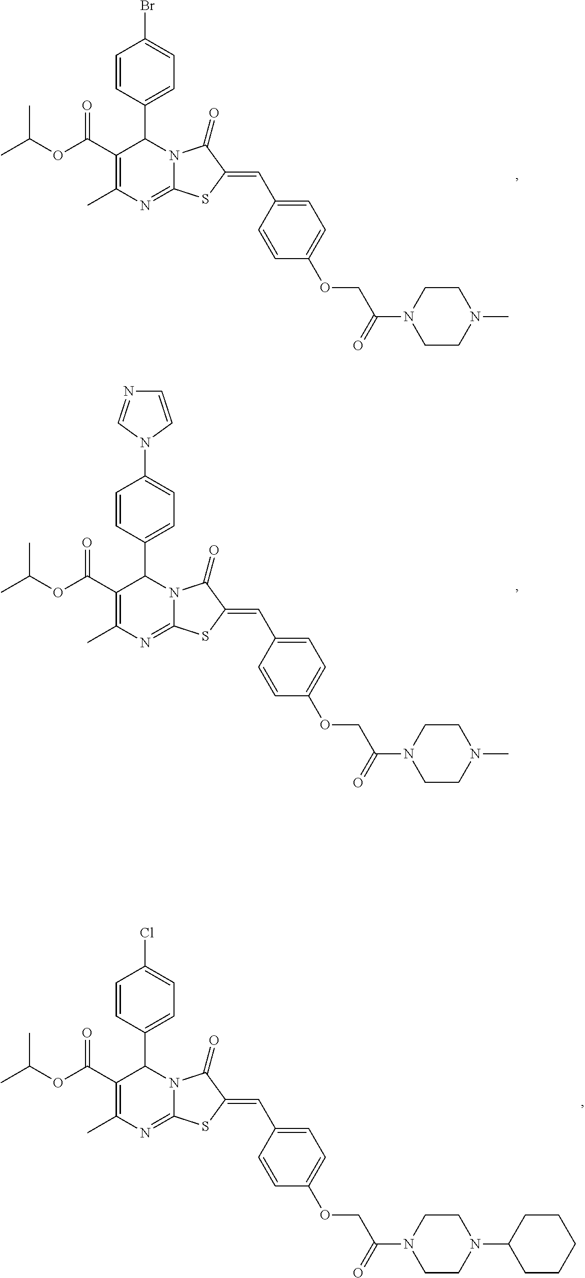

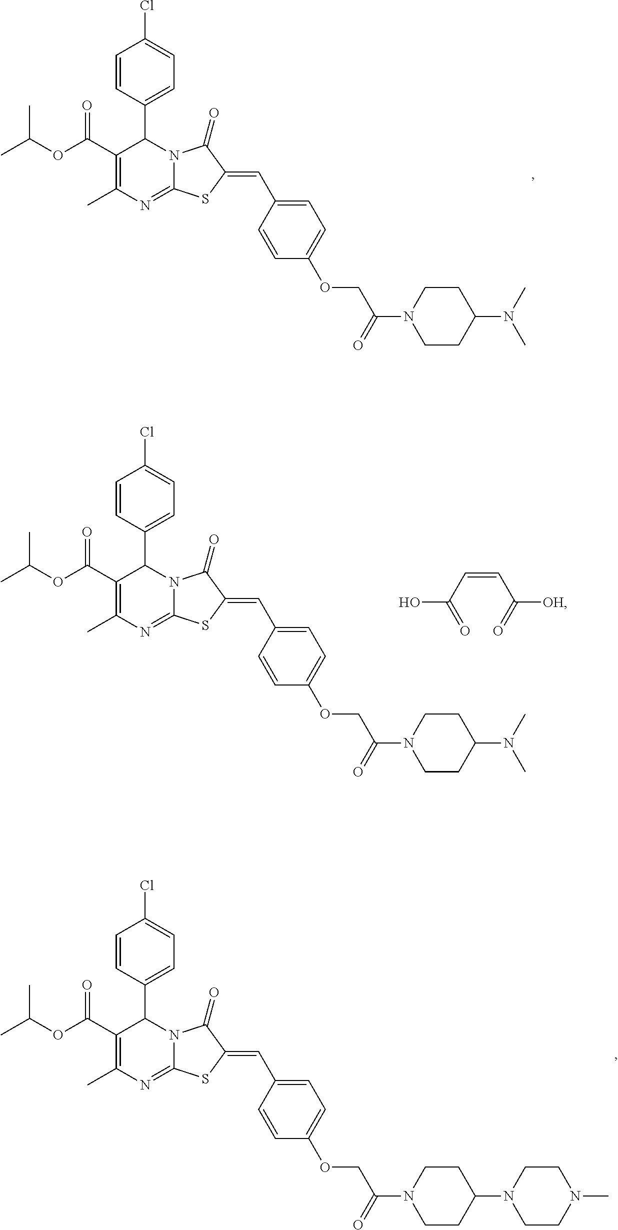

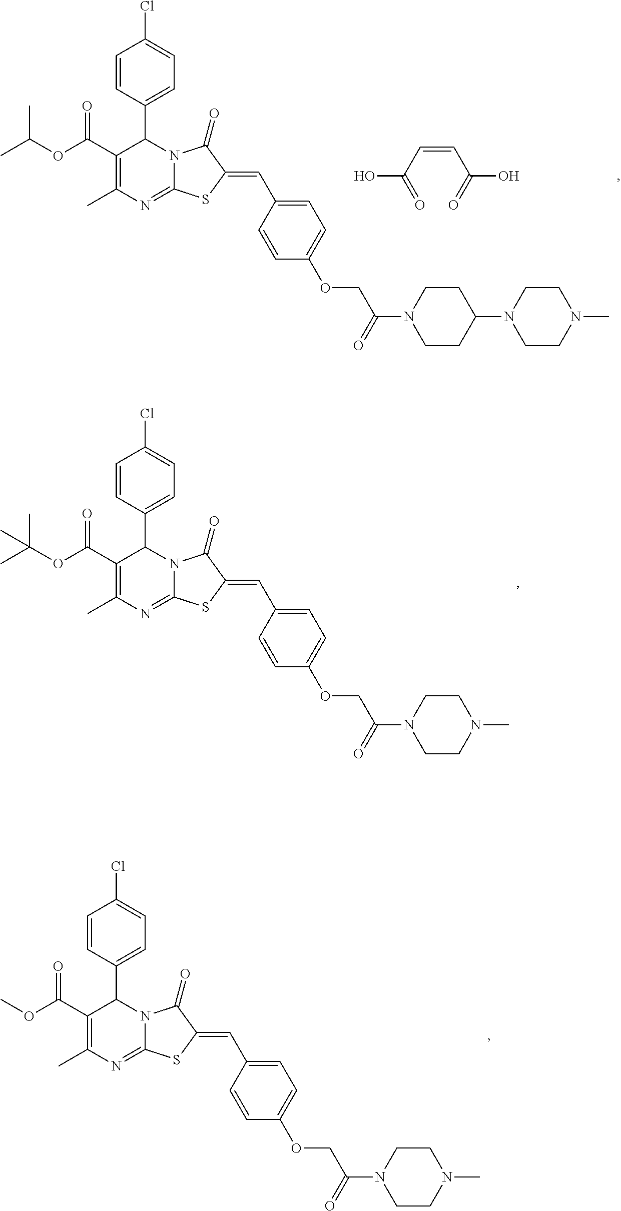

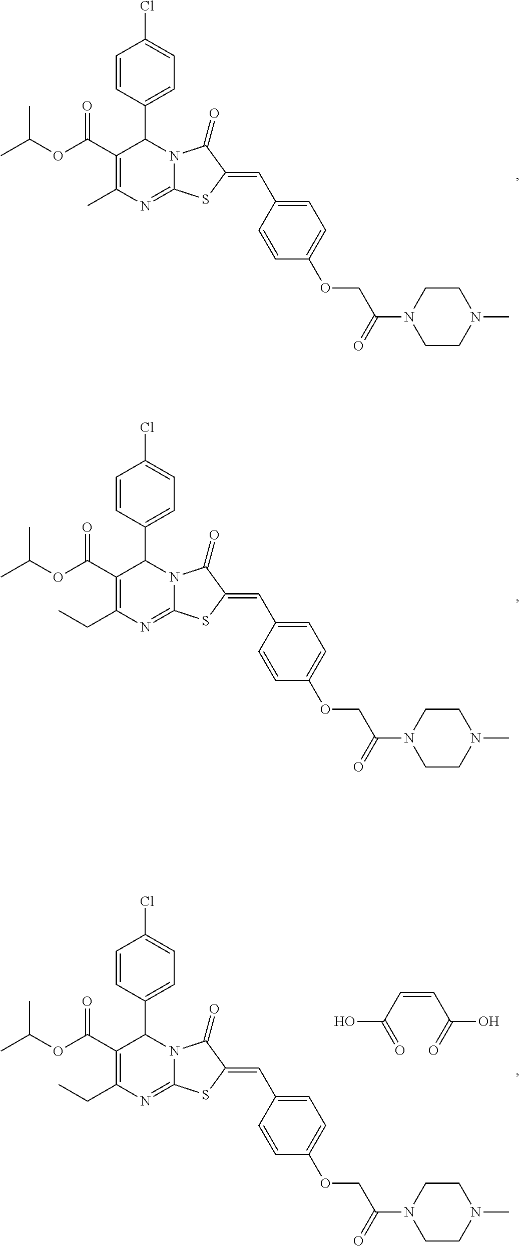

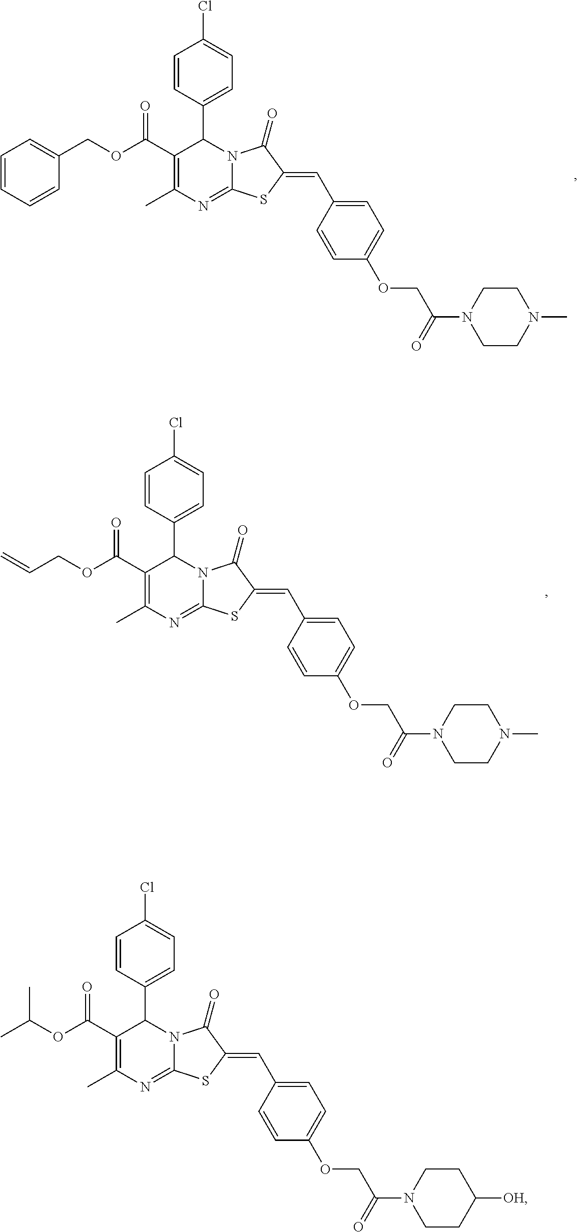

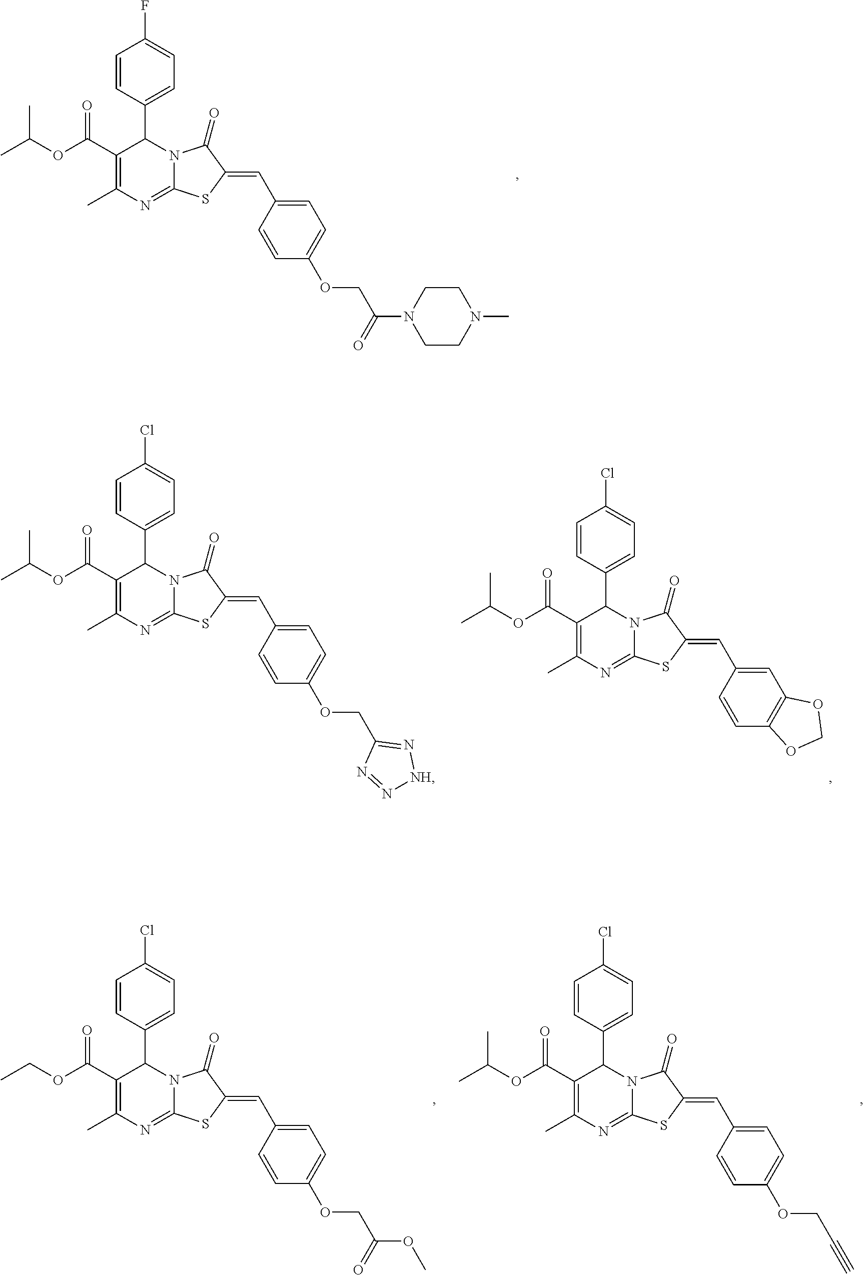 [SMILES: CC1=C(C(=O)OC(C)C)C(c2ccc(F)cc2)n2c(s/c(=C\c3ccc(OCC(=O)N4CCN(C)CC4)cc3)c2=O)=N1 ; CC1=C(C(=O)OC(C)C)C(c2ccc(Cl)cc2)n2c(s/c(=C\c3ccc(OCc4nn[nH]n4)cc3)c2=O)=N1 ; CC1=C(C(=O)OC(C)C)C(c2ccc(Cl)cc2)n2c(s/c(=C\c3ccc4c(c3)OCO4)c2=O)=N1 ; CCOC(=O)C1=C(C)N=c2s/c(=C\c3ccc(OCC(=O)OC)cc3)c(=O)n2C1c1ccc(Cl)cc1 ; C#CCOc1ccc(/C=c2\sc3n(c2=O)C(c2ccc(Cl)cc2)C(C(=O)OC(C)C)=C(C)N=3)cc1]

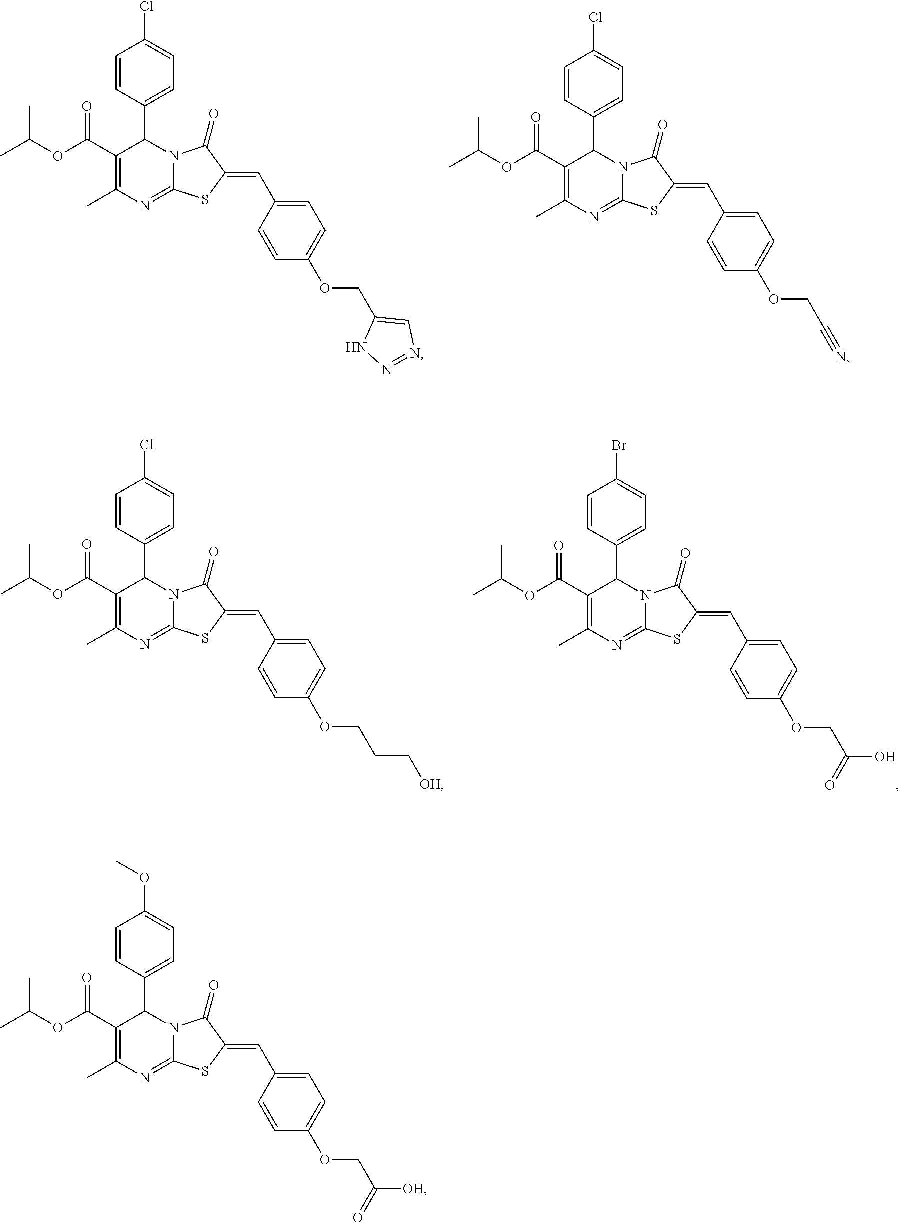 [SMILES: CC1=C(C(=O)OC(C)C)C(c2ccc(Cl)cc2)n2c(s/c(=C\c3ccc(OCc4cnn[nH]4)cc3)c2=O)=N1 ; CC1=C(C(=O)OC(C)C)C(c2ccc(Cl)cc2)n2c(s/c(=C\c3ccc(OCC#N)cc3)c2=O)=N1 ; CC1=C(C(=O)OC(C)C)C(c2ccc(Cl)cc2)n2c(s/c(=C\c3ccc(OCCCO)cc3)c2=O)=N1 ; CC1=C(C(=O)OC(C)C)C(c2ccc(Br)cc2)n2c(s/c(=C\c3ccc(OCC(=O)O)cc3)c2=O)=N1 ; COc1ccc(C2C(C(=O)OC(C)C)=C(C)N=c3s/c(=C\c4ccc(OCC(=O)O)cc4)c(=O)n32)cc1]

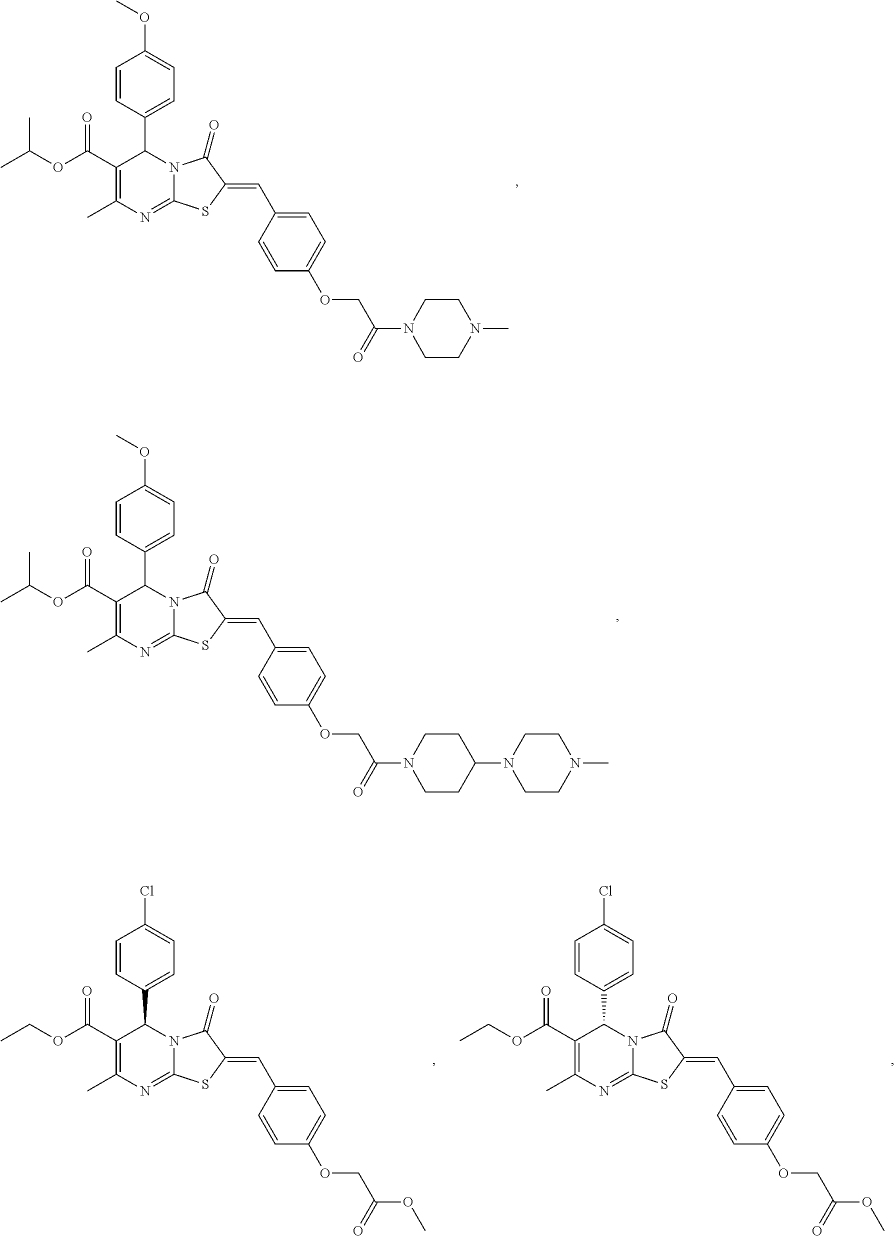

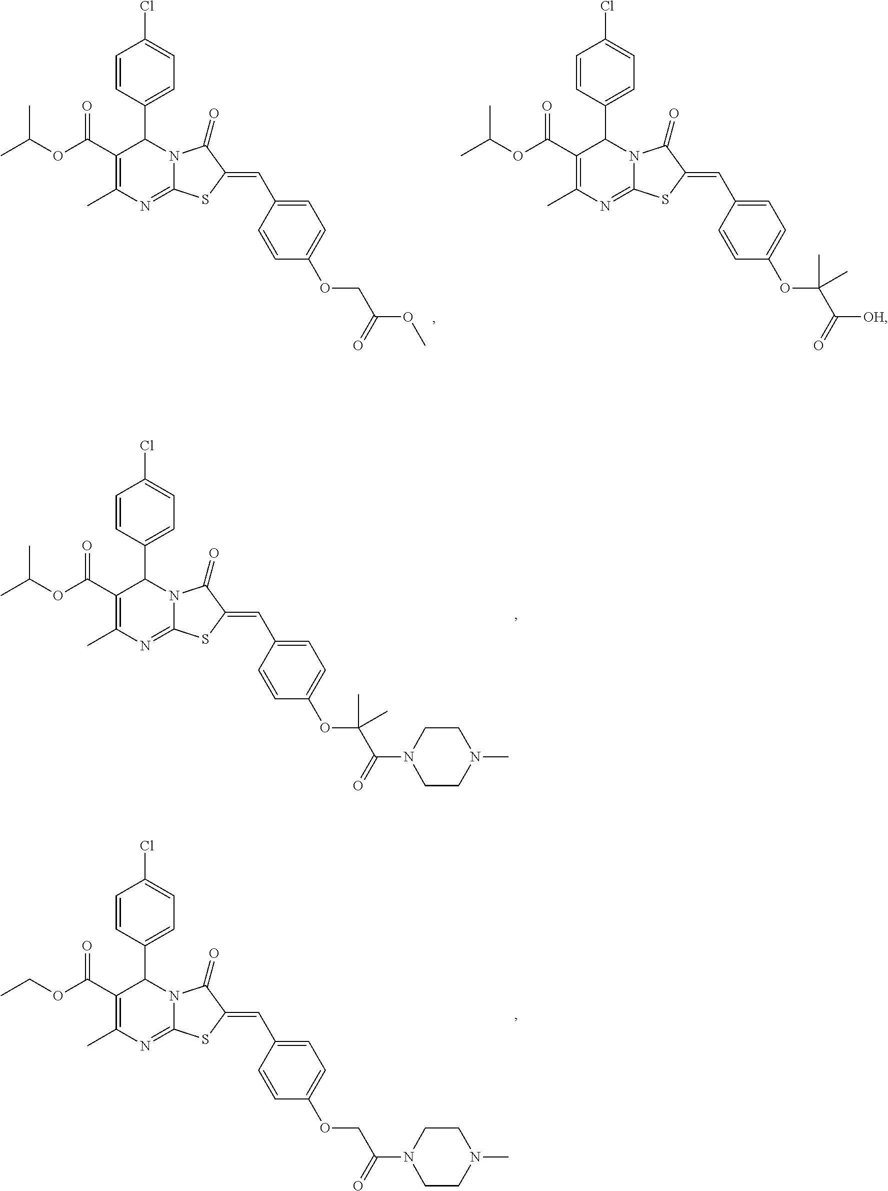 [SMILES: COC(=O)COc1ccc(/C=c2\sc3n(c2=O)C(c2ccc(Cl)cc2)C(C(=O)OC(C)C)=C(C)N=3)cc1 ; CC1=C(C(=O)OC(C)C)C(c2ccc(Cl)cc2)n2c(s/c(=C\c3ccc(OC(C)(C)C(=O)O)cc3)c2=O)=N1 ; CC1=C(C(=O)OC(C)C)C(c2ccc(Cl)cc2)n2c(s/c(=C\c3ccc(OC(C)(C)C(=O)N4CCN(C)CC4)cc3)c2=O)=N1 ; CCOC(=O)C1=C(C)N=c2s/c(=C\c3ccc(OCC(=O)N4CCN(C)CC4)cc3)c(=O)n2C1c1ccc(Cl)cc1]

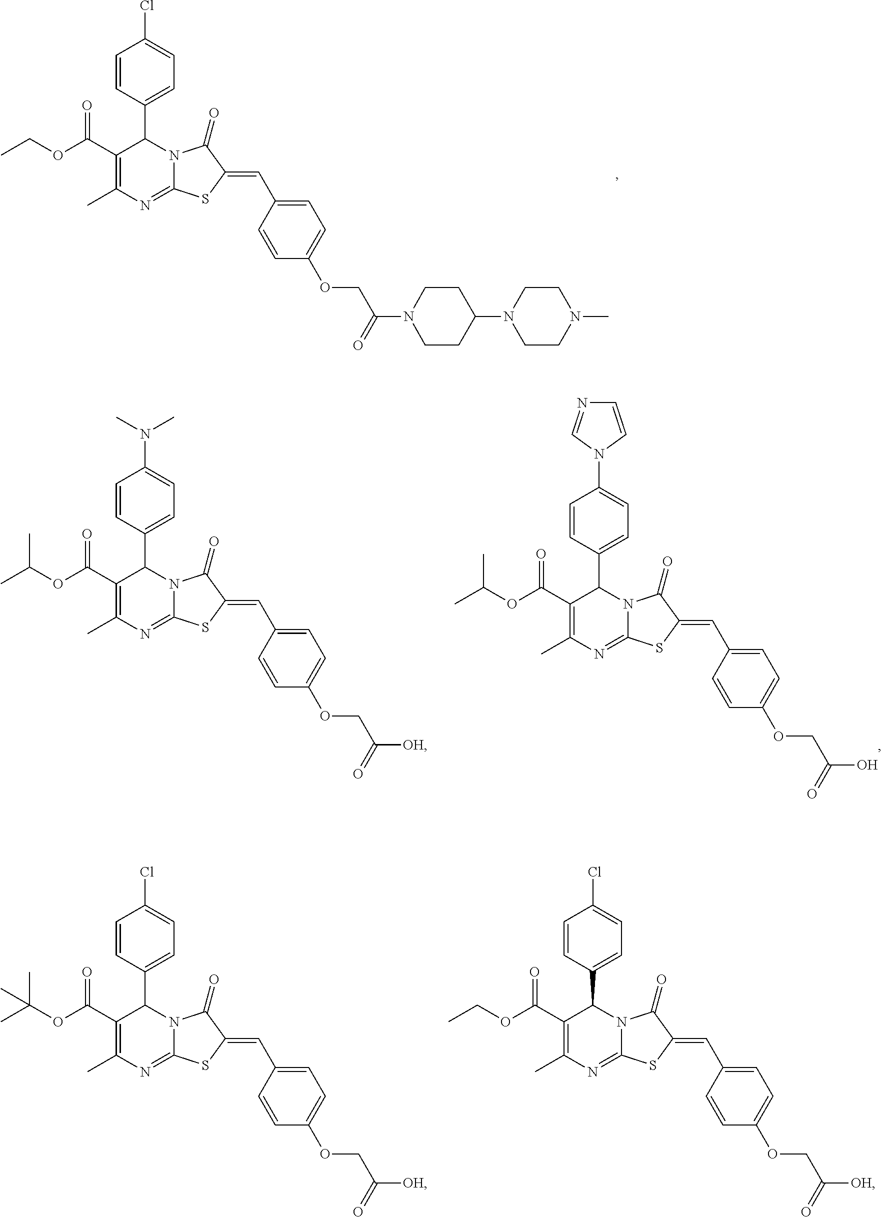

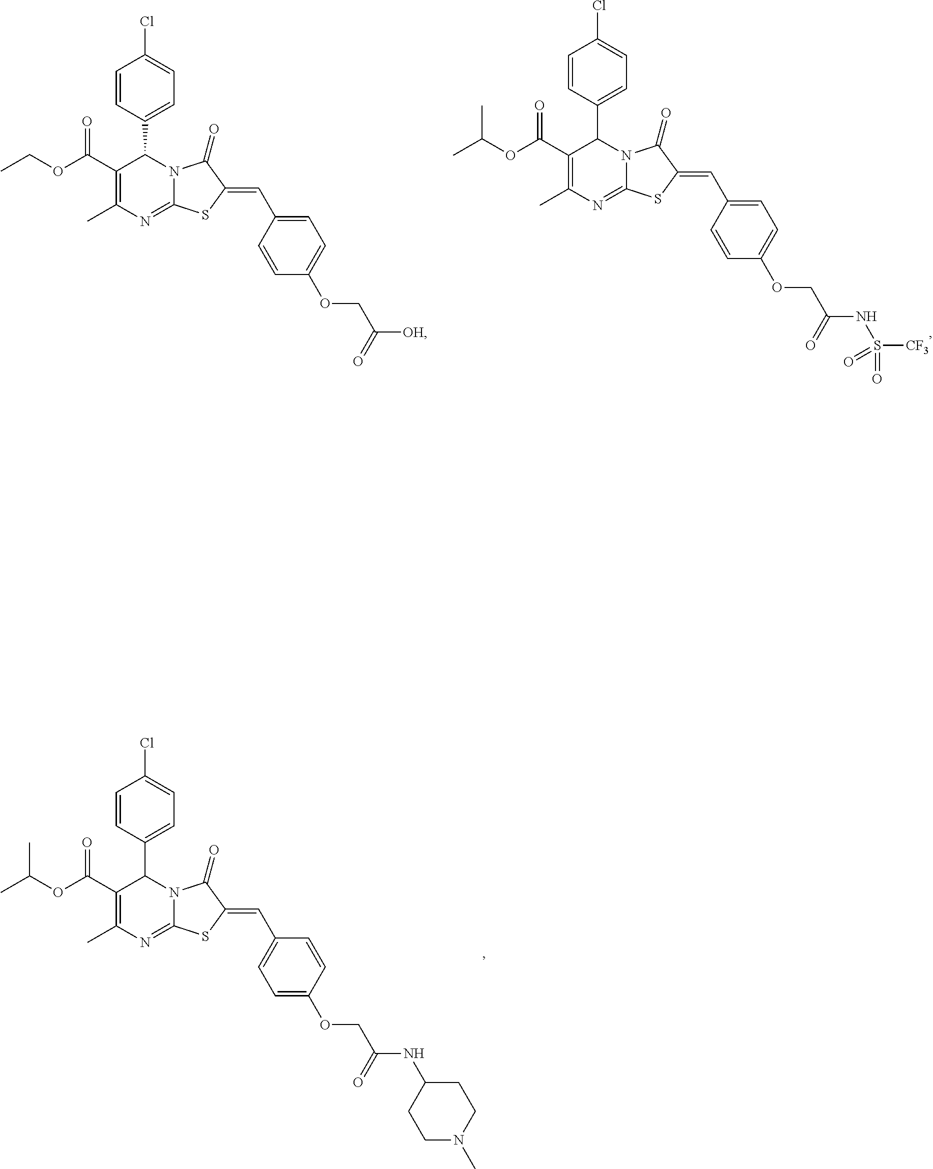 [SMILES: CCOC(=O)C1=C(C)N=c2s/c(=C\c3ccc(OCC(=O)O)cc3)c(=O)n2[C@H]1c1ccc(Cl)cc1 ; CC1=C(C(=O)OC(C)C)C(c2ccc(Cl)cc2)n2c(s/c(=C\c3ccc(OCC(=O)NS(=O)(=O)C(F)(F)F)cc3)c2=O)=N1 ; CC1=C(C(=O)OC(C)C)C(c2ccc(Cl)cc2)n2c(s/c(=C\c3ccc(OCC(=O)NC4CCN(C)CC4)cc3)c2=O)=N1]

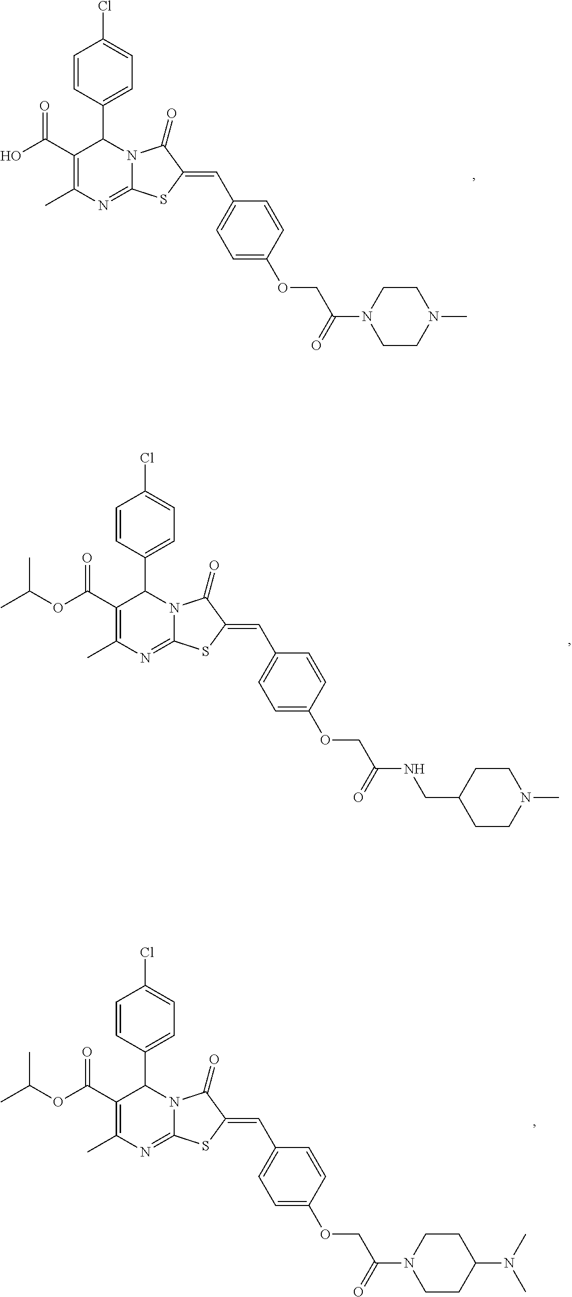

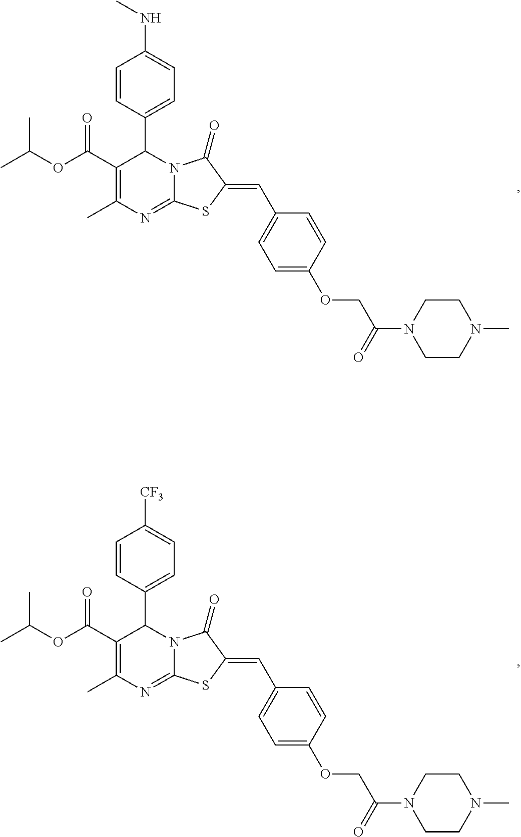

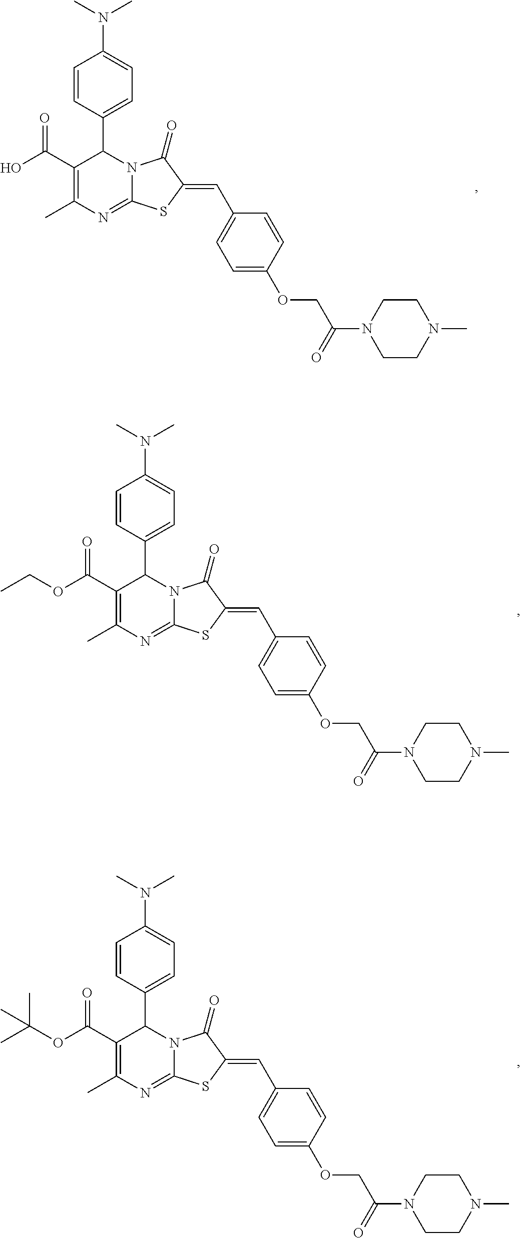

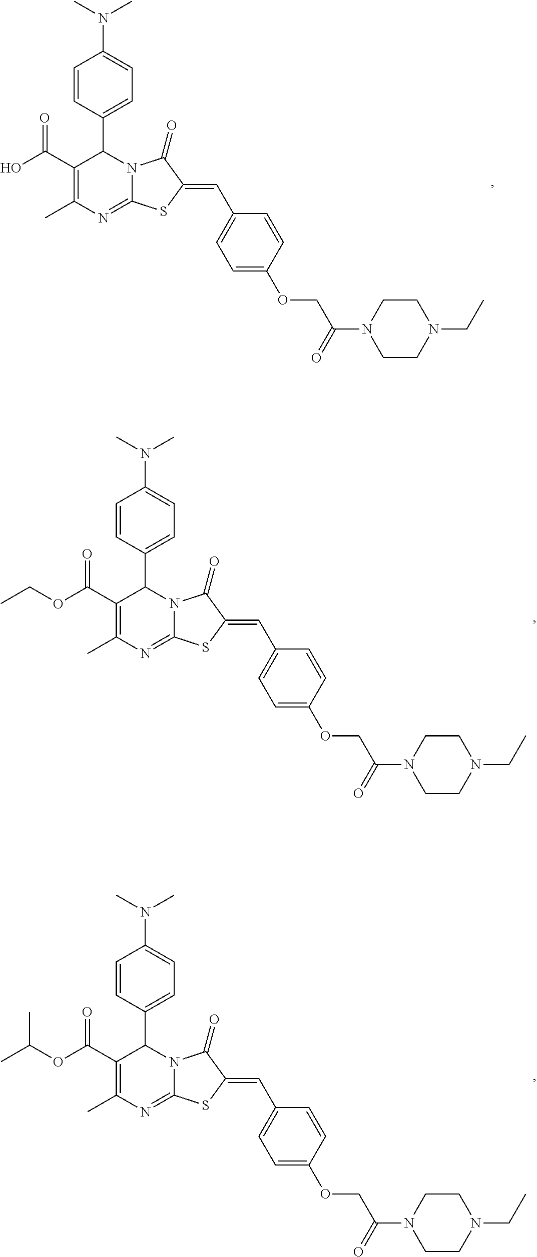

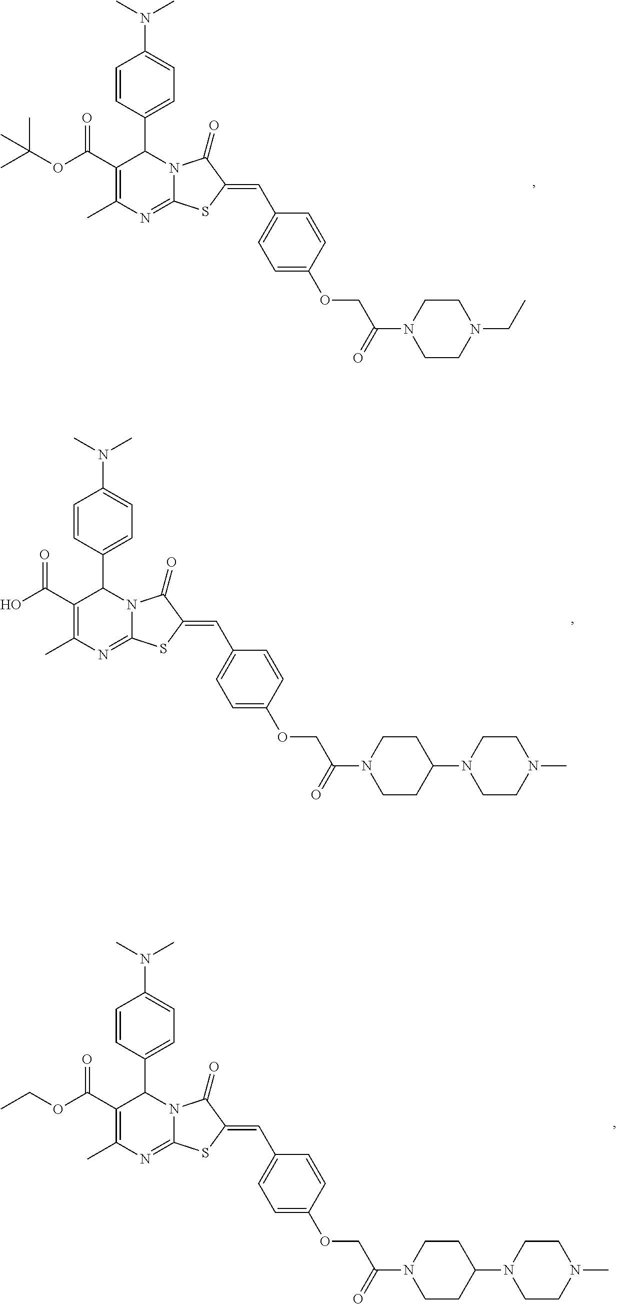

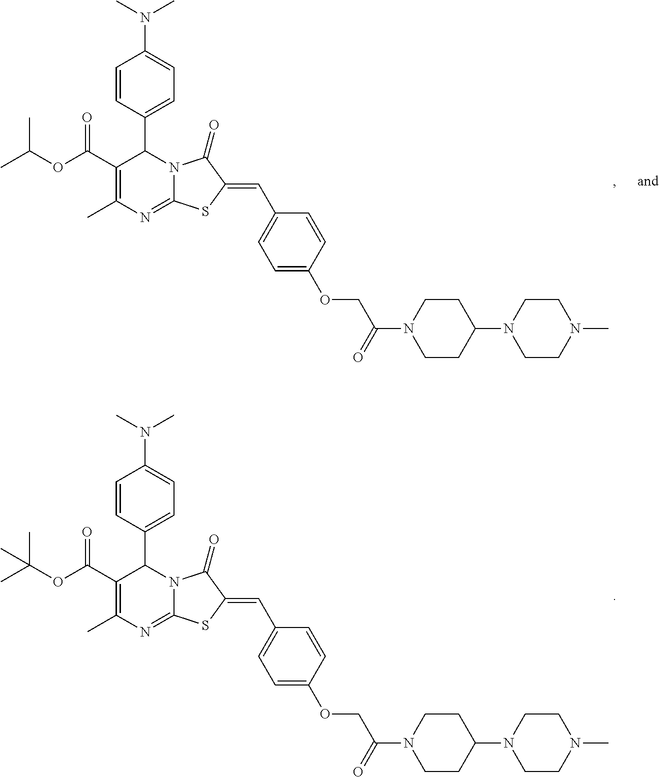

18. A pharmaceutical composition comprising
a compound of claim 1 or a pharmaceutically acceptable salt thereof; and
a pharmaceutically acceptable carrier, excipient or diluent.

19. The pharmaceutical composition of claim 18, further comprising a therapeutic agent.

20. The pharmaceutical composition of claim 19, wherein the therapeutic agent is at least one selected from the group consisting of paclitaxel, docetaxel, ixabepilone, eribulin, and vinorelbine.

21. A method for treating a cancer, comprising the following steps:
administering to a subject in need thereof an effective amount of a compound of claim 1 or a pharmaceutically acceptable salt thereof.
